(19) United States
(12) Patent Application Publication
(10) Pub. No.: US 2022/0310230 A1
(43) Pub. Date: Sep. 29, 2022

(54) BIOMARKERS FOR DETERMINING AN IMMUNO-ONOCOLOGY RESPONSE

(71) Applicant: Venn Biosciences Corporation, South San Francisco, CA (US)

(72) Inventors: Daniel SERIE, San Mateo, CA (US); Chad Eagle PICKERING, San Mateo, CA (US); Alan Nicolas MITCHELL, Fresno, CA (US); Gege XU, Redwood City, CA (US); Klaus LINDPAINTER, Concord, NH (US)

(73) Assignee: Venn Biosciences Corporation, South San Francisco, CA (US)

(21) Appl. No.: 17/688,788

(22) Filed: Mar. 7, 2022

Related U.S. Application Data
(60) Provisional application No. 63/158,283, filed on Mar. 8, 2021.
(60) Provisional application No. 63/246,293, filed on Sep. 20, 2021.
(60) Provisional application No. 63/251,023, filed on Sep. 30, 2021.

(51) Int. Cl. G16H 20/40; G16B 15/00; G16H 10/40

(52) U.S. Cl. CPC G16H 20/40; G16B 15/00; G16H 10/40

(57) ABSTRACT

Provided herein are methods, devices, and kits for identifying glycosylated polypeptide biomarkers and signatures for progression of a disease or a condition, such as cancer, or and response of the disease or condition to a treatment, such as treatment with immune checkpoint blockade for cancer. Provided herein are methods of generating glycosylated polypeptide biomarkers and methods of analyzing glycosylated polypeptides using mass spectrometry. Provided herein are methods of validating a model using glycosylated polypeptides for predicting the disease or condition or for making treatment recommendation.

### CROSS-REFERENCE TO RELATED APPLICATIONS

This application claims the benefit under 35 U.S.C. § 119(e) of U.S. Provisional Patent Application No. 63/158,283, filed 8 Mar. 2021, U.S. Provisional Patent Application No. 63/246,293, filed 20 Sep. 2021, and U.S. Provisional Patent Application No. 63/251,023, filed 30 Sep. 2021, each of which is incorporated herein by reference.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 166532000900SEQLIST.txt, date recorded: Mar. 6, 2022, size: 674,724 bytes

### BACKGROUND

Protein glycosylation and other post-translational modifications play vital roles in virtually all aspects of human physiology. Unsurprisingly, faulty or altered protein glycosylation often accompanies various disease states. The identification of aberrant glycosylation provides opportunities for early detection, intervention, and treatment of affected subjects. Current biomarker identification methods, such as those developed in the fields of proteomics and genomics, can be used to detect indicators of certain diseases, such as cancer, and to differentiate certain types of cancer from other, non-cancerous diseases. However, the use of glycoproteomic analyses has not previously been used to successfully manage treatment of a subject.

Glycoprotein analysis is fraught with challenges on several levels. For example, a single glycan composition in a glycopeptide can contain a large number of isomeric structures due to different glycosidic linkages, branching patterns, and/or multiple monosaccharides having the same mass. In addition, the presence of multiple glycans that share the same peptide backbone can lead to assay signals from various glycoforms, lowering their individual abundances compared to aglycosylated peptides. Accordingly, the development of algorithms that can identify glycan structures on peptide fragments remains elusive.

In light of the above, there is a desire for improved analytical methods that involve site-specific analysis of glycoproteins to obtain information about protein glycosylation patterns, which can in turn provide quantitative information that can be used to manage the treatment of a subject diagnosed with a particular disease or condition. Thus, it may be desirable to have methods and systems capable of addressing one or more of the above-identified issues.

### SUMMARY

In one or more embodiment, a method is provided for managing a treatment for a subject diagnosed with a melanoma condition. The method includes receiving peptide structure data corresponding to a set of glycoproteins in a biological sample obtained from the subject. A treatment score is computed using quantification data identified from the peptide structure data for a set of peptide structures. The set of peptide structures includes at least one peptide structure identified from a plurality of peptide structures listed in Table 1. A treatment output that indicates a predicted response to the treatment for the subject is generated using the treatment score.

In one or more embodiments, a method is provided for treatment management of a subject diagnosed with a melanoma condition. The method includes receiving peptide structure data corresponding to a set of peptide structures associated with a set of glycoproteins in a biological sample obtained from the subject. A plurality of treatment scores is computed using quantification data identified from the peptide structure data for a plurality of subsets of the set of peptide structures. Each treatment score of the plurality of treatment scores corresponds to a different treatment of a plurality of treatments; wherein each subset of the plurality of subsets includes at least one peptide structure identified from a plurality of peptide structures listed in Table 1. A comparison analysis of the plurality of treatment scores is performed. A treatment output is generated based on the comparison analysis. The treatment output includes a recommended treatment plan for treating the subject.

In one or more embodiments, a method is provided for treatment management of a subject diagnosed with a melanoma condition. The method includes receiving peptide structure data corresponding to a set of peptide structures associated with a set of glycoproteins in a biological sample obtained from the subject. A first treatment score is computed for a first treatment of pembrolizumab using first quantification data identified from the peptide structure data for a first subset of the set of peptide structures. The first subset includes at least one peptide structure identified from a plurality of peptide structures listed in Table 2. A second treatment score is computed for a second treatment comprised of nivolumab and ipilimumab using second quantification data identified from the peptide structure data for a second subset of the set of peptide structures. The second subset includes at least one peptide structure identified from a plurality of peptide structures listed in Table 3. A comparison analysis of the first treatment score and the second treatment score is performed. A treatment output is generated based on the comparison analysis. The treatment output identifies one of the first treatment and the second treatment as a recommended treatment for the subject.

In one or more embodiments, a method is provided for treating a subject diagnosed with a melanoma condition. The method includes receiving peptide structure data corresponding to a set of glycoproteins in a biological sample obtained from the subject. A treatment score is computed using quantification data identified from the peptide structure data for a set of peptide structures. The set of peptide structures includes at least one peptide structure identified from a plurality of peptide structures listed in Table 1. A treatment output that indicates a predicted response to a treatment for the subject is generated using the treatment score. The treatment is administered to the patient in response to the predicted response including a positive response classification. The step of administering comprises at least one of intravenous or oral administration of the recommended treatment or a derivative thereof at a therapeutic dosage. The treatment is selected as one from a group consisting of: a first treatment of pembrolizumab for which the therapeutic dosage of at least one of 200 mg every three weeks, 2 mg/kg every three weeks is administered, or 400 mg every 6 weeks; and a second treatment comprised of nivolumab and ipilimumab for which the therapeutic dosage of either 1 mg/kg nivolumab with 3 mg/kg ipilimumab or 3 mg/kg nivolumab with 1 mg/kg ipilimumab is administered.

In one or more embodiments, a method is provided for managing a treatment for a subject diagnosed with a melanoma condition. The method includes receiving sample data for a sample population. The sample data characterizes responses of a plurality of sample subjects diagnosed with the melanoma condition to the treatment and includes sample peptide structure data for a collection of peptide structures for each subject of the plurality of sample subjects. The sample data is grouped based on the responses of the plurality of sample subjects into a first group corresponding to a first response classification and a second group corresponding to a second response classification. A differential abundance analysis is performed using the sample data to compare the first group of the sample data corresponding to the first response classification and the second group of the sample data corresponding to the second response classification to identify a set of peptide structures from the collection of peptide structures. The set of peptide structures comprises a selected N most differentiating peptide structures between the first response classification and the second response classification. Peptide structure data corresponding to a set of glycoproteins in a biological sample obtained from the subject is received. A treatment score is computed for the treatment using quantification data identified from the peptide structure data for the set of peptide structures. A treatment output that indicates a predicted response to the treatment for the subject is generated using the treatment score.

In one or more embodiments, a method of treating melanoma in a subject is provided. The method includes receiving peptide structure data corresponding to a set of glycoproteins in a biological sample obtained from the subject. A treatment score is computed using quantification data identified from the peptide structure data for a set of peptide structures. The set of peptide structures includes at least one peptide structure identified from a plurality of peptide structures listed in Table 1. A treatment output is computed using the treatment score. A pembrolizumab treatment is administered to the subject if the treatment output includes at least one of a positive response classification for the pembrolizumab treatment or an identification of the pembrolizumab treatment as a recommended treatment.

In one or more embodiments, a method of treating melanoma in a subject is provided. The method includes receiving peptide structure data corresponding to a set of glycoproteins in a biological sample obtained from the subject. A treatment score is computed using quantification data identified from the peptide structure data for a set of peptide structures. The set of peptide structures includes at least one peptide structure identified from a plurality of peptide structures listed in Table 1. A treatment output is computed using the treatment score. A combination treatment comprising a combination of nivolumab and ipilimumab is administered to the subject if the treatment output includes at least one of a positive response classification for the combination treatment or an identification of the combination treatment as a recommended treatment.

In one or more embodiments, a method of identifying patients with melanoma for treatment with a pembrolizumab treatment is provided. The method includes receiving peptide structure data corresponding to a set of glycoproteins in a biological sample obtained from the subject. A treatment score is computed using quantification data identified from the peptide structure data for a set of peptide structures. The set of peptide structures includes at least one peptide structure identified from a plurality of peptide structures listed in Table 1. A treatment output is generated using the treatment score. The patient is treated with the pembrolizumab treatment if the treatment output includes at least one of a positive response classification for the pembrolizumab treatment or an identification of the pembrolizumab treatment as a recommended treatment.

In one or more embodiments, a method of identifying patients with melanoma for treatment with a combination treatment comprising nivolumab and ipilimumab is provided. The method includes receiving peptide structure data corresponding to a set of glycoproteins in a biological sample obtained from the subject. A treatment score is computed using quantification data identified from the peptide structure data for a set of peptide structures. The set of peptide structures includes at least one peptide structure identified from a plurality of peptide structures listed in Table 1. A treatment output is generated using the treatment score. The patient is treated with the combination treatment if the treatment output includes at least one of a positive response classification for the combination treatment or an identification of the combination treatment as a recommended treatment.

In one or more embodiments, a method is provided for analyzing a set of peptide structures in a sample from a patient. The method includes (a) obtaining the sample from the patient; (b) preparing the sample to form a prepared sample comprising a set of peptide structures; (c) inputting the prepared sample into a reaction monitoring mass spectrometry system to detect a set of product ions associated with each peptide structure of the set of peptide structures; and (d) generating quantification data for the set of product ions using the reaction monitoring mass spectrometry system. The set of peptide structures includes at least one peptide structure selected from peptide structures PS-1 to PS-38 identified in Table 6. The set of peptide structures includes a peptide structure that is characterized as having: (i) a precursor ion with a mass-charge (m/z) ratio within ±1.5 of the m/z ratio listed for the precursor ion in Table 6 as corresponding to the peptide structure; and (ii) a product ion having an m/z ratio within ±1.0 of the m/z ratio listed for the first product ion in Table 6 as corresponding to the peptide structure.

In one or more embodiments, a composition is provided, the composition comprising a peptide structure or a product ion, wherein: the peptide structure or product ion comprises the amino acid sequence having at least 90% sequence identity to any one of SEQ ID NOS: 21-46, corresponding to peptide structures PS-1 to PS-38 in Table 1; and the product ion is selected as one from a group consisting of product ions identified in Table 6 including product ions falling within an identified m/z range.

In one or more embodiments, a composition is provided, the composition comprising a glycopeptide structure selected as one from a group consisting of peptide structures PS-1 to PS-38 identified in Table 6. The glycopeptide structure comprises: an amino acid peptide sequence identified in Table 5 as corresponding to the glycopeptide structure; and a glycan structure identified in Table 1 as corresponding to the glycopeptide structure in which the glycan structure is linked to a residue of the amino acid peptide sequence at a corresponding position identified in Table 1. The glycan structure has a glycan composition.

In one or more embodiments, a composition is provided, the composition comprising a peptide structure selected as one from a plurality of peptide structures identified in Table 1. The peptide structure has a monoisotopic mass identified as corresponding to the peptide structure in Table 1. The peptide structure comprises the amino acid sequence of SEQ ID NOs: 21-46 identified in Table 1 as corresponding to the peptide structure.

In one or more embodiments, a kit is provided, the kit comprising at least one agent for quantifying at least one peptide structure identified in Table 1 to carry out at least a portion of any one of the methods disclosed herein.

In one or more embodiments, a kit is provided, the kit comprising at least one of a glycopeptide standard, a buffer, or a set of peptide sequences to carry out at least a portion of any one of the methods disclosed herein, a peptide sequence of the set of peptide sequences identified by a corresponding one of SEQ ID NOS: 21-46, defined in Table 1.

Provided herein are methods, devices, and kits for identifying glycoproteomic biomarkers and signatures for diagnosis of a disease or a condition, such as cancer, progression of the disease or condition, and response of the disease or condition to a treatment, such as treatment with immune checkpoint blockade for cancer.

Provided herein are methods for identifying one or more glycopeptide biomarkers predictive of a disease or a condition in a subject, the method comprising: (a) obtaining from a subject a first sample at a first timepoint and a second sample at a second timepoint, wherein the first sample and the second sample comprise a glycoprotein; (b) fragmenting the glycoprotein in the first sample or the second sample into one or more glycopeptides, wherein the one or more glycopeptides comprise one or more amino acid sequences selected from a group consisting of SEQ ID NO: 101-131, 159-207, and 21-46, and combinations thereof; (c) determining an amount of the one or more glycopeptides using multiple reaction monitoring mass spectrometry (MRM-MS); (d) associating the amount of the one or more glycopeptides with the first timepoint or the second timepoint, wherein the subject has a change in a disease or a condition from the first timepoint to the second timepoint; and (e) identifying as glycopeptide biomarkers the glycopeptide where the amount of the one or more glycopeptides changed from the first timepoint to the second timepoint.

Provided herein are methods for identifying one or more glycopeptide biomarkers predictive of a disease or a condition in a subject, the method comprising: (a) obtaining, by a computer, data of an amount of one or more glycopeptides for a set (n) of subjects, wherein the one or more glycopeptides are generated by fragmenting a glycoprotein in a sample from a subject, the amount of one or more glycopeptides are determined using multiple reaction monitoring mass spectrometry (MRM-MS), and the data for each subject comprises data from samples taken at a plurality of timepoints; (b) selecting, by the computer, a subset of the one or more glycopeptides to include in a predictive model; (c) assessing, by the computer, the predictive model using a cross-validation with n−1 subjects to generate an outcome score for a holdout subject; (d) iterating, by the computer, step (c) for each of n subjects as the holdout subject to generate an outcome score for each subject; (e) dichotomizing, by the computer, the outcome scores for each subject at a cutoff outcome score as below or above the cutoff outcome score; (f) analyzing, by the computer, the amount of one or more glycopeptides for subjects having outcome scores above the cutoff outcome score to the amount of one or more glycopeptides for subjects having outcome scores below the cutoff outcome score for each glycopeptide in the subset of the one or more glycopeptides to determine a hazard ratio and an interaction p-value for each glycopeptide; (g) identifying, by the computer, the glycopeptide having the interaction p-value ≤0.05 as a glycopeptide biomarker for predicting the disease or the condition. In some embodiments, the cross-validation is leave-one-out cross-validation (LOOCV). In some embodiments, the cutoff outcome score was determined to optimize Harrell's C-index. In some embodiments, the interaction p-value is less than or equal to 0.01, 0.005, or 0.001 in step (g).

Provided herein are methods for assessing a status of a condition and a treatment in a subject, the method comprising: (a) fragmenting a glycoprotein in a sample from a subject into one or more glycopeptides, wherein the sample comprises one or more of glycoproteins, glycans, or glycopeptides; (b) performing mass spectroscopy (MS) on the one or more glycopeptides using multiple reaction monitoring mass spectrometry (MRM-MS) to quantify an amount of the one or more glycopeptides in the sample, wherein the one or more glycopeptides comprise one or more amino acid sequences selected from a group consisting of SEQ ID NOs: 7, 9, 12, 15, 16, 18, 20, 30, 34, 37, 44, 59, 60, 61, 62, 66, 69, 70, 75, 77, 80, and 83, and combinations thereof; (c) inputting data of the amount of the one or more glycopeptides into a trained model to generate an output probability, wherein the output probability is indicative of whether a treatment positively influences an outcome of the subject having a condition; and (d) generating a treatment recommendation based on the output probability, wherein the condition is melanoma and the treatment comprises checkpoint inhibitors. In some embodiments, the outcome comprises overall survival time. In some embodiments, the outcome comprises progression-free survival time. In some embodiments, the treatment comprises one or more of ipilimumab, nivolumab, and pembrolizumab. In some embodiments, the treatment comprises one or more of PD-1-, PD-L1-, and CTLA-4-inhibitors. In some embodiments, the recommendation comprises continuing the treatment if the output probability indicates the treatment positively influences the outcome.

Furthermore, provided herein are methods for assessing a status of a condition and a treatment in a subject, the method comprising: (a) fragmenting a glycoprotein in a sample from a subject into one or more glycopeptides, wherein the sample comprises one or more of glycoproteins, glycans, or glycopeptides; (b) performing mass spectroscopy (MS) on the one or more glycopeptides using multiple reaction monitoring mass spectrometry (MRM-MS) to quantify an amount of the one or more glycopeptides in the sample, wherein the one or more glycopeptides comprise one or more amino acid sequences selected from a group consisting of SEQ ID NOs: 300-429, and combinations thereof; (c) inputting data of the amount of the one or more glycopeptides into a trained model to generate an output probability, wherein the output probability is indicative of whether a treatment positively influences an outcome of the subject having a condition; and (d) generating a treatment recommendation based on the output probability, wherein the condition is non-small cell lung cancer (NSCLC) and the treatment comprises checkpoint inhibitors. In some embodiments, the outcome comprises overall survival time. In some embodiments, the outcome comprises progression-free survival time. In some embodiments, the treatment comprises one or more of ipilimumab, nivolumab, and pembrolizumab. In some embodiments, the treatment comprises one or more of PD-1-, PD-L1-, and CTLA-4-inhibitors. In some embodiments, the treatment comprises chemotherapy. In some embodiments, the chemotherapy comprises one or more of carboplatin and pemetrexed. In some embodiments, the recommendation comprises continuing the treatment if the output probability indicates the treatment positively influences the outcome.

Provided herein are glycopeptides comprising an amino acid sequence selected from a group consisting of SEQ ID NOs: 300-429, and combinations thereof.

Described herein are kits comprising a glycopeptide standard comprising a glycopeptide comprising one or more amino acid sequences selected from a group consisting of SEQ ID NOs: 300-429, and an instruction for using the glycopeptide standard for treating cancer.

In some embodiments, fragmenting comprises protease digestion. In some embodiments, fragmenting comprises applying a mechanical force. In some embodiments, the amount of one or more glycopeptides measures multiple reaction monitoring (MRM) transitions. In some embodiments, the method comprises further generating a panel of glycopeptide biomarkers comprising one or more of the glycopeptide biomarkers identified in step (e). In some embodiments, the cross-validation is leave-one-out cross-validation (LOOCV). In some embodiments, the cutoff outcome score was determined to optimize Harrell's C-index. In some embodiments, the interaction p-value is less than or equal to 0.01, 0.005, or 0.001 in step (g). In some embodiments, the outcome comprises overall survival time. In some embodiments, the outcome comprises progression-free survival time. In some embodiments, the treatment comprises one or more of ipilimumab, nivolumab, and pembrolizumab. In some embodiments, the treatment comprises one or more of PD-1-, PD-L1-, and CTLA-4-inhibitors. In some embodiments, the treatment comprises chemotherapy. In some embodiments, the chemotherapy comprises one or more of carboplatin and pemetrexed. In some embodiments, the recommendation comprises continuing the treatment if the output probability indicates the treatment positively influences the outcome.

In one or more embodiments, a system is provided that includes one or more data processors and a non-transitory computer readable storage medium containing instructions which, when executed on the one or more data processors, cause the one or more data processors to perform part or all of one or more methods disclosed herein.

In one or more embodiments, a computer-program product is provided that is tangibly embodied in a non-transitory machine-readable storage medium and that includes instructions configured to cause one or more data processors to perform part or all of one or more methods disclosed herein.

### BRIEF DESCRIPTION OF THE DRAWINGS

The present disclosure is described in conjunction with the appended figures:

FIG. 1 is a schematic diagram of an exemplary workflow 100 for the detection of peptide structures associated with a condition for use in treatment management in accordance with one or more embodiments.

FIG. 2A is a schematic diagram of a preparation workflow in accordance with one or more embodiments.

FIG. 2B is a schematic diagram of data acquisition in accordance with one or more embodiments.

FIG. 3 is a block diagram of an analysis system in accordance with one or more embodiments.

FIG. 4 is a block diagram of a computer system in accordance with various embodiments.

FIG. 5 is a flowchart of a process for managing a treatment for a subject diagnosed with a melanoma condition in accordance with one or more embodiments.

FIG. 6 is a flowchart of a process for treatment management of a subject diagnosed with a melanoma condition in accordance with various embodiments.

FIG. 7 is a flowchart of a process for treatment management of a subject diagnosed with a melanoma condition in accordance with various embodiments.

FIG. 8 is a flowchart of a process for identifying a treatment for a subject diagnosed with a melanoma condition in accordance with one or more embodiments.

FIG. 9 is a plot showing the distribution of the treatment scores generated for those patients who were treated with pembro in accordance with one or more embodiments.

FIG. 10 is a plot showing the distribution of the treatment scores generated for those patients who were treated with ipi/nivo in accordance with one or more embodiments.

FIG. 11 is a scatterplot showing the treatment scores by treatment type in accordance with one or more embodiments.

FIG. 12 is a plot showing disruption event times for patients treated with pembro by their predicted response.

FIG. 13 is a plot showing disruption event times for patients treated with ipi/nivo by their predicted response.

FIGS. 14A and 14B show progression-free survival (PFS) Kaplan-Meier curves of patients with metastatic melanoma for various glycopeptide fragments.

FIGS. 15A and 15B show progression-free survival (PFS) Kaplan-Meier curves of patients with non-small-cell lung cancer (NSCLC) for various glycopeptide fragments.

FIGS. 16-41 show overall survival (OS) Kaplan-Meier curves of patients with metastatic melanoma for various glycopeptide fragments.

FIGS. 42-80 show progression-free survival (PFS) Kaplan-Meier curves of patients with metastatic melanoma for various glycopeptide fragments.

FIGS. 81A and 81B illustrate an algorithm development pipeline for identifying non-small-cell lung cancer (NSCLC).

FIGS. 82A and 82B illustrate a multivariate classifier development for case-control studies for identifying non-small-cell lung cancer (NSCLC).

FIGS. 83A-83D illustrate scoring prediction curves for identifying non-small-cell lung cancer (NSCLC).

### DETAILED DESCRIPTION

Objective response rates for immune-oncology therapy are low in malignant melanoma and non-small cell lung cancer patients. Subjects should avoid unnecessary exposure and toxicities if they will not respond to immune-oncology therapy. Thus, in some aspects, the present invention is directed to identifying subjects who are not likely to respond to immune-oncology therapy (such as treatment with pembrolizumab and/or treatment with nivolumab and ipilimumab). In some embodiments the methods provided herein increase the rate of responder to immune-oncology treatments by identifying non-responders. Another advantage of the present method is that it can be used to reduce the cost associated with immune-oncology therapy per indication by avoiding treatment of subjects that are not likely to respond to treatment.

In some aspects, the present methods employ models and other predictive methods to assess the likelihood of response of a subject to immunotherapy. In some aspects, the methods provided herein have a high sensitivity for non-responders (those that are not likely to respond to immune-oncology therapy). In some aspects, the methods provided herein have a >95%, >97%, >98, or >99% sensitivity for detection of non-responders.

Provided herein are methods for management of treatment for subjects diagnosed with melanomas. In some embodiments, the subject is diagnosed with advanced melanoma. In some embodiments, the subject is diagnosed with malignant melanoma. In some embodiments, the subject is diagnosed with metastatic melanoma. In some embodiments, the method comprises determining whether the subject is likely to respond to an immunotherapy. In some embodiments, the method comprises determining whether the subject is likely to respond to treatment with pembrolizumab. In some embodiments, the method comprises determining whether the subject is likely to respond to treatment with nivolumab and ipilimumab.

Provided herein are methods of treating melanoma in a subject comprising administering a treatment to the subject. In some embodiments, the melanoma is advanced melanoma. In some embodiments, the melanoma is malignant melanoma. In some embodiments, the melanoma is metastatic melanoma. In some embodiments, the treatment comprises administering pembrolizumab to the subject. In some embodiments, the treatment comprises administering nivolumab and ipilimumab to the subject.

In some embodiments, the method comprises determining the likelihood of response of a subject having melanoma to nivolumab plus ipilimumab as a first line therapy. In some embodiments, the method comprises determining the likelihood of response to nivolumab plus ipilimumab as a second line therapy.

In some embodiments, the method comprises determining the likelihood of response of a subject having non-small cell lung cancer to pembrolizumab as a first line therapy. In some embodiments, the method comprises determining the likelihood of response to pembrolizumab as a second line therapy.

In some embodiments, the methods provided herein comprises generating a treatment output that predicts a response to an immune-oncology therapy (such as pembrolizumab or nivolumab plus ipilimumab) In some embodiments, the predicted response is likely responsive, likely nonresponsive, or indeterminate. In some embodiments, the treatment output is determined based upon the presence, absence, or amount of one or more glycopeptide set forth in Table 7, Table 12, Table 14, or Table 16. In some embodiments, the methods provided herein predict overall survival in subjects with melanoma. In some embodiments, the methods provided herein predict progression free survival in subject with NSCLC.

### 1. Managing Treatment of Melanoma

### I. Overview

The embodiments described herein recognize that glycoproteomics is an emerging field that can be used in the overall treatment of subjects (e.g., patients) with various types of diseases. Glycoproteomics aims to determine the positions, identities, and quantities of glycans and glycosylated proteins in a given sample (e.g., blood sample, cell, tissue, etc.). Protein glycosylation is one of the most common and most complex forms of post-translational protein modification, and can affect protein structure, conformation, and function. For example, glycoproteins may play crucial roles in important biological processes such as cell signaling, host-pathogen interactions, and immune response and disease. Glycoproteins may therefore be important to treating different types of diseases.

Although protein glycosylation provides useful information about cancer and other diseases, analysis of protein glycosylation may be difficult as the glycan typically cannot be traced back to the protein site of origin with currently available methodologies. Glycoprotein analysis can be challenging in general due to several reasons. For example, a single glycan composition in a peptide may contain a large number of isomeric structures because of different glycosidic linkages, branching, and many monosaccharides having the same mass. Further, the presence of multiple glycans that share the same peptide sequence may cause the mass spectrometry (MS) signal to split into various glycoforms, lowering their individual abundances compared to the peptides that are not glycosylated (aglycosylated peptides).

But to understand various disease conditions and more accurately manage the treatment of such disease conditions, such as melanoma, it may be important to perform analysis of glycoproteins and to identify not only the glycan but also the linking site (e.g., the amino acid residue of attachment) within the protein. Thus, there is a need to provide a method for site-specific glycoprotein analysis to obtain detailed information about protein glycosylation patterns which may be able to provide information that can be used to treat diseases, such as melanoma.

Melanoma is a type of cancer that develops from melanocytes, cells that product pigment. Melanoma may be treated using different types of treatment including, for example, immunotherapies. Such immunotherapies include various types of immune check point inhibitor treatments (e.g., pembrolizumab, nivolumab, ipilimumab) and cytokine therapies (e.g., interferon alpha (IFN-α) and Interleukin 2 (IL-2). Immune check point inhibitors include, for example, anti-cytotoxic T-lymphocyte-associated protein 4 (CTLA-4) monoclonal antibodies (e.g., ipilimumab, tremelimumab), toll-like receptor (TLR) agonists, cluster of differentiation 40 (CD40) agonists, anti-programmed cell death protein 1 (PD-1) (e.g., pembrolizumab, pidilizumab, and nivolumab) and programmed death-ligand 1 (PD-L1) antibodies.

Different patients may respond differently to different treatments. For example, some patients may have great success with one type of treatment while other patients may have limited or no success with that same treatment. Because melanoma is an aggressive cancer and one of the most serious cancers, subjects may not have the luxury of trying different types of treatments over time. It may be important to identify those subjects who are likely to respond to a given treatment to help avoid the burden associated with adverse events (e.g., events that disrupt a subject's progression-free survival) and to avoid the cost associated with treatment subjects who are not likely to respond to certain treatments. Previous methodologies generally focused on specific mechanisms of drug efficacy of a particular treatment. For example, such methodologies focused on tumor response rather than subject survival. But the embodiments described herein provide ways in which to predict treatment response with respect to survivability for different drugs so that a better selection of treatment may be selected for a subject at the outset.

Analyzing peptide structure expression in subjects and, in particular, glycopeptide structure abundance may help predict subject response to treatment for melanoma. A peptide structure may be defined by an aglycosylated peptide sequence (e.g., a peptide or peptide fragment of a larger parent protein) or a glycosylated peptide sequence. A glycosylated peptide sequence (also referred to as a glycopeptide structure) may be a peptide sequence having a glycan structure that is attached to a linking site (e.g., an amino acid residue) of the peptide sequence, which may occur via, for example, a particular atom of the amino acid residue). Non-limiting examples of glycosylated peptides include N-linked glycopeptides and O-linked glycopeptides.

Further, with glycoproteins, there may be too many potential proteoforms to consider. Still further, analysis of peptide structure data in the manner described by the various embodiments herein may be more conducive to accurately predicting treatment response as compared to glycomic analysis that provides little to no information about what proteins and to which amino acid residue sites various glycan structures attach.

By analyzing which peptide structures are most differentiating between different treatment response classifications of interest (e.g., sustained control and early disruption) for a given treatment and then analyzing a subject's peptide structure profile of those particular peptide structures, a clearer understanding of how that subject will respond to that treatment may be achieved.

Accordingly, the embodiments described herein provide various methods and systems for analyzing proteins in subjects and, in particular, glycoproteins. In one or more embodiments, methods and systems are provided for treatment management of a subject diagnosed with a melanoma condition. For example, the embodiments described herein provide methods and systems for receiving peptide structure data corresponding to a set of glycoproteins in a biological sample obtained from the subject; computing a treatment score using quantification data identified from the peptide structure data for a set of peptide structures, wherein the set of peptide structures includes at least one peptide structure identified from a plurality of peptide structures listed in Table 1; and generating a treatment output that indicates a predicted response to the treatment for the subject using the treatment score. The predicted response may indicate whether the subject is likely to have sustained control (e.g., no disruption events that might disrupt the subject's progression-free survival within 12 months of treatment) with the treatment or to have early disruption (e.g., one or more disruption events within the first 6 months of treatment).

The description below provides exemplary implementations of the methods and systems described herein for the research and/or treatment (e.g., designing, planning, administration, etc. of a treatment) of melanoma. Descriptions and examples of various terms, as used herein, are provided in Section II below.

### II. Exemplary Descriptions of Terms

The term “ones” means more than one.

As used herein, the term “plurality” may be 2, 3, 4, 5, 6, 7, 8, 9, 10, or more.

As used herein, the term “set of” means one or more. For example, a set of items includes one or more items.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The term “amino acid,” as used herein, generally refers to any organic compound that includes an amino group (e.g. —NH2), a carboxyl group (—COOH), and a side chain group (R) which varies based on a specific amino acid. Amino acids can be linked using peptide bonds.

The term “alkylation,” as used herein, generally refers to the transfer of an alkyl group from one molecule to another. In various embodiments, alkylation is used to react with reduced cysteines to prevent the re-formation of disulfide bonds after reduction has been performed.

The term “linking site” or “glycosylation site” as used herein generally refers to the location where a sugar molecule of a glycan or glycan structure is directly bound (e.g. covalently bound) to an amino acid of a peptide, a polypeptide, or a protein. For example, the linking site may be an amino acid residue and a glycan structure may be linked via an atom of the amino acid residue. Non-limiting examples of types of glycosylation can include N-linked glycosylation, O-linked glycosylation, C-linked glycosylation, S-linked glycosylation, and glycation.

The terms “biological sample,” “biological specimen,” or “biospecimen” as used herein, generally refers to a specimen taken by sampling so as to be representative of the source of the specimen, typically, from a subject. A biological sample can be representative of an organism as a whole, specific tissue, cell type, or category or sub-category of interest. The biological sample can include a macromolecule. The biological sample can include a small molecule. The biological sample can include a virus. The biological sample can include a cell or derivative of a cell. The biological sample can include an organelle. The biological sample can include a cell nucleus. The biological sample can include a rare cell from a population of cells. The biological sample can include any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological sample can include a constituent of a cell. The biological sample can include nucleotides (e.g. ssDNA, dsDNA, RNA), organelles, amino acids, peptides, proteins, carbohydrates, glycoproteins, or any combination thereof. The biological sample can include a matrix (e.g., a gel or polymer matrix) comprising a cell or one or more constituents from a cell (e.g., cell bead), such as DNA, RNA, organelles, proteins, or any combination thereof, from the cell. The biological sample may be obtained from a tissue of a subject. The biological sample can include a hardened cell. Such hardened cells may or may not include a cell wall or cell membrane. The biological sample can include one or more constituents of a cell but may not include other constituents of the cell. An example of such constituents may include a nucleus or an organelle. The biological sample may include a live cell. The live cell can be capable of being cultured.

The term “denaturation,” as used herein, generally refers to any molecule that loses quaternary structure, tertiary structure, and secondary structure which is present in their native state. Non-limiting examples include proteins or nucleic acids being exposed to an external compound or environmental condition such as acid, base, temperature, pressure, radiation, etc.

The term “denatured protein,” as used herein, generally refers to a protein that loses quaternary structure, tertiary structure, and secondary structure which is present in their native state.

The terms “digestion” or “enzymatic digestion,” as used herein, generally refer to breaking apart a polymer (e.g. cutting a polypeptide at a cut site). Proteins may be digested in preparation for mass spectrometry using trypsin digestion protocols. Proteins may be digested using other proteases in preparation for mass spectrometry if access is limited to cleavage sites.

The term “treatment” may generally refer to any number of drugs, therapeutics, lifestyle modifications, behavioral modifications, dietary modifications, or combination thereof that can be used to treat a subject suffering form a disease condition.

The term “therapeutic” may refer generally to any drug that can be administered to a subject physically (e.g., via oral, intravenous injection, topical treatment, exposure, etc.).

The terms “immune checkpoint inhibitor,” “immune checkpoint inhibitor therapeutic,” and “immune checkpoint inhibitor drug,” as used herein, generally refer to drugs or therapeutics that can target immune checkpoint molecules (e.g. molecules on immune cells that need to be activated (or inactivated) to start an immune response). Non-limiting examples of immune checkpoint inhibitor therapeutics can include pembrolizumab, nivolumab, and ipilimumab.

The terms “glycan” or “polysaccharide” as used herein, both generally refer to a carbohydrate residue of a glycoconjugate, such as the carbohydrate portion of a glycopeptide, glycoprotein, glycolipid, or proteoglycan. Glycans can include monosaccharides.

The term “glycopeptide” or “glycopolypeptide” as used herein, generally refer to a peptide or polypeptide comprising at least one glycan residue. In various embodiments, glycopeptides comprise carbohydrate moieties (e.g. one or more glycans) covalently attached to a side chain (i.e. R group) of an amino acid residue.

The term “glycoprotein,” as used herein, generally refers to a protein having at least one glycan residue bonded thereto. In some examples, a glycoprotein is a protein with at least one oligosaccharide chain covalently bonded thereto. Examples of glycoproteins, include but are not limited to apolipoprotein C-III (APOC3), alpha-1-antichymotrypsin (AACT), afamin (AFAM), alpha-1-acid glycoprotein 1 & 2 (AGP12), apolipoprotein B-100 (APOB), apolipoprotein D (APOD), complement C1s subcomponent (C1S), calpain-3 (CAN3), clusterin (CLUS), complement component C8AChain (CO8A), alpha-2-HS-glycoprotein (FETUA), haptoglobin (HPT), immunoglobulin heavy constant gamma 1 (IgG1), immunoglobulin J chain (IgJ), plasma kallikrein (KLKB1), serum paraoxonase/arylesterase 1 (PON1), prothrombin (THRB), serotransferrin (TRFE), protein unc-13 homologA (UN13A), and zinc-alpha-2-glycoprotein (ZA2G). A glycopeptide, as used herein, refers to a fragment of a glycoprotein, unless specified otherwise to the contrary.

The term “liquid chromatography,” as used herein, generally refers to a technique used to separate a sample into parts. Liquid chromatography can be used to separate, identify, and quantify components.

The term “mass spectrometry,” as used herein, generally refers to an analytical technique used to identify molecules. In various embodiments described herein, mass spectrometry can be involved in characterization and sequencing of proteins.

The term “peptide,” as used herein, generally refers to amino acids linked by peptide bonds. Peptides can include amino acid chains between 10 and 50 residues. Peptides can include amino acid chains shorter than 10 residues, including, oligopeptides, dipeptides, tripeptides, and tetrapeptides. Peptides can include chains longer than 50 residues and may be referred to as “polypeptides” or “proteins.”

The terms “protein” or “polypeptide” or “peptide” may be used interchangeably herein and generally refer to a molecule including at least three amino acid residues. Proteins can include polymer chains made of amino acid sequences linked together by peptide bonds. Proteins may be digested in preparation for mass spectrometry using trypsin digestion protocols. Proteins may be digested using other proteases in preparation for mass spectrometry if access is limited to cleavage sites.

The term “peptide structure,” as used herein, generally refers to peptides or a portion thereof or glycopeptides or a portion thereof. In various embodiments described herein, a peptide structure can include any molecule comprising at least two amino acids in sequence.

The term “reduction,” as used herein, generally refers to the gain of an electron by a substance. In various embodiments described herein, a sugar can directly bind to a protein, thereby, reducing the amino acid to which it binds. Such reducing reactions can occur in glycosylation. In various embodiments, reduction may be used to break disulfide bonds between two cysteines.

The term “sample,” as used herein, generally refers to a sample from a subject of interest and may include a biological sample of a subject. The sample may include a cell sample. The sample may include a cell line or cell culture sample. The sample can include one or more cells. The sample can include one or more microbes. The sample may include a nucleic acid sample or protein sample. The sample may also include a carbohydrate sample or a lipid sample. The sample may be derived from another sample. The sample may include a tissue sample, such as a biopsy, core biopsy, needle aspirate, or fine needle aspirate. The sample may include a fluid sample, such as a blood sample, urine sample, or saliva sample. The sample may include a skin sample. The sample may include a cheek swab. The sample may include a plasma or serum sample. The sample may include a cell-free or cell free sample. A cell-free sample may include extracellular polynucleotides. The sample may originate from blood, plasma, serum, urine, saliva, mucosal excretions, sputum, stool, or tears. The sample may originate from red blood cells or white blood cells. The sample may originate from feces, spinal fluid, CNS fluid, gastric fluid, amniotic fluid, cyst fluid, peritoneal fluid, marrow, bile, other body fluids, tissue obtained from a biopsy, skin, or hair.

The term “sequence,” as used herein, generally refers to a biological sequence including one-dimensional monomers that can be assembled to generate a polymer. Non-limiting examples of sequences include nucleotide sequences (e.g. ssDNA, dsDNA, and RNA), amino acid sequences (e.g. proteins, peptides, and polypeptides), and carbohydrates (e.g. compounds including Cₘ(H₂O))ₙ).

The term “subject,” as used herein, generally refers to an animal, such as a mammal (e.g., human) or avian (e.g., bird), or other organism, such as a plant. For example, the subject can include a vertebrate, a mammal, a rodent (e.g., a mouse), a primate, a simian or a human. Animals may include, but are not limited to, farm animals, sport animals, and pets. A subject can include a healthy or asymptomatic individual, an individual that has or is suspected of having a disease (e.g., cancer) or a pre-disposition to the disease, and/or an individual that is in need of therapy or suspected of needing therapy. A subject can be a patient. A subject can include a microorganism or microbe (e.g., bacteria, fungi, archaea, viruses).

As used herein, a “model” may include one or more algorithms, one or more functions, one or more equations, one or more statistical tests, one or more mathematical techniques, one or more machine-learning algorithms, or a combination thereof.

As used herein, “abundance,” may refer to a quantitative value generated using mass spectrometry. The quantitative value may relate to the amount of a particular peptide structure. In one or more embodiments, the quantitative value may include an amount of an ion produced using mass spectrometry. The quantitative value may be expressed as an m/z value, in atomic mass units, or in some other manner.

As used herein, “relative abundance,” may refer to a comparison of two or more abundances. In one or more embodiments, the comparison may include comparing one peptide structure to a total number of a set of peptide structures (e.g., the total number of all peptide structures). In some embodiments, the comparison may include comparing one peptide glycoform (e.g., two identical peptides differing by one or more glycans) to a set of peptide glycoforms. In one or more embodiments, the comparison may include comparing a number of ions having a particular m/z ratio versus a total number of ions detected. In one or more embodiments, a relative abundance can be expressed as a ratio, as a percentage, or in some other manner.

The terms “determining”, “measuring”, “evaluating”, “assessing,” “assaying,” and “analyzing” are often used interchangeably herein to refer to forms of measurement, and include determining if an element is present or not (for example, detection). These terms can include quantitative, qualitative or quantitative and qualitative determinations. Assessing is alternatively relative or absolute. “Detecting the presence of” includes determining the amount of something present, as well as determining whether it is present or absent.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. In some embodiments, the mammal is a mouse, rat, simian, canine, feline, bovine, equine, or ovine. The subject may be diagnosed or suspected of being at high risk for a disease. The disease can be cancer. In some cases, the subject is not necessarily diagnosed or suspected of being at high risk for the disease or the condition.

As used herein, the terms “cancer” and “cancerous” refer to or describe the physiological condition in a subject that is typically characterized by unregulated cell growth. Examples of cancer include, but are not limited to, melanoma, carcinoma, lymphoma, blastoma, sarcoma, and leukemia and metastases thereof. The term “metastasis” refers to the transference of disease-producing organisms or of malignant or cancerous cells to other parts of the body by way of the blood or lymphatic vessels or membranous surfaces. Non-limiting examples of such cancers include small-cell lung cancer, non-small cell lung cancer, adenocarcinoma of the lung, squamous carcinoma of the lung, melanoma, squamous cell cancer, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, thyroid cancer, hepatic carcinoma and various types of head and neck cancer.

As used herein, the phrase “stage of disease” refers to the stages of cancer progression referred to as Stage I, II, III, or IV. Stage of disease indicates if metastasis has occurred in the subject.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term “protein” or “polypeptide” or “peptide” may be used interchangeably herein and refers to a molecule comprising at least three amino acid residues. As used herein, the term “protein” or “polypeptide” or “peptide” includes glycopeptides unless stated otherwise.

The term “polysaccharide” is used to describe any polymer made up of subunit monosaccharides, oligomers, or modified monosaccharides. In some embodiments, the polymer may be a homopolymer or a heteropolymer. The linkages between the subunits may include but are not limited to acetal linkages, such as glycosidic bonds; ester linkages such as phosphodiester linkages; amide linkages; and ether linkages.

The term “glycan” is used to describe a carbohydrate residue of a glycoconjugate, such as the carbohydrate portion of a glycopeptide, glycoprotein, glycolipid or proteoglycan. Glycan structures may be described by a glycan reference code number.

As used herein, the term “glycoform” refers to a unique primary, secondary, tertiary and quaternary structure of a protein with an attached glycan of a specific structure.

As used herein, the term “glycopeptide” or “glycopolypeptide” refers to a polypeptide having at least one glycan residue bonded thereto.

As used herein, the phrase “glycosylated peptides” or “glycosylated polypeptides” refers to a polypeptide bonded to a glycan residue.

As used herein, the term “glycoprotein,” refers to a protein having at least one glycan residue bonded thereto. In some examples, a glycoprotein is a protein with at least one oligosaccharide chain covalently bonded thereto. Examples of glycoproteins, include but are not limited to apolipoprotein C-III (APOC3), alpha-1-antichymotrypsin (AACT), afamin (AFAM), alpha-1-acid glycoprotein 1 & 2 (AGP12), apolipoprotein B-100 (APOB), apolipoprotein D (APOD), complement C1s subcomponent (C1S), calpain-3 (CAN3), clusterin (CLUS), complement component C8AChain (CO8A), alpha-2-HS-glycoprotein (FETUA), haptoglobin (HPT), immunoglobulin heavy constant gamma 1 (IgG1), immunoglobulin J chain (IgJ), plasma kallikrein (KLKB1), serum paraoxonase/arylesterase 1 (PON1), prothrombin (THRB), serotransferrin (TRFE), protein unc-13 homologA (UN13A), and zinc-alpha-2-glycoprotein (ZA2G). A glycopeptide, as used herein, refers to a fragment of a glycoprotein, unless specified otherwise to the contrary.

As used herein, the phrase “glycopeptide fragment,” “glycosylated peptide fragment,” “glycopolypeptide fragment”, and “glycosylated polypeptide fragment” refer to a glycosylated polypeptide or glycopeptide having an amino acid sequence that is the same as part (but not all) of the amino acid sequence of the glycosylated protein from which the glycosylated peptide is obtained by digestion, e.g., with one or more protease(s) or by fragmentation, e.g., ion fragmentation within a MRM-MS instrument. MRM refers to multiple-reaction-monitoring. Unless specified otherwise, “glycopeptide fragments” or “fragments of a glycopeptide” refer to the fragments produced directly by using a mass spectrometer optionally after the glycoprotein has been digested enzymatically to produce the glycopeptides.

As used herein, the phrase “multiple reaction monitoring mass spectrometry (MRM-MS),” refers to a highly sensitive and selective method for the targeted quantification of glycans and peptides in biological samples. Unlike traditional mass spectrometry, MRM-MS is highly selective (targeted), allowing researchers to fine tune an instrument to specifically look for certain peptides fragments of interest. MRM allows for greater sensitivity, specificity, speed and quantitation of peptides fragments of interest, such as a potential biomarker. MRM-MS involves using one or more of a triple quadrupole (QQQ) mass spectrometer and a quadrupole time-of-flight (qTOF) mass spectrometer.

As used herein, the phrase “digesting a glycopeptide,” refers to a biological process that employs enzymes to break specific amino acid peptide bonds. For example, digesting a glycopeptide includes contacting a glycopeptide with a digesting enzyme, e.g., trypsin, to produce fragments of the glycopeptide. In some examples, a protease enzyme is used to digest a glycopeptide. The term “protease” refers to an enzyme that performs proteolysis or breakdown of large peptides into smaller polypeptides or individual amino acids. Examples of a protease include, but are not limited to, one or more of a serine protease, threonine protease, cysteine protease, aspartate protease, glutamic acid protease, metalloprotease, asparagine peptide lyase, and any combinations of the foregoing.

As used herein, the phrase “fragmenting a glycopeptide,” refers to the ion fragmentation process which occurs in an MRM-MS instrument. Fragmenting may produce various fragments having the same mass but varying with respect to their charge.

As used herein, the phrase “multiple-reaction-monitoring (MRM) transition,” refers to the mass to charge (m/z) peaks or signals observed when a glycopeptide, or a fragment thereof, is detected by MRM-MS. The MRM transition is detected as the transition of the precursor and product ion.

As used herein, the phrase “detecting a multiple-reaction-monitoring (MRM) transition,” refers to the process in which a mass spectrometer analyzes a sample using tandem mass spectrometer ion fragmentation methods and identifies the mass to charge ratio for ion fragments in a sample. The absolute value of these identified mass to charge ratios are referred to as transitions. In the context of the methods set forth herein, the mass to charge ratio transitions are the values indicative of glycan, peptide or glycopeptide ion fragments. For some glycopeptides set forth herein, there is a single transition peak or signal. For some other glycopeptides set forth herein, there is more than one transition peak or signal. Background information on MRM mass spectrometry can be found in Introduction to Mass Spectrometry: Instrumentation, Applications, and Strategies for Data Interpretation, 4th Edition, J. Throck Watson, O. David Sparkman, ISBN: 978-0-470-51634-8, November 2007, the entire contents of which are here incorporated by reference in its entirety for all purposes.

As used herein, the phrase “detecting a multiple-reaction-monitoring (MRM) transition indicative of a glycopeptide,” refers to a MS process in which an MRM-MS transition is detected and then compare to a calculated mass to charge ratio (m/z) of a glycopeptide, or fragment thereof, in order to identify the glycopeptide. In some examples, herein, a single transition may be indicative of two more glycopeptides, if those glycopeptides have identical MRM-MS fragmentation patterns. A transition peak or signal includes, but is not limited to, those transitions set forth herein were are associated with a glycopeptide consisting essentially of an amino acid sequence selected from SEQ ID NO: 101-131, 159-207, and 21-46, and combinations thereof, according to Tables 1-5. A transition peak or signal includes, but is not limited to, those transitions set forth herein were are associated with a glycopeptide consisting of an amino acid sequence selected from SEQ ID NO: 101-131, 159-207, and 21-46, and combinations thereof, according to Tables 1-5.

As used herein, the term “reference value” refers to a value obtained from a population of individual(s) whose disease state is known. The reference value may be in n-dimensional feature space and may be defined by a maximum-margin hyperplane. A reference value can be determined for any particular population, subpopulation, or group of individuals according to standard methods well known to those of skill in the art.

As used herein, the term “population of individuals” means one or more individuals. In one embodiment, the population of individuals consists of one individual. In one embodiment, the population of individuals comprises multiple individuals. As used herein, the term “multiple” means at least 2 (such as at least 4, 6, 8, 10, 12, 14, 16, 18, 20, 22, 24, 26, 28, or 30) individuals. In one embodiment, the population of individuals comprises at least 10 individuals.

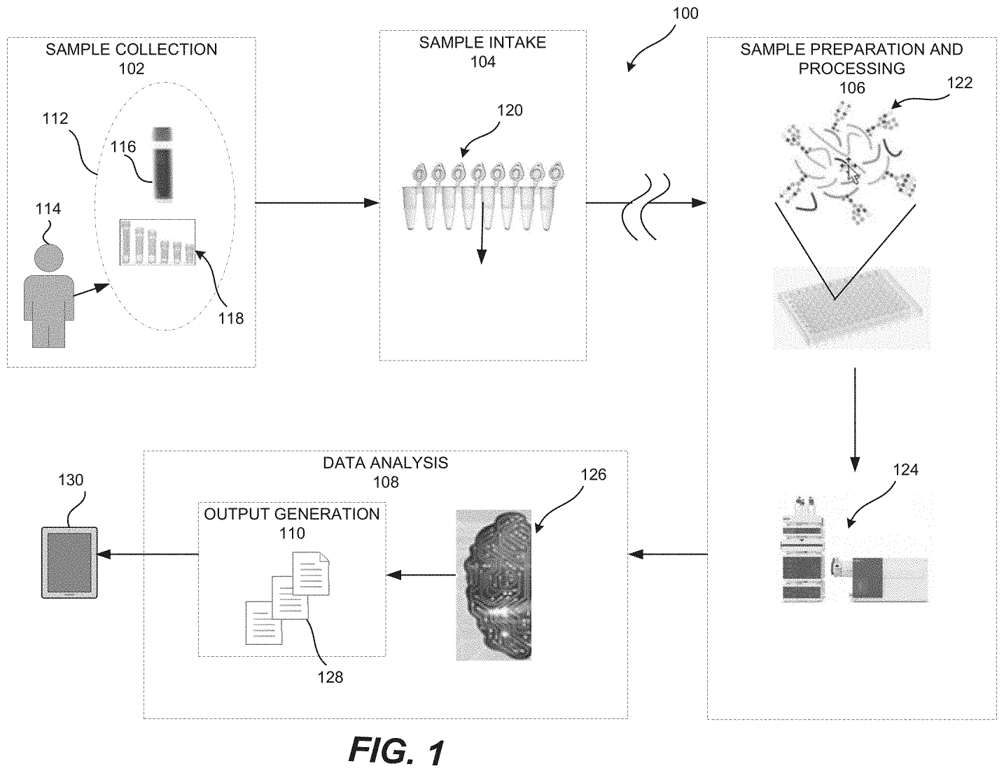

Glycans are referenced herein using the Symbol Nomenclature for Glycans (SNFG) for illustrating glycans. An explanation of this illustration system is available on the internet at www.ncbi.nlm.nih.gov/glycans/snfg.html, the entire contents of which are herein incorporated by reference in its entirety for all purposes. Symbol Nomenclature for Graphical Representation of Glycans as published in Glycobiology 25: 1323-1324, 2015. Additional information showing illustrations of the SNFG system are. Within this system, the term, Hex_i: is interpreted as follows: i indicates the number of green circles (mannose) and the number of yellow circles (galactose). The term, HexNAC_j, uses j to indicate the number of blue squares (G1cNAC's). The term Fuc_d, uses d to indicate the number of red triangles (fucose). The term Neu₅AC_1, uses 1 to indicate the number of purple diamonds (sialic acid). The glycan reference codes used herein combine these i, j, d, and l terms to make a composite 4-5 number glycan reference code, e.g., 5300 or 5320. See, for example, FIGS. 1 through 14 of PCT Patent Application No. PCT/US2020/0162861, filed Jan. 31, 2020, which are herein incorporated by reference in their entirety for all purposes.

The term “in vivo” is used to describe an event that takes place in a subject's body.

The term “ex vivo” is used to describe an event that takes place outside of a subject's body. An “ex vivo” assay is not performed on a subject. Rather, it is performed upon a sample separate from a subject. An example of an “ex vivo” assay performed on a sample is an “in vitro” assay.

The term “in vitro” is used to describe an event that takes places contained in a container for holding laboratory reagent such that it is separated from the living biological source organism from which the material is obtained. In vitro assays can encompass cell-based assays in which cells alive or dead are employed. In vitro assays can also encompass a cell-free assay in which no intact cells are employed.

As used herein, the term ‘about’ a number refers to that number plus or minus 10% of that number. The term ‘about’ a range refers to that range minus 10% of its lowest value and plus 10% of its greatest value.

### III. Overview of Exemplary Workflow

FIG. 1 is a schematic diagram of an exemplary workflow 100 for the detection of peptide structures associated with a condition for use in treatment management in accordance with one or more embodiments. Workflow 100 may include various operations including, for example, sample collection 102, sample intake 104, sample preparation and processing 106, data analysis 108, and output generation 110.

Sample collection 102 may include, for example, obtaining a biological sample 112 of one or more subjects, such as subject 114. Biological sample 112 may take the form of a specimen obtained via one or more sampling methods. Biological sample 112 may be representative of subject 114 as a whole or of a specific tissue, cell type, or other category or sub-category of interest. Biological sample 112 may be obtained in any of a number of different ways. In various embodiments, biological sample 112 includes whole blood sample 116 obtained via a blood draw. In other embodiments, biological sample 112 includes set of aliquoted samples 118 that includes, for example, a serum sample, a plasma sample, a blood cell (e.g., white blood cell (WBC), red blood cell (RBC) sample, another type of sample, or a combination thereof. Biological samples 112 may include nucleotides (e.g., ssDNA, dsDNA, RNA), organelles, amino acids, peptides, proteins, carbohydrates, glycoproteins, or any combination thereof.

Sample intake 104 may include one or more various operations such as, for example, aliquoting, registering, processing, storing, thawing, and/or other types of operations. In one or more embodiments, when biological sample 112 includes whole blood sample 116, sample intake 104 includes aliquoting whole blood sample 116 to form a set of aliquoted samples that can then be sub-aliquoted to form set of samples 120.

Sample preparation and processing 106 may include, for example, one or more operations to form set of peptide structures 122. In various embodiments, set of peptide structures 122 may include various fragments of unfolded proteins that have undergone digestion and may be ready for analysis.

Further, sample preparation and processing 106 may include, for example, data acquisition 124 based on set of peptide structures 122. For example, data acquisition 124 may include use of, for example, but is not limited to, a liquid chromatography/mass spectrometry (LC/MS) system.

Data analysis 108 may include, for example, peptide structure analysis 126. In some embodiments, data analysis 108 also includes output generation 110. In other embodiments, output generation 110 may be considered a separate operation from data analysis 108. Output generation 110 may include, for example, generating final output 128 based on the results of peptide structure analysis 126. Final output 128 may be used for the research, and/or treatment of disease, such as, for example, melanoma.

In various embodiments, final output 128 is comprised of one or more outputs. Final output 128 may take various forms. For example, final output 128 may be a report that includes, for example, a treatment output (e.g., a treatment design output, a treatment plan output, or combination thereof). In some embodiments, final output 128 may be an alert (e.g., a visual alert, an audible alert, etc.), a notification (e.g., a visual notification, an audible notification, an email notification, etc.), an email output, or a combination thereof. In some embodiments, final output 128 may be sent to remote system 130 for processing. Remote system 130 may include, for example, a computer system, a server, a processor, a cloud computing platform, cloud storage, a laptop, a tablet, a smartphone, some other type of mobile computing device, or a combination thereof.

In other embodiments, workflow 100 may optionally exclude one or more of the operations described herein and/or may optionally include one or more other steps or operations other than those described herein (e.g., in addition to and/or instead of those described herein). For example, in one or more embodiments, final output 128 may not be sent to remote system 130 for processing. Instead, a notification or a communication (e.g., email) may be sent to remote system 130 to notify a user(s) or entity that final output 128 is available for retrieval (e.g., download). Accordingly, workflow 100 may be implemented in any of a number of different ways for use in the research and/or treatment of melanoma.

### I. Detection and Quantification of Peptide Structures

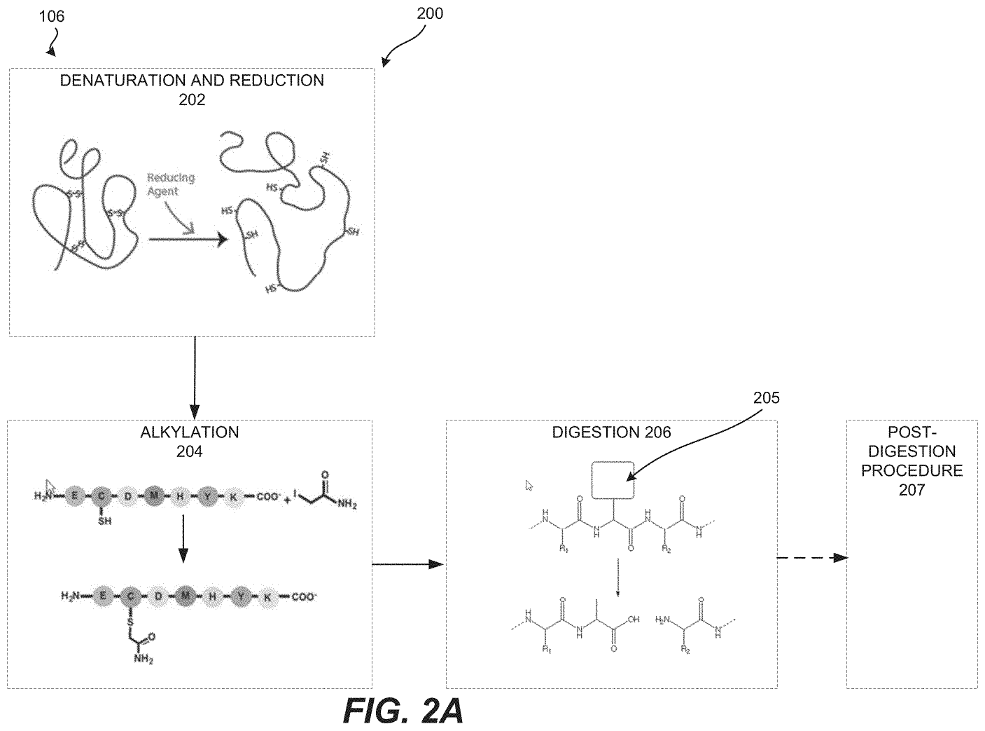

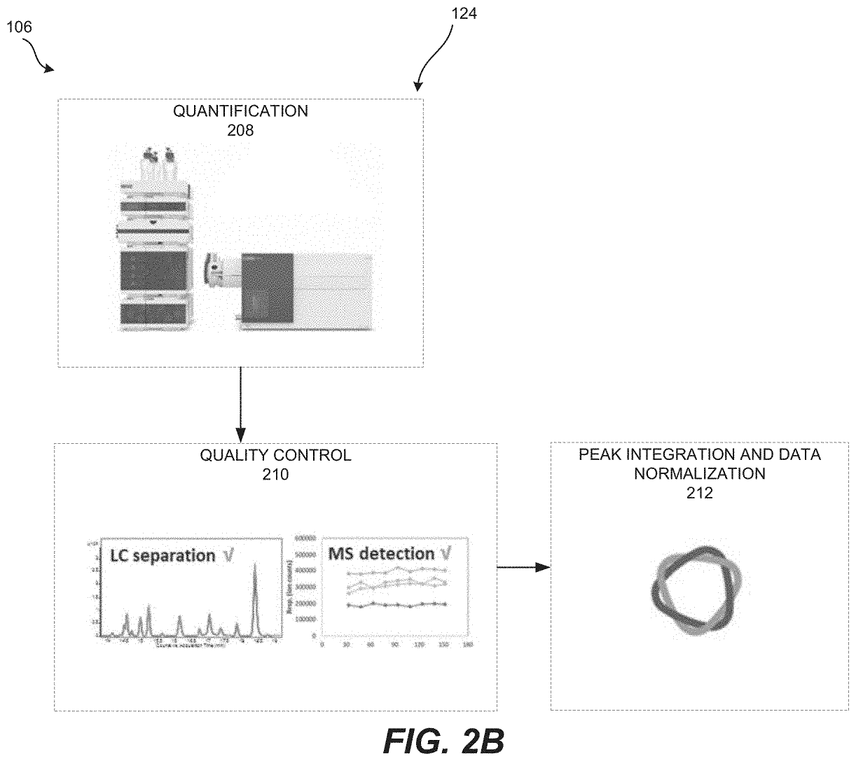

FIGS. 2A and 2B are schematic diagrams of a workflow for sample preparation and processing 106 in accordance with one or more embodiments. FIGS. 2A and 2B are described with continuing reference to FIG. 1. Sample preparation and processing 106 may include, for example, preparation workflow 200 shown in FIG. 2A and data acquisition 124 shown in FIG. 2B.

I.A. Sample Preparation and Processing

FIG. 2A is a schematic diagram of preparation workflow 200 in accordance with one or more embodiments. Preparation workflow 200 may be used to prepare a sample, such as a sample of set of samples 120 in FIG. 1, for analysis via data acquisition 124. For example, this analysis may be performed via mass spectrometry. In various embodiments, preparation workflow 200 may include denaturation and reduction 202, alkylation 204, and digestion 206.

In general, polymers, such as proteins, in their native form, can fold to include secondary, tertiary, and/or other higher order structures. Such higher order structures may functionalize proteins to complete tasks (e.g., enable enzymatic activity) in a subject. Further, such higher order structures of polymers may be maintained via various interactions between side chains of amino acids within the polymers. Such interactions can include ionic bonding, hydrophobic interactions, hydrogen bonding, and disulfide linkages between cysteine residues. However, when using analytic systems and methods, including mass spectrometry, unfolding such polymers (e.g., peptide/protein molecules) may be desired to obtain sequence information. In some embodiments, unfolding a polymer may include denaturing the polymer, which may include, for example, linearizing the polymer.

In one or more embodiments, denaturation and reduction 202 can be used to disrupt higher order structures (e.g., secondary, tertiary, quaternary, etc.) of one or more proteins (e.g., polypeptides and peptides) in a sample (e.g., one of set of samples 120 in FIG. 1). Denaturation and reduction 202 may include, for example, a denaturation procedure and a reduction procedure. In some embodiments, the denaturation procedure may be performed using, for example, thermal denaturation, where heat is used as a denaturing agent. The thermal denaturation can disrupt ionic bonding, hydrophobic interactions, and/or hydrogen bonding.

In one or more embodiments, the denaturation procedure may include using one or more denaturing agents in combination with heat. These one or more denaturing agents may include, for example, but are not limited to, any number of chaotropic salts (e.g., urea, guanidine), surfactants (e.g., sodium dodecyl sulfate (SDS), beta octyl glucoside, Triton X-100), or combination thereof. In some cases, such denaturing agents may be used in combination with heat when sample preparation workflow further includes a cleanup procedure.

The resulting one or more denatured (e.g., unfolded, linearized) proteins may then undergo further processing in preparation of analysis. For example, a reduction procedure may be performed in which one or more reducing agents are applied. A reducing agent may take the form of, for example, without limitation, dithiothreitol (DTT), tris(2-carboxyethyl)phosphine (TCEP), or some other reducing agent. The reducing agent may reduce (e.g., cleave) the disulfide linkages between cysteine residues of the one or more denatured proteins to form one or more reduced proteins.

In various embodiments, the one or more reduced proteins resulting from denaturation and reduction 202 may undergo a process to prevent the reformation of disulfide linkages between, for example, the cysteine residues of the one or more reduced proteins. This process may be implemented using alkylation 204 to form one or more alkylated proteins. For example, alkylation 204 may be used add an acetamide group to a sulfur on each cysteine residue to prevent disulfide linkages from reforming. In various embodiments, an acetamide group can be added by reacting one or more alkylating agents with a reduced protein. The one or more alkylating agents may include, for example, one or more acetamide salts. An alkylating agent may take the form of, for example, iodoacetamide (IAA), 2-chloroacetamide, some other type of acetamide salt, or some other type of alkylating agent.

In some embodiments, alkylation 204 may include a quenching procedure. The quenching procedure may be performed using one or more reducing agents (e.g., one or more of the reducing agents described above).

In various embodiments, the one or more alkylated formed via alkylation 204 can then undergo digestion 206 in preparation for analysis (e.g., mass spectrometry analysis). Digestion 206 of a protein may include cleaving the protein at or around one or more cleavage sites (e.g., site 205 which may be one or more amino acid residues). For example, without limitation, an alkylated protein may be cleaved at the carboxyl side of the lysine or arginine residues. This type of cleavage may break the protein into various segments, which include one or more peptide structures (e.g., glycosylated or aglycosylated).

In various embodiments, digestion 206 is performed using one or more proteolysis catalysts. For example, an enzyme can be used in digestion 206. In some embodiments, the enzyme takes the form of trypsin. In other embodiments, one or more other types of enzymes (e.g., proteases) may be used in addition to or in place of trypsin. These one or more other enzymes include, but are not limited to, LysC, LysN, AspN, GluC, and ArgC. In some embodiments, digestion 206 may be performed using tosyl phenylalanyl chloromethyl ketone (TPCK)-treated trypsin, one or more engineered forms of trypsin, one or more other formulations of trypsin, or a combination thereof. In some embodiments, digestion 206 may be performed in multiple steps, with each involving the use of one or more digestion agents. For example, a secondary digestion, tertiary digestion, etc. may be performed. In one or more embodiments, trypsin is used to digest serum samples. In one or more embodiments, trypsin/LysC cocktails are used to digest plasma samples.

In one or more embodiments, digestion 206 further includes a quenching procedure. The quenching procedure may be performed by acidifying the sample (e.g., to a pH<3). In one or more embodiments, formic acid may be used to perform this acidification.

In various embodiments, preparation workflow 200 further includes post-digestion procedure 207. Post-digestion procedure 207 may include, for example, a cleanup procedure. The cleanup procedure may include, for example, the removal of unwanted components in the sample that results from digestion 206. For example, unwanted components may include, but are not limited to, inorganic ions, surfactants, etc. In some embodiments, post-digestion procedure 207 further includes a procedure for the addition of heavy-labeled peptide internal standards.

Although preparation workflow 200 has been described with respect to a sample created or taken from biological sample 112 that is blood-based (e.g., a whole blood sample, a plasma sample, a serum sample, etc.), sample preparation workflow 200 may be similarly implemented for other types of samples (e.g., tears, urine, tissue, interstitial fluids, sputum, etc.) to produce set of peptides structures 122.

I.B. Peptide Structure Identification and Quantitation

FIG. 2B is a schematic diagram of data acquisition 124 in accordance with one or more embodiments. In various embodiments, data acquisition 124 can commence following sample preparation 200 described in FIG. 2A. In various embodiments, data acquisition 124 can comprise quantification 208, quality control 210, and peak integration and normalization 212.

In various embodiments, targeted quantification 208 of peptides and glycopeptides can incorporate use of liquid chromatography-mass spectrometry LC/MS instrumentation. For example, LC-MS/MS, or tandem MS may be used. In general, LC/MS (e.g., LC-MS/MS) can combine the physical separation capabilities of liquid chromatograph (LC) with the mass analysis capabilities of mass spectrometry (MS). According to some embodiments described herein, this technique allows for the separation of digested peptides to be fed from the LC column into the MS ion source through an interface.

In various embodiments, any LC/MS device can be incorporated into the workflow described herein. In various embodiments, a Triple Quadrupole LC/MS™ includes example instruments suited for identification and targeted quantification 208. In various embodiments, targeted quantification 208 is performed using multiple reaction monitoring mass spectrometry (MRM-MS).

In various embodiments described herein, identification of a particular protein or peptide and an associated quantity can be assessed. In various embodiments described herein, identification of a particular glycan and an associated quantity can be assessed. In various embodiments described herein, particular glycans can be matched to a glycosylation site on a protein or peptide and their absolute or relative quantities assessed.

In some cases, targeted quantification 208 includes using a specific collision energy associated for the appropriate fragmentation to consistently see an abundant product ion. Glycopeptide structures may have a lower collision energy than aglycosylated peptide structures. When analyzing a sample that includes glycopeptide structures, the source voltage and gas temperature may be lowered as compared to generic proteomic analysis.

In various embodiments, quality control 210 procedures can be put in place to optimize data quality. In various embodiments, measures can be put in place allowing only errors within acceptable ranges outside of an expected value. In various embodiments, employing statistical models (e.g. using Westgard rules) can assist in quality control 210. For example, quality control 210 may include, for example, assessing the retention time and abundance of representative peptide structures (e.g., glycosylated and/or aglycosylated) and spiked-in internal standards, in either every sample, or in each quality control sample (e.g., pooled serum digest).

Peak integration and normalization 212 may be performed to process the data that has been generated and transform the data into a format for analysis. For example, peak integration and normalization 212 may include converting abundance data for various product ions that were detected for a selected peptide structure into a single quantification metric (e.g., a relative quantity, an adjusted quantity, a normalized quantity, a relative concentration, an adjusted concentration, a normalized concentration, etc.) for that peptide structure. In some embodiments, peak integration and normalization 212 may be performed using one or more of the techniques described in U.S. Patent Publication No. 2020/0372973A1 and/or US Patent Publication No. 2020/0240996, the disclosures of which are incorporated by reference herein in their entireties.

### II. Peptide Structure Data Analysis and Melanoma Treatment Management

II.A. Exemplary System

II.A.1. System for Analyzing Peptide Structure Data and Managing Melanoma Treatment

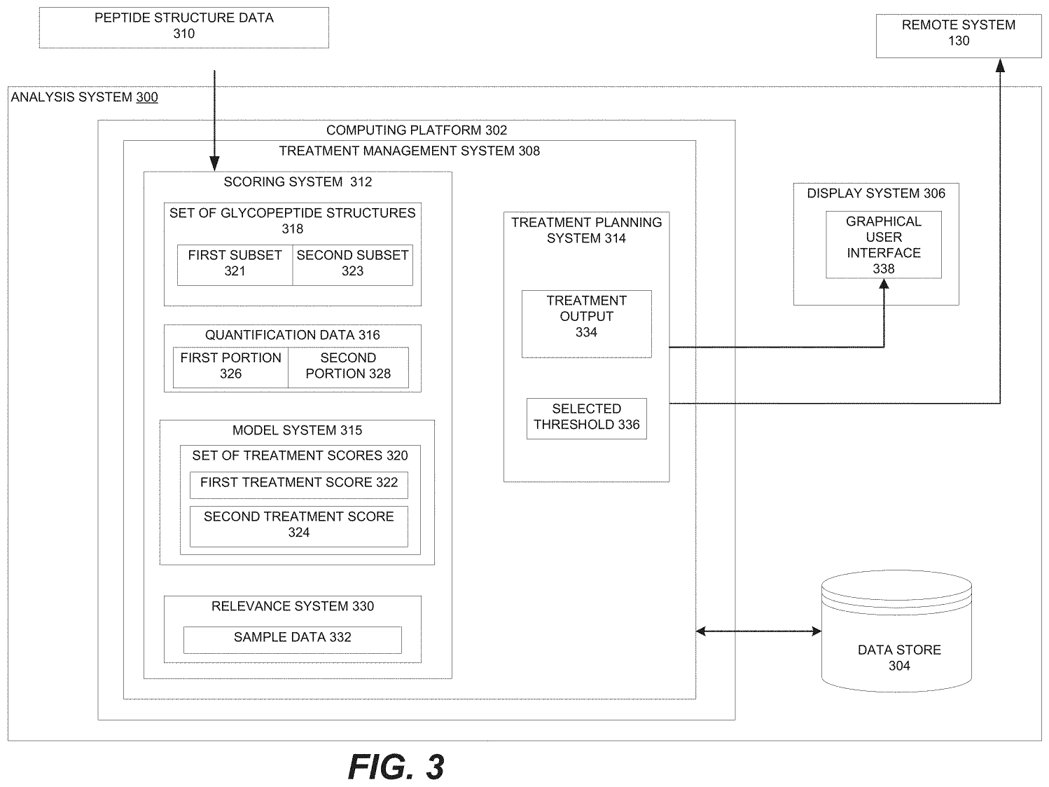

FIG. 3 is a block diagram of an analysis system 300 in accordance with one or more embodiments. Analysis system 300 can be used to both detect and analyze various peptide structures that have been associated with melanoma treatments. Analysis system 300 is one example of an implementation for a system that may be used to perform data analysis 108 in FIG. 1. Thus, analysis system 300 is described with continuing reference to workflow 100 as described in FIGS. 1, 2A, and/or 2B.

Analysis system 300 may include computing platform 302 and data store 304. In some embodiments, analysis system 300 also includes display system 306. Computing platform 302 may take various forms. In one or more embodiments, computing platform 302 includes a single computer (or computer system) or multiple computers in communication with each other. In other examples, computing platform 302 takes the form of a cloud computing platform. In still other examples, computing platform 302 may include any number of or combination computers, cloud computing platforms, servers, or mobile devices.

Data store 304 and display system 306 may each be in communication with computing platform 302. In some examples, data store 304, display system 306, or both may be considered part of or otherwise integrated with computing platform 302. Thus, in some examples, computing platform 302, data store 304, and display system 306 may be separate components in communication with each other, but in other examples, some combination of these components may be integrated together. Communication between these different components may be implemented using any number of wired communications links, wireless communications links, optical communications links, or a combination thereof.

Analysis system 300 includes, for example, treatment management system 308, which may be implemented using hardware, software, firmware, or a combination thereof. In one or more embodiments, peptide structure analyzer 308 is implemented using computing platform 302.

Treatment management system 308 may be used to manage the treatment of a subject diagnosed with a melanoma condition (i.e., malignant melanoma). Treatment management system 308 may be used to predict the subject's response to one or more treatments for the melanoma condition, select a treatment to be administered to the subject to prevent the progression (or advancement) of the melanoma condition and/or otherwise improve the condition of the subject, and/or otherwise plan the treatment of the subject.

Treatment management system 308 receives peptide structure data 310 for processing. Peptide structure data 310 may have been generated using multiple reaction monitoring mass spectrometry. Peptide structure data 310 may be, for example, the peptide structure data that is output from sample preparation and processing 106 in FIGS. 1, 2A, and 2B. Accordingly, peptide structure data 310 may correspond to set of peptide structures 122 identified for biological sample 112 and may thereby correspond to biological sample 112. Further, as set of peptide structures 122 corresponds to a set of glycoproteins (e.g., each peptide structure of set of peptide structures 122 being derived from a corresponding glycoprotein), peptide structure data 310 therefore corresponds to the set of glycoproteins. In some cases, two or more peptide structures may correspond to a same glycoprotein and these two or more peptide structures may be referred to as glycoforms of that same glycoprotein.

Peptide structure data 310 can be sent as input into treatment management system 308, retrieved from data store 304 or some other type of storage (e.g., cloud storage), accessed from cloud storage, or obtained in some other manner. In some cases, peptide structure data 310 may be retrieved from data store 304 in response to (e.g., directly or indirectly based on) receiving user input entered by a user via an input device.

Treatment management system 308 may include scoring system 312. In one or more embodiments, treatment management system 308 further includes and treatment planning system 314. Scoring system 312 may be used to predict the response of a subject (e.g., subject 114) to one or more types of treatment. Treatment planning system 314 may be used to plan how to treat the subject based on the predicted response(s) for the subject.

Scoring system 312 may include, for example, model system 315 that is configured to receive peptide structure data 310 for processing. Model system 315 may be implemented in any of a number of different ways. Model system 315 may be a computational model system that may be implemented using any number of models, functions, equations, algorithms, and/or other mathematical techniques.

In one or more embodiments, scoring system 312 receives peptide structure data 310 for processing and inputs quantification data 316 identified from peptide structure data 310 for set of peptide structures 318 into model system 315. Model system 315 analyzes quantification data 316 to generate set of treatment scores 320 corresponding to a set of treatments. Peptide structure data 310 may comprise a set of quantification metrics for each peptide structure of, for example, set of peptide structures 122 in FIG. 1. A quantification metric for a peptide structure may be comprised of at least one of a relative abundance, a normalized abundance, an adjusted abundance, an absolute abundance, a relative quantity, an adjusted quantity, a normalized quantity, a relative concentration, an adjusted concentration, or a normalized concentration. Accordingly, quantification data 316 may include one or more quantification metrics for each peptide structure of set of peptide structures 318.

A peptide structure of set of peptide structures 318 may be a glycosylated peptide structure, or glycopeptide structure, that is defined by a peptide sequence and a glycan structure attached to a linking site of the peptide sequence quantity. For example, the peptide structure may be a glycopeptide or a portion of a glycopeptide. Alternatively, a peptide structure of set of peptide structures 318 may be an aglycosylated peptide structure that is defined by a peptide sequence. For example, the peptide structure may be a peptide or a portion of a peptide and may be referred to as a quantification peptide.

Set of peptide structures 318 may be identified as being those most predictive or relevant to the response of a subject to a corresponding treatment(s) based on training of model 312. In one or more embodiments, set of peptide structures 318 includes at least one, at least three, at least five, or at least some other number of the peptide structures identified in Table 1 below in Section V.B. The number of peptide structures selected from Table 1 for inclusion in set of peptide structures 318 may be based on, for example, a desired level of accuracy, the number of treatments for which set of treatment scores 320 are being generated, one or more other factors, or a combination thereof.

In one or more embodiments, model system 315 may be used to analyze the response of a subject to a pembrolizumab treatment (“pembro”), the response of the subject to a combination treatment comprised of the combination of nivolumab and ipilimumab (“ipi/nivo”). Both pembro and ipi/nivo are treatments used to treat melanoma. For example, model system 315 may use quantification data 316 for set of peptide structures 318 to generate set of treatment scores 320 that includes a first treatment score 322 for pembro and a second treatment score 324 for ipi/nivo. In one or more embodiments, set of peptide structures 318 may include first subset 321 of set of peptide structures 318 used to compute first treatment score 322 and second subset 323 of set of peptide structures 318 used to compute second treatment score 324. In one or more embodiments, first subset 321 and the second subset 323 of set of peptide structures 318 may partially overlap (e.g., have one, two, three, four, five, some other number of peptide structures in common.

First portion 326 of quantification data 316 used to compute first treatment score 322 may correspond to first subset 321. Second portion 328 of quantification data 316 used to compute second treatment score 324 may correspond to second subset 323. First portion 326 and second portion 328 may be referred to as first quantification data and second quantification data, respectively. When first subset 321 and second subset 323 partially overlap, first portion 326 and second portion 328 similarly overlap. As one example, first portion 326 of quantification data 316 corresponding to first portion 321 used to compute first treatment score 322 and second portion 328 of quantification data 316 corresponding to second subset 323 of set of peptide structures 318 used to compute second treatment score 324 may have two peptide structures in common.

In one or more embodiments, first subset 321 of set of peptide structures 318 includes at least one, at least three, at least five, or at least some other number of the peptide structures identified in Table 2 below in Section V.B. In one or more embodiments, second subset 323 of set of peptide structures 318 includes at least one, at least three, at least five, or at least some other number of the peptide structures identified in Table 3 below in Section V.B.

In one or more embodiments, set of peptide structures 318 may have been identified by treatment management system 308 using relevance system 330. Relevance system 330 may include any number of computational models to analyze sample data 332 to determine which peptide structures to include in set of peptide structures 318. Sample data 332 may be retrieved from data store 304 or received in some other manner. Sample data 332 may include data capturing multiple subjects' responses to one or more treatments. For example, sample data 332 may include data capturing subjects' responses to pembro and to subjects' responses to ipi/nivo.

In one or more embodiments, relevance system 330 includes a first algorithm that uses a Wilcoxon rank-sum test to determine first subset 321 and a second algorithm that uses the Wilcoxon rank-sum test to determine second subset 323. For example, relevance system 330 includes a first algorithm that uses a Wilcoxon rank-sum test to determine which peptide structures to include in first subset 321 to compute first treatment score 322 (e.g., for pembro) and a second algorithm that uses the Wilcoxon rank-sum test to determine which peptide structures to include in second subset 323 to compute second treatment score 324 (e.g., for ipi/nivo).

Treatment planning system 314 receives set of treatment scores 320 from scoring system 312. Treatment planning system 314 uses set of treatment scores 320 to generate treatment output 334. Treatment output 334 may include, for example, an identification or categorization of the response of the subject to the one or more treatments for which the subject's response is being predicted, at least one of an identification of a therapeutic to treat the subject, a design for the therapeutic, a treatment plan for administering the therapeutic, or a combination thereof. In some embodiments, the therapeutic is an immune checkpoint inhibitor. In various embodiments, treatment output 326 includes a therapeutic dosage for each therapeutic to be used in treating the subject.

In one or more embodiments, treatment output 334 identifies a response classification that indicates a predicted response for the subject to a treatment. For example, set of treatment scores 320 may include a treatment score that can be used to classify a subject's response to a melanoma treatment as either early disruption or sustained control.

The response classification may be, for example, a positive response classification, a negative response classification, or some other type of response classification. A positive response classification may, for example, indicate that the subject is predicted to have a relatively positive or otherwise successful response to treatment. A negative response classification may, for example, indicate that the subject is predicted to have a relatively poor or otherwise unsuccessful response to treatment. In one or more embodiments, the response classification predicts response to treatment with respect to survivability (e.g., overall survival, progression-free survival, etc.).

“Early disruption” may be an example of a negative response classification. “Early disruption” may indicate that the subject is predicted to have a relatively poor response to the treatment. For example, a prediction of “early disruption” may mean that the subject is predicted to have a disruption event within an initial period of time (e.g., 6 months) after treatment. A disruption event may be any event that disrupts the subject's “progression-free survival” (PFS). A disruption event may be also referred to as a progression event or an advancement event as such an event indicates disease progression or advancement. In some cases, the progression event may be a final level of progression or disease advancement, such as death. Thus, “early disruption” may also be referred to as “progression,” “disease progression,” or “disease advancement.” A disruption event may include, for example, at least one of a new melanoma (e.g., malignant mole), an increase in the size of an existing melanoma, or some other type of event. A disruption event may be detected using any number of progression criteria. For example, a disruption event may be considered “detected” in response to a selected number or proportion of a set of progression criteria being met. The set of progression criteria may include, for example, but is not limited to, one or more immune-related response criteria (irRC), one or more response evaluation criteria in solid tumors (RECIST), one or more other types of criteria, or a combination thereof.

“Sustained control” may be one example of a positive response classification. “Sustained control” may be a response classification that indicates that the subject is predicted to have a relatively successful response to the treatment. For example, a prediction of “sustained control” may mean that the subject is predicted to have no disruption events within a sustained period of time (e.g., 12 months) after treatment. The sustained period of time may be longer than the initial period of time.

In one or more embodiments, treatment planning system 314 uses one or more selected thresholds to classify set of treatment scores 320. In one or more embodiments, a different selected threshold is used for each treatment. In other embodiments, a same threshold is used for all treatments being considered. For example, treatment planning system 314 may use selected threshold 336. In one or more embodiments, selected threshold is 0.5. In other embodiments, selected threshold is 0.6, 0.7, 0.75, 0.8, or some other threshold.

As one example, when selected threshold is 0.5, treatment planning system 314 may generate a first predicted response based on a determination that a treatment score is above (or is at and above) the selected threshold and may generate a second predicted response based on a determination that the treatment score is not above (or is below) the selected threshold. The first predicted response may be, for example, a first predicted response classification (e.g., sustained control); the second predicted response may be a second predicted response classification (e.g., early disruption).

Treatment output 334 may include the response classification that is predicted such that a user (e.g., a medical professional) can determine whether a corresponding treatment should be or should not be administered to a subject. For example, when first treatment score 322 is generated for pembro, and treatment output 334 indicates that a subject's predicted response is “early disruption,” a medical professional may determine to administer a different treatment, a higher dosage of pembro, or change the treatment plan for the subject in some other way.

When set of treatment scores 320 includes at least two treatment scores, treatment planning system 314 may analyze the at least two treatment scores and determine which treatment score indicates a best response to the corresponding treatment for the subject. As one example, treatment planning system 314 may compare the at least treatment scores and select the treatment corresponding to the highest treatment score for the subject. This selected treatment may then be identified in treatment output 334. In some cases, treatment output 334 may further include a therapeutic dosage (e.g., an approved dosage) for selected treatment for the subject. In some cases, treatment output 334 may further include a response classification for the selected treatment. For example, while first treatment score 322 may be higher than second treatment score 324, both first treatment score 322 and second treatment score 324 may indicate that the predicted response for the subject is “early disruption” with both treatments. In this example, treatment output 336 may identify the treatment corresponding to first treatment score 322 with an indication that the predicted response “early disruption” and a recommendation to either select a different treatment, alter (e.g., increase/decrease) a dosage of the treatment corresponding to first treatment score 322, combine the treatment with at least one other treatment, or change the treatment plan for the subject in some other manner.

Treatment output 334 may be sent to remote system 130 for processing in some examples. In other embodiments, treatment output 334 may be displayed on graphical user interface 338 in display system 306 for viewing by a human operator. The human operator may use treatment output 334 to manage the melanoma treatment of the subject.

II.A.2. Computer Implemented System

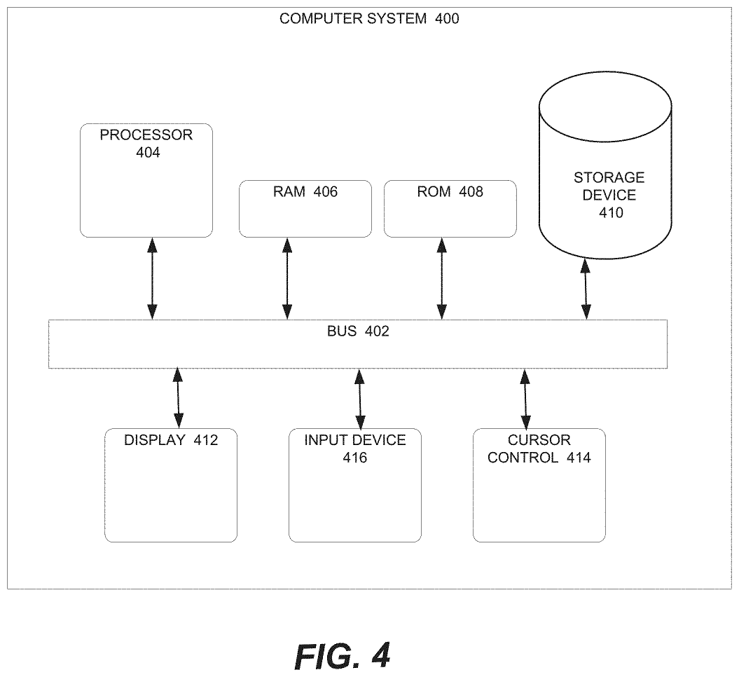

FIG. 4 is a block diagram of a computer system in accordance with various embodiments. Computer system 400 may be an example of one implementation for computing platform 302 described above in FIG. 3.

In one or more examples, computer system 400 can include a bus 402 or other communication mechanism for communicating information, and a processor 404 coupled with bus 402 for processing information. In various embodiments, computer system 400 can also include a memory, which can be a random-access memory (RAM) 406 or other dynamic storage device, coupled to bus 402 for determining instructions to be executed by processor 404. Memory also can be used for storing temporary variables or other intermediate information during execution of instructions to be executed by processor 404. In various embodiments, computer system 400 can further include a read only memory (ROM) 408 or other static storage device coupled to bus 402 for storing static information and instructions for processor 404. A storage device 410, such as a magnetic disk or optical disk, can be provided and coupled to bus 402 for storing information and instructions.

In various embodiments, computer system 400 can be coupled via bus 402 to a display 412, such as a cathode ray tube (CRT) or liquid crystal display (LCD), for displaying information to a computer user. An input device 414, including alphanumeric and other keys, can be coupled to bus 402 for communicating information and command selections to processor 404. Another type of user input device is a cursor control 416, such as a mouse, a joystick, a trackball, a gesture input device, a gaze-based input device, or cursor direction keys for communicating direction information and command selections to processor 404 and for controlling cursor movement on display 412. This input device 414 typically has two degrees of freedom in two axes, a first axis (e.g., x) and a second axis (e.g., y), that allows the device to specify positions in a plane. However, it should be understood that input devices 414 allowing for three-dimensional (e.g., x, y, and z) cursor movement are also contemplated herein.

Consistent with certain implementations of the present teachings, results can be provided by computer system 400 in response to processor 404 executing one or more sequences of one or more instructions contained in RAM 406. Such instructions can be read into RAM 406 from another computer-readable medium or computer-readable storage medium, such as storage device 410. Execution of the sequences of instructions contained in RAM 406 can cause processor 404 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, storage device, data storage device, etc.) or “computer-readable storage medium” as used herein refers to any media that participates in providing instructions to processor 404 for execution. Such a medium can take many forms, including but not limited to, non-volatile media, volatile media, and transmission media. Examples of non-volatile media can include, but are not limited to, optical, solid state, magnetic disks, such as storage device 410. Examples of volatile media can include, but are not limited to, dynamic memory, such as RAM 406. Examples of transmission media can include, but are not limited to, coaxial cables, copper wire, and fiber optics, including the wires that comprise bus 402.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 404 of computer system 400 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, optical communications connections, etc.

It should be appreciated that the methodologies described herein, flow charts, diagrams, and accompanying disclosure can be implemented using computer system 400 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as R, C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 400, whereby processor 404 would execute the analyses and determinations provided by these engines, subject to instructions provided by any one of, or a combination of, the memory components RAM 406, ROM, 408, or storage device 410 and user input provided via input device 414.

II.B. Exemplary Methodologies for Analyzing Peptide Structure Data and Managing Melanoma Treatment

II.B.1. Predicting Treatment Response

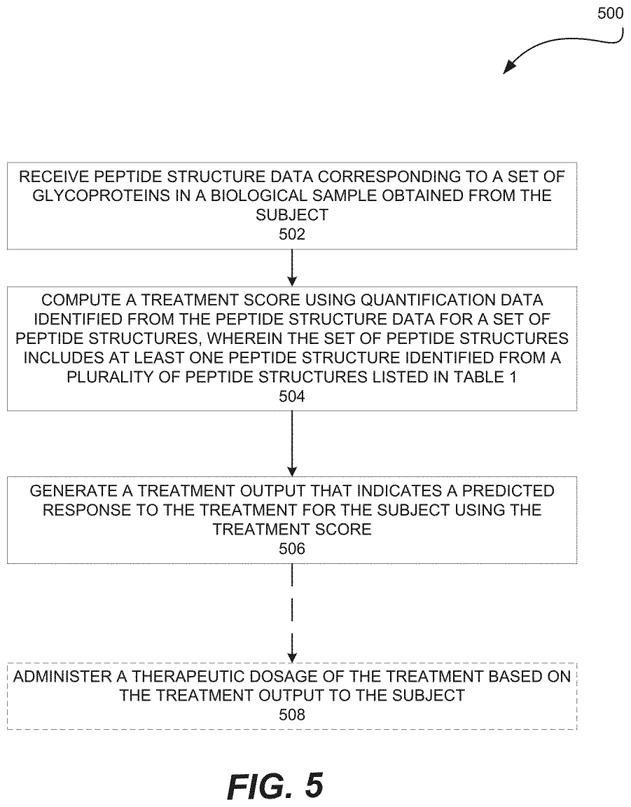

FIG. 5 is a flowchart of a process for managing a treatment for a subject diagnosed with a melanoma condition in accordance with one or more embodiments. Process 500 may be implemented using, for example, at least a portion of workflow 100 as described in FIGS. 1, 2A, and 2B and/or analysis system 300 as described in FIG. 3. Process 500 may be used to generate, for example, a treatment output such as treatment output 334 in FIG. 3 to aid in the treatment of a subject diagnosed with a melanoma condition (e.g., malignant melanoma).

Step 502 includes receiving peptide structure data corresponding to a set of glycoproteins in a biological sample obtained from the subject. The peptide structure data may be, for example, one example of an implementation of peptide structure data 310 in FIG. 3. The peptide structure data may have been generated using multiple reaction monitoring mass spectrometry. The peptide structure data may include quantification data for each peptide structure of a plurality of peptide structures. The quantification data may include, for example, one or more quantification metrics for each peptide structure of the plurality of peptide structures. A quantification metric for a peptide structure may include, for example, but is not limited to, at least one of a relative abundance, an absolute abundance, an adjusted abundance, a normalized abundance, a relative quantity, an adjusted quantity, a normalized quantity, a relative concentration, an adjusted concentration, or a normalized concentration. In this manner, the quantification data for a given peptide structure provides an indication of the abundance of the peptide structure in the biological sample.

Step 504 includes computing a treatment score using quantification data identified from the peptide structure data for a set of peptide structures, wherein the set of peptide structures includes at least one peptide structure identified from a plurality of peptide structures listed in Table 1. In step 504, the set of peptide structures may include, for example, at least two peptide structures from a selected group of peptide structures identified in Table 1 below. The selected group of peptide structures may be, for example, a portion of the peptide structure identified in Table 1. The selected group of peptide structures may be, for example, those peptide structures identified in Table 2 below or those peptide structures identified in Table 3 below. For example, when the treatment being considered includes pembrolizumab, the selected group of peptide structures includes the peptide structures listed in Table 2. When the treatment being considered includes a combination of nivolumab and ipilimumab, the selected group of peptide structures includes the peptide structures listed in Table 3. In step 504, the set of peptide structures may include at least one glycopeptide structure defined by a peptide sequence and a glycan structure linked to a linking site of the peptide sequence, as identified in Table 1.

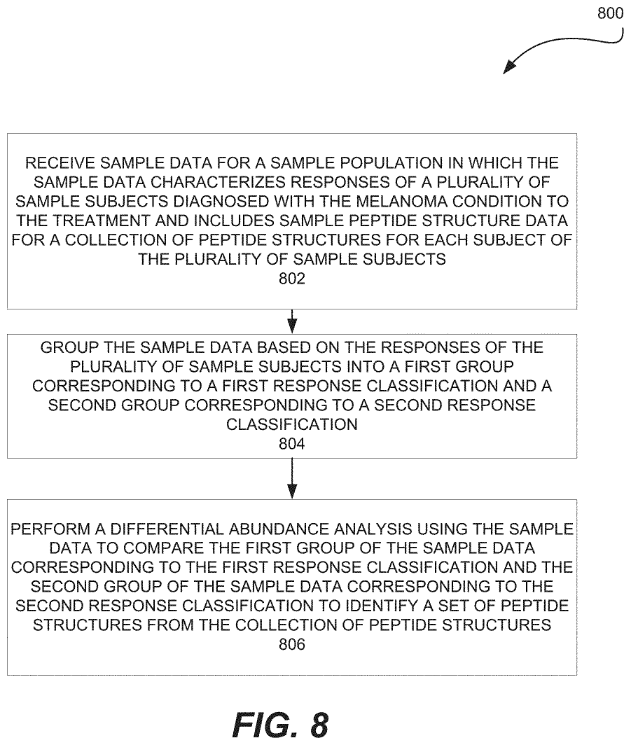

In one or more embodiments, the set of peptide structures may have been identified using sample data for a sample population (e.g., subjects diagnosed with melanoma in which at least a portion of the subjects have been treated using the treatment being considered in process 500) and a statistical algorithm that identifies a relative significance for each peptide structure of a collection of peptide structures corresponding to the sample data. The statistical algorithm may include, for example, a Wilcoxon rank-sum test. In one or more embodiments, the identification of the set of peptide structures is performed using process 800 described below in FIG. 8.

Step 504 may be performed by, for example, computing a proportion of the set of peptide structures having a certain type of abundance (e.g., relative abundance for glycopeptide structures and absolute abundance for aglycosylated peptide structures) greater than a reference abundance as the treatment score. In one or more embodiments, the reference abundance for a given peptide structure may be, for example, a median abundance of a plurality of abundances for that peptide structure across a sample population (e.g., as identified during training). The relative abundance for a given peptide structure is the abundance of that peptide structure relative to the corresponding aglycosylated peptide structure (e.g., the peptide structure having the same peptide sequence but without a glycan structure being bound to the peptide sequence).

Step 506 includes generating a treatment output that indicates a predicted response to the treatment for the subject using the treatment score. The treatment output may be one example of an implementation for treatment output 334 in FIG. 3. In one or more embodiments, step 506 may be performed by generating the predicted response to the treatment based on whether the treatment score is above a selected threshold. The selected threshold may be, for example, 0.5. For example, step 506 may include identifying a first predicted response classification for the subject when the treatment score is above 0.5 or identifying a second predicted response classification for the subject when the treatment score is not above 0. The first predicted response classification may be “sustained control” and the second predicted response classification may be “early disruption.” Sustained control may indicate that an absence of disruption events is predicted during a sustained period of time (e.g., 6 months) after treatment administration. Early disruption may indicate that a presence of at least one disruption event is predicted during an initial period of time (e.g., 12 months) after treatment.

The treatment outcome may include, for example, a recommendation to modify a treatment plan for the subject. For example, in some cases, the treatment output may indicate that early disruption is predicted for the subject. Accordingly, it may be desirable to modify the treatment plan. For example, the recommendation for modifying the treatment plan may include at least one of selecting a different treatment for the subject, alter (e.g., increase/decrease) a dosage for the treatment, or combining the treatment with at least one other treatment.

In one or more embodiments, the treatment output includes at least one of a design for the treatment or a therapeutic dosage for the treatment. For example, in some cases when the treatment score indicates that the subject will respond well (e.g., sustained control) to the treatment, the treatment outcome may identify the therapeutic dosage for the treatment. In this manner, a medical professional that receives the treatment output at a remote system (e.g., phone, tablet, laptop, etc.) may be able to more quickly administer the treatment to the subject.

In one or more embodiments, process 500 may optionally include step 508. Step 508 may include administering a therapeutic dosage of the treatment based on the treatment output to the subject. For example, the treatment may be administered (e.g., via intravenous or oral administration) based on the predicted response being a predicted response classification that indicates the treatment will be successful. For example, a predicted response classification of “sustained control” may indicate that the subject is predicted to respond well to treatment.

TABLE 1
Peptide Structures associated with Melanoma Treatments

| | | | | Mono- | Linking | Linking | |
|---|---|---|---|---|---|---|---|
| | Peptide | (Protein) | (Peptide) | isotopic | Site Pos. | Site Pos. | Glycan |
| PS-ID | Structure (PS) | SEQ ID | SEQ ID | mass | in Protein | in Peptide | Structure |
| NO. | NAME | NO. | NO. | (Da) | Sequence | Sequence | GL NO. |
| PS-1 | IGG1_297_5400 | 1 | 45 | 2811.09 | 180 | 5 | 5400 |
| PS-2 | IGG2_297_5411 | 2 | 46 | 3216.25 | 176 | 5 | 5411 |
| PS-3 | IGG1_297_5510 | 1 | 45 | 3160.22 | 180 | 5 | 5510 |
| PS-4 | IGG2_297_5410 | 2 | 46 | 2925.15 | 176 | 5 | 5410 |
| PS-5 | IGG1_297_5410 | 1 | 45 | 2957.14 | 180 | 5 | 5410 |
| PS-6 | IGG2_297_4411 | 2 | 46 | 3054.20 | 176 | 5 | 4411 |
| PS-7 | THBG_36_5402 | 3 | 44 | 3880.57 | 36 | 10 | 5402 |
| PS-8 | IGG2_297_5510 | 2 | 46 | 3128.23 | 176 | 5 | 5510 |
| PS-9 | AGP1_33_6503 | 4 | 21 | 5436.40 | 33 | 15 | 6503 |
| PS-10 | CO8B_243_6610 | 5 | 22 | 4231.67 | 243 | 11 | 6610 |
| PS-11 | IGA12_144_5502 | 6, 20 | 23 | 5370.44 | 144 | 18 | 5502 |
| PS-12 | KLKB1_494_5410 | 7 | 24 | 4014.82 | 494 | 6 | 5410 |
| PS-13 | IGG1_297_4400 | 1 | 45 | 2649.03 | 180 | 5 | 4400 |
| PS-14 | AACT_271_7602 | 8 | 25 | 4686.91 | 271 | 4 | 7602 |
| PS-15 | CO8B_553_5410 | 5 | 26 | 3454.29 | 553 | 6 | 5410 |
| PS-16 | FETUA_156_5402.5421 | 9 | 27 | 3975.61 | 156 | 12 | 5402 |
| PS-17 | IGA12_144_5501 | 6, 20 | 23 | 5079.35 | 144 | 18 | 5501 |
| PS-18 | IGG2_297_4500 | 2 | 46 | 2820.12 | 176 | 5 | 4500 |
| PS-19 | AGP1_33_6502 | 4 | 21 | 5145.31 | 33 | 15 | 6502 |
| PS-20 | CLUS_374_6520.6501 | 10 | 28 | 3961.64 | 374 | 3 | 6501 |
| PS-21 | A2MG_869_5200 | 11 | 29 | 4629.04 | 869 | 6 | 5200 |
| PS-22 | CFAH_882_5420.5401 | 12 | 30 | 3933.66 | 882 | 15 | 5401 |
| PS-23 | CFAH_911_5420.5401 | 12 | 31 | 3474.32 | 911 | 5 | 5401 |
| PS-24 | HEMO_453_5420.5401 | 13 | 32 | 3648.55 | 453 | 7 | 5401 |
| PS-25 | IGG34_297_4410 | 14, 19 | 33 | 2779.10 | 227 | 5 | 4410 |
| | | | | | (IGG3)/177 | | |
| | | | | | (IGG4) | | |
| PS-26 | KLKB1_127_5410 | 7 | 34 | 4014.82 | 127 | 5 | 5410 |
| PS-27 | TRFE_432_5401 | 15 | 35 | 3389.42 | 432 | 12 | 5401 |
| PS-28 | QUANTPEP.IGG4_ | 19 | 36 | 1900.92 | N/A | N/A | N/A |
| | TTPPVLDSDGSFFLYSR | | | | | | |
| PS-29 | NEWQUANTPEP-IGG3_ | 14 | 37 | 2413.15 | N/A | N/A | N/A |
| | TPEVTCVVVDVSHEDPEVQFK | | | | | | |
| PS-30 | A2MG_869_6200 | 11 | 29 | 4791.10 | 869 | 6 | 6200 |
| PS-31 | HPT_184_5511 | 16 | 38 | 4941.20 | 184 | 6 | 5511 |
| PS-32 | VTNC_169_5401 | 17 | 39 | 2824.14 | 169 | 1 | 5401 |
| PS-33 | AACT_271_7603 | 8 | 25 | 4978.01 | 271 | 4 | 7603 |
| PS-34 | HPT_207_10803 | 16 | 40 | 5576.18 | 207 & 211 | 5 & 9 | 5401 & |
| | | | | | | | 5402 |
| PS-35 | HPT_241_5401.5420 | 16 | 41 | 3707.68 | 241 | 6 | 5401 |
| PS-36 | IGG34_297_4411 | 14, 19 | 33 | 3070.19 | 227 | 5 | 4411 |
| | | | | | (IGG3)/177 | | |
| | | | | | (IGG4) | | |
| PS-37 | ITIH4_517_5420.5401 | 18 | 42 | 4722.02 | 517 | 5 | 5401 |
| PS-38 | AACT_127_5401 | 8 | 43 | 4125.73 | 127 | 3 | 5401 |

TABLE 2
Peptide Structures associated with a First Treatment (e.g., Pembrolizumab Tx)

| | | | | Mono- | Linking | Linking | |
|---|---|---|---|---|---|---|---|
| | Peptide | (Protein) | (Peptide) | isotopic | Site Pos. | Site Pos. | Glycan |
| PS-ID | Structure (PS) | SEQ ID | SEQ ID | mass | in Protein | in Peptide | Structure |
| NO. | NAME | NO. | NO. | (Da) | Sequence | Sequence | GL NO. |
| PS-1 | IGG1_297_5400 | 1 | 45 | 2811.09 | 180 | 5 | 5400 |
| PS-2 | IGG2_297_5411 | 2 | 46 | 3216.25 | 176 | 5 | 5411 |
| PS-3 | IGG1_297_5510 | 1 | 45 | 3160.22 | 180 | 5 | 5510 |
| PS-4 | IGG2_297_5410 | 2 | 46 | 2925.15 | 176 | 5 | 5410 |
| PS-5 | IGG1_297_5410 | 1 | 45 | 2957.14 | 180 | 5 | 5410 |
| PS-6 | IGG2_297_4411 | 2 | 46 | 3054.20 | 176 | 5 | 4411 |
| PS-7 | THBG_36_5402 | 3 | 44 | 3880.57 | 36 | 10 | 5402 |
| PS-8 | IGG2_297_5510 | 2 | 46 | 3128.23 | 176 | 5 | 5510 |
| PS-9 | AGP1_33_6503 | 4 | 21 | 5436.40 | 33 | 15 | 6503 |
| PS-10 | CO8B_243_6610 | 5 | 22 | 4231.67 | 243 | 11 | 6610 |
| PS-11 | IGA12_144_5502 | 6, 20 | 23 | 5370.44 | 144 | 18 | 5502 |
| PS-12 | KLKB1_494_5410 | 7 | 24 | 4014.82 | 494 | 6 | 5410 |
| PS-13 | IGG1_297_4400 | 1 | 45 | 2649.03 | 180 | 5 | 4400 |
| PS-14 | AACT_271_7602 | 8 | 25 | 4686.91 | 271 | 4 | 7602 |
| PS-15 | CO8B_553_5410 | 5 | 26 | 3454.29 | 553 | 6 | 5410 |
| PS-16 | FETUA_156_ | 9 | 27 | 3975.61 | 156 | 12 | 5402 |
| | 5402.5421 | | | | | | |
| PS-17 | IGA12_144_5501 | 6, 20 | 23 | 5079.35 | 144 | 18 | 5501 |
| PS-18 | IGG2_297_4500 | 2 | 46 | 2820.12 | 176 | 5 | 4500 |
| PS-19 | AGP1_33_6502 | 4 | 21 | 5145.31 | 33 | 15 | 6502 |
| PS-20 | CLUS_374_ | 10 | 28 | 3961.64 | 374 | 3 | 6501 |
| | 6520.6501 | | | | | | |

TABLE 3
Peptide Structures associated with a Second treatment
(e.g., Ipilimumab/Nivolumab Tx)

| | | | | Mono- | Linking | Linking | |
|---|---|---|---|---|---|---|---|
| | Peptide | (Protein) | (Peptide) | isotopic | Site Pos. | Site Pos. | Glycan |
| PS-ID | Structure | SEQ ID | SEQ ID | mass | in Protein | in Peptide | Structure |
| NO. | (PS) NAME | NO. | NO. | (Da) | Sequence | Sequence | GL NO. |
| PS-21 | A2MG_869_5200 | 11 | 29 | 4629.04 | 869 | 6 | 5200 |
| PS-9 | AGP1_33_6503 | 4 | 21 | 5436.40 | 33 | 15 | 6503 |
| PS-22 | CFAH_882_5420.5401 | 12 | 30 | 3933.66 | 882 | 15 | 5401 |
| PS-23 | CFAH_911_5420.5401 | 12 | 31 | 3474.32 | 911 | 5 | 5401 |
| PS-24 | HEMO_453_5420.5401 | 13 | 32 | 3648.55 | 453 | 7 | 5401 |
| PS-25 | IGG34_297_4410 | 14, 19 | 33 | 2779.10 | 227 | 5 | 4410 |
| | | | | | (IGG3)/177 | | |
| | | | | | (IGG4) | | |
| PS-26 | KLKB1_127_5410 | 7 | 34 | 4014.82 | 127 | 5 | 5410 |
| PS-27 | TRFE_432_5401 | 15 | 35 | 3389.42 | 432 | 12 | 5401 |
| PS-28 | QUANTPEP.IGG4_ | 19 | 36 | 1900.92 | N/A | N/A | N/A |
| | TTPPVLDSDGSFF | | | | | | |
| | LYSR | | | | | | |
| PS-29 | NEWQUANTPEP- | 14 | 37 | 2413.15 | N/A | N/A | N/A |
| | IGG3_TPEVTCVV | | | | | | |
| | VDVSHEDPEVQFK | | | | | | |
| PS-30 | A2MG_869_6200 | 11 | 29 | 4791.10 | 869 | 6 | 6200 |
| PS-31 | HPT_184_5511 | 16 | 38 | 4941.20 | 184 | 6 | 5511 |
| PS-32 | VTNC_169_5401 | 17 | 39 | 2824.14 | 169 | 1 | 5401 |
| PS-33 | AACT_271_7603 | 8 | 25 | 4978.01 | 271 | 4 | 7603 |
| PS-34 | HPT_207_10803 | 16 | 40 | 5576.18 | 207 & 211 | 5 & 9 | 5401 & |
| | | | | | | | 5402 |
| PS-35 | HPT_241_5401.5420 | 16 | 41 | 3707.68 | 241 | 6 | 5401 |
| PS-36 | IGG34_297_4411 | 14, 19 | 33 | 3070.19 | 227 | 5 | 4411 |
| | | | | | (IGG3)/177 | | |
| | | | | | (IGG4) | | |
| PS-37 | ITIH4_517_5420.5401 | 18 | 42 | 4722.02 | 517 | 5 | 5401 |
| PS-12 | KLKB1_494_5410 | 7 | 24 | 4014.82 | 494 | 6 | 5410 |
| PS-38 | AACT_127_5401 | 8 | 43 | 4125.73 | 127 | 3 | 5401 |

II.B.2. Selecting Between Multiple Treatments

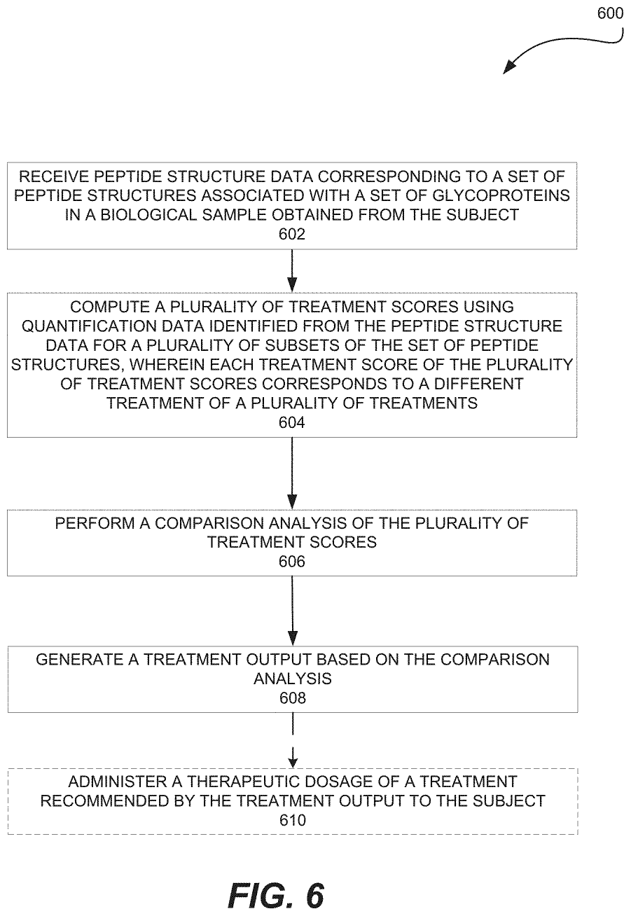

FIG. 6 is a flowchart of a process for treatment management of a subject diagnosed with a melanoma condition in accordance with various embodiments. Process 600 may be implemented using, for example, at least a portion of workflow 100 as described in FIGS. 1, 2A, and 2B and/or analysis system 300 as described in FIG. 3. In some embodiments, process 600 may be one example that includes and expands upon process 500 in FIG. 5.

Step 602 may include receiving peptide structure data corresponding to a set of peptide structures associated with a set of glycoproteins in a biological sample obtained from the subject. Step 602 may be performed in a manner similar to step 502 as described above with respect to FIG. 5.

Step 604 may include computing a plurality of treatment scores using quantification data identified from the peptide structure data for a plurality of subsets of the set of peptide structures, wherein each treatment score of the plurality of treatment scores corresponds to a different treatment of a plurality of treatments. Each subset of the plurality of subsets may include at least one peptide structure identified from a plurality of peptide structures listed in Table 1. Computing a treatment score of the plurality of treatment scores may be performed in a manner similar to step 504 as described above with respect to FIG. 5. Each treatment score may be computed using, for example, a proportion of a subset of the plurality of subsets of the set of peptide structures having a selected abundance (e.g., relative abundance for glycopeptide structures and absolute abundance for aglycosylated peptide structures) greater than a reference abundance for that peptide structure as a treatment score of the plurality of treatment scores.

In one or more embodiments, the plurality of subsets includes a first subset and a second subset. For example, step 604 may include computing a first treatment score for a first treatment of using a first portion of the quantification data identified from the peptide structure data for a first subset of the plurality of subsets of the set of peptide structures. Step 604 may further include computing a second treatment score for the second treatment using a second portion of the quantification data identified from the peptide structure data for a second subset of the plurality of subsets of the set of peptide structures. The first subset may include one or more peptide structures from those listed in Table 2. The second subset may include one or more peptide structures from those listed in Table 3.

In one or more embodiments, a subset of the plurality of subsets may have been previously identified using sample data for a sample population (e.g., subjects diagnosed with melanoma, in which at least a portion of the sample population has been treated with the plurality of treatments) and a statistical algorithm that identifies a relative significance for each peptide structure of a collection of peptide structures corresponding to the sample data with respect to a response to a selected treatment of the plurality of treatments. For example, identifying the subset may include performing a differential abundance analysis using the sample data to compare a first portion of the sample data corresponding to a first response classification (e.g., a positive response classification such as, for example, sustained control) for the selected treatment and a second portion of the sample data corresponding to a second response classification (e.g., a negative response classification such as, for example, early disruption) for the selected treatment to identify a selected N most differentiating peptide structures (e.g., the 20 most differentiating peptide structures) between the first response classification and the second response classification. The statistical algorithm may include, for example, a Wilcoxon rank-sum test.

Step 606 may include performing a comparison analysis of the plurality of treatment scores. Step 606 may be performed by, for example, determining which of the plurality of treatment scores is a highest-scoring treatment score. In some embodiments, step 606 may include determining that a treatment of the plurality of treatments has a treatment score below a selected threshold and excluding that treatment from the comparison analysis. The selected threshold may be, for example, 0.5.

Step 608 may include generating a treatment output based on the comparison analysis. The treatment output includes a recommended treatment plan for treating the subject. For example, step 608 may include identifying the treatment of the plurality of treatments having a highest treatment score as a recommended treatment for treating the subject.

In one or more embodiments, step 608 may include identifying a predicted response classification for the subject for each treatment of the plurality of treatments using a corresponding treatment score of the plurality of treatment scores. The predicted response classification may be, for example, a positive response classification, a negative response classification, or another type of response classification. In one or more embodiments, the predicted response classification for a particular treatment may be, for example, sustained control when the corresponding treatment score is above a selected threshold and may be, for example, early disruption when the corresponding treatment score is not above the selected threshold. The selected threshold may be, for example, 0.5.

In one or more embodiments, step 608 includes identifying a treatment of the plurality of treatments having a highest treatment score as a highest-scored treatment; determining that the highest treatment score is not above a selected threshold (e.g., 0.5); and generating the treatment output such that the recommended treatment plan includes a recommendation to modify an existing treatment plan for the subject. The recommendation for modifying the treatment plan may include at least one of selecting a different treatment for the subject, altering a dosage for a treatment that is part of the existing treatment plan, or combining the treatment with at least one other treatment.

In one or more embodiments, when the treatment output includes a recommended treatment, process 600 may optionally include step 610. Step 610 may include administering a therapeutic dosage of a treatment recommended by the treatment output to the subject.

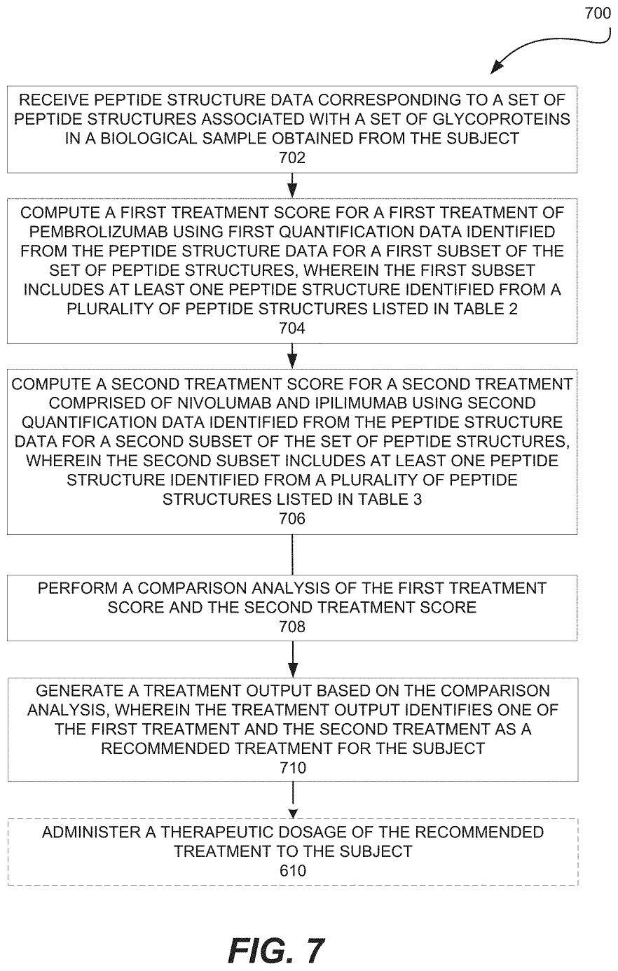

FIG. 7 is a flowchart of a process for treatment management of a subject diagnosed with a melanoma condition in accordance with various embodiments. Process 700 may be implemented using, for example, at least a portion of workflow 100 as described in FIGS. 1, 2A, and 2B and/or analysis system 300 as described in FIG. 3. In some embodiments, process 700 may be one example that includes and expands upon process 500 in FIG. 5. Further, process 700 may be one example of an implementation of process 600 in FIG. 6.

Step 702 may include receiving peptide structure data corresponding to a set of peptide structures associated with a set of glycoproteins in a biological sample obtained from the subject. Step 702 may be performed in a manner similar to step 502 as described above with respect to FIG. 5.

Step 704 may include computing a first treatment score for a first treatment of pembrolizumab using first quantification data identified from the peptide structure data for a first subset of the set of peptide structures, wherein the first subset includes at least one peptide structure identified from a plurality of peptide structures listed in Table 2. The treatment score may be computed using, for example, a proportion of a subset of the plurality of subsets of the set of peptide structures having a selected abundance (e.g., relative abundance for glycopeptide structures and absolute abundance for aglycosylated peptide structures) greater than a reference abundance for that peptide structure as a treatment score of the plurality of treatment scores. In one or more embodiments, the first subset includes all of or a majority of (e.g., more than 15) the peptide structures listed in Table 2.

Step 706 may include computing a second treatment score for a second treatment comprised of nivolumab and ipilimumab using second quantification data identified from the peptide structure data for a second subset of the set of peptide structures, wherein the second subset includes at least one peptide structure identified from a plurality of peptide structures listed in Table 3. In one or more embodiments, the first subset includes all of or a majority of (e.g., more than 15) the peptide structures listed in Table 3.

Step 708 may include performing a comparison analysis of the first treatment score and the second treatment score. Step 708 may include, for example, determining which of the first treatment score and the second treatment score is a highest score.

Step 710 may include generating a treatment output based on the comparison analysis, wherein the treatment output identifies one of the first treatment and the second treatment as a recommended treatment for the subject. For example, step 710 may include identifying the highest-scoring treatment as a recommended treatment for treating the subject. The recommended treatment may then be administered to the subject to treat the subject's melanoma. For example, the treatment may be administered via at least one of intravenous or oral administration at a therapeutic dosage.

In one or more embodiments, process 700 may optionally include step 712. Step 712 may include administering a therapeutic dosage of the recommended treatment to the subject.

II.C. Exemplary Methodology for Identifying a Set of Peptide Structures Corresponding to a Treatment

FIG. 8 is a flowchart of a process for identifying a treatment for a subject diagnosed with a melanoma condition in accordance with one or more embodiments. Process 800 may be implemented using, for example, at least a portion of workflow 100 as described in FIGS. 1, 2A, and 2B and/or analysis system 300 as described in FIG. 3. In some embodiments, process 800 may be one example that includes and expands upon process 500 in FIG. 5.

Step 802 includes receiving sample data for a sample population in which the sample data characterizes responses of a plurality of sample subjects diagnosed with the melanoma condition to the treatment and includes sample peptide structure data for a collection of peptide structures for each subject of the plurality of sample subjects.

Step 804 includes grouping the sample data based on the responses of the plurality of sample subjects into a first group corresponding to a first response classification and a second group corresponding to a second response classification.

Step 806 includes performing a differential abundance analysis using the sample data to compare the first group of the sample data corresponding to the first response classification and the second group of the sample data corresponding to the second response classification to identify a set of peptide structures from the collection of peptide structures. The set of peptide structures may be identified as a selected N most differentiating peptide structures (e.g., the 20 most significant peptide structures for differentiation) between the first response classification and the second response classification. The first response classification may be, for example, sustained control, which indicates an absence of disruption events during a sustained period of time (e.g., 12 months) after treatment administration. The second response classification may be, for example, early disruption, which indicates a presence of at least one disruption event during an initial period of time (e.g., 6 months) after treatment.

This set of peptide structure that is identified in step 806 may then be used in future analysis (e.g., in process 500 in FIG. 5, in process 600 in FIG. 6, in process 700 in FIG. 7) to compute a treatment score for a subject using the subject's peptide structure profile that indicates the likelihood of a successful response (e.g., sustained control) of the subject to the treatment.

Step 806 may be performed using, for example, a Wilcoxon rank-sum test in one or more embodiments. Exemplary results of the differential abundance analysis performed using the Wilcoxon rank-sum test are presented below in Tables 5 and 6.

TABLE 4
Wilcoxon Analysis of Peptide Structures associated with Pembrolizumab Tx

| PS-ID | Median | Median | Differential | Wilcoxon | |
|---|---|---|---|---|---|
| NO. | SC | EF | (SC-EF) | p-value | FDR |
| PS-1 | 0.5016406 | −0.3477531 | 0.8493937 | 0.0017802 | 0.3761093 |
| PS-2 | 0.5490382 | −0.6903325 | 1.2393706 | 0.0022447 | 0.3761093 |
| PS-3 | 0.6102916 | −0.4022977 | 1.0125893 | 0.0028112 | 0.3761093 |
| PS-4 | 0.4726799 | −0.8630625 | 1.3357424 | 0.0034924 | 0.3761093 |
| PS-5 | 0.9085908 | −0.820044 | 1.7286347 | 0.0043126 | 0.3761093 |
| PS-6 | −0.0540671 | −0.3156836 | 0.2616165 | 0.0052867 | 0.3761093 |
| PS-7 | 0.2843746 | −0.31304 | 0.5974146 | 0.0052867 | 0.3761093 |
| PS-8 | 0.3041313 | −0.568187 | 0.8723183 | 0.0064434 | 0.4011026 |
| PS-9 | 0.3805894 | −0.3185274 | 0.6991168 | 0.0078028 | 0.4317552 |
| PS-10 | 0.6412248 | −0.2431649 | 0.8843898 | 0.0093974 | 0.4679899 |
| PS-11 | −0.0136785 | −0.6949529 | 0.6812744 | 0.0112501 | 0.5093223 |
| PS-12 | 0.2518882 | −0.4929206 | 0.7448088 | 0.0134001 | 0.5561048 |
| PS-13 | 0.6384324 | −0.1801925 | 0.8186249 | 0.0158719 | 0.582317 |
| PS-14 | 0.3603753 | −0.1926793 | 0.5530546 | 0.018709 | 0.582317 |
| PS-15 | 0.5414354 | −0.2145807 | 0.7560161 | 0.018709 | 0.582317 |
| PS-16 | −0.0702782 | −0.4763048 | 0.4060266 | 0.018709 | 0.582317 |
| PS-17 | 0.4330799 | −0.4610782 | 0.8941581 | 0.0219396 | 0.6069946 |
| PS-18 | 0.2377877 | −0.5018914 | 0.7396791 | 0.0219396 | 0.6069946 |
| PS-19 | 0.4095444 | −0.313772 | 0.7233164 | 0.029749 | 0.6590555 |
| PS-20 | 0.1573811 | −0.2217593 | 0.3791404 | 0.029749 | 0.6590555 |

TABLE 5
Wilcoxon Analysis of Peptide Structures associated with Ipilimumab/NivolumabTx

| PS-ID | Median | Median | Differential | Wilcoxon | |
|---|---|---|---|---|---|
| NO. | SC | EF | (SC-EF) | p-value | FDR |
| PS-21 | 0.3328389 | −0.6312886 | 0.9641274 | 0.0021645 | 0.3761093 |
| PS-9 | 0.5024846 | 0.0758823 | 0.4266023 | 0.0021645 | 0.3761093 |
| PS-22 | 1.0534081 | −0.6860991 | 1.7395073 | 0.0021645 | 0.3761093 |
| PS-23 | 0.7030683 | −0.5793093 | 1.2823776 | 0.0021645 | 0.3761093 |
| PS-24 | 0.5131039 | −0.792533 | 1.3056369 | 0.0021645 | 0.3761093 |
| PS-25 | 0.4540561 | −0.9637756 | 1.4178318 | 0.0021645 | 0.3761093 |
| PS-26 | 0.6041198 | −0.8676916 | 1.4718114 | 0.0021645 | 0.3761093 |
| PS-27 | 0.3696252 | −0.8139757 | 1.1836009 | 0.0021645 | 0.4011026 |
| PS-28 | 1.0638627 | −1.0730903 | 2.1369529 | 0.0021645 | 0.4317552 |
| PS-29 | 0.938314 | −1.056397 | 1.994711 | 0.0021645 | 0.4679899 |
| PS-30 | 0.1958926 | −0.7169942 | 0.9128868 | 0.004329 | 0.5093223 |
| PS-31 | 0.3090463 | −1.5388815 | 1.8479278 | 0.004329 | 0.5561048 |
| PS-32 | 0.9161205 | −0.7184875 | 1.634608 | 0.004329 | 0.582317 |
| PS-33 | 0.1694553 | −1.6309309 | 1.8003861 | 0.008658 | 0.582317 |
| PS-34 | 0.3946123 | −0.5476397 | 0.942252 | 0.0151515 | 0.582317 |
| PS-35 | 0.320616 | −0.4720598 | 0.7926757 | 0.0151515 | 0.582317 |
| PS-36 | 0.4591413 | −0.6433692 | 1.1025105 | 0.0151515 | 0.6069946 |
| PS-37 | 0.0750044 | −1.5985227 | 1.6735272 | 0.0151515 | 0.6069946 |
| PS-12 | 0.3832391 | −0.6207699 | 1.0040091 | 0.0151515 | 0.6590555 |
| PS-38 | 0.6264716 | 0.1222803 | 0.5041913 | 0.025974 | 0.6590555 |

### III. Peptide Structure and Product Ion Compositions, Kits and Reagents

Aspects of the disclosure include compositions comprising one or more of the peptide structures listed in Table 1. In some embodiments, a composition comprises a plurality of the peptide structures listed in Table 1. In some embodiments, a composition comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, or 38 of the peptide structures listed in Table 1. In some embodiments, a composition comprises a peptide structure having an amino acid sequence with at least 80% sequence identity, such as, for example, at least 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 21-46, listed in Table 1 and defined in Table 7 below.

Aspects of the disclosure include compositions comprising one or more precursor ions having a defined charge and/or defined mass-to-charge (m/z) ratio, as listed in Table 6. Aspects of the disclosure include compositions comprising one or more product ions having a defined mass-to-charge (m/z) ratio, which product ions are produced by converting a peptide structure described herein (e.g., a peptide structure listed in Table 1) into a gas phase ion in a mass spectrometry system. Conversion of the peptide structure into a gas phase ion can take place using any of a variety of techniques, including, but not limited to, matrix assisted laser desorption ionization (MALDI); electron ionization (EI); electrospray ionization (ESI); atmospheric pressure chemical ionization (APCI); and/or atmospheric pressure photo ionization (APPI).

Aspects of the disclosure include compositions comprising one or more product ions produced from one or more of the peptide structures described herein (e.g., a peptide structure listed in Table 1). In some embodiments, a composition comprises a set of the product ions listed in Table 1, having an m/z ratio selected from the list provided for each peptide structure in Table 1.

In some embodiments, a composition comprises at least one of peptide structures PS-1 to PS-38 identified in Table 1.

In some embodiments, a composition comprises a peptide structure or a product ion. The peptide structure or product ion comprises an amino acid sequence having at least 90% sequence identity to any one of SEQ NOs: 21-46, as identified in Table 7, corresponding to peptide structures PS-1 to PS-38 in Table 1.

In some embodiments, a composition comprises a peptide structure having a monoisotopic mass identified in Table 1 as corresponding to the peptide structure.

In some embodiments, the product ion is selected as one from a group consisting of product ions identified in Table 6, including product ions falling within an identified m/z range of the m/z ratio identified in Table 6 and characterized as having a precursor ion having an m/z ratio within an identified m/z range of the m/z ratio identified in Table 6. A first range for the product ion m/z ratio may be ±0.5. A second range for the product ion m/z ratio may be ±0.8. A third range for the product ion m/z ratio may be ±1.0. A first range for the precursor ion m/z ratio may be ±0.5; a second range for the precursor ion m/z ratio may be ±1.0; a third range for the precursor ion m/z ratio may be ±1.5. Thus, a composition may include a product ion having an m/z ratio that falls within at least one of the first range (±0.5), the second range (±0.8), or the third range (±1.0) of the product ion m/z ratio identified in Table 6, and characterized as having a precursor ion having an m/z ratio that falls within at least one of a first range (±0.5), a second range (±1.0), or a third range (±1.5) of the precursor ion m/z ratio identified in Table 6.

TABLE 6
Mass Spectrometry-Related Characteristics for the Peptide Structures associated
with Melanoma Treatments

| | | | | | 1st | 1st | 2nd | 2nd |
|---|---|---|---|---|---|---|---|---|
| PS-ID | RT | Collision | Precursor | Precursor | Product | Product | Product | Product |
| NO. | (min) | Energy | m/z | Charge | m/z | Charge | m/z | Charge |
| PS-1 | 7.8 | 22 | 938.4 | 3 | 366.1 | 1 | 1392.6 | 1 |
| PS-2 | 13.6 | 26 | 1073.1 | 3 | 366.1 | 1 | 1360.6 | 1 |
| PS-3 | 8 | 20 | 1054.7 | 3 | 366.1 | 1 | 1392.6 | 1 |
| PS-4 | 12.7 | 20 | 976.1 | 3 | 366.1 | 1 | 1360.6 | 1 |
| PS-5 | 7.8 | 24 | 987.1 | 3 | 366.1 | 1 | 1392.6 | 1 |
| PS-6 | 13.9 | 30 | 1019.4 | 3 | 204.1 | 1 | 1360.6 | 1 |
| PS-7 | 11.1 | 38 | 1295.2 | 3 | 366.1 | 1 | N/A | N/A |
| PS-8 | 12.8 | 25 | 1043.8 | 3 | 366.1 | 1 | 1360.6 | 1 |
| PS-9 | 39 | 27 | 1088.6 | 5 | 366.1 | 1 | N/A | N/A |
| PS-10 | 13.5 | 33 | 1073.4 | 4 | 366.1 | 1 | N/A | N/A |
| PS-11 | 41.5 | 26 | 1075.1 | 5 | 366.1 | 1 | 1056.2 | 3 |
| PS-12 | 30.4 | 20 | 1004.7 | 4 | 366.1 | 1 | N/A | N/A |
| PS-13 | 7.9 | 21 | 884.4 | 3 | 204.1 | 1 | 1392.6 | 1 |
| PS-14 | 30.2 | 28 | 1173.2 | 4 | 366.1 | 1 | 978.5 | 2 |
| PS-15 | 25 | 35 | 1152.4 | 3 | 366.1 | 1 | N/A | N/A |
| PS-16 | 27.3 | 24 | 995.4 | 4 | 366.1 | 1 | N/A | N/A |
| PS-17 | 40.8 | 20 | 1017.3 | 5 | 366.1 | 1 | 1584.4 | 2 |
| PS-18 | 13 | 23 | 941.1 | 3 | 204.1 | 1 | 1360.6 | 1 |
| PS-19 | 38 | 32 | 1287.7 | 4 | 366.1 | 1 | N/A | N/A |
| PS-20 | 23.3 | 33 | 991.4 | 4 | 366.1 | 1 | N/A | N/A |
| PS-21 | 34.4 | 23 | 1158.8 | 4 | 1206.9 | 3 | 366.1 | 1 |
| PS-22 | 14.8 | 25 | 984.7 | 4 | 366.1 | 1 | N/A | N/A |
| PS-23 | 12.1 | 35 | 1159.4 | 3 | 366.1 | 1 | N/A | N/A |
| PS-24 | 29.7 | 15 | 913.4 | 4 | 366.1 | 1 | 1234 | 2 |
| PS-25 | 9.9 | 15 | 927.4 | 3 | 204.1 | 1 | 1376.6 | 1 |
| PS-26 | 30.9 | 31 | 1004.7 | 4 | 366.1 | 1 | N/A | N/A |
| PS-27 | 26.2 | 28 | 1131.1 | 3 | 366.1 | 1 | 840.4 | 2 |
| PS-28 | 36.5 | 45 | 951.5 | 2 | 1178.5 | 1 | 1293.6 | 1 |
| PS-29 | 31.9 | 25 | 805.4 | 3 | 994.5 | 2 | 1044 | 2 |
| PS-30 | 34.4 | 30 | 1199.3 | 4 | 1206.9 | 3 | 366.1 | 1 |
| PS-31 | 34 | 30 | 1236.1 | 4 | 366.1 | 1 | N/A | N/A |
| PS-32 | 23.8 | 23 | 942.4 | 3 | 366.1 | 1 | 1114.6 | 1 |
| PS-33 | 31.1 | 28 | 1246 | 4 | 366.1 | 1 | 978.5 | 2 |
| PS-34 | 13 | 27 | 1116.4 | 5 | 366.1 | 1 | N/A | N/A |
| PS-35 | 29.1 | 31 | 1237.3 | 3 | 366.1 | 1 | 999.5 | 2 |
| PS-36 | 10.5 | 25 | 1024.5 | 3 | 204.1 | 1 | 1376.6 | 1 |
| PS-37 | 32.7 | 30 | 1182 | 4 | 366.1 | 1 | N/A | N/A |
| PS-38 | 33 | 20 | 1032.9 | 4 | 366.1 | 1 | 1208.6 | 2 |

Table 7 defines the peptide sequences for SEQ ID NOS: 21-46 from Table 1. Table 7 further identifies a corresponding protein SEQ ID NO for each peptide sequence. Each peptide sequence in Table 7 is defined as an amino acid sequence.

TABLE 7
Peptide SEQ ID NOS

| SEQ | | Corresponding |
|---|---|---|
| ID | | Protein |
| NO: | Peptide Sequence | SEQ ID NO: |
| 21 | QIPLCANLVPVPITNATLDQITGK | 4 |
| 22 | EYESYSDFERNVTEK | 5 |
| 23 | LSLHRPALEDLLLGSEANLTCTLTGLR | 6,45 |
| 24 | LQAPLNYTEFQKPICLPSK | 7 |
| 25 | YTGNASALFILPDQDK | 8 |
| 26 | WNCWSNWSSCSGR | 5 |
| 27 | VCQDCPLLAPLNDTR | 9 |
| 28 | LANLTQGEDQYYLR | 10 |
| 29 | SLGNVNFTVSAEALESQELCGTEVPSVPEHGR | 11 |
| 30 | IPCSQPPQIEHGTINSSR | 12 |
| 31 | ISEENETTCYMGK | 12 |
| 32 | ALPQPQNVTSLLGCTH | 13 |
| 33 | EEQYNSTFR | 14,44 |
| 34 | GVNFNVSK | 7 |
| 35 | CGLVPVLAENYNK | 15 |
| 36 | TTPPVLDSDGSFFLYSR | 44 |
| 37 | TPEVTCVVVDVSHEDPEVQFK | 14 |
| 38 | MVSHHNLTTGATLINEQWLLTTAK | 16 |
| 39 | NGSLFAFR | 17 |
| 40 | NLFLNHSENATAK | 16 |
| 41 | VVLHPNYSQVDIGLIK | 16 |
| 42 | LPTQNITFQTESSVAEQEAEFQSPK | 18 |
| 43 | TLNQSSDELQLSMGNAMFVK | 8 |
| 44 | VTACHSSQPNATLYK | 3 |
| 45 | EEQYNSTYR | 1 |
| 46 | EEQFNSTFR | 2 |

Table 8 identifies the proteins of SEQ ID NOS: 1-20 from Table 1. Table 8 identifies a corresponding protein abbreviation and protein name for each of protein SEQ ID NOS: 1-20. Further, Table 8 identifies a corresponding Uniprot ID for each of protein SEQ ID NOS: 1-20.

TABLE 8
Protein SEQ ID NOS

| SEQ | Protein | | |
|---|---|---|---|
| ID | Abbre- | | Uniprot |
| NO. | viation | Protein Name | ID |
| 1 | IGG1 | Immunoglobulin heavy constant gamma 1 | P01857 |
| 2 | IGG2 | Immunoglobulin heavy constant gamma 2 | P01859 |
| 3 | THBG | Thyroxine-binding globulin | P05543 |
| 4 | AGP1 | Alpha-1-acid glycoprotein 1 | P02763 |
| 5 | CO8B | Complement component C8 beta chain | P07358 |
| 6 | IGAI | Immunoglobulin heavy constant alpha l | P01876 |
| 7 | KLKB1 | Plasma kallikrein | P03952 |
| 8 | AACT | Alpha-1-antichymotrypsin | P01011 |
| 9 | FETUA | Alpha-2-HS-glycoprotein | P02765 |
| 10 | CLUS | Clusterin | P10909 |
| 11 | A2MG | Alpha-2-macroglobulin | P01023 |
| 12 | CFAH | Complement factor H | P08603 |
| 13 | HEMO | Hemopexin | P02790 |
| 14 | IGG3 | Immunoglobulin heavy constant gamma 3 | P01860 |
| 15 | TREE | Serotransferrin | P02787 |
| 16 | HPT | Haptoglobin | P00738 |
| 17 | VTNC | Vitronectin | P04004 |
| 18 | ITIH4 | Inter-alpha-trypsin inhibitor heavy chain H4 | Q14624 |
| 19 | IGG4 | Immunoglobulin heavy constant gamma 4 | P01861 |
| 20 | IGA2 | Immunoglobulin heavy constant alpha 2 | P01877 |

Table 9 identifies and defines the glycan structures from Table 1. Table 9 identifies a graphical representation of the structure and a coded representation of the composition for each glycan structure included in Table 1. As used herein, the 4-digit GL NO. is a designation that represents the number of hexoses, the number of HexNAcs, the number of Fucoses, and the number of Neuraminic Acids.

Aspects of the disclosure include kits comprising one or more compositions, each comprising one or more peptide structures of the disclosure that can be used as assay standards, and instructions for use. Kits in accordance with one or more embodiments described herein may include a label indicating the intended use of the contents of the kit. The term “label” as used herein with respect to a kit includes any writing, or recorded material supplied on or with a kit, or that otherwise accompanies a kit.

The peptide structures and the transitions produced therefrom, as described herein, may be useful for treatment management of melanoma. A transition includes a precursor ion and at least one product ion grouping. As reviewed herein, the peptide structures in Table 1, as well as their corresponding precursor ion and product ion groupings (these ions having defined m/z ratios or m/z ratios that fall within the m/z ranges identified herein), can be used in mass spectrometry-based analyses to predict treatment response, select a treatment for administration, determine whether to alter a treatment plan or dosage, or a combination thereof.

Aspects of the disclosure include methods for analyzing one or more peptide structures, as described herein. In some embodiments, the methods involve processing a sample from a patient to generate a prepared sample that can be inputted into a mass spectrometry system (e.g., a reaction monitoring mass spectrometry system). In certain embodiments, processing the sample can comprise performing one or more of: a denaturation procedure, a reduction procedure, an alkylation procedure, and a digestion procedure. The denaturation and reduction procedures may be implemented in a manner similar to, for example, denaturation and reduction 202 in FIG. 2A. The alkylation procedure may be implemented in a manner similar to, for example, alkylation procedure 204 in FIG. 2A. The digestion procedure may be implemented in a manner similar to, for example, digestion procedure 206 in FIG. 2A.

In some embodiments, the methods for analyzing one or more peptide structures involve detecting a set of product ions generated by a reaction monitoring mass spectrometry system in which one or more product ions may correspond to each of the one or more peptide structures that have been inputted into the mass spectrometry system. As described herein, each peptide structure can be converted into a set of product ions having a defined m/z ratio, as provided in Table 6 or an m/z ratio within an identified m/z ratio as provided in Table 6. In some embodiments, the methods involve generating quantification (e.g., abundance) data for the one or more product ions detected using the reaction monitoring mass spectrometry system.

In some embodiments, the methods further comprise generating a diagnosis output using the quantification data and a model that has been trained using supervised or unsupervised machine-learning. In certain embodiments, the reaction monitoring mass spectrometry system may include multiple/selected reaction monitoring mass spectrometry (MRM/SRM-MS) to detect the one or more product ions and generate the quantification data.

### IV. Representative Experimental Results

### Samples:

Sample data via glycoproteomic analysis of pretreatment blood samples was compiled for a sample population comprising advanced malignant melanoma patients treated with pembrolizumab (Pembro; n=24) or nivolumab-ipilimumab (ipi/nivo; n=11). Samples were analyzed using an advanced glycoproteomics platform that combines ultra-high-performance liquid chromatography coupled to triple quadrupole mass spectrometry and a neural-network-based data processing engine. Individual glycopeptide signatures derived from 67 abundant serum proteins were analyzed and correlated with treatment, progression-free survival (PFS, and other clinical outcome metrics).

### Analysis:

Two response groups were defined based on PFS: early disruption (e.g., early failure) (EF; PFS event within 6 months) and sustained control (SC; no events for ≥12 months). Differential relative/absolute abundances for 498 serum glycopeptides and aglycosylated peptides were calculated between SC and EF patients for each treatment group to determine a set of peptide structures more abundant in each SC versus EF by treatment group. A score was developed for each treatment group based on the 20 markers within each treatment group identified as the most statistically significant ones based on one-sided Wilcoxon test comparing EF and SC. For a given patient, the score was computed as the proportion of glycopeptides/aglycosylated peptides with relative/absolute abundance exceeding their median abundance. A low score was associated with high risk for early failure.

Table 10 and Table 11 below show the median abundances identified for the set of peptide structures. These median abundances are examples of what may be used as reference abundances for these peptide structures.

TABLE 10
Median Abundances for Peptide Structures associated with Pembro

| PS-ID | | |
|---|---|---|
| NO. | Peptide Structure (PS) NAME | Median Abundance |
| PS-1 | IGG1_297_5400 | 0.1044605 |
| PS-2 | IGG2_297_5411 | 0.1214551 |
| PS-3 | IGG1_297_5510 | 0.1032259 |
| PS-4 | IGG2_297_5410 | 0.0993292 |
| PS-5 | IGG1_297_5410 | 0.0525704 |
| PS-6 | IGG2_297_4411 | −0.0737209 |
| PS-7 | THBG_36_5402 | 0.1421563 |
| PS-8 | IGG2_297_5510 | 0.1248705 |
| PS-9 | AGP1_33_6503 | 0.3600423 |
| PS-10 | CO8B_243_6610 | 0.1909322 |
| PS-11 | IGA12_144_5502 | −0.1191828 |
| PS-12 | KLKB1_494_5410 | 0.0492207 |
| PS-13 | IGG1_297_4400 | 0.1883168 |
| PS-14 | AACT_271_7602 | 0.0923715 |
| PS-15 | CO8B_553_5410 | −0.014819 |
| PS-16 | FETUA_156_5402.5421 | −0.2216863 |
| PS-17 | IGA12_144_5501 | 0.0029389 |
| PS-18 | IGG2_297_4500 | 0.0712961 |
| PS-19 | AGP1_33_6502 | 0.0921509 |
| PS-20 | CLUS_374_6520.6501 | 0.0556276 |

TABLE 11
Median Abundances for Peptide Structures
associated with Ipi/Nivo

| | | Glycan |
|---|---|---|
| PS-ID | | Structure |
| NO. | Peptide Structure (PS) NAME | GL NO. |
| PS-21 | A2MG_869_5200 | 0.0754619 |
| PS-9 | AGP1_33_6503 | 0.3600423 |
| PS-22 | CFAH_882_5420.5401 | 0.1460826 |
| PS-23 | CFAH_911_5420.5401 | 0.1281516 |
| PS-24 | HEMO_453_5420.5401 | 0.2013525 |
| PS-25 | IGG34_297_4410 | 0.2134462 |
| PS-26 | KLKB1_127_5410 | -0.0022041 |
| PS-27 | TRFE_432_5401 | -0.0482695 |
| PS-28 | QUANTPEP.IGG4_TTPPVLDSDGSFFLYSR | 0.0439244 |
| PS-29 | NEWQUANTPEP- | -0.0280153 |
| | IGG3_TPEVTCVVVDVSHEDPEVQFK | |
| PS-30 | A2MG_869_6200 | -0.0430135 |
| PS-31 | HPT_184_5511 | -0.2843536 |
| PS-32 | VTNC_169_5401 | -0.0248306 |
| PS-33 | AACT_271_7603 | 0.1237403 |
| PS-34 | HPT_207_10803 | 0.0825476 |
| PS-35 | HPT_241_5401.5420 | -0.0008547 |
| PS-36 | IGG34_297_4411 | 0.2102183 |
| PS-37 | ITIH4_517_5420.5401 | 0.0750044 |
| PS-12 | KLKB1_494_5410 | 0.0492207 |
| PS-38 | AACT_127_5401 | 0.0715 |

### Results:

When examined in all patients in the cohort (regardless of treatment), both treatment scores isolated EF from SC. Algorithmic assignment was performed by choosing the treatment with the highest treatment-specific score (e.g., if ipi/nivo score>pembro score, then assign to ipi/nivo). PFS was superior for cases where the assigned treatment matched the treatment received. Log-rank p-values comparing PFS by assigned treatment within pembro- and ipi/nivo-treated cases were 0.009 and 0.0004, respectively. Our results show that serum glycoproteomic analysis allows targeted treatment assignment not only to immune checkpoint inhibitor treatment in general, but specifically to the most likely successful agent among different drugs for melanoma. This may fundamentally improve the clinical use of immuno-therapy in subjects with melanoma.

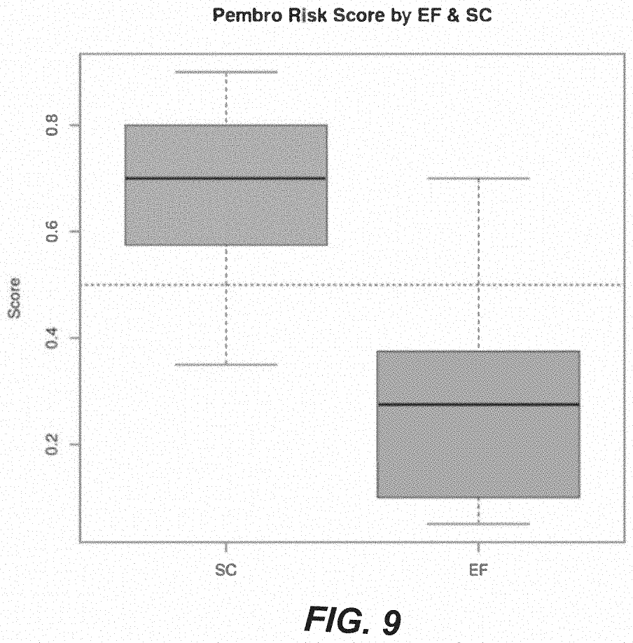

FIG. 9 is a plot showing the distribution of the treatment scores generated for those patients who were treated with pembro in accordance with one or more embodiments.

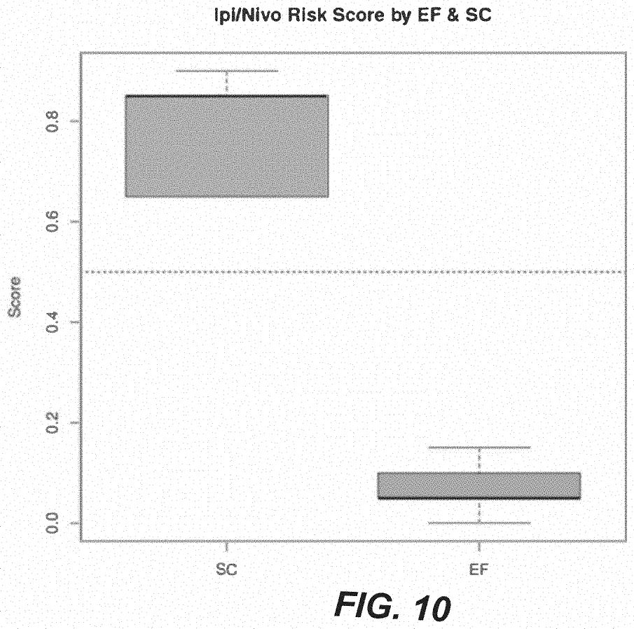

FIG. 10 is a plot showing the distribution of the treatment scores generated for those patients who were treated with ipi/nivo in accordance with one or more embodiments.

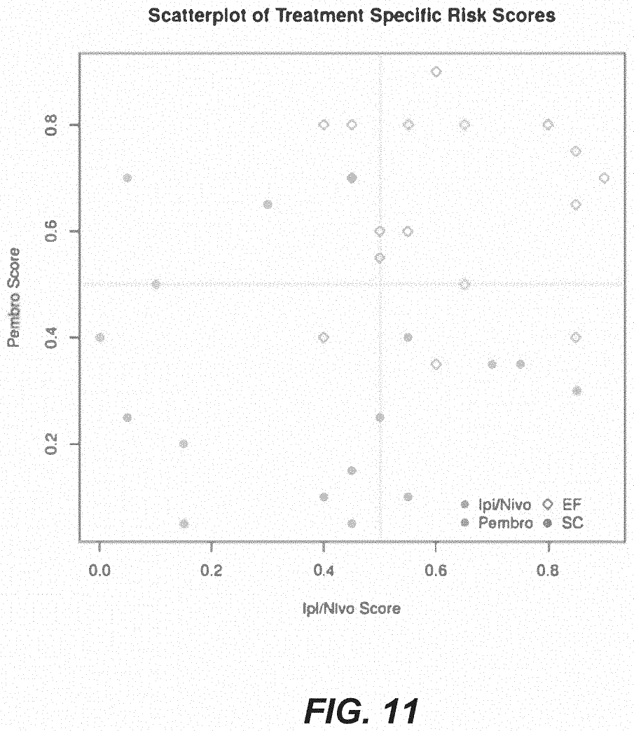

FIG. 11 is a scatterplot showing the treatment scores by treatment type in accordance with one or more embodiments.

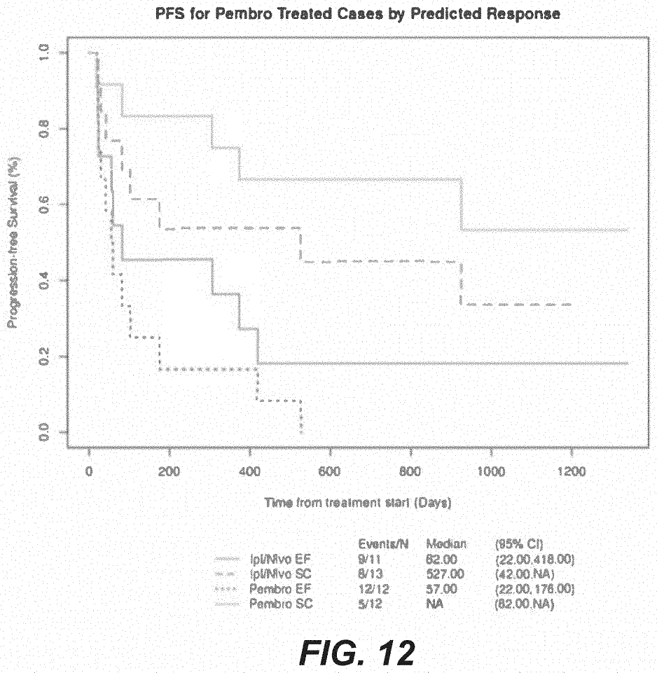

FIG. 12 is a plot showing disruption event times for patients treated with pembro by their predicted response.

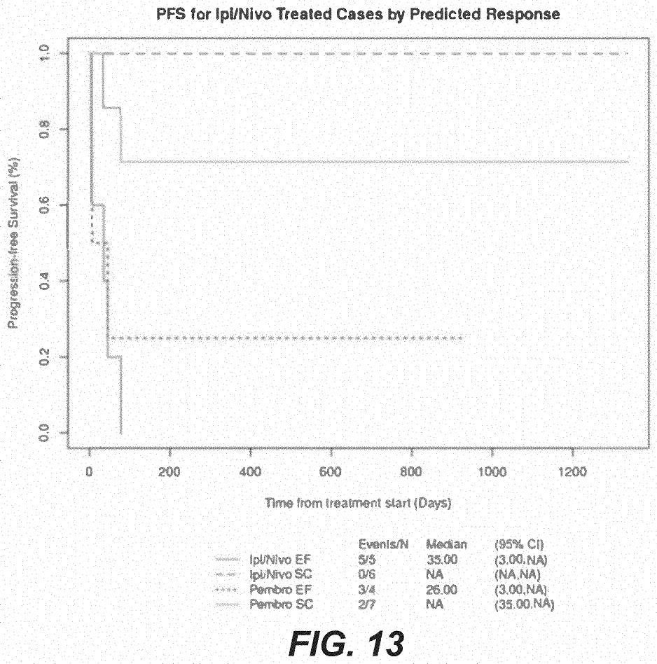

FIG. 13 is a plot showing disruption event times for patients treated with ipi/nivo by their predicted response.

### 2. Biomarkers for Determining Immuno-Oncology Response

Provided herein are methods, devices, glycopeptides, and kits for identifying glycoproteomic biomarkers and signatures for risk of having a disease or a condition, progression of the disease or condition, and response of the disease or condition to a treatment, such as treatment with immune checkpoint blockade for cancer. In some cases, the disease or condition may be cancer. In some cases, the progression of the disease or condition includes but is not limited to stage of cancer or size of tumor or a surrogate endpoint. Such information may be used to provide actionable recommendations for treatment to a healthcare provider, including but not limited to initiation of a new treatment, continuation of ongoing treatment, adding a new therapy, or changing the dosage and/or frequency of ongoing treatment.

Protein glycosylation is one of the abundant and most complex form of post-translational protein modification. Glycosylation profoundly can affect structure, conformation, and function of a polypeptide. The elucidation of the potential role of differential polypeptide glycosylation as biomarkers has so far been limited by the technical complexity of generating and interpreting this information. A novel, powerful platform has been established that combines ultra-high-performance liquid chromatography (LC) coupled to triple quadrupole mass spectrometry (MS) with a machine-learning and neural-network-based data processing engine that allows for high-throughput, highly scalable interrogation of the glycoproteome. The glycoproteomic biomarkers and signatures may be used to predict which cancer patients may respond to immune checkpoint blockade treatment, such as PD1/PDL1 checkpoint inhibitors.

Changes in glycosylation have been described in relationship to disease states such as cancer. See, e.g., Dube, D. H.; Bertozzi, C. R. Glycans in Cancer and Inflammation—Potential for Therapeutics and Diagnostics. Nature Rev. Drug Disc. 2005, 4, 477-88, the entire contents of which are herein incorporated by reference in its entirety for all purposes. However, clinically relevant, non-invasive assays for diagnosing cancer in a patient based on glycosylation changes in a sample from that patient are still needed.

Mass spectroscopy (MS) offers sensitive and precise measurement of cancer-specific biomarkers including glycopeptides. See, for example, Ruhaak, L. R., et al., Protein-Specific Differential Glycosylation of Immunoglobulins in Serum of Ovarian Cancer Patients DOI: 10.1021/acs.jproteome.5b01071; J. Proteome Res., 2016, 15, 1002-1010 (2016); also Miyamoto, S., et al., Multiple Reaction Monitoring for the Quantitation of Serum Protein Glycosylation Profiles: Application to Ovarian Cancer, DOI: 10.1021/acs.jproteome.7b00541, J. Proteome Res. 2018, 17, 222-233 (2017), the entire contents of which are herein incorporated by reference in its entirety for all purposes. However, using MS to diagnose cancer has not been demonstrated to date in a clinically relevant manner. What is needed are new biomarkers and new methods of using MS to assess a diagnosis for a disease or a condition, a risk of having a disease or a condition, progression of the disease or condition, and response of the disease or condition to a treatment.

I. Overview

Provided herein are methods for identifying one or more glycopeptide biomarkers predictive of a disease or a condition in a subject, the method comprising: (a) obtaining from a subject a first sample at a first timepoint and a second sample at a second timepoint, wherein the first sample and the second sample comprise a glycoprotein; (b) fragmenting the glycoprotein in the first sample or the second sample into one or more glycopeptides, wherein the one or more glycopeptides comprise one or more amino acid sequences selected from a group consisting of SEQ ID NO: 101-131, 159-207, and 21-46, and combinations thereof; (c) determining an amount of the one or more glycopeptides using multiple reaction monitoring mass spectrometry (MRM-MS); (d) associating the amount of the one or more glycopeptides with the first timepoint or the second timepoint, wherein the subject has a change in a disease or a condition from the first timepoint to the second timepoint; and (e) identifying as glycopeptide biomarkers the glycopeptide where the amount of the one or more glycopeptides changed from the first timepoint to the second timepoint.

Described herein are methods for identifying one or more glycopeptide biomarkers predictive of a disease or a condition in a subject, the method comprising: (a) obtaining, by a computer, data of an amount of one or more glycopeptides for a set (n) of subjects, wherein the one or more glycopeptides are generated by fragmenting a glycoprotein in a sample from a subject, the amount of one or more glycopeptides are determined using multiple reaction monitoring mass spectrometry (MRM-MS), and the data for each subject comprises data from samples taken at a plurality of timepoints; (b) selecting, by the computer, a subset of the one or more glycopeptides to include in a predictive model; (c) assessing, by the computer, the predictive model using a cross-validation with n−1 subjects to generate an outcome score for a holdout subject; (d) iterating, by the computer, step (c) for each of n subjects as the holdout subject to generate an outcome score for each subject; (e) dichotomizing, by the computer, the outcome scores for each subject at a cutoff outcome score as below or above the cutoff outcome score; (f) analyzing, by the computer, the amount of one or more glycopeptides for subjects having outcome scores above the cutoff outcome score to the amount of one or more glycopeptides for subjects having outcome scores below the cutoff outcome score for each glycopeptide in the subset of the one or more glycopeptides to determine a hazard ratio and an interaction p-value for each glycopeptide; (g) identifying, by the computer, the glycopeptide having the interaction p-value ≤0.05 as a glycopeptide biomarker for predicting the disease or the condition. In some embodiments, the cross-validation is leave-one-out cross-validation (LOOCV). In some embodiments, the cutoff outcome score was determined to optimize Harrell's C-index. In some embodiments, the interaction p-value is less than or equal to 0.01, 0.005, or 0.001 in step (g).

Provided herein are method for identifying one or more glycopeptide biomarkers predictive of a disease or a condition in a subject, the method comprising: (a) obtaining, by a computer, data of an amount of one or more glycopeptides for a set (n) of subjects, wherein the one or more glycopeptides are generated by fragmenting a glycoprotein in a sample from a subject, the amount of one or more glycopeptides are determined using multiple reaction monitoring mass spectrometry (MRM-MS), and the data for each subject comprises data from samples taken at a plurality of timepoints; (b) selecting, by the computer, a subset of the one or more glycopeptides to include in a predictive model; (c) assessing, by the computer, the predictive model using a cross-validation with n−1 subjects to generate an outcome score for a holdout subject; (d) iterating, by the computer, step (c) for each of n subjects as the holdout subject to generate an outcome score for each subject; (e) dichotomizing, by the computer, the outcome scores for each subject at a cutoff outcome score as below or above the cutoff outcome score; (f) analyzing, by the computer, the amount of one or more glycopeptides for subjects having outcome scores above the cutoff outcome score to the amount of one or more glycopeptides for subjects having outcome scores below the cutoff outcome score for each glycopeptide in the subset of the one or more glycopeptides to determine a hazard ratio and an interaction p-value for each glycopeptide; (g) identifying, by the computer, the glycopeptide having the interaction p-value ≤0.05 as a glycopeptide biomarker for predicting the disease or the condition.

Described herein are methods for assessing a status of a condition and a treatment in a subject, the method comprising: (a) fragmenting a glycoprotein in a sample from a subject into one or more glycopeptides, wherein the sample comprises one or more of glycoproteins, glycans, or glycopeptides; (b) performing mass spectroscopy (MS) on the one or more glycopeptides using multiple reaction monitoring mass spectrometry (MRM-MS) to quantify an amount of the one or more glycopeptides in the sample, wherein the one or more glycopeptides comprise one or more amino acid sequences selected from a group consisting of SEQ ID NOs:101-131, 159-207, and 21-46, and combinations thereof; (c) inputting data of the amount of the one or more glycopeptides into a trained model to generate an output probability, wherein the output probability is indicative of whether a treatment positively influences an outcome of the subject having a condition; and (d) generating a treatment recommendation based on the output probability, wherein the condition is melanoma and the treatment comprises checkpoint inhibitors. In some embodiments, the outcome comprises overall survival time. In some embodiments, the outcome comprises progression-free survival time. In some embodiments, the treatment comprises one or more of ipilimumab, nivolumab, and pembrolizumab. In some embodiments, the treatment comprises one or more of PD-1-, PD-L1-, and CTLA-4-inhibitors. In some embodiments, the treatment comprises chemotherapy. In some embodiments, the chemotherapy comprises one or more of carboplatin and pemetrexed. In some embodiments, the recommendation comprises continuing the treatment if the output probability indicates the treatment positively influences the outcome.

Provided herein are methods for assessing a status of a condition and a treatment in a subject, the method comprising: (a) fragmenting a glycoprotein in a sample from a subject into one or more glycopeptides, wherein the sample comprises one or more of glycoproteins, glycans, or glycopeptides; (b) performing mass spectroscopy (MS) on the one or more glycopeptides using multiple reaction monitoring mass spectrometry (MRM-MS) to quantify an amount of the one or more glycopeptides in the sample, wherein the one or more glycopeptides comprise one or more amino acid sequences selected from a group consisting of SEQ ID NOs: 101-131, 159-207, and 21-46, and combinations thereof; (c) inputting data of the amount of the one or more glycopeptides into a trained model to generate an output probability, wherein the output probability is indicative of whether a treatment positively influences an outcome of the subject having a condition; and (d) generating a treatment recommendation based on the output probability, wherein the condition is non-small cell lung cancer (NSCLC) and the treatment comprises checkpoint inhibitors. In some embodiments, the outcome comprises overall survival time. In some embodiments, the outcome comprises progression-free survival time. In some embodiments, the treatment comprises one or more of ipilimumab, nivolumab, and pembrolizumab. In some embodiments, the treatment comprises one or more of PD-1-, PD-L1-, and CTLA-4-inhibitors. In some embodiments, the treatment comprises chemotherapy. In some embodiments, the chemotherapy comprises one or more of carboplatin and pemetrexed. In some embodiments, the recommendation comprises continuing the treatment if the output probability indicates the treatment positively influences the outcome.

In some embodiments, provided herein are methods for identifying a classification for a sample, the method comprising: quantifying by mass spectroscopy (MS) one or more glycopeptides in a sample wherein the glycopeptides each, individually in each instance, comprises a glycopeptide consisting essentially of an amino acid sequence selected from the group consisting of SEQ ID NO: 101-131, 159-207, and 21-46, and combinations thereof; and inputting the quantification into a trained model to generate an output probability; determining if the output probability is above or below a threshold for a classification; and identifying a classification for the sample based on whether the output probability is above or below a threshold for a classification.

In some embodiments, provided herein are methods for training a machine-learning algorithm, comprising: providing a first data set of MRM transition signals indicative of a sample comprising a glycopeptide consisting of, or consisting essentially of, an amino acid sequence selected from the group consisting of SEQ ID NO: 101-131, 159-207, and 21-46; providing a second data set of MRM transition signals indicative of a control sample; and comparing the first data set with the second data set using a machine-learning algorithm.

In some embodiments, provided herein are methods for diagnosing a patient having cancer; the method comprising: obtaining a biological sample from the patient; performing mass spectroscopy of the biological sample using MRM-MS with a QQQ and/or qTOF spectrometer to detect and quantify one or more glycopeptides consisting essentially of an amino acid sequence selected from the group consisting of SEQ ID NO: 101-131, 159-207, and 21-46; or to detect and quantify one or more MRM transitions; inputting the quantification of the detected glycopeptides or the MRM transitions into a trained model to generate an output probability, determining if the output probability is above or below a threshold for a classification; identifying a diagnostic classification for the patient based on whether the output probability is above or below a threshold for a classification; and providing a recommendation for treatment. In some examples, the method includes performing mass spectroscopy of the biological sample using MRM-MS with a QQQ.

II. Biomarkers

Provided herein are glycopeptide biomarkers. These biomarkers are useful for a variety of applications, including, but not limited to, diagnosing diseases and conditions. For example, certain biomarkers set forth herein, or combinations thereof, are useful for diagnosing cancer. In some embodiments, the cancer is melanoma. In some embodiments, the cancer is non-small cell lung cancer (NSCLC). In some embodiments, the biomarkers are useful for diagnosing and screening patients having cancer, an autoimmune disease, or fibrosis. In some embodiments, the biomarkers are useful for classifying a patient so that the patient receives the appropriate medical treatment. In some embodiments, the biomarkers are useful for treating or ameliorating a disease or condition in patient by, for example, identifying a therapeutic agent with which to treat a patient. In some embodiments, the biomarkers are useful for determining a prognosis of treatment for a patient or a likelihood of success or survivability for a treatment regimen.

in some embodiments, a sample from a patient is analyzed by MS and the results are used to determine the presence, absolute amount, and/or relative amount of a glycopeptide consisting of an amino acid sequence selected from SEQ ID NO: 101-131, 159-207, and 21-46 in the sample. In some embodiments, a sample from a patient, is analyzed by MS and the results are used to determine the presence, absolute amount, and/or relative amount of a glycopeptide consisting essentially of an amino acid sequence selected from SEQ ID NO: 101-131, 159-207, and 21-46 in the sample. In some embodiments, a sample from a patient is analyzed by MS and the results are used to determine the presence, absolute amount, and/or relative amount of a glycopeptide consisting of, or consisting essentially of, an amino acid sequence selected from SEQ ID NO: 101-131, 159-207, and 21-46 in the sample. In some embodiments, a sample from a patient is analyzed by MS and the results are used to determine the presence, absolute amount, and/or relative amount of a glycopeptide consisting of, or consisting essentially of, an amino acid sequence selected from SEQ ID NO: 101-131, 159-207, and 21-46 in the sample. In some embodiments, the presence, absolute amount, and/or relative amount of a glycopeptide is determined by analyzing the MS results. In some embodiments, the MS results are analyzed using machine-learning.

Provided herein are biomarkers selected from glycans, peptides, glycopeptides, fragments thereof, and combinations thereof. In some embodiments, the glycopeptide comprise an amino acid sequence selected from SEQ ID NO: 101-131, 159-207, and 21-46. In some embodiments, the glycopeptide consists essentially of an amino acid sequence selected from SEQ ID NO: 101-131, 159-207, and 21-46.

### O-Glycosylation

In some examples, the glycopeptides set forth herein include O-glycosylated peptides. These peptides include glycopeptides in which a glycan is bonded to the peptide through an oxygen atom of an amino acid. Typically, the amino acid to which the glycan is bonded is threonine (T) or serine (S). In some examples, the amino acid to which the glycan is bonded is threonine (T). In some examples, the amino acid to which the glycan is bonded is serine (S).

In certain examples, the 0-glycosylated peptides include those peptides from the group selected from Apolipoprotein C-Ill (APOC3), Alpha-2-HS-glycoprotein (FETUA.), and combinations thereof. In certain examples, the O-glycosylated peptide, set forth herein, is an Apolipoprotein (APOC3) peptide. In certain examples, the O-glycosylated peptide, set forth herein, is an Alpha-2-HS-glycoprotein (FETUA).

### N-Glycosylation

In some examples, the glycopeptides set forth herein include N-glycosylated peptides. These peptides include glycopeptides in which a glycan is bonded to the peptide through a nitrogen atom of an amino acid. Typically, the amino acid to which the glycan is bonded is asparagine (N) or arginine (R). In some examples, the amino acid to which the glycan is bonded is asparagine (N). In some examples, the amino acid to which the glycan is bonded is arginine (R).

In certain examples, the N-glycosylated peptides include members selected from the group consisting of Alpha-1-antitrypsin (A1AT), Alpha-1B-glycoprotein (A1BG), Leucine-richAlpha-2-glycoprotein (A2GL), Alpha-2-macroglobulin (A2MG), Alpha-1-antichymotrypsin (AACT), Afamin (AFAM), Alpha-1-acid glycoprotein 1 & 2 (AGP12), Alpha-1-acid glycoprotein 1 (AGP1), Alpha-1-acid glycoprotein 2 (AGP2), Apolipoprotein A-I (APOA1), Apolipoprotein B-100 (APOB), Apolipoprotein D (APOD), Beta-2-glycoprotein-1 (APOH), Apolipoprotein M (APOM), Attractin (ATRN), Calpain-3 (CAN3), Ceruloplasmin (CERU), Complement Factor H (CFAH), Complement Factor I (CFAI), Clusterin (CLU), ComplementC3 (CO3), ComplementC4-A&B (CO4A&CO4B), ComplementcomponentE6 (CO6),

ComplementComponentC8AChain (CO8A), Coagulation factor XII (FA12),

Haptoglobin (HPT), Histidine-rich Glycoprotein (HRG), Immunoglobulin heavy constant alpha 1&2 (IgA12), Immunoglobulin heavy constant alpha 2 (IgA2).

Immunoglobulin heavy constant gamma 2 (IgG2), Immunoglobulin heavy constant mu (IgM), Inter-alpha-trypsin inhibitor heavy chain H1 (ITIH1), Plasma Kallikrein (KLKB1),

Kininogen-1 (KNG1), Serum paraoxonase/arylesterase 1 (PON1), Selenoprotein P (SEPP1), Prothrombin (THRB), Serotransferrin (TREE), Transthyretin (TTR), Protein unc-13HomologA (UN13A), Vitronectin (VTNC), Zinc-alpha-2-glycoprotein (ZA2G), growth factor-II (IGF2), Apolipoprotein C-I (APOC1), Hemopexin (HEMO), Immunoglobulin heavy constant gamma 1 (IgG1), Immunoglobulin J chain (0.10), and combinations thereof.

### Peptides and Glycopeptides

In some examples, set forth herein is a glycopeptide or peptide consisting of an amino acid sequence selected from the group consisting of SEQ ID NO: 101-131, 159-207, and 21-46, and combinations thereof.

In some examples, set forth herein is a glycopeptide or peptide consisting essentially of an amino acid sequence selected from the group consisting of SEQ ID NO: 101-131, 159-207, and 21-46, and combinations thereof.

III. Methods

Provided herein are methods of identifying the glycoproteomic biomarkers and signatures that may be used to predict which cancer patients respond to immune checkpoint blockade treatment, such as PD1/PDL1 checkpoint inhibitors, and have an improvement or a positive change in their condition.

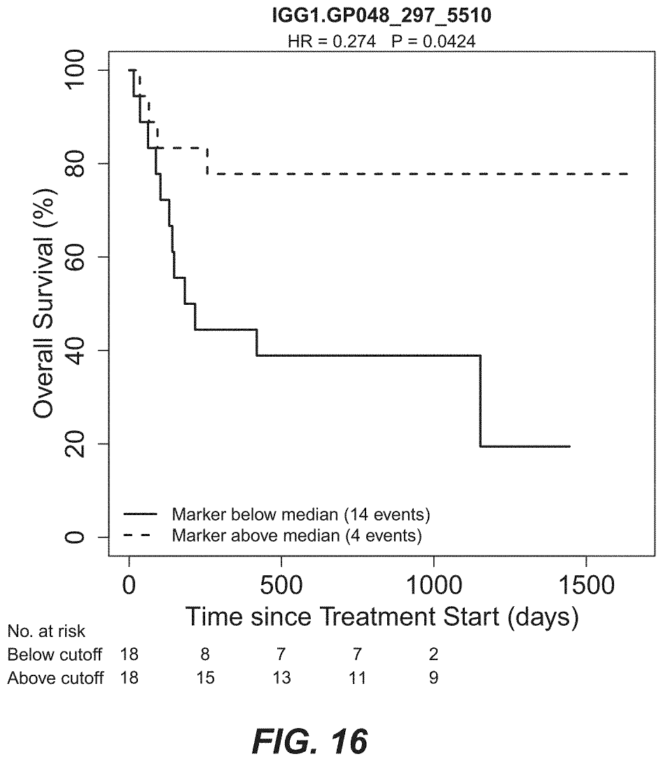

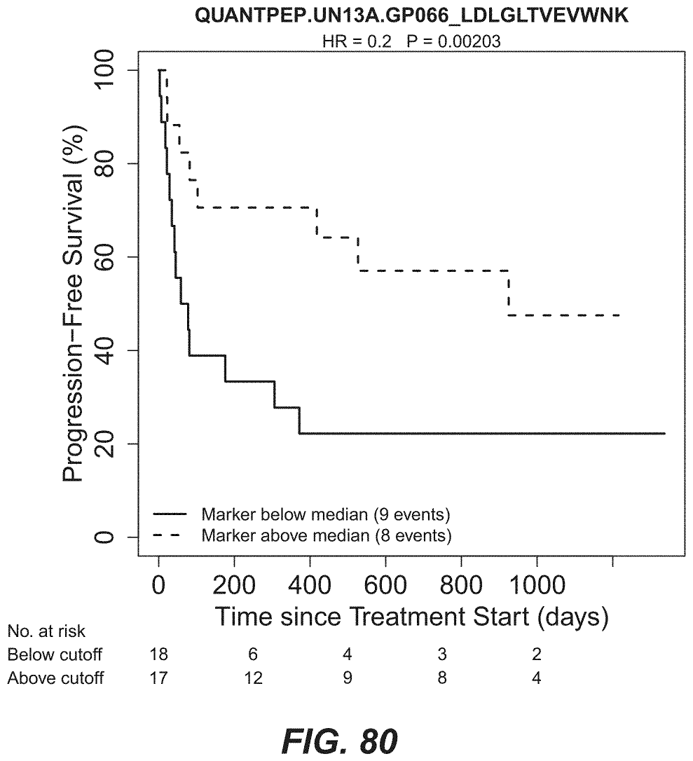

In some embodiments, individual glycopeptide expression levels are associated with various timepoints to determine which glycopeptides changed with events, such as death or metastasis, at the various timepoints. In some embodiments, individual glycopeptide expression levels are associated with time from treatment initiation to progression/metastasis (progression-free survival, PFS) or death (overall survival, OS) in the patient cohorts. In some embodiments, examples of individual glycopeptide expression levels are shown in FIGS. 16-80.

In some embodiments, multivariable models are used predict OS and PFS in cancer patients. In some embodiments, the cancer patients have NSCLC or melanoma. In some embodiments, a small subset of glycopeptides for modeling are selected, a model with n−1 patients from a total of n patients is built, a survival score on the one holdout patient is predicted, and the step are iterated over all patients as individual holdouts, to generate unbiased prediction scores for everyone (a leave-one-out cross-validation approach, LOOCV). In some embodiments, the resulting scores are dichotomized at a cutoff which optimizes Harrell's C-index. In some embodiments, Kaplan-Meier (KM) curves were plotted for each glycopeptide.

In some embodiments, hazard ratio (HR), p-value, and interaction P-value were calculated. In some embodiments, hazard ratio (HR) is calculated from a Cox Proportional Hazards model, representing the multiplicative increase in odds of death or progression-free survival time for each increase of the biomarker by 1 unit. In some embodiments, p-value is associated with the HR above. In some embodiments, P<0.01 was considered significant. In some embodiments, P≤0.05, P≤0.01, P≤0.005, or P≤0.001 was considered significant. In some embodiments, interaction P-value is associated with the biomarker x treatment interaction; significance indicates potential for use in treatment selection.

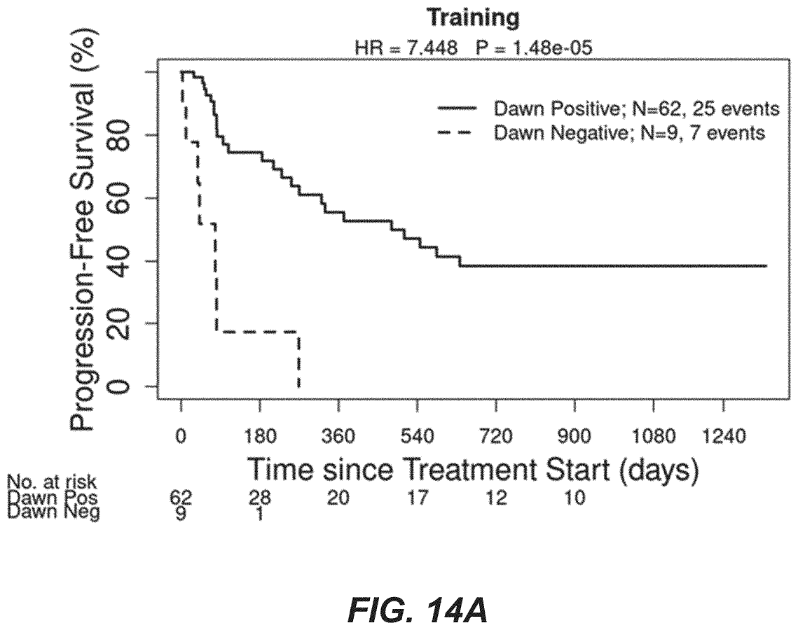

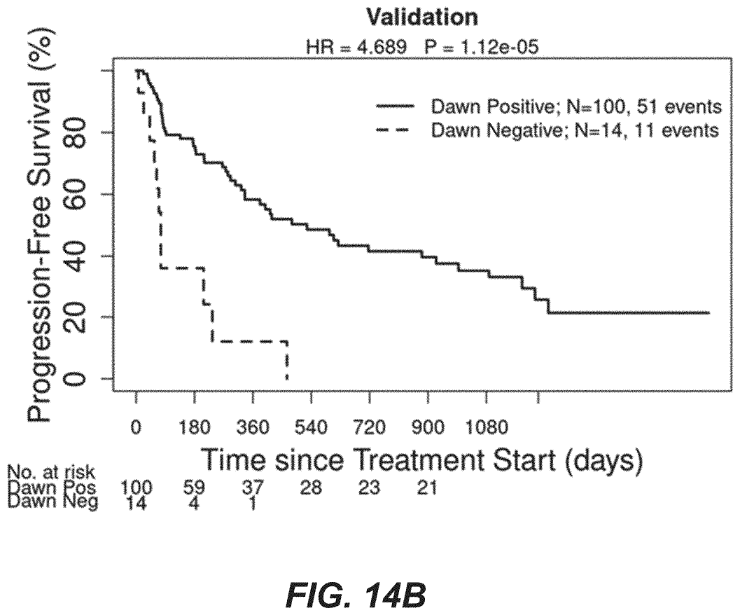

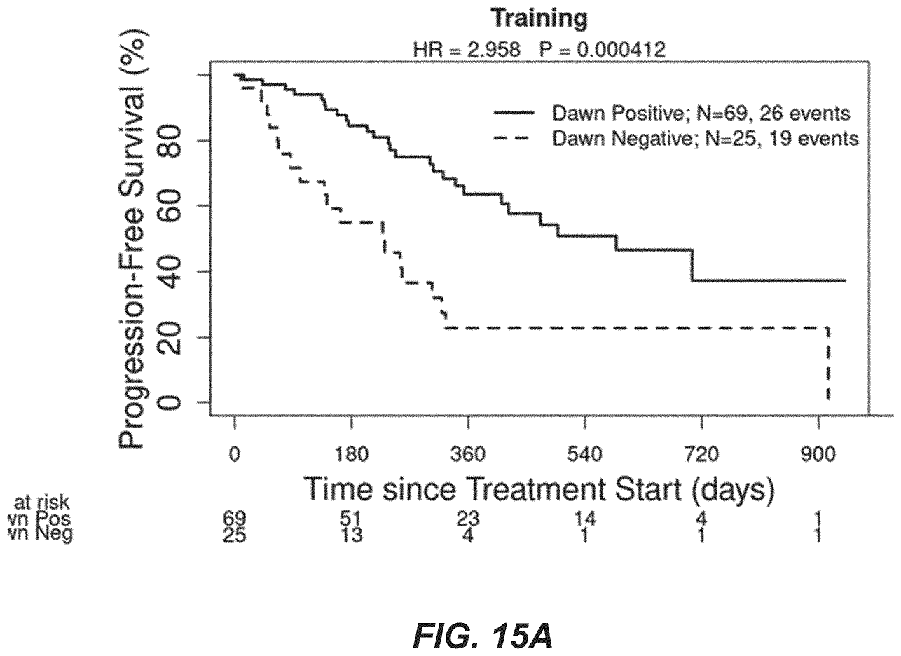

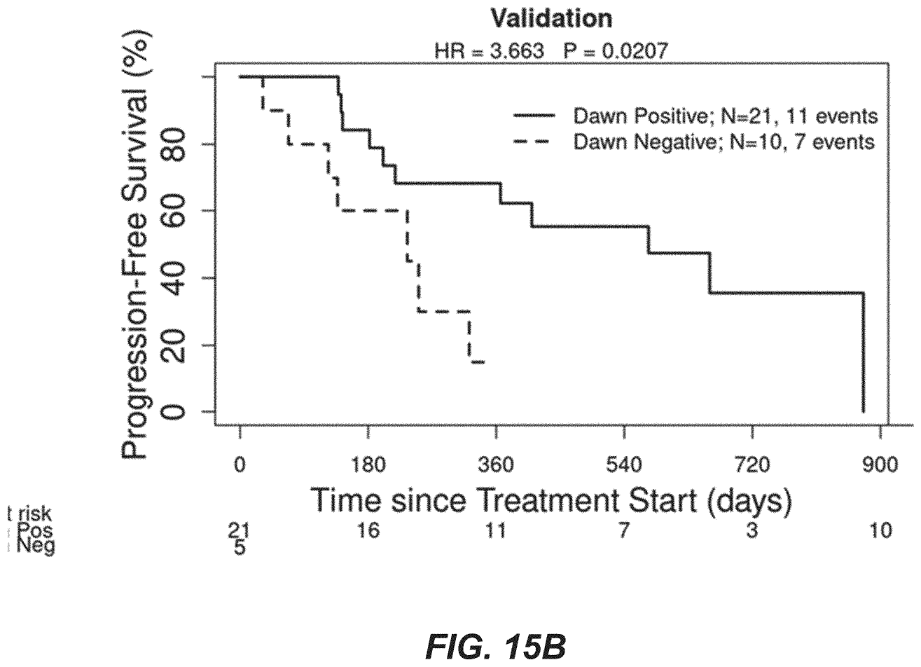

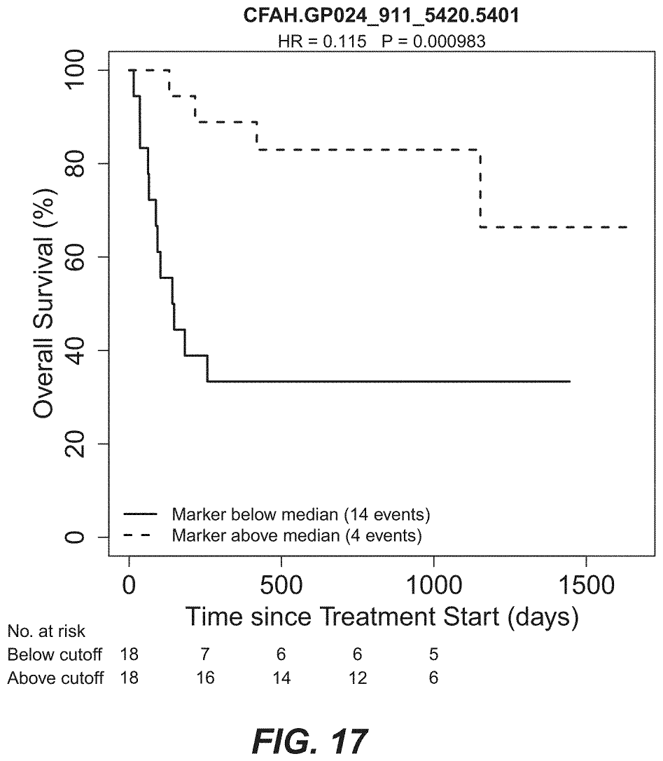

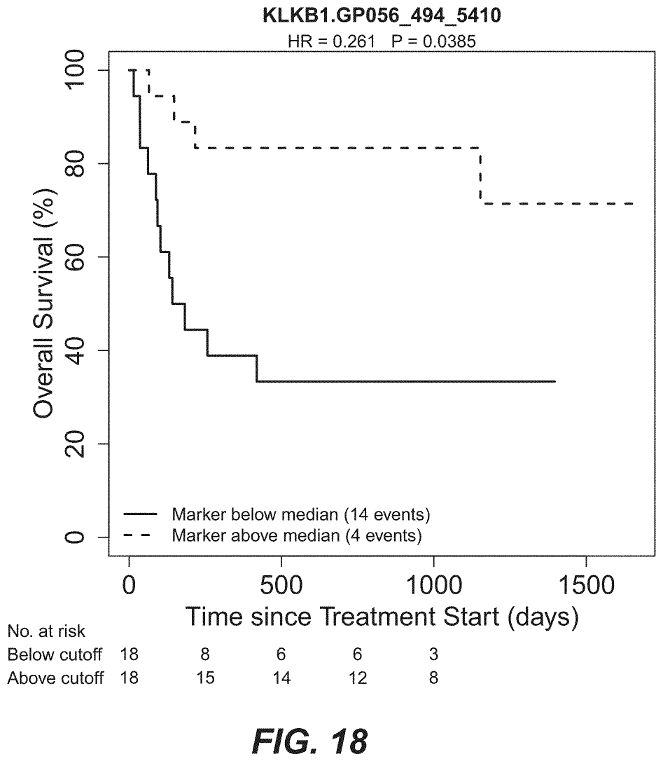

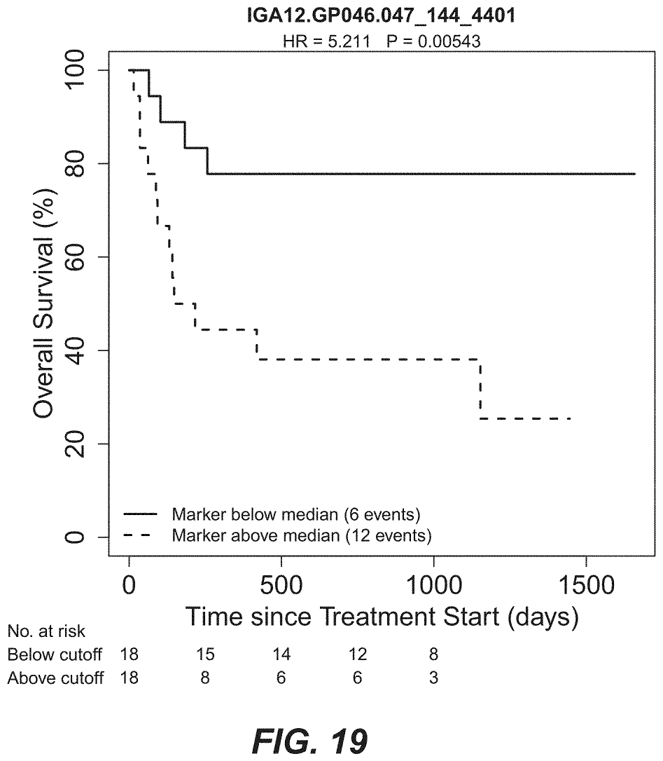

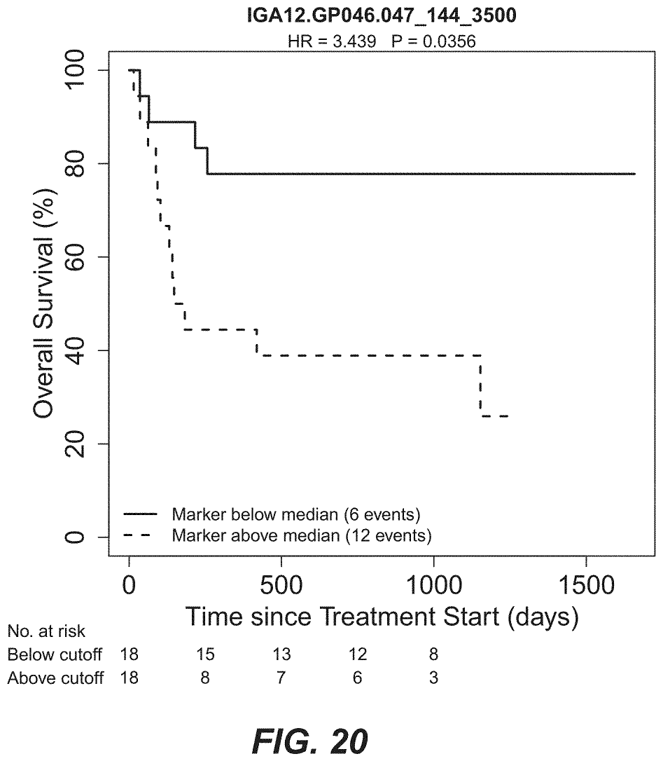

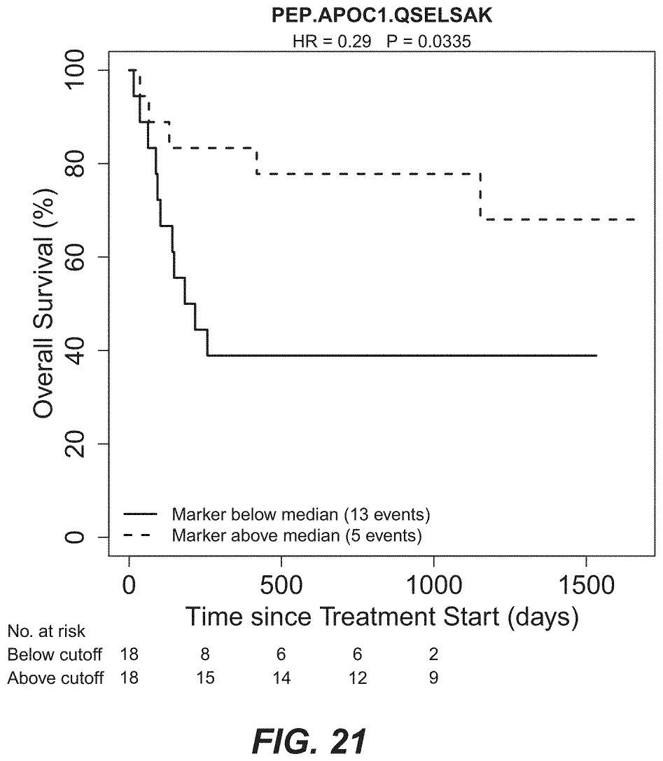

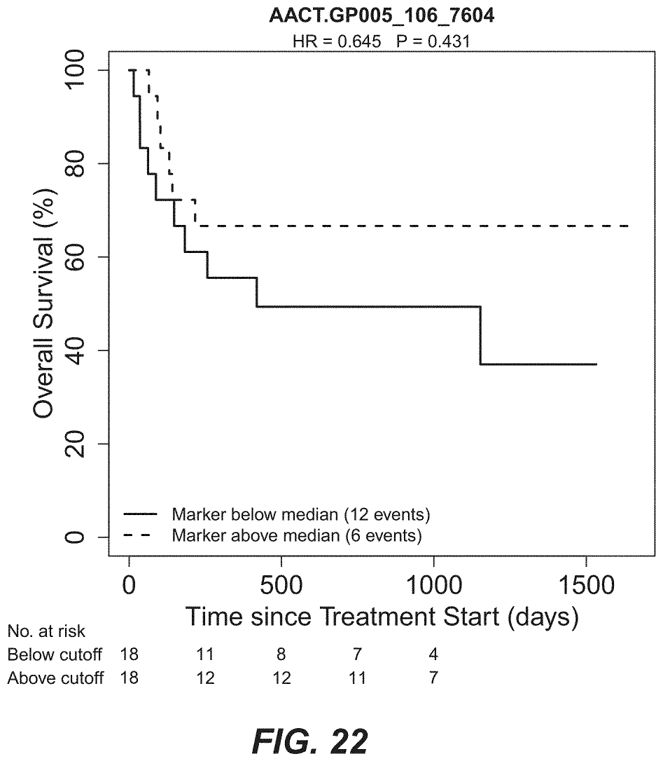

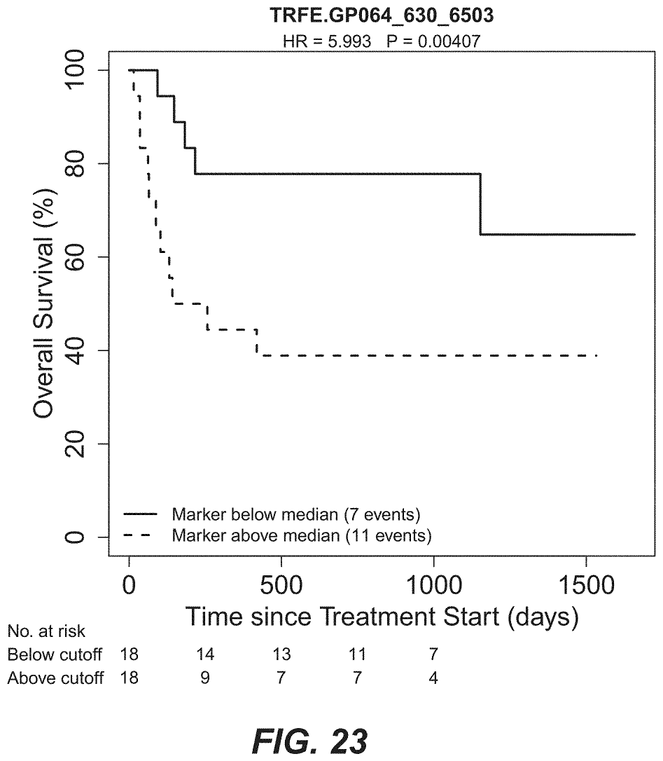

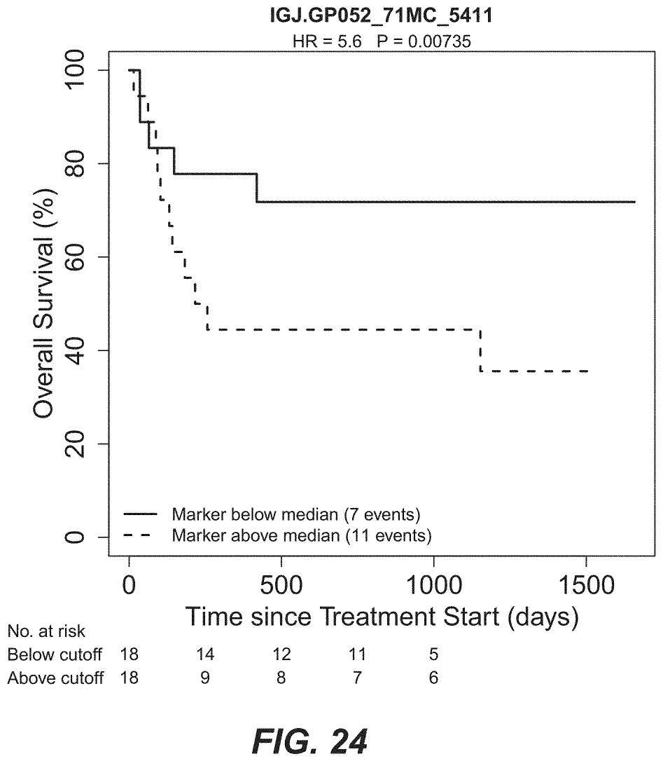

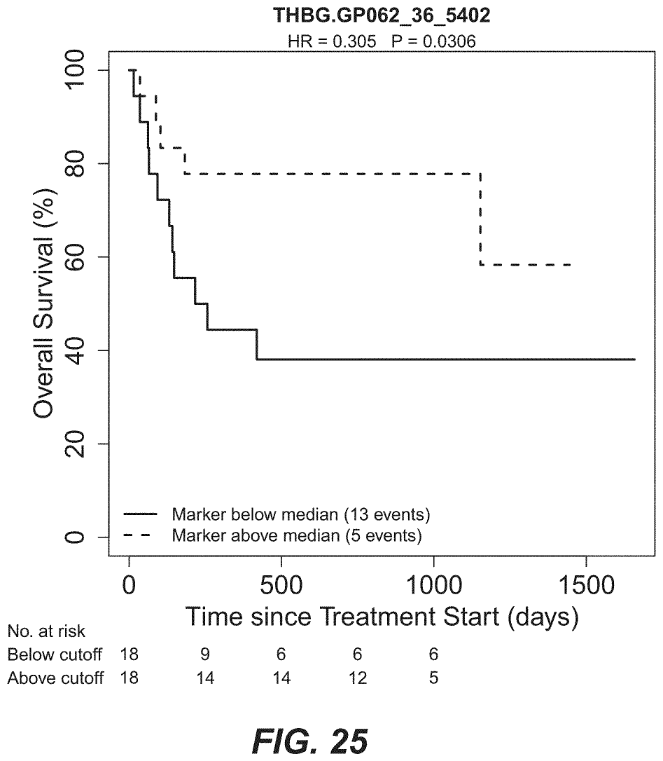

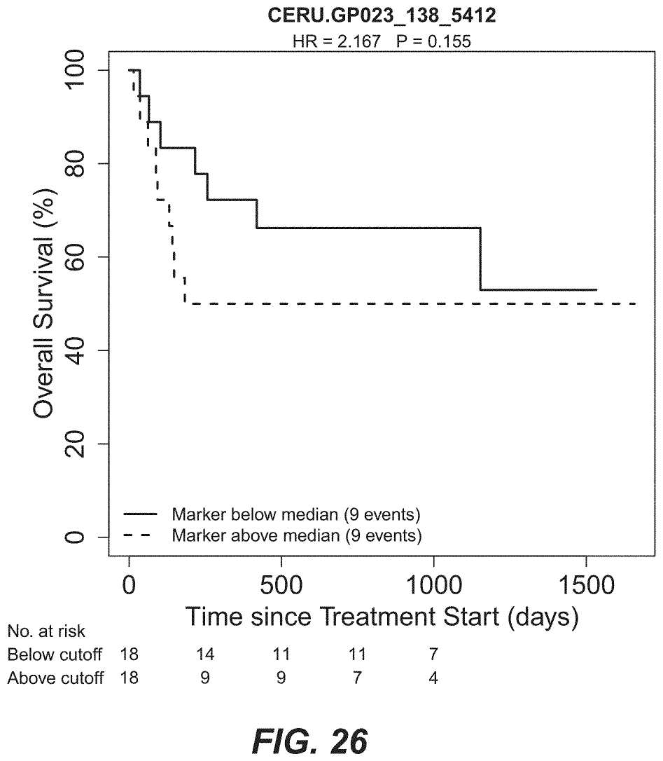

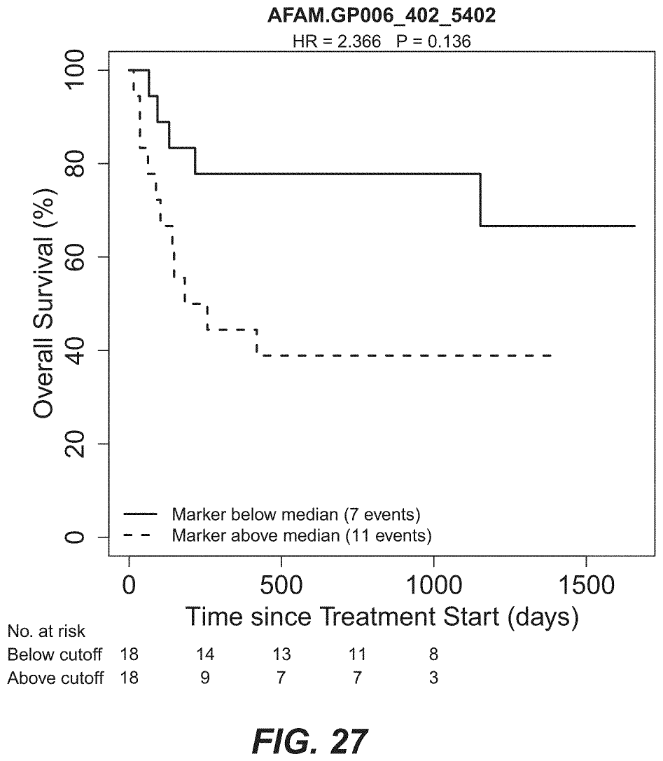

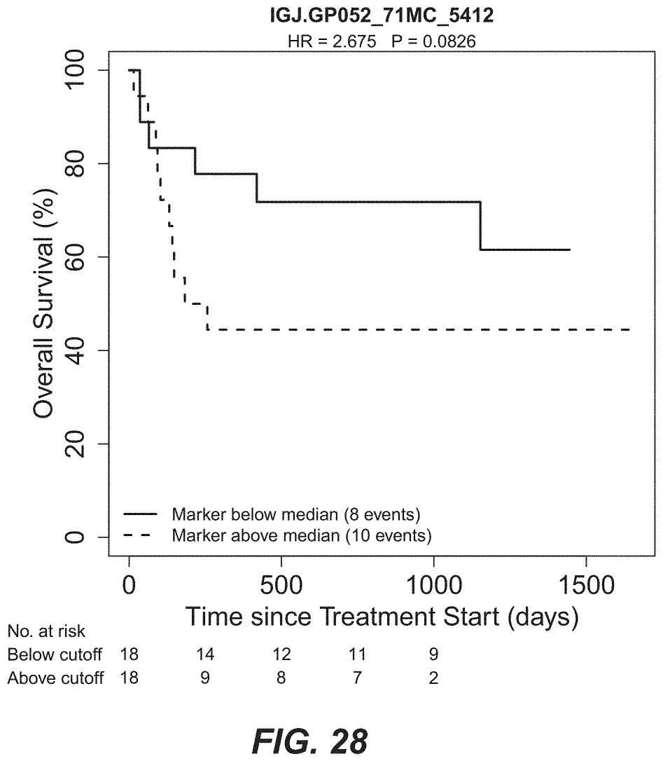

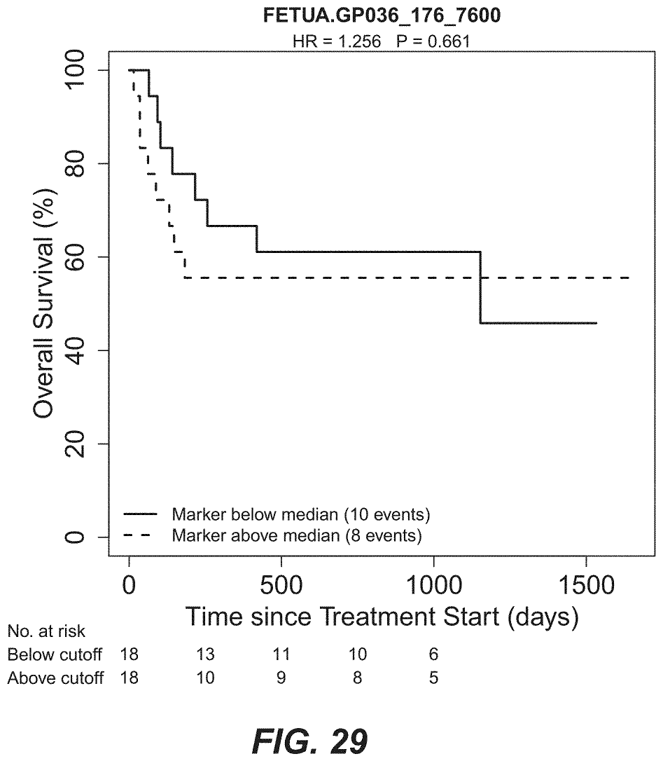

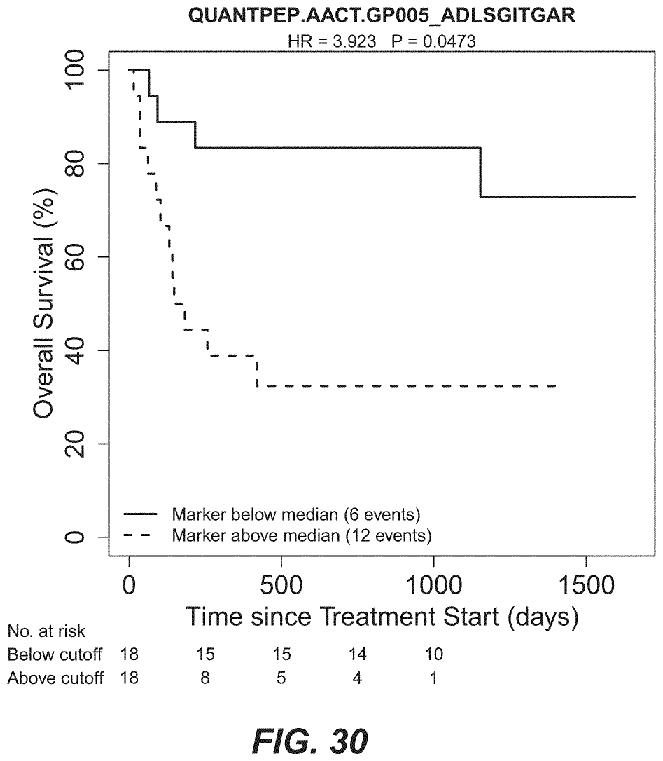

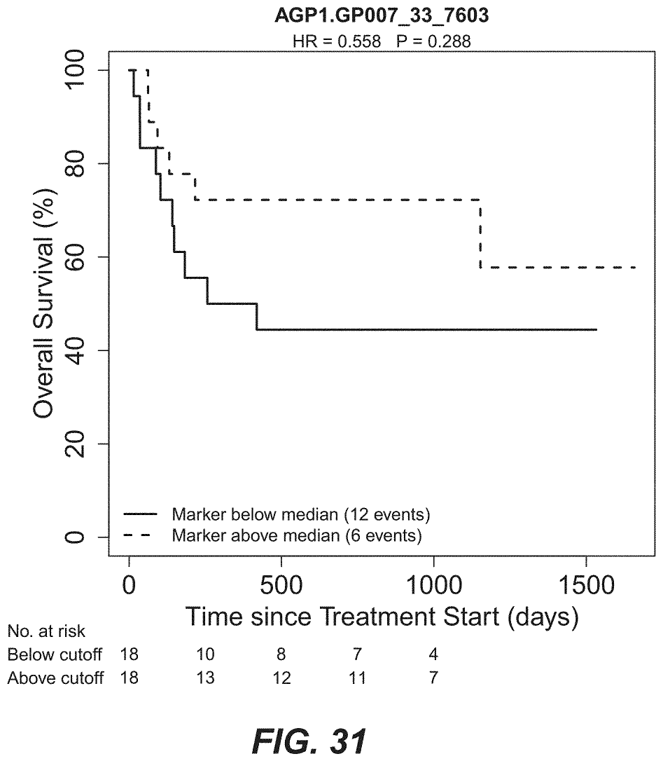

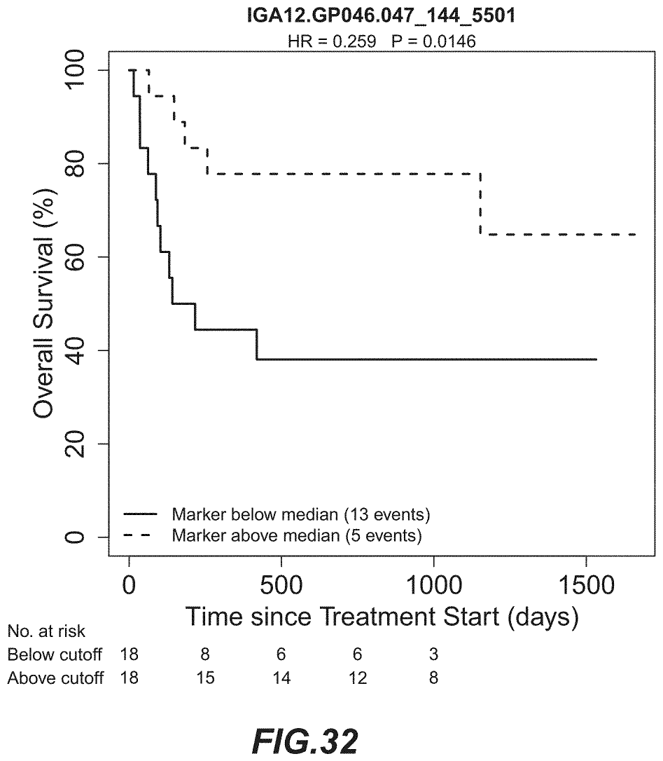

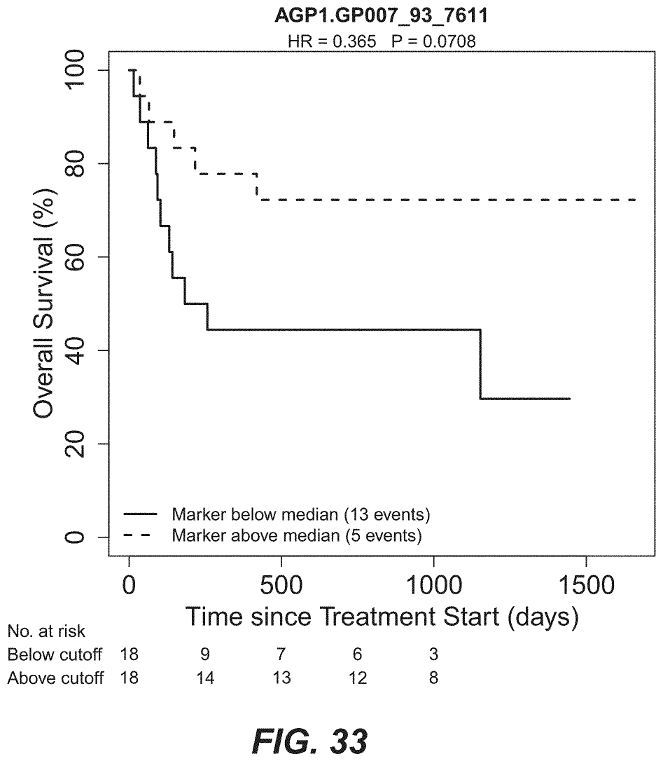

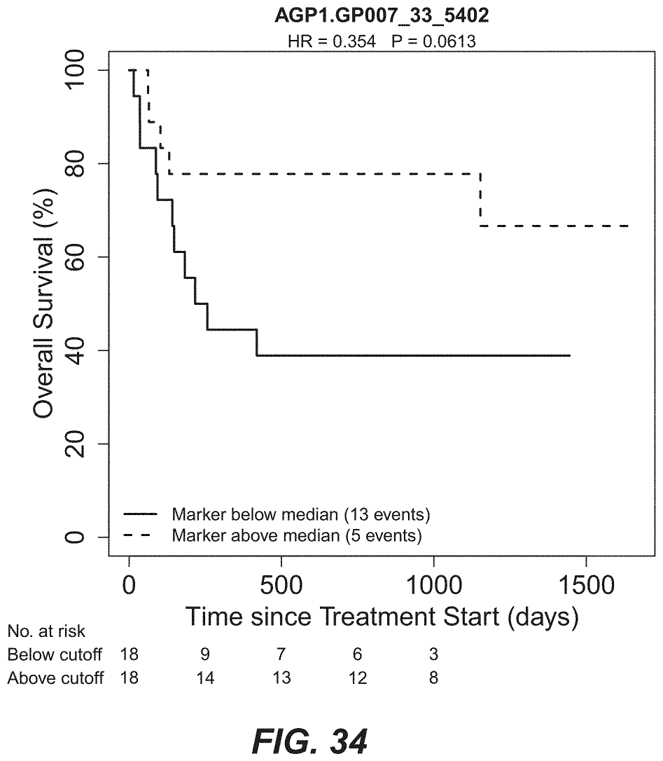

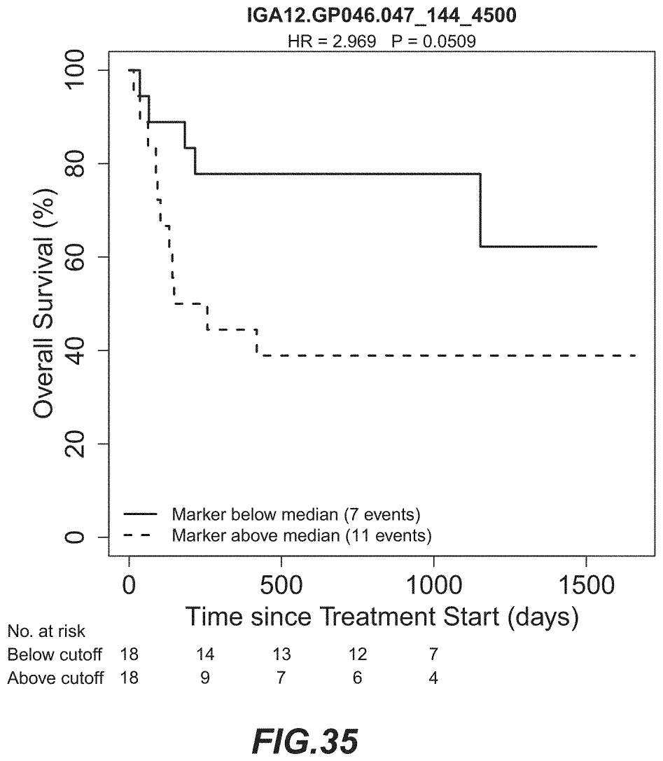

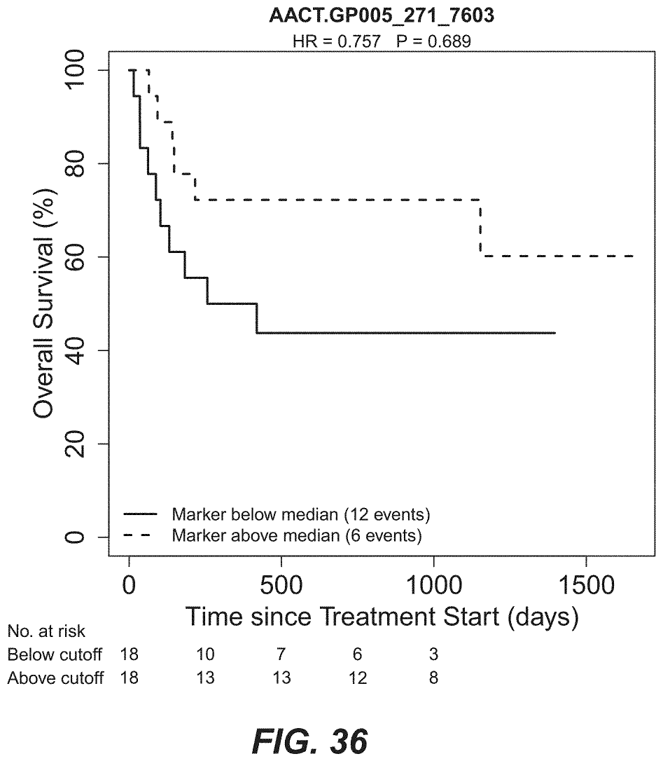

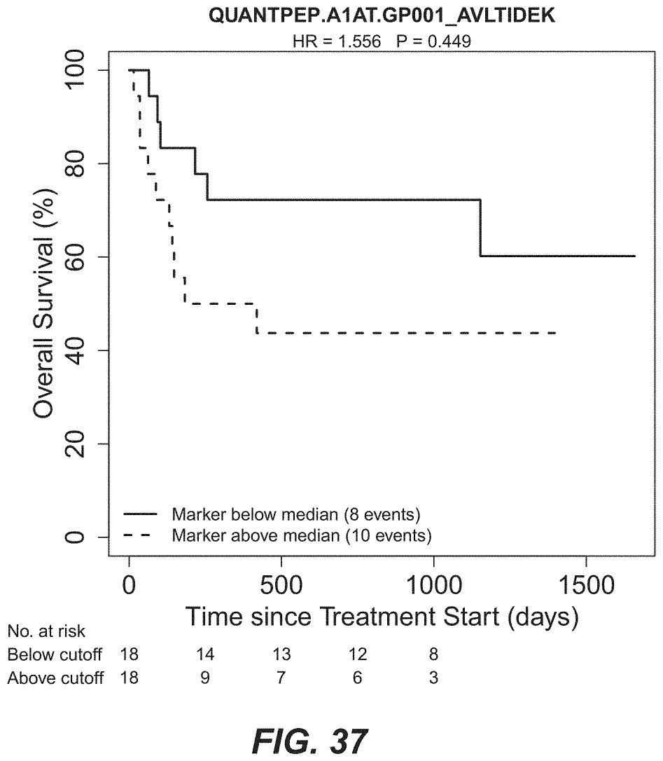

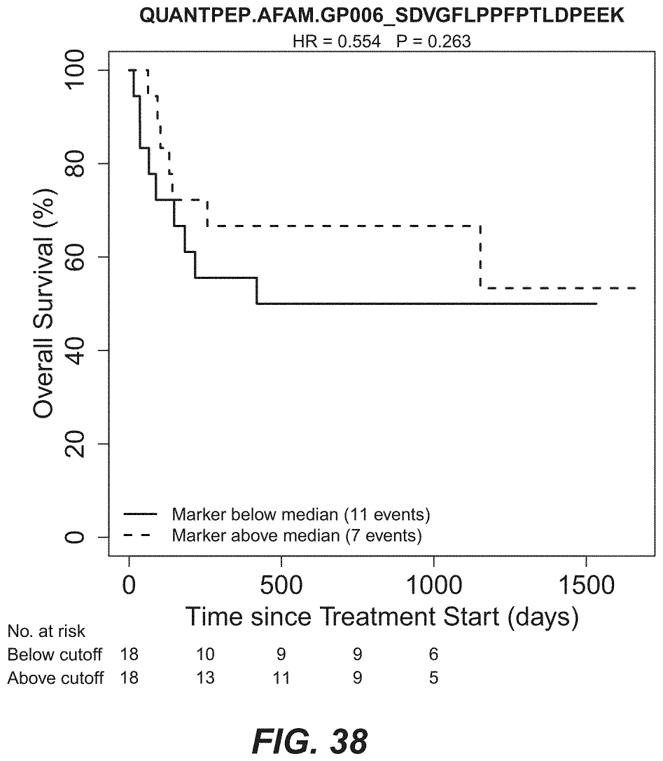

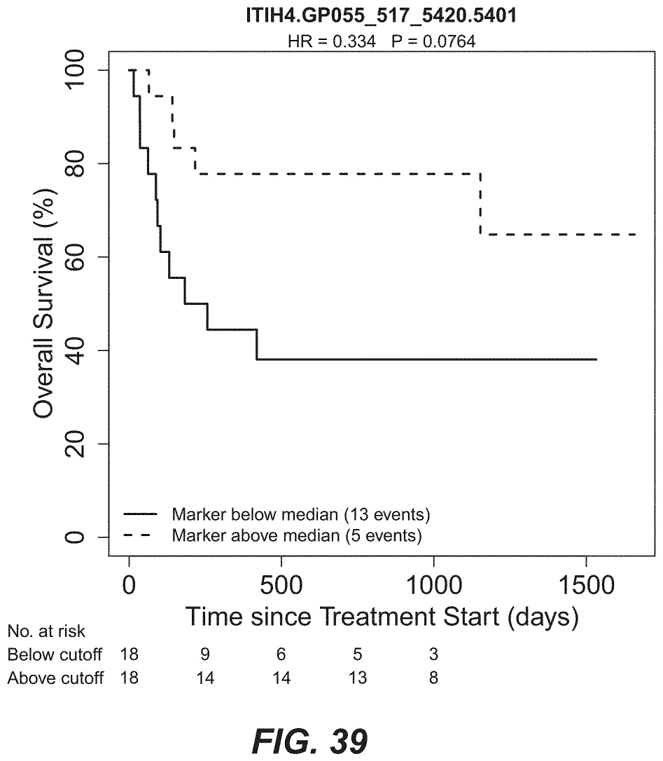

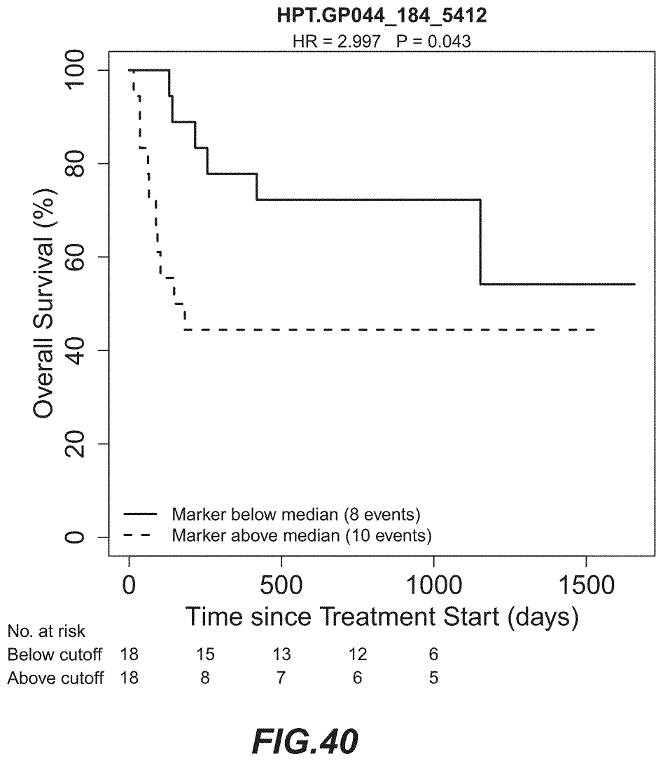

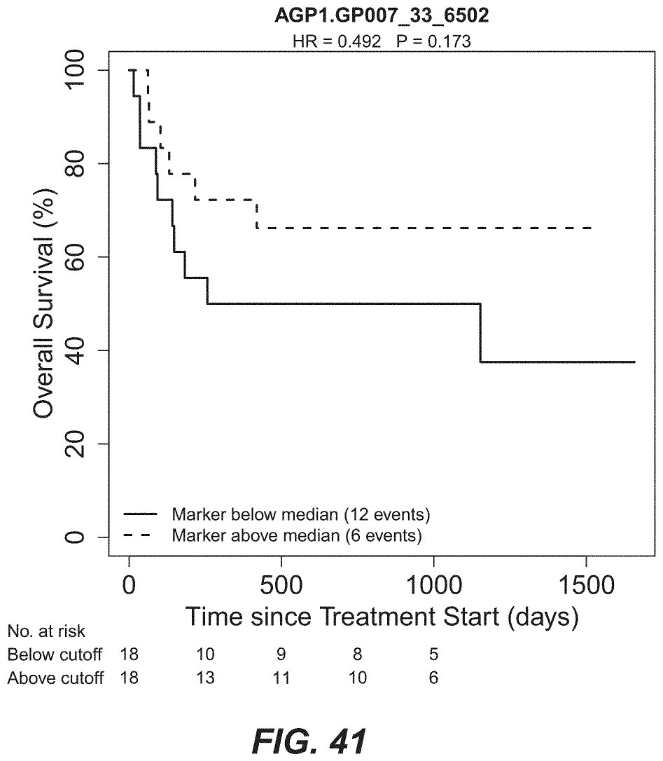

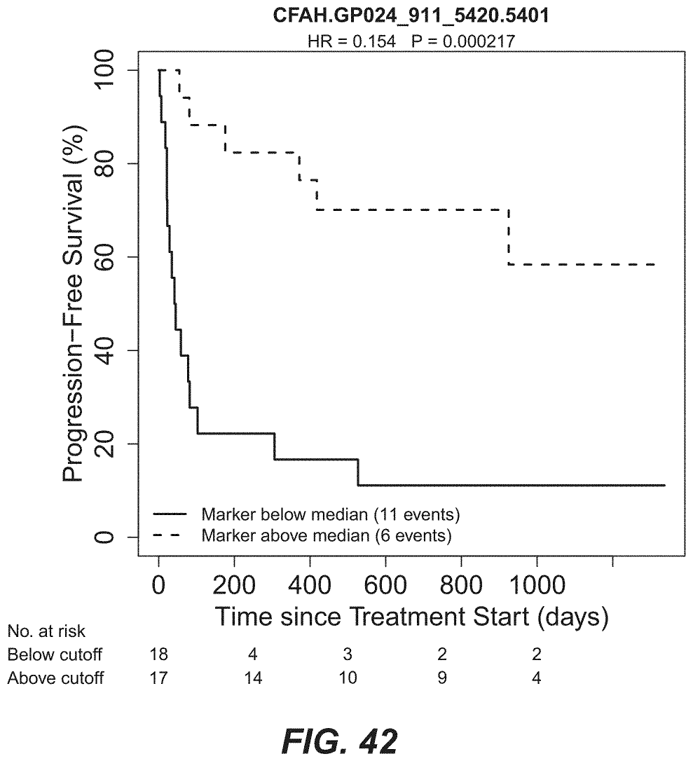

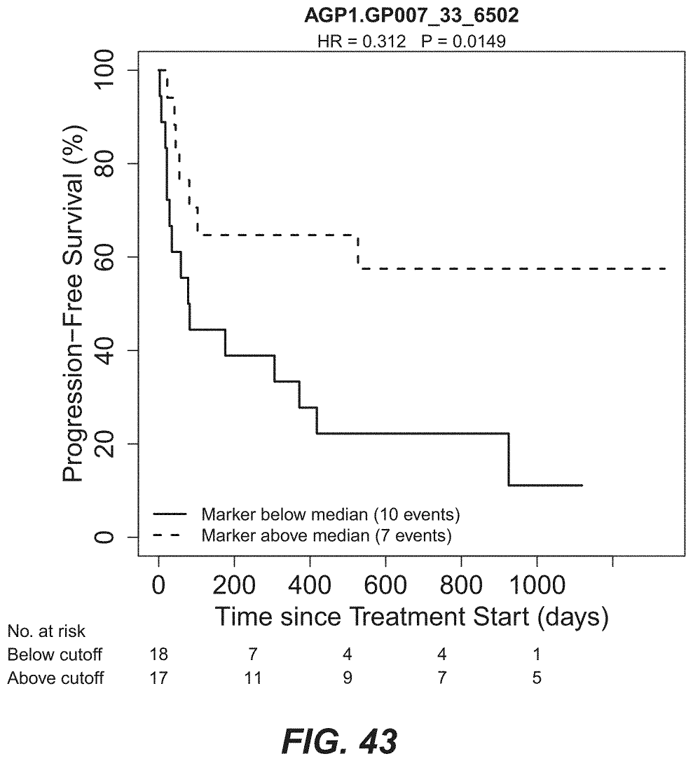

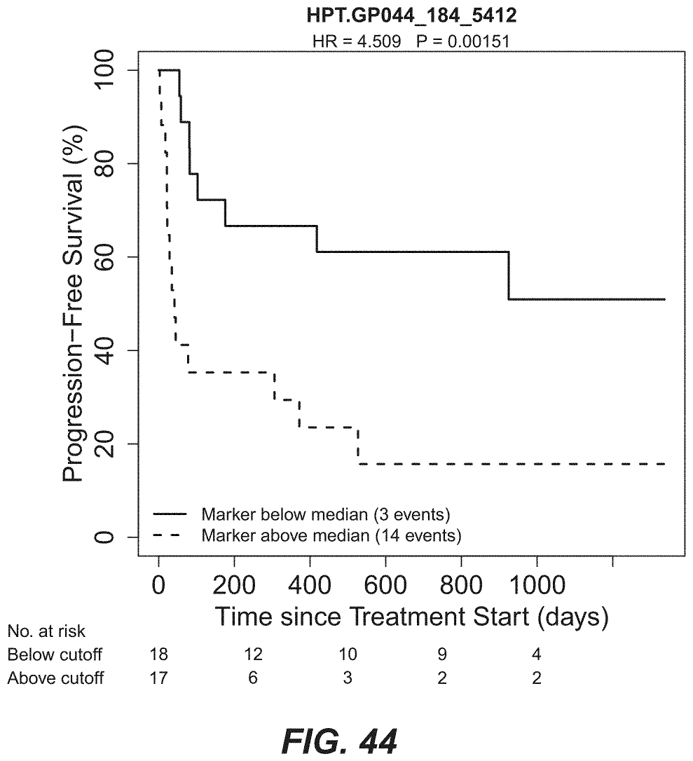

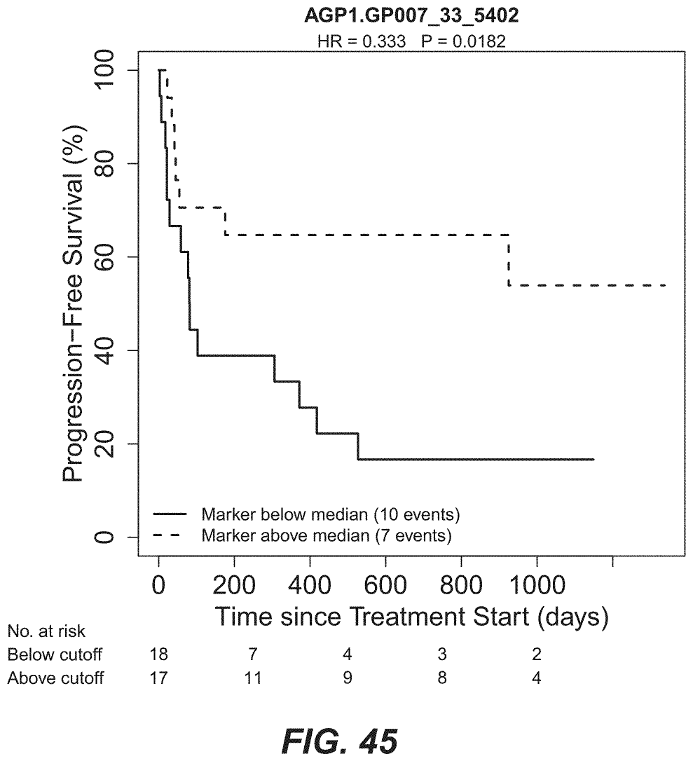

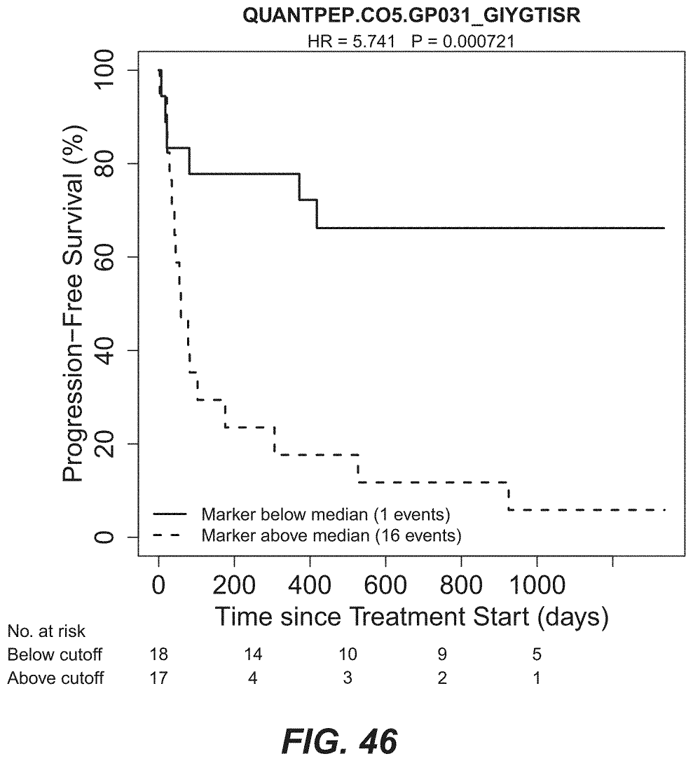

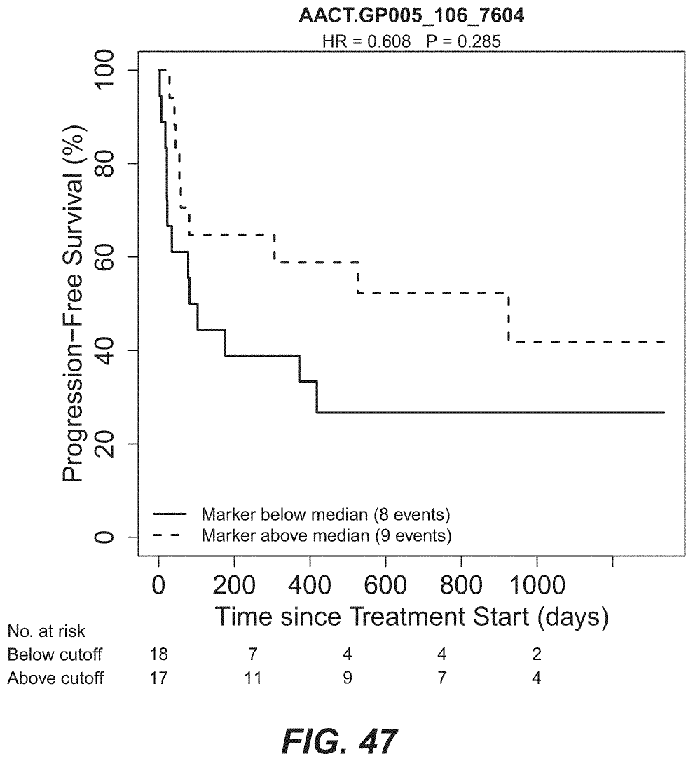

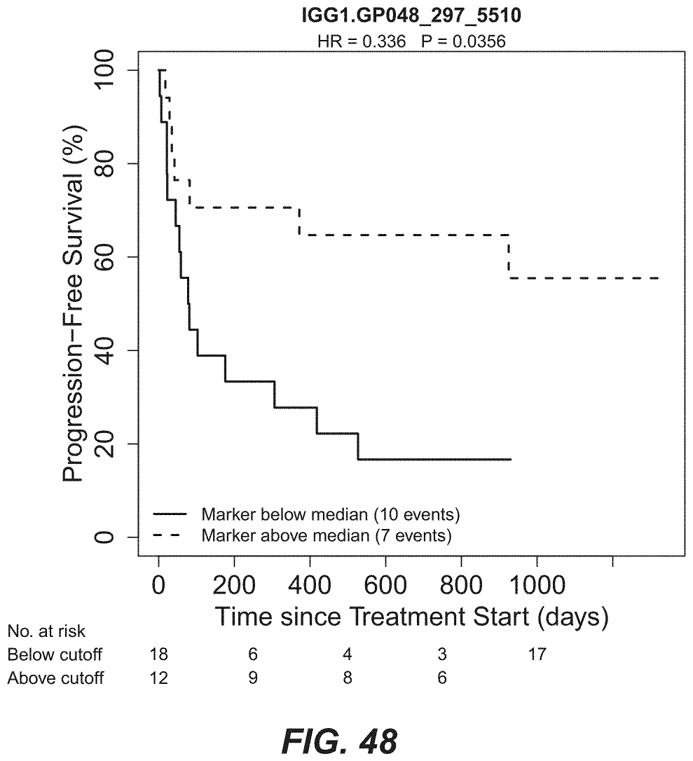

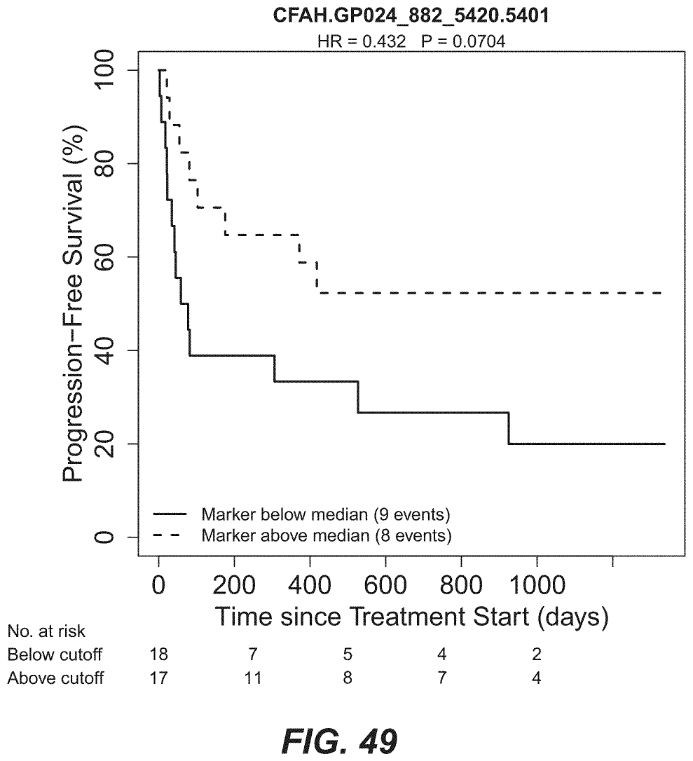

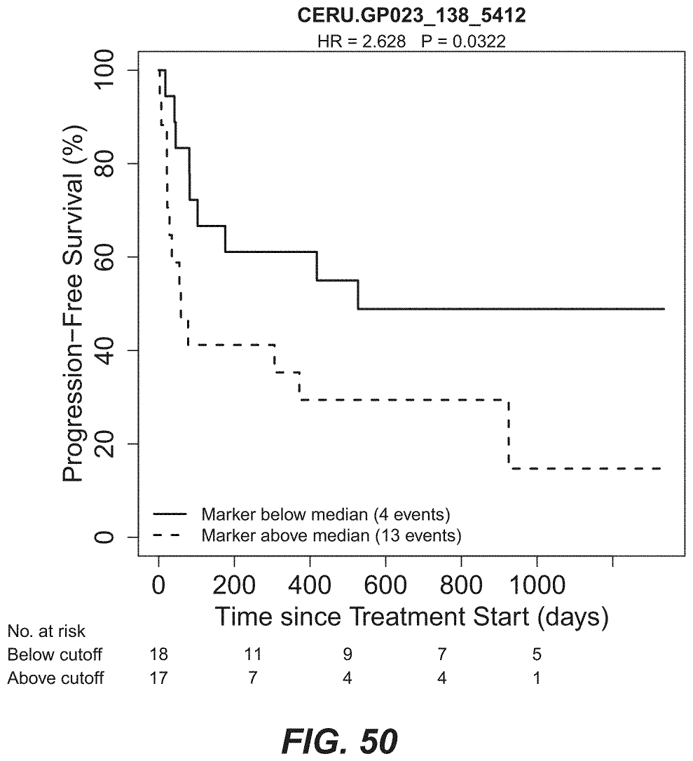

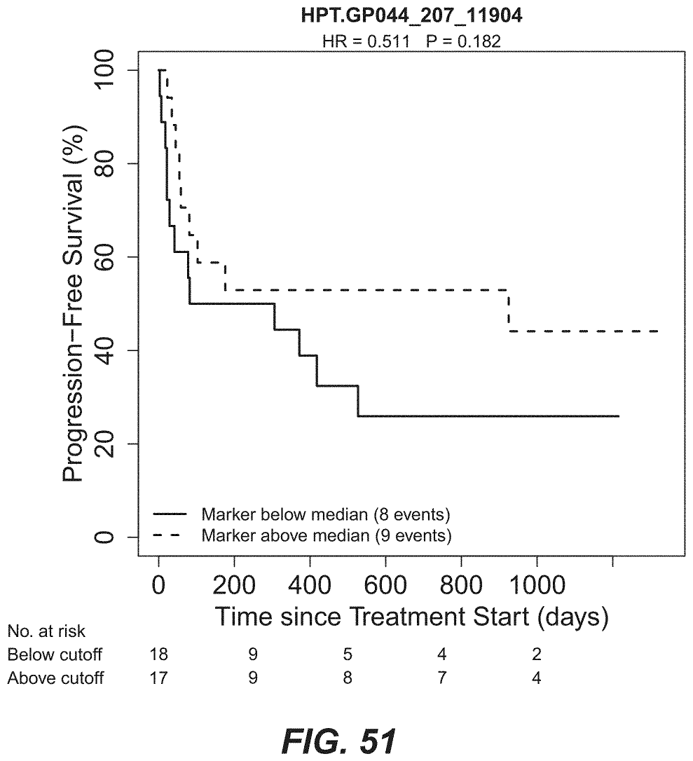

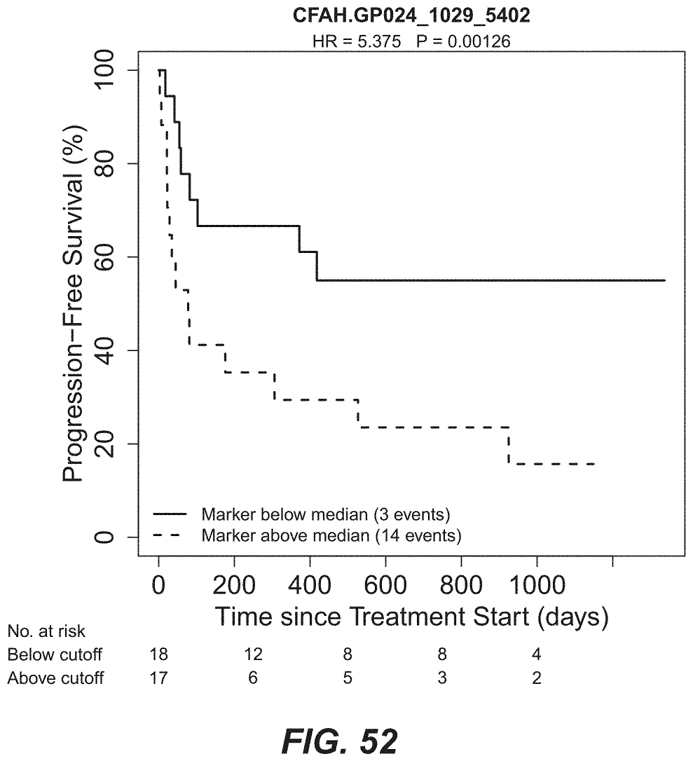

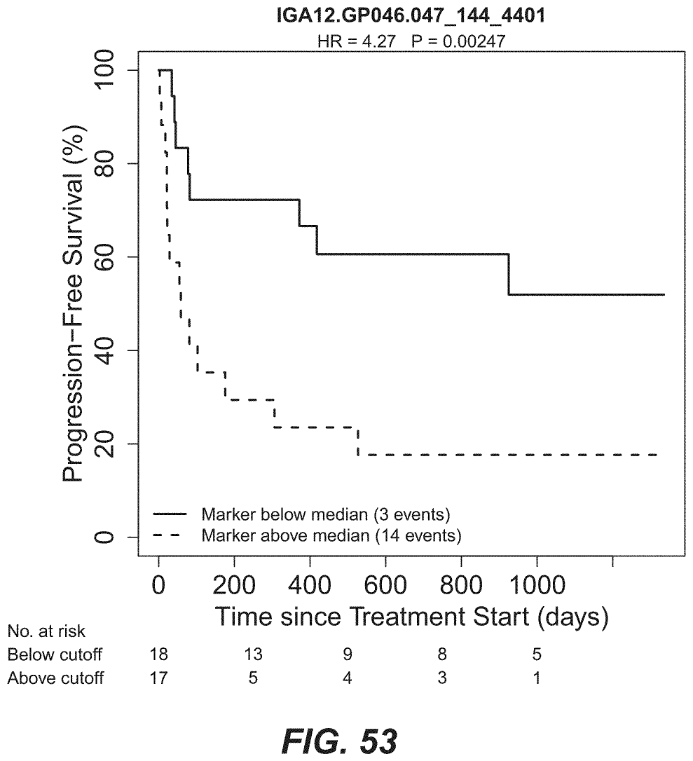

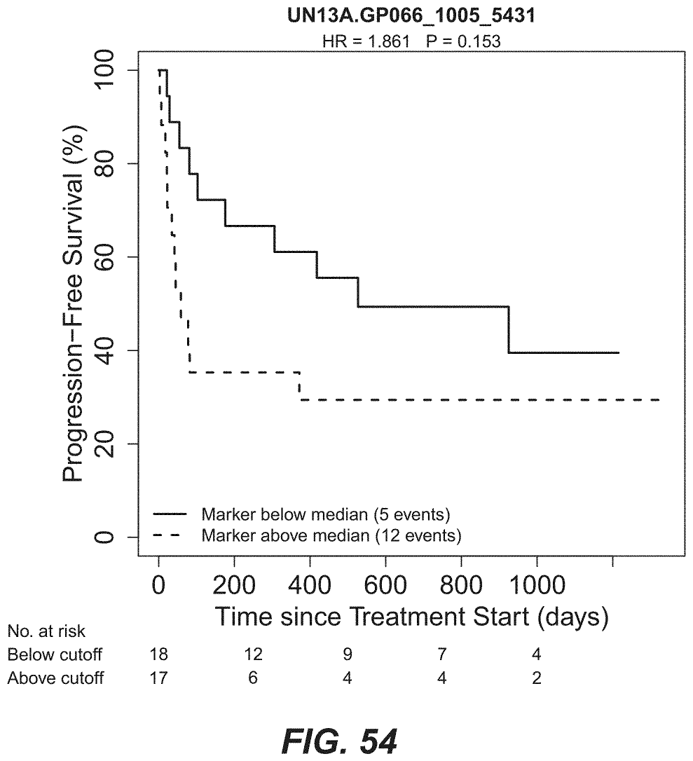

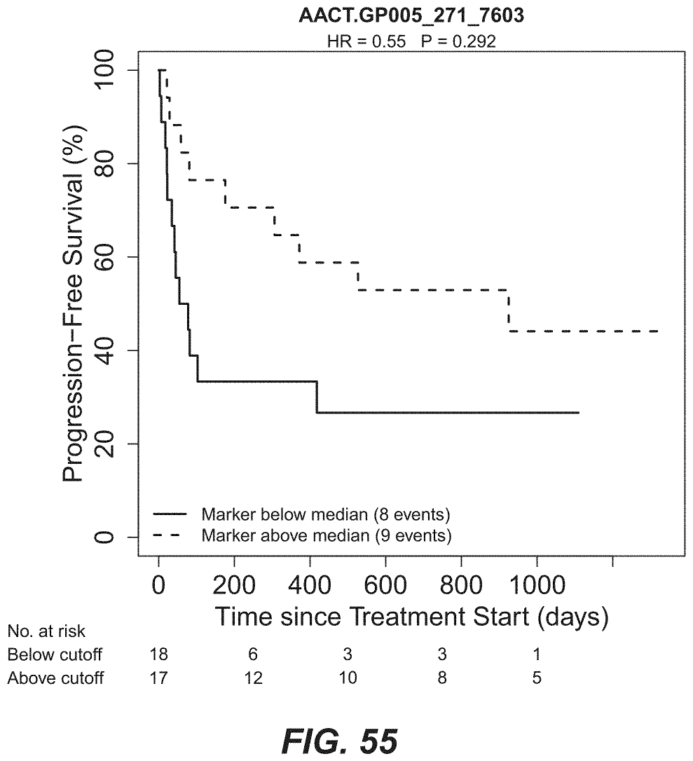

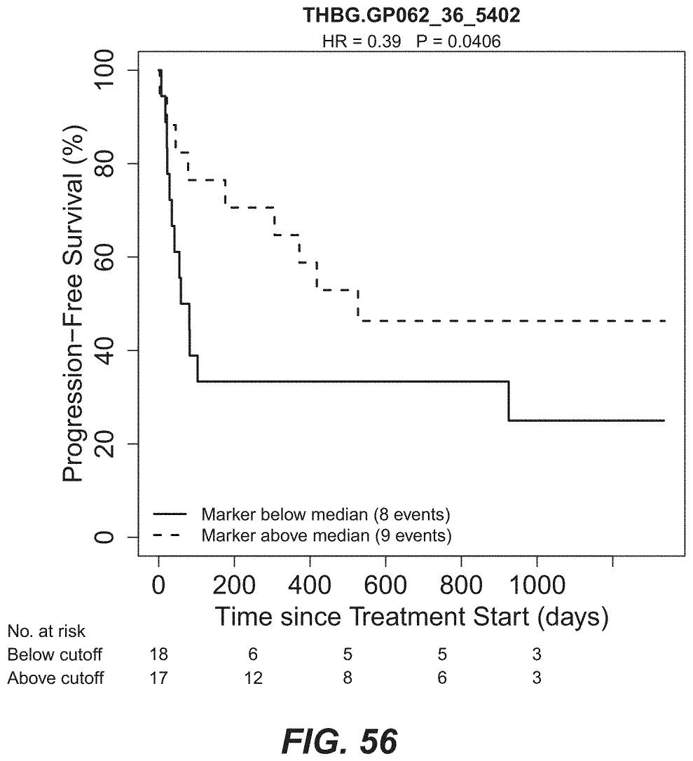

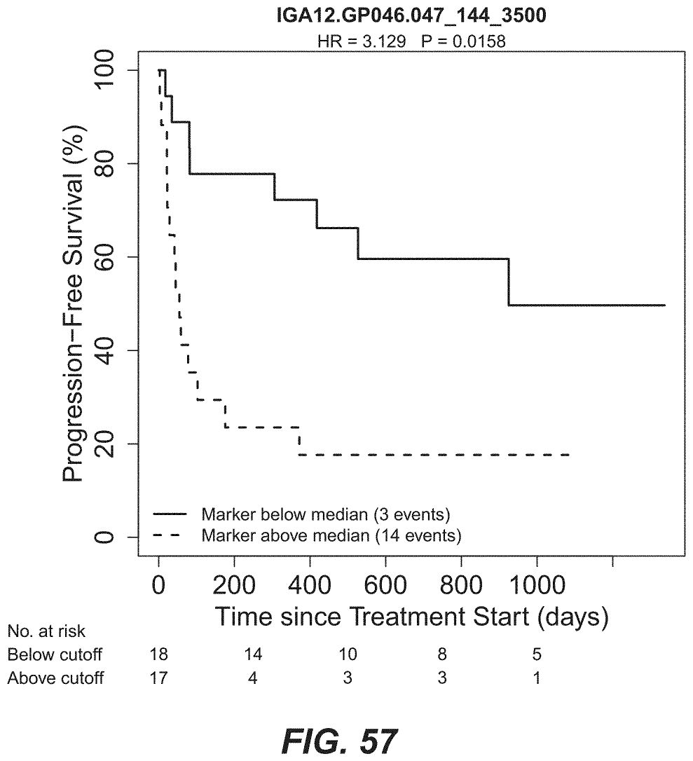

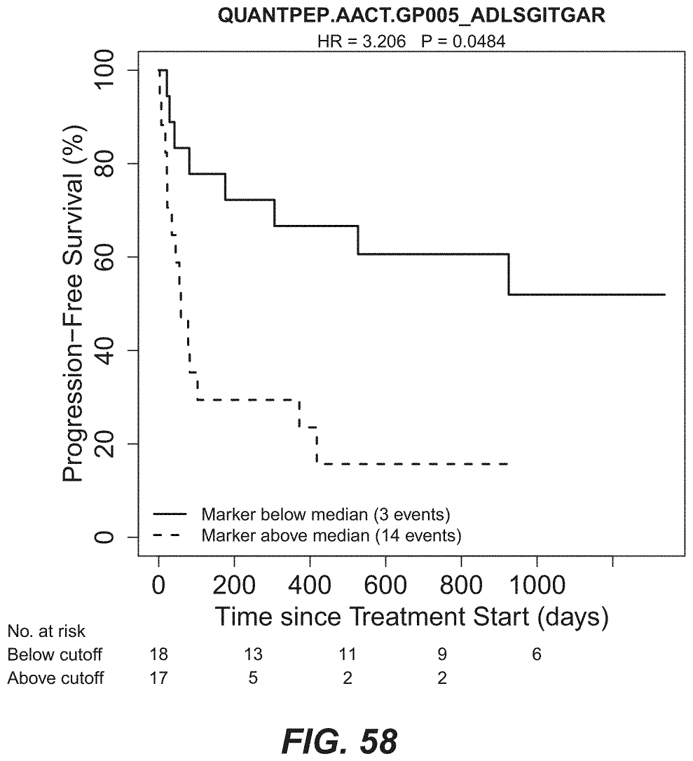

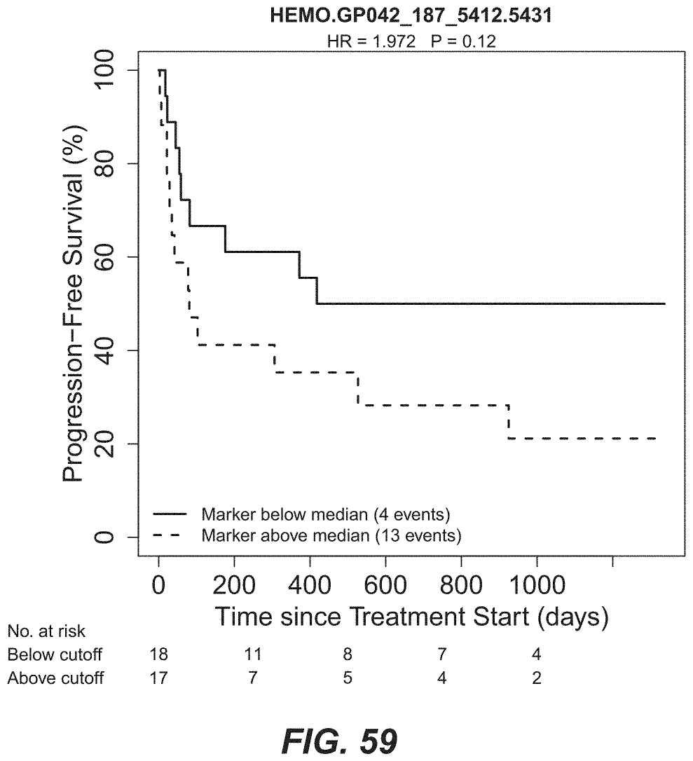

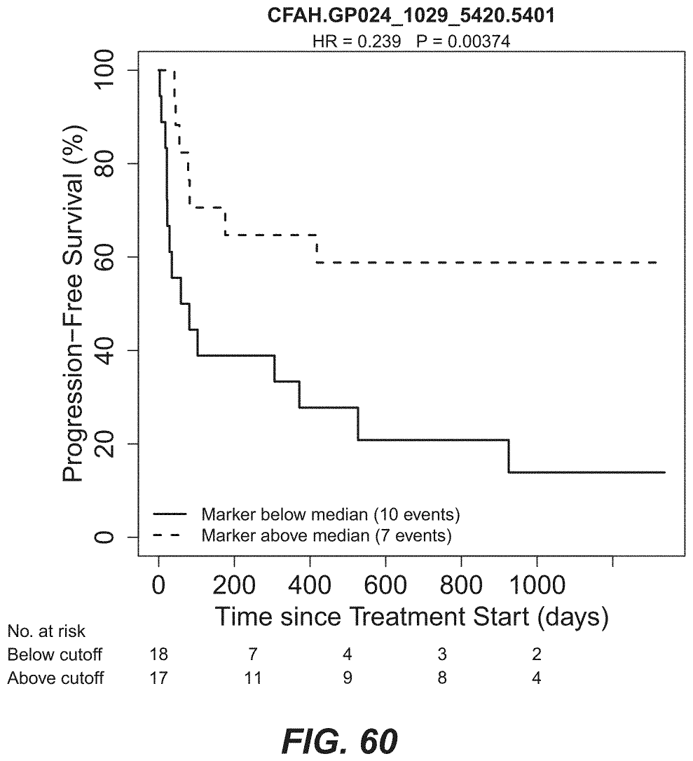

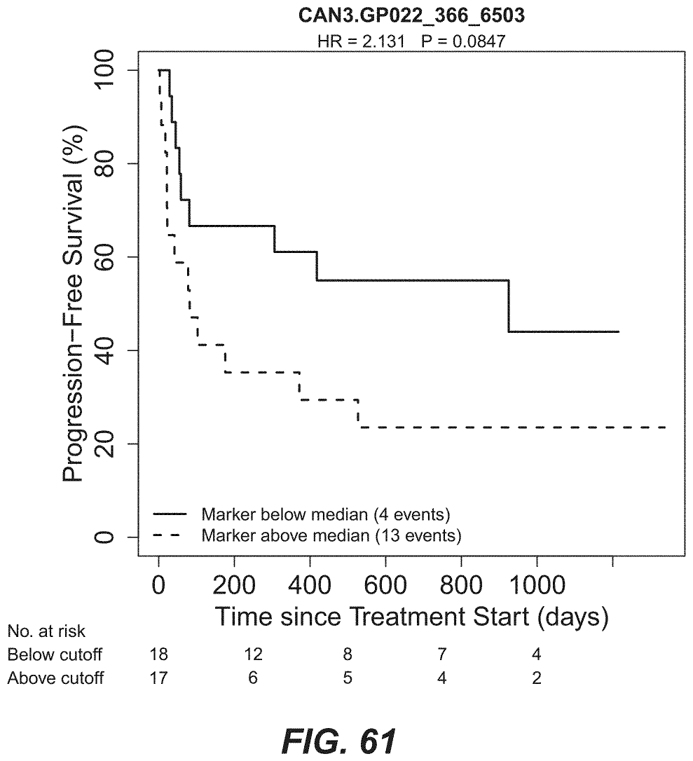

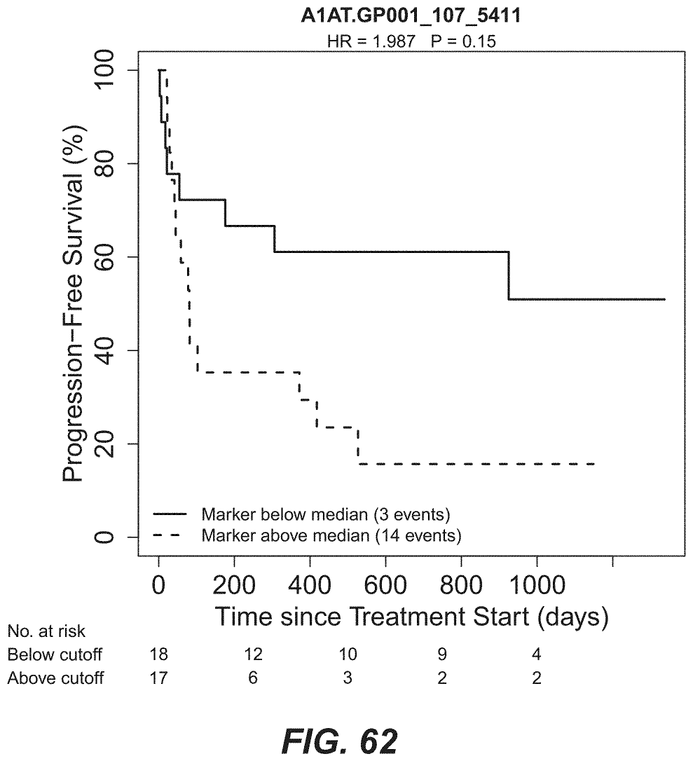

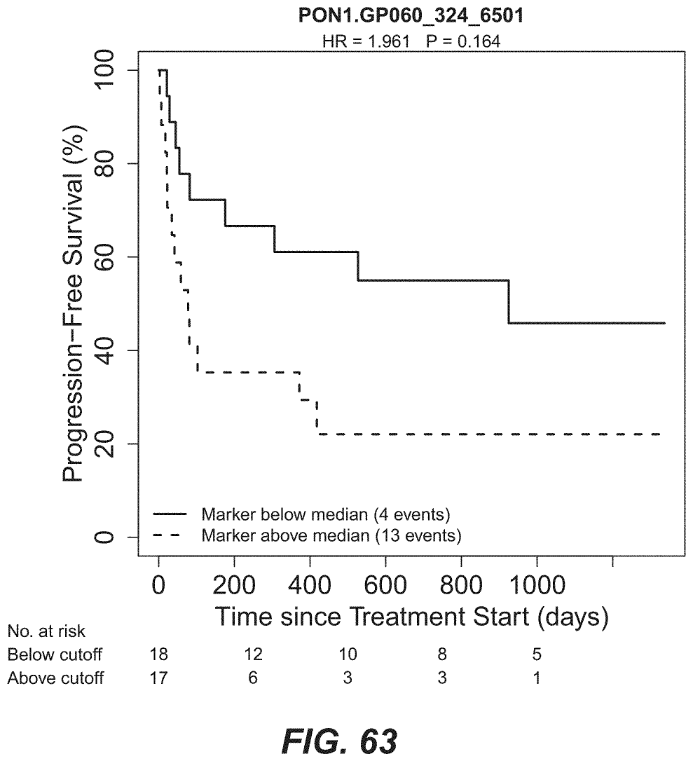

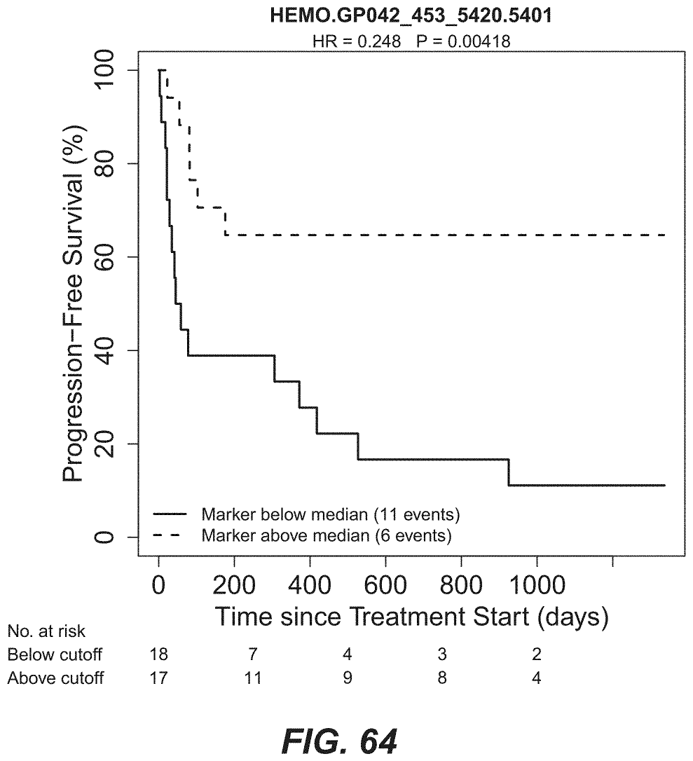

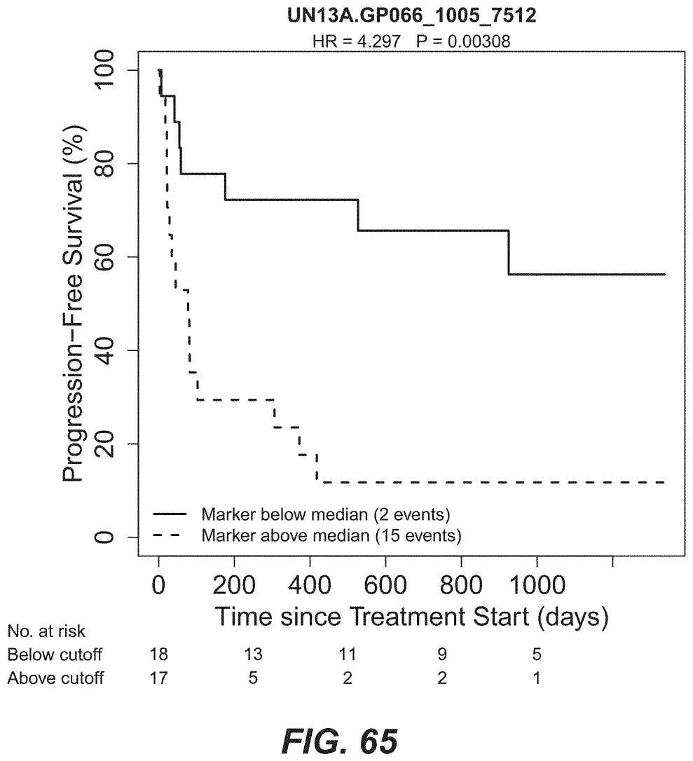

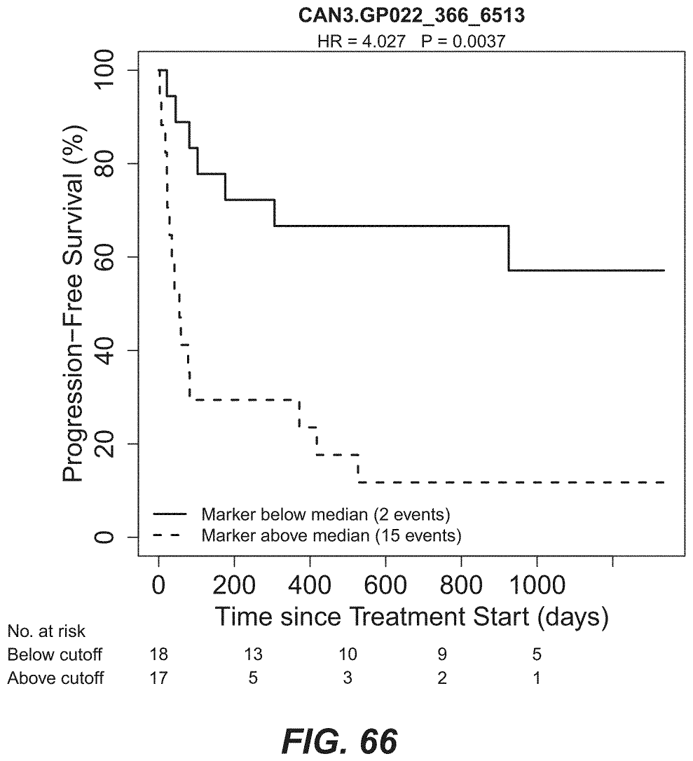

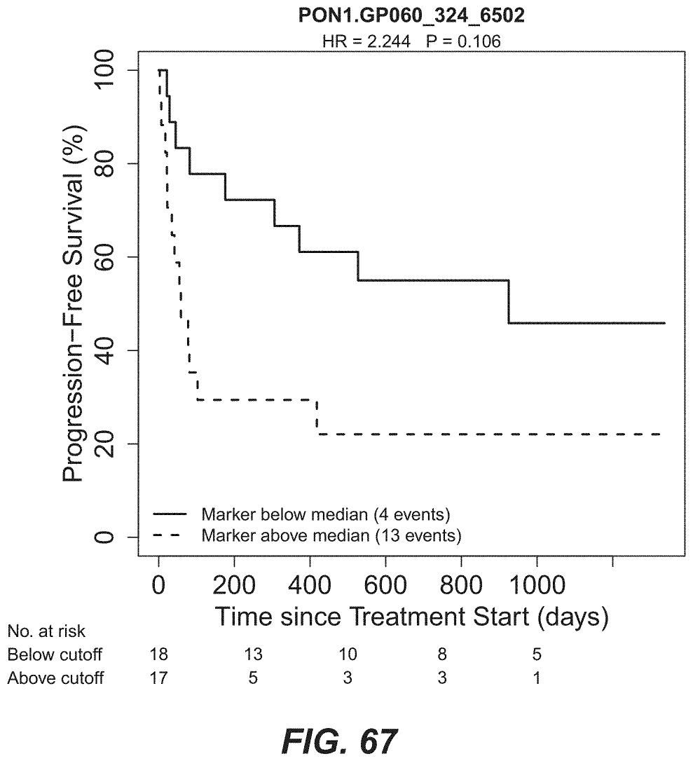

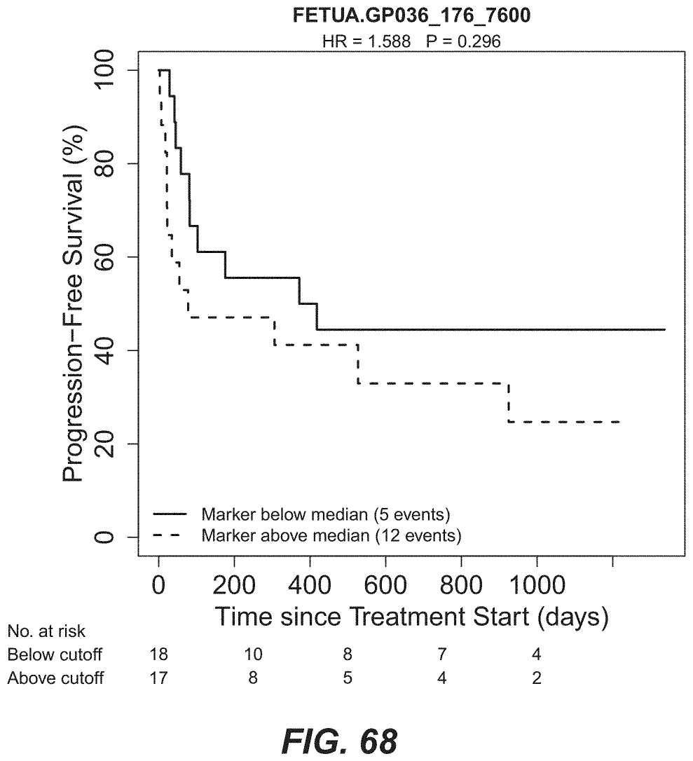

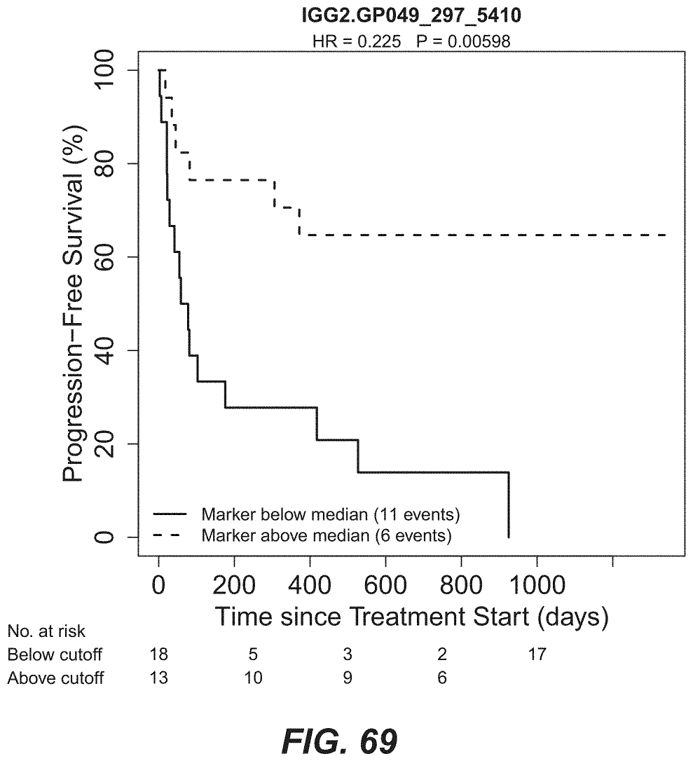

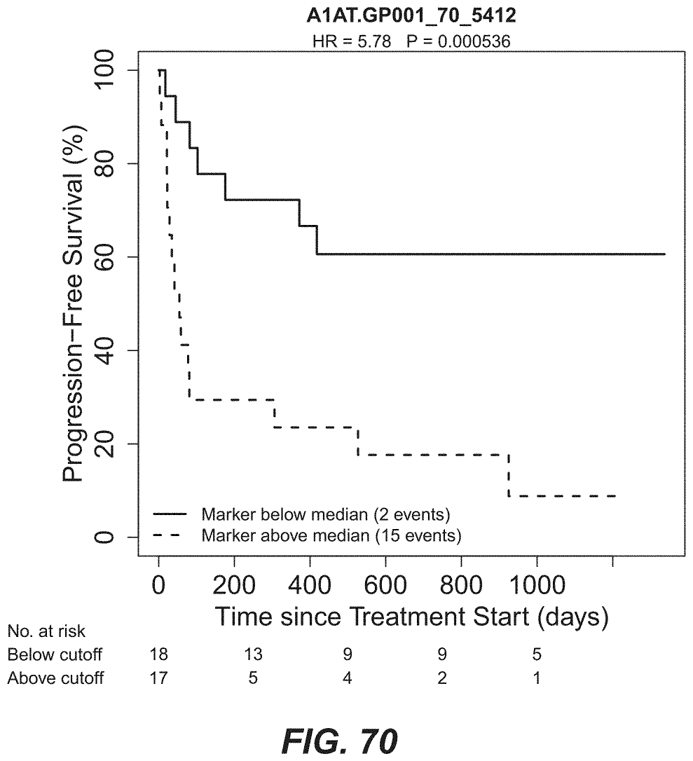

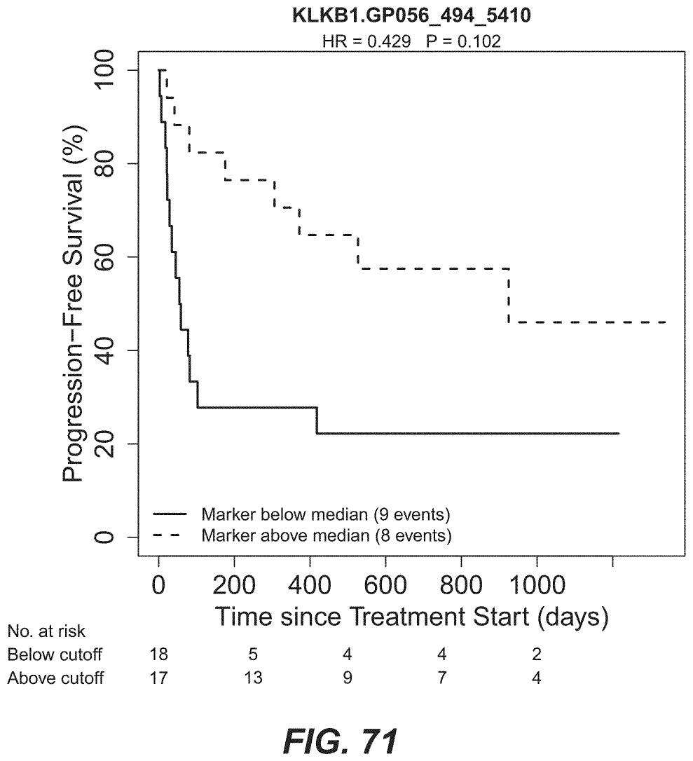

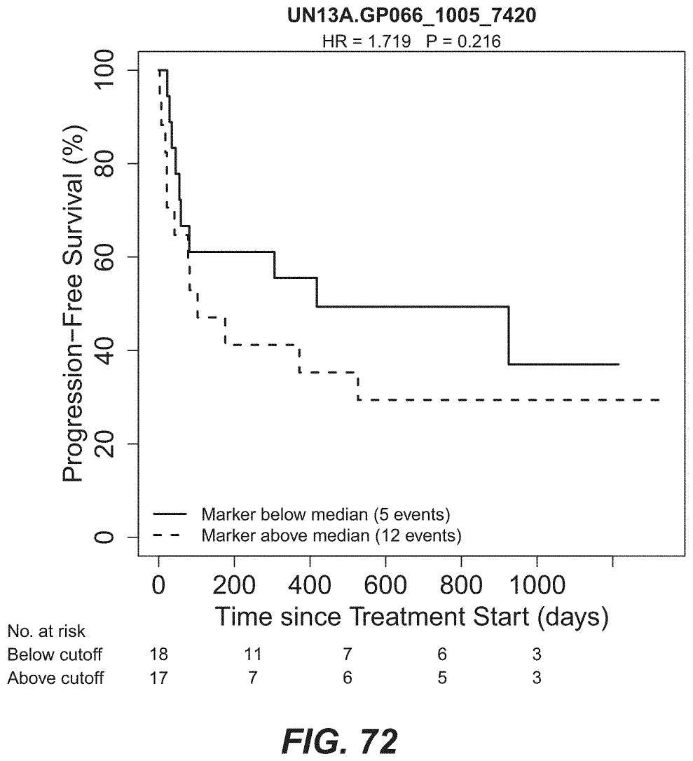

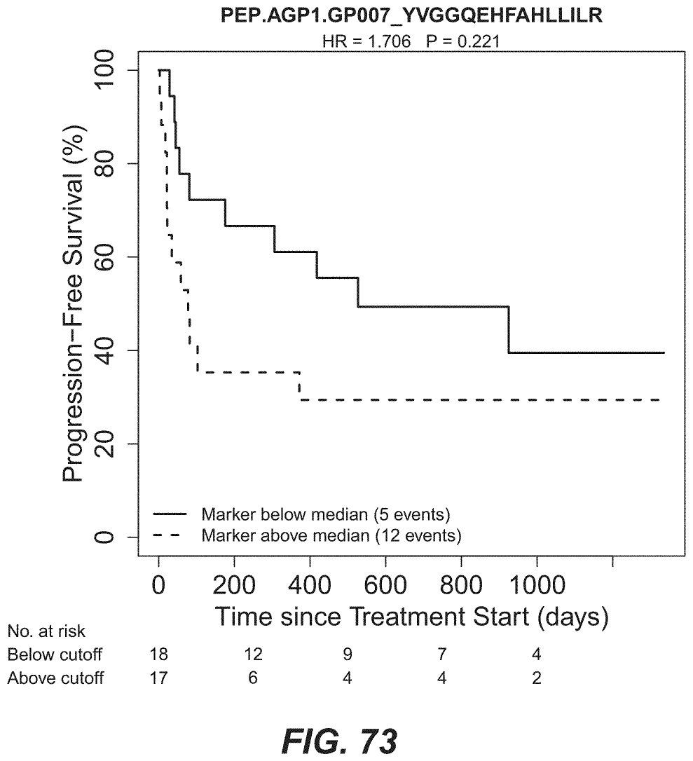

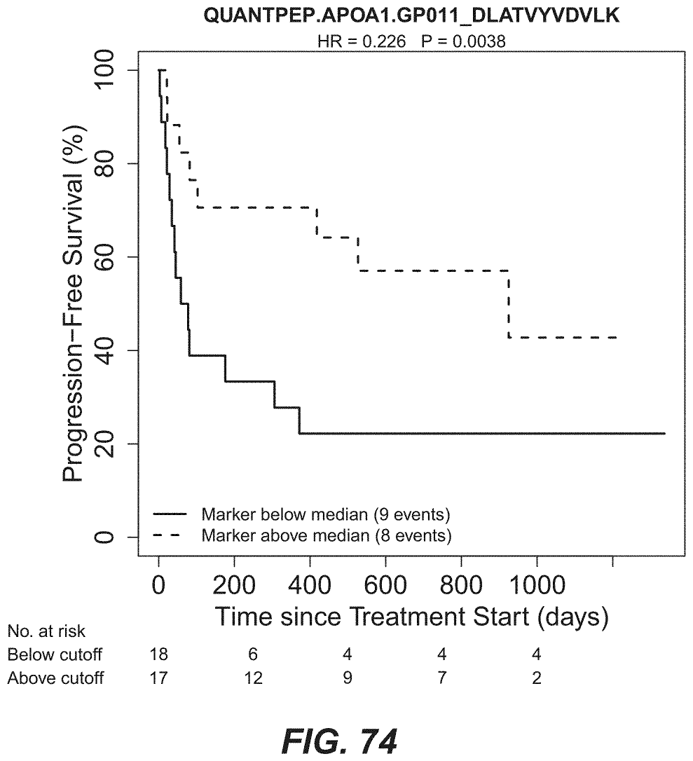

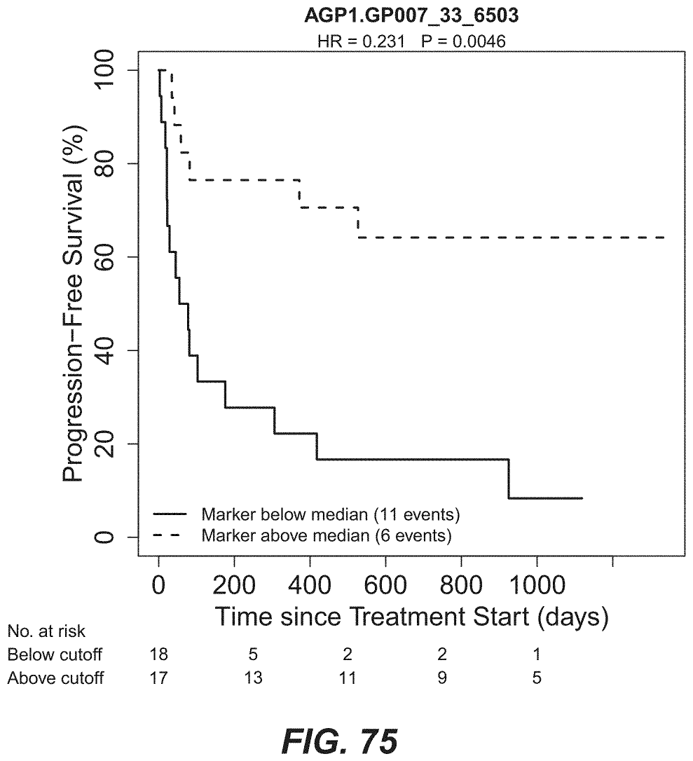

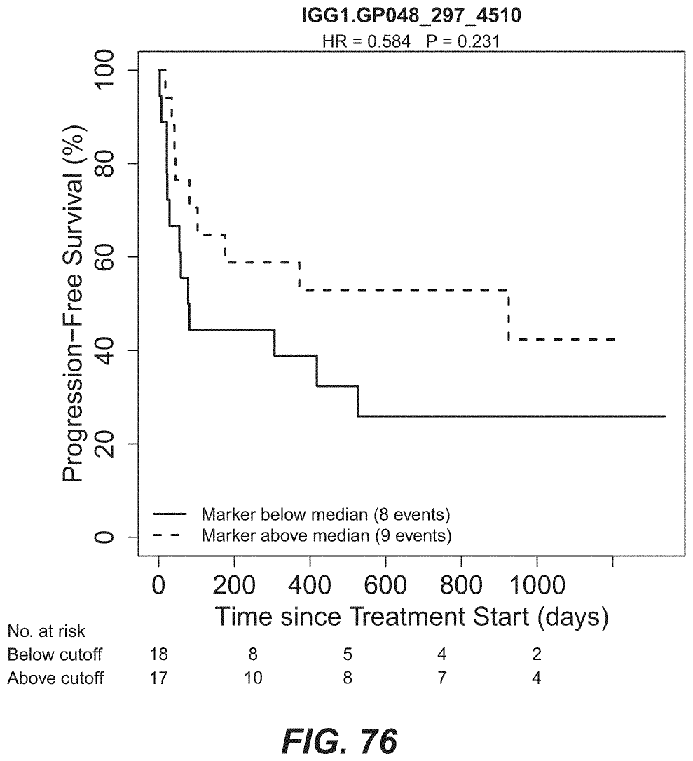

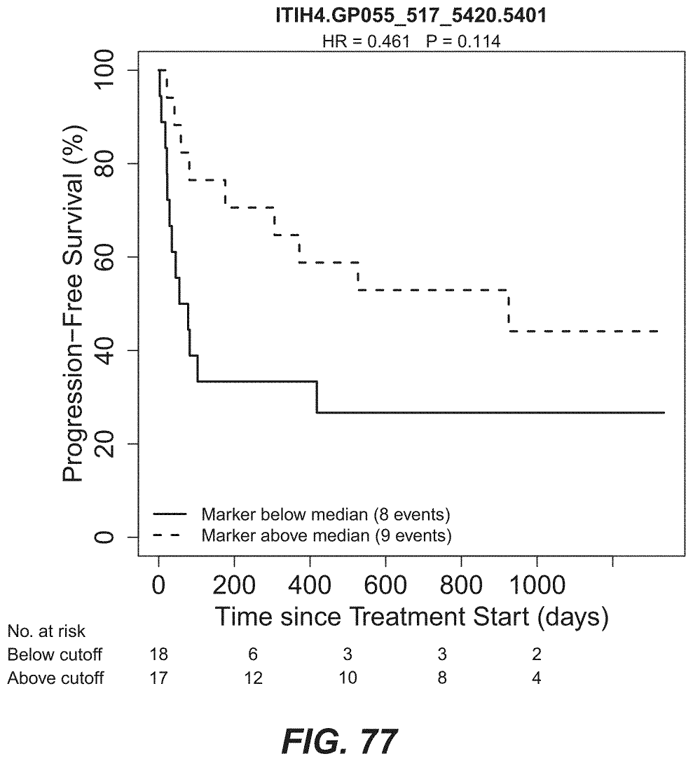

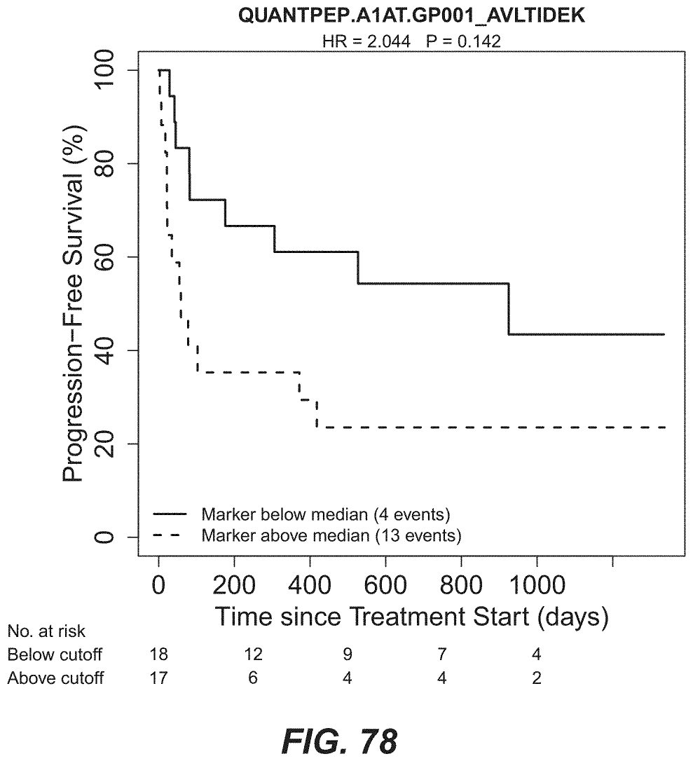

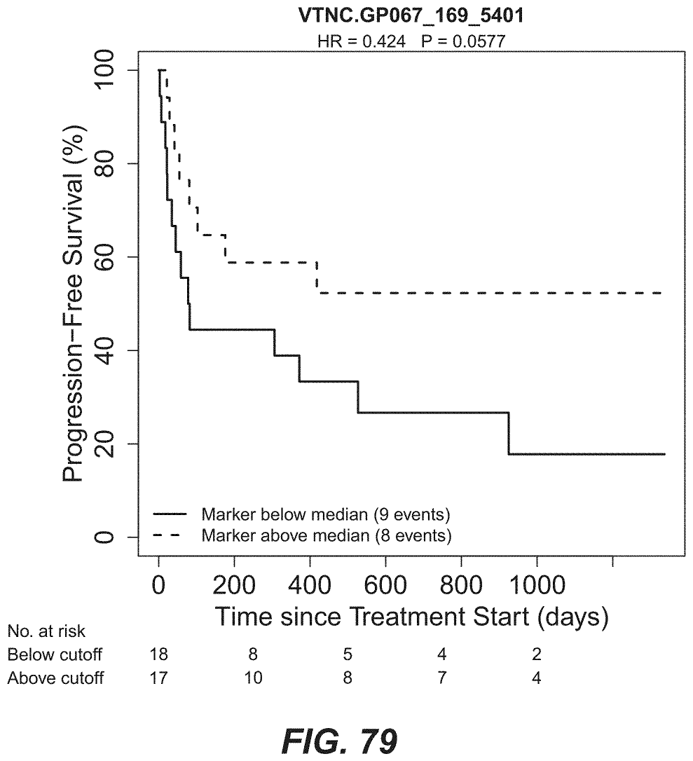

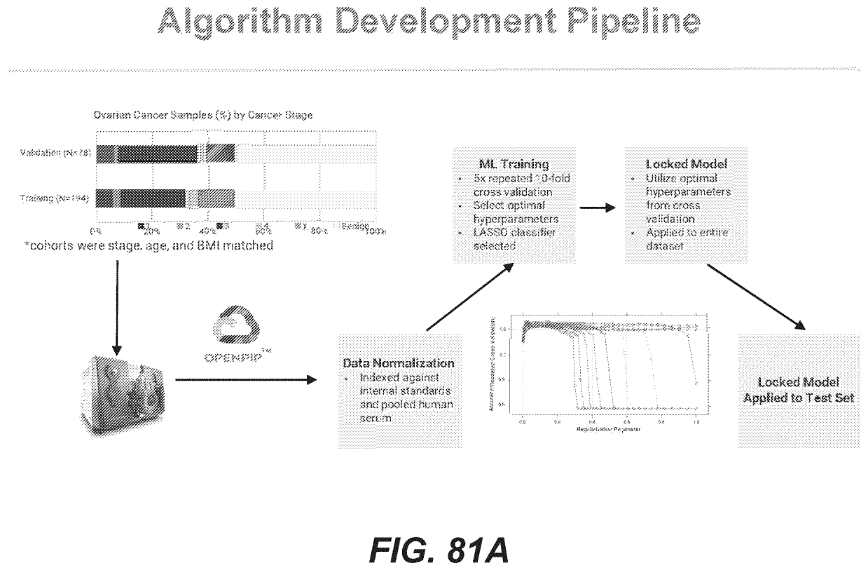

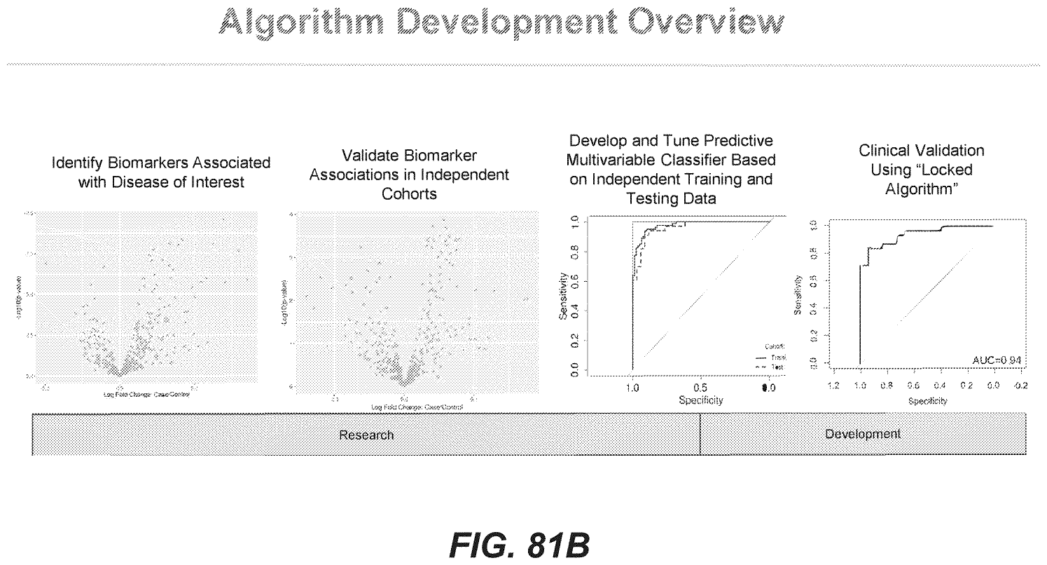

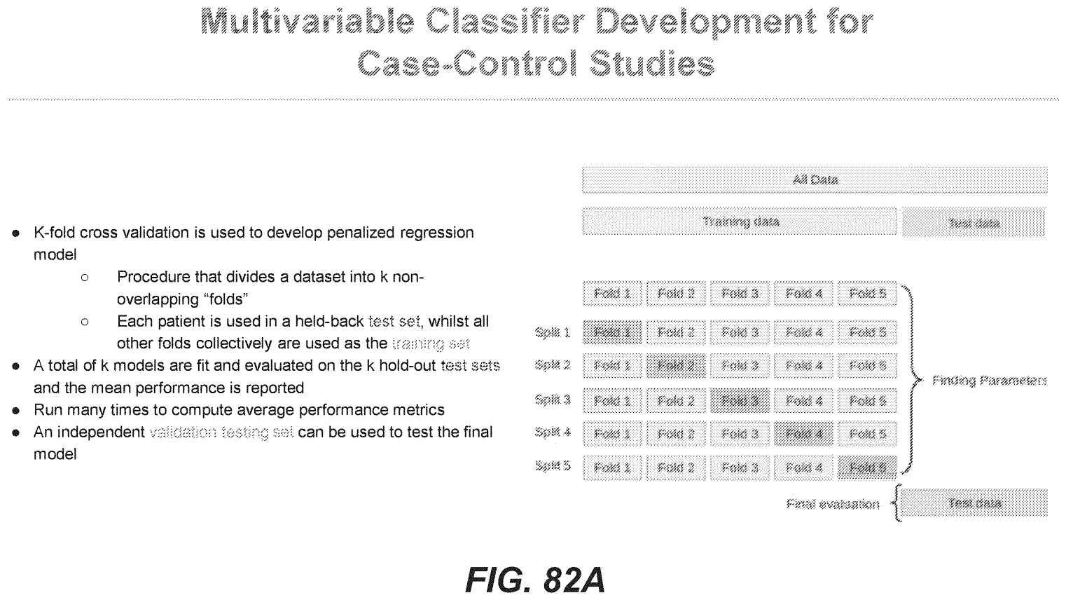

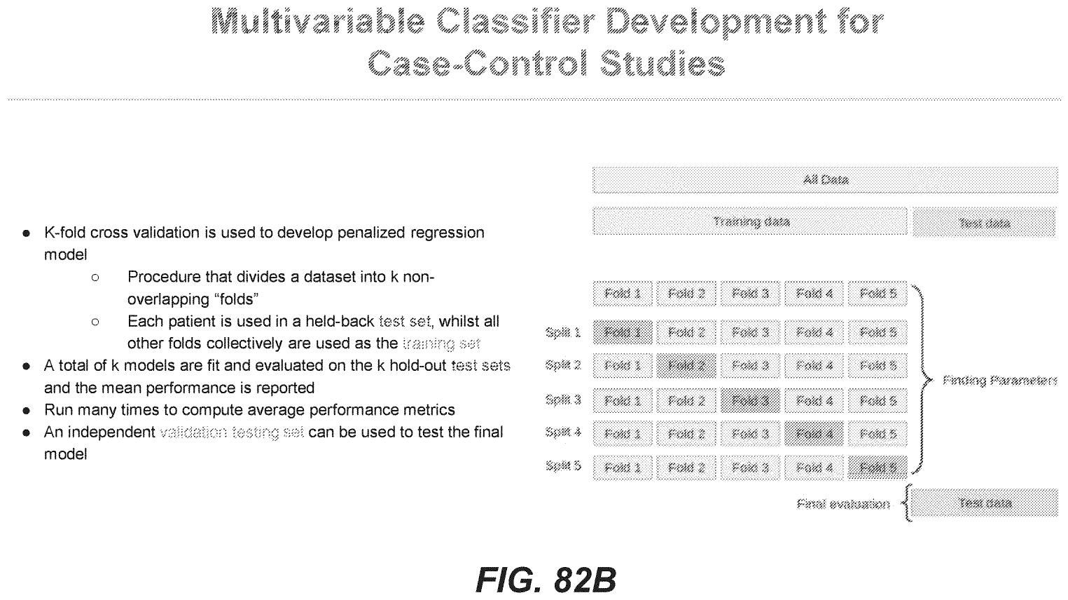

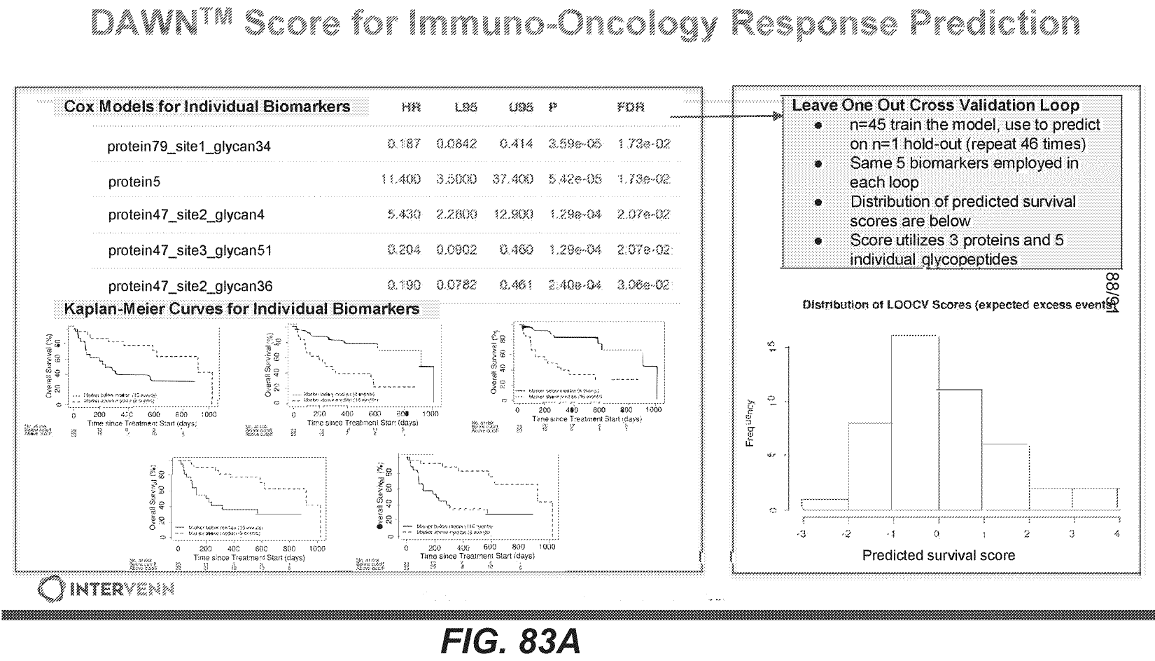

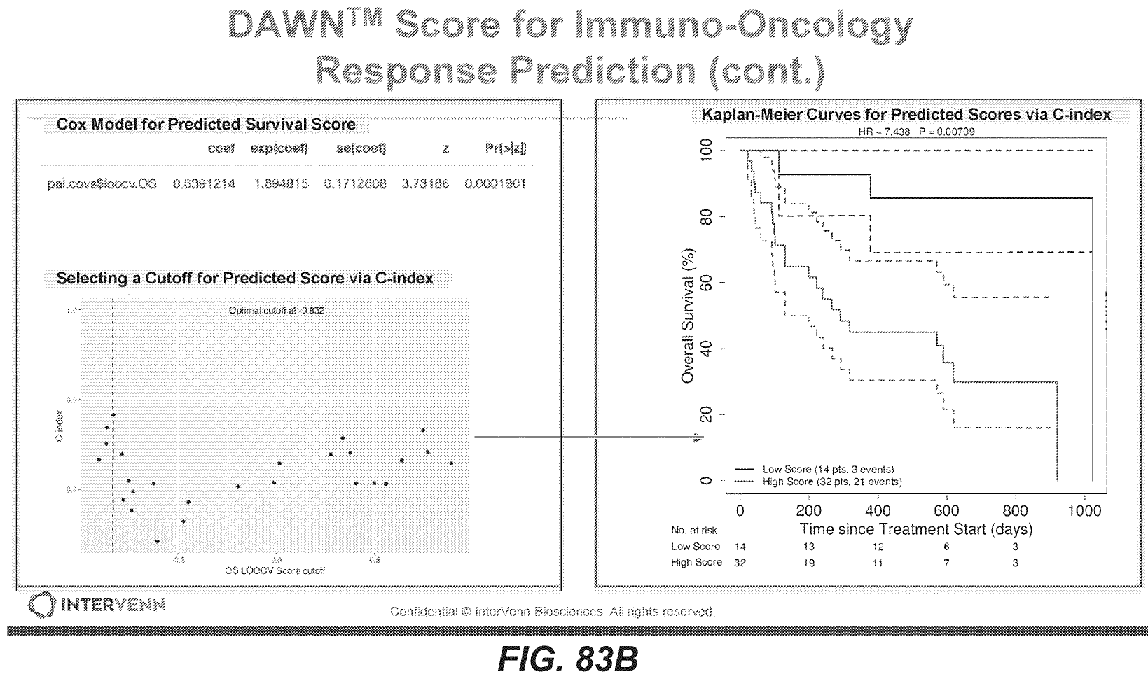

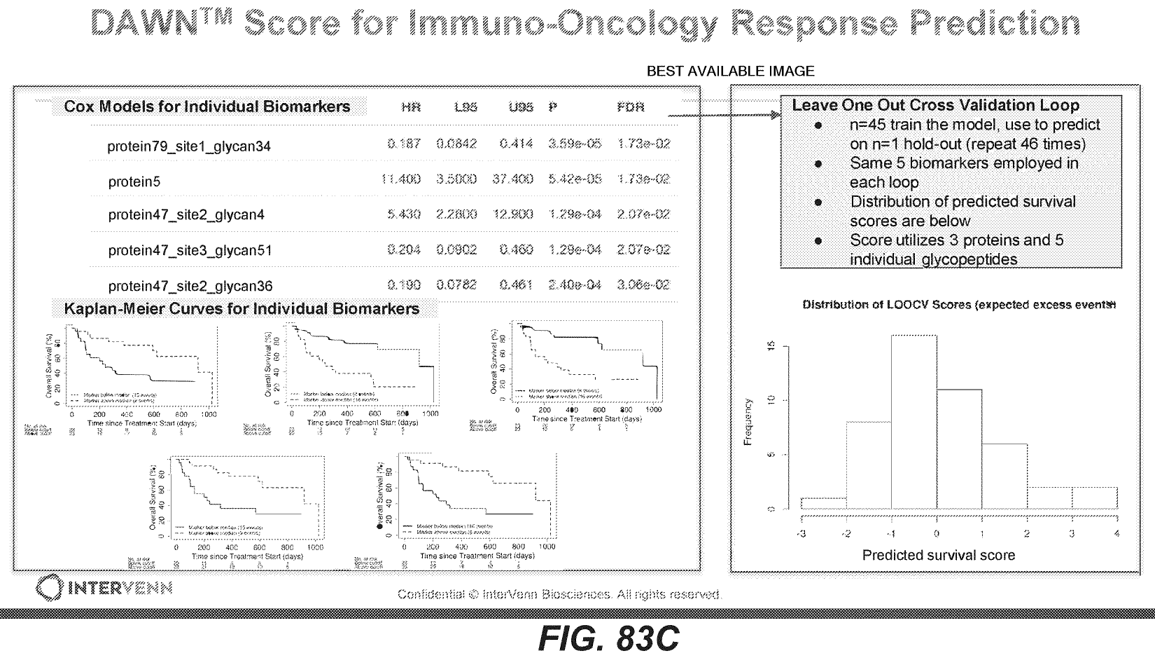

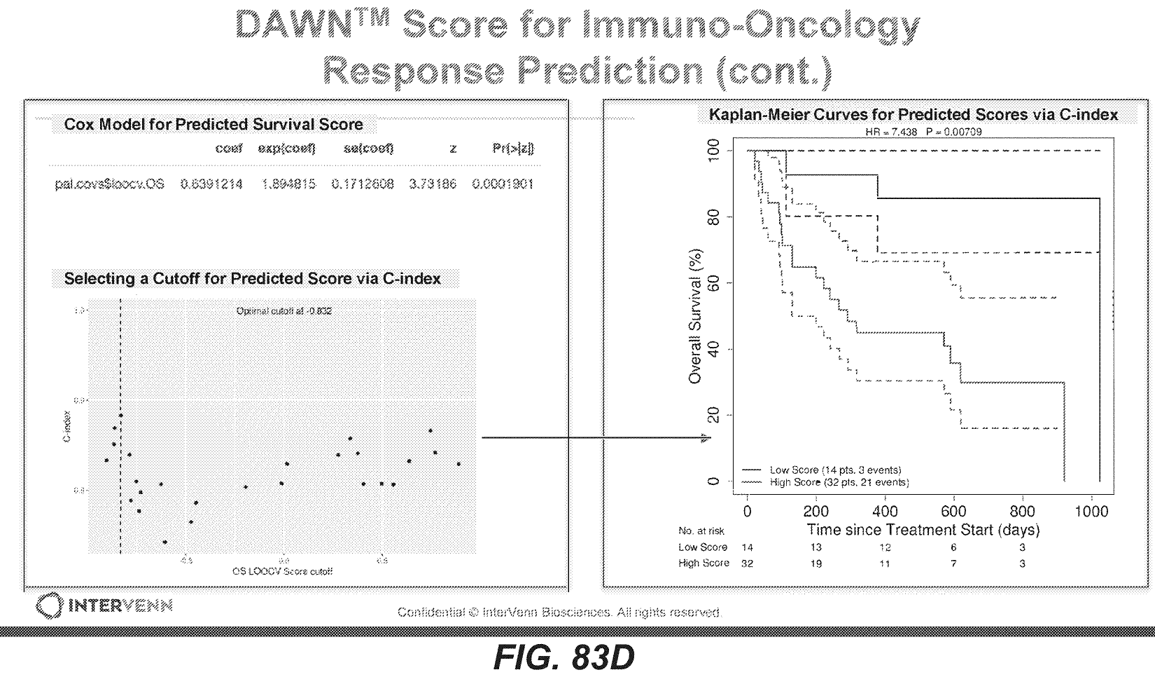

In some embodiments, the model helped to determine whether the glycopeptide marker individually predictive of OS. In some embodiments, the model helped to determine whether the glycopeptide marker individually predictive of PFS. In some embodiments, the model helped to determine whether the glycopeptide marker individually is of use in treatment selection or varied with and without treatment. In some embodiments, individual Kaplan-Meier (KM) curves are plotted for the markers relevant in each disease for each outcome, such as OS or PFS. In some embodiments, hazard ratios and p-values on the plots are representative of the plotted high/low split at median biomarker expression. Examples of individual KM curves are shown in FIGS. 16-80 for melanoma and NSCLC. FIGS. 16-41 show overall survival (OS) Kaplan-Meier curves of patients with metastatic melanoma for various glycopeptide fragments. FIGS. 42-80 show progression-free survival (PFS) Kaplan-Meier curves of patients with metastatic melanoma for various glycopeptide fragments. Examples of such multivariate KM curves generated from the individual KM curves are seen in FIGS. 14A, 14B, 15A, and 15B. FIGS. 81A and 81B illustrate an algorithm development pipeline for identifying non-small-cell lung cancer (NSCLC), in accordance with the presently disclosed embodiments. FIGS. 82A and 82B illustrate a multivariate classifier development for case-control studies for identifying non-small-cell lung cancer (NSCLC), in accordance with the presently disclosed embodiments. FIGS. 83A-83D illustrate scoring prediction curves for identifying non-small-cell lung cancer (NSCLC), in accordance with the presently disclosed embodiments.

In some embodiments, patients are treated with a therapeutically effective amount of an immune-therapeutic. In some embodiments, the immune-therapeutic comprises an immune checkpoint inhibitor. In some embodiments, the checkpoint inhibitor comprises PD-1 inhibitors, PD-L1 inhibitors, or CTLA-4 inhibitors, or combinations thereof.

In some embodiments, patients are treated with a therapeutically effective amount of a targeted therapeutic agent. In some embodiments, the targeted therapeutic agent is a drug that targets blood vessel that targets vascular endothelial growth factor (VEGF) such as bevacizumab, ramucirumab, and ziv-aflibercept. In some embodiments, the targeted therapeutic agent comprises an epidermal growth factor receptor (EGFR). In some embodiments, the EGFR comprises cetuximab or panitumumab. In some embodiments, the targeted therapeutic agent comprises a kinase inhibitor. In some embodiments, the kinase inhibitor comprises regorafenib.

In some embodiments, the patient is treated with a targeted therapy. In some embodiments, the methods herein include administering a therapeutically effective amount of one or more of 5-fluorouracil (5-FU); capecitabine, irinotecan, oxaliplatin, trifluridine, or tipiracil.

### Methods for Detecting Glycopeptides

In some embodiments, provided herein are methods for detecting one or more a multiple-reaction-monitoring (MRM) transition, comprising: obtaining a biological sample from a patient, wherein the biological sample comprises one or more glycopeptides; digesting and/or fragmenting a glycopeptide in the sample; and detecting a multiple-reaction-monitoring (MRM) transition.

In some embodiments, provided herein are methods of detecting one or more glycopeptides, wherein each glycopeptide is individually in each instance selected from a glycopeptide consisting of an amino acid sequence selected from the group consisting of SEQ ID NOs: 21-46, 101-131, and 159-207, and combinations thereof. In some embodiments, provided herein are methods of detecting one or more glycopeptides, wherein each glycopeptide is individually in each instance selected from a glycopeptide consisting essentially of an amino acid sequence selected from the group consisting of SEQ ID NO: 101-131, 159-207, and 21-46, and combinations thereof.

In some embodiments, provided herein are methods of detecting one or more glycopeptides. In some examples, set forth herein is a method of detecting one or more glycopeptide fragments. In certain examples, the method includes detecting the glycopeptide group to which the glycopeptide, or fragment thereof, belongs. In some of these examples, the glycopeptide group is selected from Alpha-1-antitrypsin (A1AT), Alpha-1B-glycoprotein (A1BG), Leucine-richAlpha-2-glycoprotein (A2GL), Alpha-2-macroglobulin (A2MG), Alpha-1-antichymotrypsin (AACT), Afamin (AFAM), Alpha-1-acid glycoprotein 1 & 2 (AGP12), Alpha-1-acid glycoprotein 1 (AGP1), Alpha-1-acid glycoprotein 2 (AGP2), Apolipoprotein A-I (APOA1), Apolipoprotein C-III (APOC3), Apolipoprotein B-100 (APOB), Apolipoprotein D (APOD), Beta-2-glycoprotein-1 (APOH), Apolipoprotein M (APOM), Attractin (ATRN), Calpain-3 (CAN3), Ceruloplasmin (CERU), Complement Factor H (CFAH), Complement Factor I (CFAI), Clusterin (CLUS), ComplementC3 (CO3), ComplementC4-A&B (CO4A&CO4B), ComplementcomponentC6 (CO6), ComplementComponentC8AChain (CO8A), Coagulation factor XII (FA12), Alpha-2-HS-glycoprotein (FETUA), Haptoglobin (HPT), Histidine-rich Glycoprotein (HRG), Immunoglobulin heavy constant alpha 1&2 (IgA12), Immunoglobulin heavy constant alpha 2 (IgA2), Immunoglobulin heavy constant gamma 2 (IgG2), Immunoglobulin heavy constant mu (IgM), Inter-alpha-trypsin inhibitor heavy chain H1 (ITIH1), Plasma Kallikrein (KLKB1), Kininogen-1 (KNG1), Serum paraoxonase/arylesterase 1 (PON1), Selenoprotein P (SEPP1), Prothrombin (THRB), Serotransferrin (TRFE), Transthyretin (TTR), Protein unc-13HomologA (UN13A), Vitronectin (VTNC), Zinc-alpha-2-glycoprotein (ZA2G), Insulin-like growth factor-II (IGF2), Apolipoprotein C-I (APOC1), and combinations thereof.

In some embodiments, provided herein are methods comprising detecting a glycopeptide, a glycan on the glycopeptide and the glycosylation site residue where the glycan bonds to the glycopeptide. In some embodiments, the method includes detecting a glycan residue. In some embodiments, the method includes detecting a glycosylation site on a glycopeptide. In some embodiments, this process is accomplished with mass spectroscopy used in tandem with liquid chromatography.

In some embodiments, provided herein are methods comprising obtaining a biological sample from a patient. In some examples, the biological sample is synovial fluid, whole blood, blood serum, blood plasma, urine, sputum, tissue, saliva, tears, spinal fluid, tissue section(s) obtained by biopsy; cell(s) that are placed in or adapted to tissue culture; sweat, mucous, fecal material, gastric fluid, abdominal fluid, amniotic fluid, cyst fluid, peritoneal fluid, pancreatic juice, breast milk, lung lavage, bone marrow, gastric acid, bile, semen, pus, aqueous humor, transudate, or combinations of the foregoing. In some examples, the biological sample is selected from the group consisting of blood, plasma, saliva, mucus, urine, stool, tissue, sweat, tears, hair, or a combination thereof. In some examples, the biological sample is a blood sample. In some examples, the biological sample is a plasma sample. In some examples, the biological sample is a saliva sample. In some examples, the biological sample is a mucus sample. In some examples, the biological sample is a urine sample. In some examples, the biological sample is a stool sample. In some examples, the biological sample is a sweat sample. In some examples, the biological sample is a tear sample. In some examples, the biological sample is a hair sample.

In some examples, the method comprises digesting and/or fragmenting a glycopeptide in the sample. In some examples, the method includes digesting a glycopeptide in the sample. In some examples, the method includes fragmenting a glycopeptide in the sample. In some examples, the digested or fragmented glycopeptide is analyzed using mass spectroscopy. In some examples, the glycopeptide is digested or fragmented in the solution phase using digestive enzymes. In some examples, the glycopeptide is digested or fragmented in the gaseous phase inside a mass spectrometer, or the instrumentation associated with a mass spectrometer. In some examples, the mass spectroscopy results are analyzed using machine-learning algorithms. In some examples, the mass spectroscopy results are the quantification of the glycopeptides, glycans, peptides, and fragments thereof. In some examples, this quantification is used as an input in a trained model to generate an output probability. The output probability is a probability of being within a given category or classification, e.g., the classification of having cancer or the classification of not having cancer. In some other examples, the output probability is a probability of being within a given category or classification, e.g., the classification of having cancer or the classification of not having cancer. In some examples, the output probability is a probability of being within a given category or classification, e.g., the classification of having an autoimmune disease or the classification of not having an autoimmune disease. In some examples, the output probability is a probability of being within a given category or classification, e.g., the classification of having fibrosis or the classification of not having fibrosis.

In some examples, the mass spectroscopy is performed using multiple reaction monitoring (MRM) mode. In some examples, the mass spectroscopy is performed using qTOF MS in data-dependent acquisition. In some examples, the mass spectroscopy is performed using or MS-only mode.

In some examples, the method comprises introducing the sample, or a portion thereof, into a mass spectrometer. In some examples, the method comprises fragmenting a glycopeptide in the sample after introducing the sample, or a portion thereof, into the mass spectrometer. In some examples, the method includes digesting a glycopeptide in the sample occurs before introducing the sample, or a portion thereof, into the mass spectrometer. In some examples, the method comprises fragmenting a glycopeptide in the sample to provide a glycopeptide ion, a peptide ion, a glycan ion, a glycan adduct ion, or a glycan fragment ion. In some examples, the method comprises digesting and/or fragmenting a glycopeptide in the sample to provide one or more glycopeptides consisting of an amino acid sequence selected from the group consisting of SEQ ID NO: 101-131, 159-207, and 21-46, and combinations thereof. In some examples, the method comprises digesting and/or fragmenting a glycopeptide in the sample to provide one or more glycopeptides consisting essentially of an amino acid sequence selected from the group consisting of SEQ ID NO: 101-131, 159-207, and 21-46, and combinations thereof.

In some examples, the method includes detecting an MRM transition indicative of a glycopeptide or glycan residue, wherein the glycopeptide consists essentially of an amino acid sequence selected from the group consisting of SEQ ID NO: 101-131, 159-207, and 21-46 and combinations thereof. In some examples, the method includes detecting an MRM transition indicative of a glycopeptide consisting essentially of an amino acid sequence selected from the group consisting of SEQ ID NO: 101-131, 159-207, and 21-46 and combinations thereof.. In some examples, the method includes detecting more than one MRM transition indicative of a combination of glycopeptides having amino acid sequences selected from a combination of SEQ ID NO: 101-131, 159-207, and 21-46.

In some examples, the method includes detecting a MRM transition indicative of a glycopeptide or glycan residue, wherein the glycopeptide consists essentially of an amino acid sequence selected from the group consisting of SEQ ID NO: 101-131, 159-207, and 21-46, and combinations thereof. In some examples, the method includes detecting a MRM transition indicative of a glycopeptide consisting essentially of an amino acid sequence selected from the group consisting of SEQ ID NO: 101-131, and combinations thereof. In some examples, the method includes detecting a MRM transition indicative of a glycopeptide consisting essentially of an amino acid sequence selected from the group consisting of SEQ ID NO: 159-207, and combinations thereof. In some examples, the method includes detecting a MRM transition indicative of a glycopeptide consisting essentially of an amino acid sequence selected from the group consisting of SEQ ID NO: 21-46, and combinations thereof.

In some examples, the method includes detecting a MRM transition indicative of a glycopeptide or glycan residue, wherein the glycopeptide consists essentially of an amino acid sequence selected from the group consisting of SEQ ID NOs: 221-46, and combinations thereof. In some examples, the method includes detecting a MRM transition indicative of a glycopeptide consisting essentially of an amino acid sequence selected from the group consisting of SEQ ID NOs: 101-131, and combinations thereof.. In some examples, the method includes detecting a MRM transition indicative of a glycopeptide or glycan residue, wherein the glycopeptide consists essentially of an amino acid sequence selected from the group consisting of 159-207.

In some examples, the method comprises performing mass spectroscopy on the biological sample using multiple-reaction-monitoring mass spectroscopy (MRM-MS).

In some examples, the method includes digesting a glycoprotein in the sample to provide one or more glycopeptides consisting essentially of an amino acid sequence selected from the group consisting of SEQ ID NO: 101-131, 159-207, and 21-46, and combinations thereof. In some examples, the biological sample is combined with chemical reagents. In some examples, the biological sample is combined with enzymes. In some examples, the enzymes are lipases. In some examples, the enzymes are proteases. In some examples, the enzymes are serine proteases. In some examples, the enzyme is selected from the group consisting of trypsin, chymotrypsin, thrombin, elastase, and subtilisin. In some examples, the enzyme is trypsin. In some examples, the methods comprises contacting at least two proteases with a glycopeptide in a sample. In some examples, the at least two proteases are selected from the group consisting of serine protease, threonine protease, cysteine protease, aspartate protease. In some examples, the at least two proteases are selected from the group consisting of trypsin, chymotrypsin, endoproteinase, Asp-N, Arg-C, Glu-C, Lys-C, pepsin, thermolysin, elastase, papain, proteinase K, subtilisin, clostripain, and carboxypeptidase protease, glutamic acid protease, metalloprotease, and asparagine peptide lyase.

In some examples, the method includes detecting an MRM transition indicative of a glycopeptide or glycan residue, wherein the glycopeptide consisting of, or consisting essentially of, an amino acid sequence selected from the group consisting of SEQ ID NO: 101-131, 159-207, and 21-46 and combinations thereof. In some examples, the method includes detecting an MRM transition indicative of a glycopeptide or glycan residue, wherein the glycopeptide consists essentially of an amino acid sequence selected from the group consisting of SEQ ID NO: 101-131, 159-207, and 21-46 and combinations thereof. In some examples, the method includes detecting an MRM transition indicative of a glycopeptide consisting essentially of an amino acid sequence selected from the group consisting of SEQ ID NO: 101-131, 159-207, and 21-46 and combinations thereof. In some examples, the method includes detecting more than one MRM transition indicative of a combination of glycopeptides having amino acid sequences selected from a combination of SEQ ID NO: 101-131, 159-207, and 21-46.

In some examples, the method includes detecting a MRM transition indicative of a glycopeptide or glycan residue, wherein the glycopeptide consists essentially of an amino acid sequence selected from the group consisting of SEQ ID NO: 101-131, 159-207, and 21-46, and combinations thereof.

In some examples, the method comprises performing mass spectroscopy on the biological sample using multiple-reaction-monitoring mass spectroscopy (MRM-MS).

In some examples, the method comprises digesting a glycopeptide in the sample to provide a glycopeptide consisting of an amino acid sequence selected from the group consisting of SEQ ID NO: 101-131, 159-207, and 21-46, and combinations thereof. In some examples, the biological sample is contacted with one or more chemical reagents. In some examples, the biological sample is contacted with one or more enzymes. In some examples, the enzymes are lipases. In some examples, the enzymes are proteases. In some examples, the enzymes are serine proteases. In some examples, the enzyme is selected from the group consisting of trypsin, chymotrypsin, thrombin, elastase, and subtilisin. In some of these examples, the enzyme is trypsin. In some examples, the methods include contacting at least two proteases with a glycopeptide in a sample. In some examples, the at least two proteases are selected from the group consisting of serine protease, threonine protease, cysteine protease, aspartate protease. In some examples, the at least two proteases are selected from the group consisting of trypsin, chymotrypsin, endoproteinase, Asp-N, Arg-C, Glu-C, Lys-C, pepsin, thermolysin, elastase, papain, proteinase K, subtilisin, clostripain, and carboxypeptidase protease, glutamic acid protease, metalloprotease, and asparagine peptide lyase.

In some examples, the method includes conducting tandem liquid chromatography-mass spectroscopy on the biological sample. In some examples, the method includes multiple-reaction-monitoring mass spectroscopy (MRM-MS) mass spectroscopy on the biological sample. In some examples, the method includes detecting an MRM transition using a triple quadrupole (QQQ) and/or a quadrupole time-of-flight (qTOF) mass spectrometer. In some examples, the method includes detecting an MRM transition using a QQQ mass spectrometer. In some examples, the method includes detecting using a qTOF mass spectrometer. In some examples, a suitable instrument for use with the instant methods is an Agilent 6495B Triple Quadrupole LC/MS. In some examples, the method includes detecting using a QQQ mass spectrometer. In some examples, a suitable instrument for use with the instant methods is an Agilent 6545 LC/Q-TOF.

In some examples, the method comprises detecting more than one MRM transition using a QQQ and/or qTOF mass spectrometer. In some examples, the method includes detecting more than one MRM transition using a QQQ mass spectrometer. In some examples, the method includes detecting more than one MRM transition using a qTOF mass spectrometer. In some examples, the method includes detecting more than one MRM transition using a QQQ mass spectrometer.

In some examples, the methods herein include quantifying one or more glycomic parameters of the one or more biological samples comprises employing a coupled chromatography procedure. In some examples, these glycomic parameters include the identification of a glycopeptide group, identification of glycans on the glycopeptide, identification of a glycosylation site, identification of part of an amino acid sequence which the glycopeptide includes. In some examples, the coupled chromatography procedure comprises: performing or effectuating a liquid chromatography-mass spectrometry (LC-MS) operation. In some examples, the coupled chromatography procedure comprises: performing or effectuating a multiple reaction monitoring mass spectrometry (MRM-MS) operation. In some examples, the methods herein include a coupled chromatography procedure which comprises: performing or effectuating a liquid chromatography-mass spectrometry (LC-MS) operation; and effectuating a multiple reaction monitoring mass spectrometry (MRM-MS) operation. In some examples, the methods include training a machine-learning algorithm using one or more glycomic parameters of the one or more biological samples obtained by one or more of a triple quadrupole (QQQ) mass spectrometry operation and/or a quadrupole time-of-flight (qTOF) mass spectrometry operation. In some examples, the methods include training a machine-learning algorithm using one or more glycomic parameters of the one or more biological samples obtained by a triple quadrupole (QQQ) mass spectrometry operation. In some examples, the methods include training a machine-learning algorithm using one or more glycomic parameters of the one or more biological samples obtained by a quadrupole time-of-flight (qTOF) mass spectrometry operation. In some examples, the methods include quantifying one or more glycomic parameters of the one or more biological samples comprises employing one or more of a triple quadrupole (QQQ) mass spectrometry operation and a quadrupole time-of-flight (qTOF) mass spectrometry operation. In some examples, machine-learning algorithms are used to quantify these glycomic parameters. In some examples, including any of the foregoing, the mass spectroscopy is performed using multiple reaction monitoring (MRM) mode. In some examples, the mass spectroscopy is performed using qTOF MS in data-dependent acquisition. In some examples, the mass spectroscopy is performed using or MS-only mode.

In some examples, the method includes detecting one or more MRM transitions indicative of glycans. In some examples, the method comprises quantifying a glycan. In some examples, the method comprises quantifying a first glycan and quantifying a second glycan; and further comprising comparing the quantification of the first glycan with the quantification of the second glycan. In some examples, the method comprises associating the detected glycan with a peptide residue site, whence the glycan was bonded. In some examples, the method comprises generating a glycosylation profile of the sample. In some examples, the method comprises associating the detected glycan with a timepoint.

In some examples, the method includes spatially profiling glycans on a tissue section associated with the sample. In some examples, including any of the foregoing, the method includes spatially profiling glycopeptides on a tissue section associated with the sample. In some examples, the method includes matrix-assisted laser desorption ionization time-of-flight mass spectrometry (MALDI-TOF) mass spectroscopy in combination with the methods herein.

In some examples, the method includes quantifying relative abundance of a glycan and/or a peptide.

In some examples, the method includes normalizing the amount of a glycopeptide by quantifying a glycopeptide consisting essentially of an amino acid sequence selected from the group consisting of SEQ ID NO: 101-131, 159-207, and 21-46, and combinations thereof and comparing that quantification to the amount of another chemical species. In some examples, the method includes normalizing the amount of a peptide by quantifying a glycopeptide consisting of an amino acid sequence selected from the group consisting of SEQ ID NO: 101-131, 159-207, and 21-46, and combinations thereof, and comparing that quantification to the amount of another glycopeptide consisting of an amino acid sequence selected from the group consisting of SEQ ID NO: 101-131, 159-207, and 21-46. In some examples, the method includes normalizing the amount of a peptide by quantifying a glycopeptide consisting essentially of an amino acid sequence selected from the group consisting of SEQ ID NO: 101-131, 159-207, and 21-46, and combinations thereof, and comparing that quantification to the amount of another glycopeptide consisting essentially of an amino acid sequence selected from the group consisting of SEQ ID NO: 101-131, 159-207, and 21-46.

### Methods for Classifying Samples Comprising Glycopeptides

In some embodiment, provided herein are methods for identifying a classification for a sample, the method comprising: quantifying by mass spectroscopy (MS) one or more glycopeptides in a sample wherein the glycopeptides each, individually in each instance, comprises a glycopeptide consisting essentially of an amino acid sequence selected from the group consisting of, or consisting essentially of SEQ ID NO: 101-131, 159-207, and 21-46, and combinations thereof; and inputting the quantification into a trained model to generate a output probability; determining if the output probability is above or below a threshold for a classification; and identifying a classification for the sample based on whether the output probability is above or below a threshold for a classification.

In some examples, provided herein are methods for identifying glycopeptide biomarkers, comprising: obtaining a biological sample from a patient; digesting and/or fragmenting a glycopeptide in the sample; detecting a multiple-reaction-monitoring (MRM) transition; and classifying the glycopeptides based on the MRM transitions detected. In some examples, a machine-learning algorithm is used to train a model using the analyzed the MRM transitions as inputs. In some examples, a machine-learning algorithm is trained using the MRM transitions as a training data set. In some examples, the methods herein include identifying glycopeptides, peptides, and glycans based on their mass spectroscopy relative abundance. In some examples, a machine-learning algorithm or algorithms select and/or identify peaks in a mass spectroscopy spectrum. In some examples, the MS is MRM-MS with a QQQ and/or qTOF mass spectrometer.

In some examples, including any of the foregoing, the mass spectroscopy is performed using multiple reaction monitoring (MRM) mode. In some examples, the mass spectroscopy is performed using qTOF MS in data-dependent acquisition. In some examples, the mass spectroscopy is performed using or MS-only mode.

In some examples, the machine-learning algorithm is selected from the group consisting of a deep learning algorithm, a neural network algorithm, an artificial neural network algorithm, a supervised machine-learning algorithm, a linear discriminant analysis algorithm, a quadratic discriminant analysis algorithm, a support vector machine algorithm, a linear basis function kernel support vector algorithm, a radial basis function kernel support vector algorithm, a random forest algorithm, a genetic algorithm, a nearest neighbor algorithm, k-nearest neighbors, a naive Bayes classifier algorithm, a logistic regression algorithm, or a combination thereof. In certain examples, the machine-learning algorithm is lasso regression.

In some examples, the method includes classifying a sample as within, or embraced by, a disease classification or a disease severity classification.

In some examples, the classification is identified with 80% confidence, 85% confidence, 90% confidence, 95% confidence, 99% confidence, or 99.9999% confidence.

In some examples, the method includes quantifying by MS the glycopeptide in a sample at a first time point; quantifying by MS the glycopeptide in a sample at a second time point; and comparing the quantification at the first time point with the quantification at the second time point.

In some examples, the method includes quantifying by MS a different glycopeptide in a sample at a third time point; quantifying by MS the different glycopeptide in a sample at a fourth time point; and comparing the quantification at the fourth time point with the quantification at the third time point.

In some examples, the method includes monitoring the health status of a patient.

In some examples, monitoring the health status of a patient includes monitoring the onset and progression of disease in a patient with risk factors such as genetic mutations, as well as detecting cancer recurrence.

In some examples, the method includes diagnosing a patient with a disease or condition based on the quantification. In some examples, the method includes treating the patient with a therapeutically effective amount of a therapeutic agent comprising one or more of a chemotherapeutic, an immunotherapy, a hormone therapy, a targeted therapy, a neoadjuvant therapy, and surgery. In some embodiments, the treatment comprises checkpoint inhibitors. In some examples, the method includes diagnosing an individual with a disease or condition based on the quantification. In some examples, the method includes treating the individual with a therapeutically effective amount of a treatment.

### Methods for Diagnosing Patients

In some examples, provided herein are methods for assessing a patient having a disease or condition, comprising measuring by mass spectroscopy a glycopeptide in a sample from the patient.

In another embodiment, provided herein are methods for assessing a patient having cancer; the method comprising: obtaining a biological sample from the patient; performing mass spectroscopy of the biological sample using MRM-MS with a QQQ and/or qTOF spectrometer to detect and quantify one or more glycopeptides consisting essentially of an amino acid sequence selected from the group consisting of SEQ ID NO: 101-131, 159-207, and 21-46; inputting the quantification of the detected glycopeptides or the MRM transitions into a trained model to generate an output probability, determining if the output probability is above or below a threshold for a classification; and identifying a diagnostic classification for the patient based on whether the output probability is above or below a threshold for a classification; and assessing the patient as having cancer based on the classification.

In another embodiment, set forth herein is a method for diagnosing a patient having cancer; the method comprising: inputting the quantification of detected glycopeptides or MRM transitions into a trained model to generate an output probability, determining if the output probability is above or below a threshold for a classification; and identifying a diagnostic classification for the patient based on whether the output probability is above or below a threshold for a classification; and assessing the patient as based on the classification. In some examples, the method includes obtaining a biological sample from the patient; performing mass spectroscopy of the biological sample using MRM-MS with a QQQ and/or qTOF spectrometer to detect and quantify one or more glycopeptides consisting essentially of an amino acid sequence selected from the group consisting of 21-46, 101-131, and 159-207.

In some examples, set forth herein is a method for assessing a patient having cancer; the method comprising: obtaining a biological sample from the patient; performing mass spectroscopy of the biological sample using MRM-MS with a QQQ and/or qTOF spectrometer to detect one or more glycopeptides consisting or, or consisting essentially of, an amino acid sequence selected from the group consisting of SEQ ID NO: 101-131, 159-207, and 21-46; analyzing the detected glycopeptides or the MRM transitions to identify a classification; and assessing the patient based on the diagnostic classification.

In some examples, set forth herein is a method for assessing a patient having cancer; the method comprising: analyzing detected or quantified glycopeptides or MRM transitions to identify a classification; and assessing the patient based on the classification. In some examples, the method includes obtaining a biological sample from the patient; and performing mass spectroscopy of the biological sample using MRM-MS with a QQQ and/or qTOF spectrometer to detect one or more glycopeptides consisting or, or consisting essentially of, an amino acid sequence selected from the group consisting of SEQ ID NO: 101-131, 159-207, and 21-46.

In some examples, set forth herein is a method for diagnosing, monitoring, or classifying aging in an individual; the method comprising: obtaining a biological sample from the patient; performing mass spectroscopy of the biological sample using MRM-MS with a QQQ and/or qTOF spectrometer to detect one or more glycopeptides consisting or, or consisting essentially of, an amino acid sequence selected from the group consisting of SEQ ID NO: 101-131, 159-207, and 21-46; analyzing the detected glycopeptides or the MRM transitions to identify a diagnostic classification; and diagnosing, monitoring, or classifying the individual as having an aging classification based on the diagnostic classification.

### Diseases and Conditions

Provided herein are biomarkers for diagnosing a variety of diseases and conditions. In some examples, the diseases and conditions include cancer. In some examples, the diseases and conditions are not limited to cancer.

In some embodiments, cancer refers to a physiological condition in a subject that is typically characterized by unregulated cell growth. Examples of cancer include, but are not limited to, melanoma, carcinoma, lymphoma, blastoma, sarcoma, and leukemia and metastases thereof. The term “metastasis” refers to the transference of disease-producing organisms or of malignant or cancerous cells to other parts of the body by way of the blood or lymphatic vessels or membranous surfaces. Non-limiting examples of such cancers include small-cell lung cancer, non-small cell lung cancer, adenocarcinoma of the lung, squamous carcinoma of the lung, melanoma, squamous cell cancer, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, thyroid cancer, hepatic carcinoma and various types of head and neck cancer. The phrase “stage of disease” refers to the stages of cancer progression referred to as Stage I, II, III, or IV. Stage of disease indicates if metastasis has occurred in the subject.

In some examples, the “patient” described herein is equivalently described as an “individual.” For example, in some methods herein, set forth are biomarkers for monitoring or diagnosing a disease or a condition in an individual. In some of these examples, the individual is not necessarily a patient who has a medical condition in need of therapy.

### Machine-Learning Model

In some examples, the methods herein comprise quantifying one or more glycopeptides consisting essentially of an amino acid sequence selected from the group consisting of SEQ ID NO: 101-131, 159-207, and 21-46 using mass spectroscopy and/or liquid chromatography. In some examples, the quantification results are used as inputs in a trained model. In some examples, the quantification results are classified or categorized with a predictive algorithm based on the absolute amount, relative amount, and/or type of each glycan or glycopeptide quantified in the test sample, wherein the predictive algorithm is trained on corresponding values for each marker obtained from a population of individuals having known diseases or conditions. In some examples, the disease or condition is cancer. In some cases, the disease or condition is melanoma. In some cases, the disease or condition is NSCLC.

In some examples, including any of the foregoing, set forth herein is a method for training a machine-learning algorithm, comprising: providing a first data set of MRM transition signals indicative of a sample comprising a glycopeptide consisting essentially of an amino acid sequence selected from the group consisting of SEQ ID NO: 101-131, 159-207, and 21-46; providing a second data set of MRM transition signals indicative of a control sample; and comparing the first data set with the second data set using a machine-learning algorithm.

In some examples, the methods herein include using a sample comprising a glycopeptide consisting of an amino acid sequence selected from the group consisting of SEQ ID NO: 101-131, 159-207, and 21-46 is a sample from a patient having the disease or condition. In some examples, the methods herein include using a sample comprising a glycopeptide consisting essentially of an amino acid sequence selected from the group consisting of SEQ ID NO: 101-131, 159-207, and 21-46 is a sample from a patient having cancer. In some examples, the methods herein include using a control sample, wherein the control sample is a sample from a patient not having the disease or condition.

In some examples, the methods herein include using a sample comprising a glycopeptide consisting essentially of an amino acid sequence selected from the group consisting of SEQ ID NO: 101-131, 159-207, and 21-46, which is a pooled sample from one or more patients having the disease or condition. In some examples, the methods herein include using a control sample, which is a pooled sample from one or more patients not having the disease or condition.

In some examples, the methods include generating machine-learning models trained using mass spectrometry data (e.g., MRM-MS transition signals) from patients having a disease or condition and patients not having a disease or condition. In some examples, the disease or condition is cancer. In some examples, the methods include optimizing the machine-learning models by cross-validation with known standards or other samples. In some examples, the methods include qualifying the performance using the mass spectrometry data to form panels of glycans and glycopeptides with individual sensitivities and specificities. In certain examples, the methods include determining a confidence percent in relation to a diagnosis. In some examples, one to ten glycopeptides consisting essentially of an amino acid sequence selected from the group consisting of SEQ ID NO: 101-131, 159-207, and 21-46 may be useful for diagnosing a patient with the disease or condition with a certain confidence percent. In some examples, ten to fifty glycopeptides consisting essentially of an amino acid sequence selected from the group consisting of SEQ ID NO: 101-131, 159-207, and 21-46 may be useful for diagnosing a patient with the disease or condition with a higher confidence percent.

In some examples, including any of the foregoing, the methods include performing MRM-MS and/or LC-MS on a biological sample. In some examples, the methods include constructing, by a computing device, theoretical mass spectra data representing a plurality of mass spectra, wherein each of the plurality of mass spectra corresponds to one or more glycopeptides consisting essentially of an amino acid sequence selected from the group consisting of SEQ ID NO: 101-131, 159-207, and 21-46. In some examples, the methods include comparing, by the computing device, the mass spectra data with the theoretical mass spectra data to generate comparison data indicative of a similarity of each of the plurality of mass spectra to each of the plurality of theoretical target mass spectra associated with a corresponding glycopeptide of the plurality of glycopeptides.

In some examples, machine-learning algorithms are used to determine, by the computing device and based on the MRM-MS data, a distribution of a plurality of characteristic ions in the plurality of mass spectra; and determining, by the computing device and based on the distribution, whether one or more of the plurality of characteristic ions is a glycopeptide ion.

In some examples, the methods herein include training a predictive algorithm. Herein, training the predictive algorithm may refer to supervised learning of a predictive algorithm on the basis of values for one or more glycopeptides consisting of, or consisting essentially of, an amino acid sequence selected from the group consisting of SEQ ID NO: 101-131, 159-207, and 21-46. Training the predictive algorithm may refer to variable selection in a statistical model on the basis of values for one or more glycopeptides consisting essentially of an amino acid sequence selected from the group consisting of SEQ ID NO: 101-131, 159-207, and 21-46. Training a predictive algorithm may for example include determining a weighting vector in feature space for each category, or determining a function or function parameters.

In some examples, the machine-learning algorithm is selected from the group consisting of a deep learning algorithm, a neural network algorithm, an artificial neural network algorithm, a supervised machine-learning algorithm, a linear discriminant analysis algorithm, a quadratic discriminant analysis algorithm, a support vector machine algorithm, a linear basis function kernel support vector algorithm, a radial basis function kernel support vector algorithm, a random forest algorithm, a genetic algorithm, a nearest neighbor algorithm, k-nearest neighbors, a naive Bayes classifier algorithm, a logistic regression algorithm, or a combination thereof. In certain examples, the machine-learning algorithm is lasso regression.

In some examples, the machine-learning algorithm is LASSO, Ridge Regression, Random Forests, K-nearest Neighbors (KNN), Deep Neural Networks (DNN), and Principal Components Analysis (PCA). In certain examples, DNN's are used to process mass spec data into analysis-ready forms. In some examples, DNN's are used for peak picking from a mass spectra. In some examples, PCA is useful in feature detection.

In some examples, LASSO is used to provide feature selection.

In some examples, machine-learning algorithms are used to quantify peptides from each protein that are representative of the protein abundance. In some examples, this quantification includes quantifying proteins for which glycosylation is not measured.

In some examples, glycopeptide sequences are identified by fragmentation in the mass spectrometer and database search using Byonic software (Protein Metrics Inc).

In some examples, the methods herein include unsupervised learning to detect features of MRMS-MS data that represent known biological quantities, such as protein function or glycan motifs. In certain examples, these features are used as input for classifying by machine-learning. In some examples, the classification is performed using LASSO, Ridge Regression, or Random Forest nature.

In some examples, the methods herein include mapping input data (e.g., MRM transition peaks) to a value (e.g., a scale based on 0-100) before processing the value in an algorithm. For example, after an MRM transition is identified and the peak characterized, the methods herein include assessing the MS scans in an m/z and retention time window around the peak for a given patient. In some examples, the resulting chromatogram is integrated by a machine-learning algorithm that determines the peak start and stop points, and calculates the area bounded by those points and the intensity (height). The resulting integrated value is the abundance, which then feeds into machine-learning and statistical analyses training and data sets.

In some examples, machine-learning output, in one instance, is used as machine-learning input in another instance. For example, in addition to the PCA being used for a classification process, the DNN data processing feeds into PCA and other analyses. This results in at least three levels of algorithmic processing. Other hierarchical structures are contemplated within the scope of the instant disclosure.

In some examples, the methods include comparing the amount of each glycan or glycopeptide quantified in the sample to corresponding reference values for each glycan or glycopeptide in a predictive algorithm. In some examples, the methods include a comparative process by which the amount of a glycan or glycopeptide quantified in the sample is compared to a reference value for the same glycan or glycopeptide using a predictive algorithm. The comparative process may be part of a classification by a predictive algorithm. The comparative process may occur at an abstract level, e.g., in n-dimensional feature space or in a higher dimensional space.

In some examples, the methods herein include classifying a patient's sample based on the amount of each glycan or glycopeptide quantified in the sample with a predictive algorithm. In some examples, the methods include using statistical or machine-learning classification processes by which the amount of a glycan or glycopeptide quantified in the test sample is used to determine a category of health with a predictive algorithm. In some examples, the predictive algorithm is a statistical or machine-learning classification algorithm.

In some examples, classification by a predictive algorithm may include scoring likelihood of a panel of glycan or glycopeptide values belonging to each possible category, and determining the highest-scoring category. Classification by a predictive algorithm may include comparing a panel of marker values to previous observations by means of a distance function. Examples of predictive algorithms suitable for classification include random forests, support vector machines, logistic regression (e.g. multiclass or multinomial logistic regression, and/or algorithms adapted for sparse logistic regression). A wide variety of other predictive algorithms that are suitable for classification may be used, as known to a person skilled in the art.

In some examples, the methods herein include supervised learning of a predictive algorithm on the basis of values for each glycan or glycopeptide obtained from a population of individuals having a disease or condition (e.g., melanoma or NSCLS). In some examples, the methods include variable selection in a statistical model on the basis of values for each glycan or glycopeptide obtained from a population of individuals having the disease or condition. Training a predictive algorithm may for example include determining a weighting vector in feature space for each category, or determining a function or function parameters.

In one embodiment, the reference value is the amount of a glycan or glycopeptide in a sample or samples derived from one individual. Alternatively, the reference value may be derived by pooling data obtained from multiple individuals, and calculating an average (for example, mean or median) amount for a glycan or glycopeptide. Thus, the reference value may reflect the average amount of a glycan or glycopeptide in multiple individuals. Said amounts may be expressed in absolute or relative terms, in the same manner as described herein.

In some examples, the reference value may be derived from the same sample as the sample that is being tested, thus allowing for an appropriate comparison between the two. For example, if the sample is derived from urine, the reference value is also derived from urine. In some examples, if the sample is a blood sample (e.g. a plasma or a serum sample), then the reference value will also be a blood sample (e.g. a plasma sample or a serum sample, as appropriate). When comparing between the sample and the reference value, the way in which the amounts are expressed is matched between the sample and the reference value. Thus, an absolute amount can be compared with an absolute amount, and a relative amount can be compared with a relative amount. Similarly, the way in which the amounts are expressed for classification with the predictive algorithm is matched to the way in which the amounts are expressed for training the predictive algorithm.

When the amounts of the glycan or glycopeptide are determined, the method may comprise comparing the amount of each glycan or glycopeptide to its corresponding reference value. When the cumulative amount of one, some or all the glycan or glycopeptides are determined, the method may comprise comparing the cumulative amount to a corresponding reference value. When the amounts of the glycan or glycopeptides are combined with each other in a formula to form an index value, the index value can be compared to a corresponding reference index value derived in the same manner.

The reference values may be obtained either within (i.e., constituting a step of) or external to the (i.e., not constituting a step of) methods described herein. In some examples, the methods include a step of establishing a reference value for the quantity of the markers. In other examples, the reference values are obtained externally to the method described herein and accessed during the comparison step of the invention.

In certain embodiments, the lasso regression machine-learning model may be a regression model or other classification model that may be evaluated utilizing receiver operating characteristic (ROC) evaluation and/or area under curve (AOC) evaluation. For example, in certain embodiments, as will be further illustrated with respect to FIGS. 14A, 14B, 15A, and 15B, the ROC model evaluation may represent a plot of sensitivity rate (e.g., patient likely not responsive) against a plot of specificity rate (patient likely to be responsive) and may be further optimized based on an iterative tuning of hyperparameters of the lasso regression machine-learning model. The trained the lasso regression machine-learning model may be then utilized to predict patient overall survival (OS) and progression-free survival (PFS) patients with metastatic melanoma for various glycopeptide fragments and patients with non-small-cell lung cancer (NSCLC) for various glycopeptide fragments, in accordance with the presently disclosed embodiments.

In some examples, including any of the foregoing, training of a predictive algorithm may be obtained either within (i.e., constituting a step of) or external to (i.e., not constituting a step of) the methods set forth herein. In some examples, the methods include a step of training of a predictive algorithm. In some examples, the predictive algorithm is trained externally to the method herein and accessed during the classification step of the invention. The reference value may be determined by quantifying the amount of a glycan or glycopeptide in a sample obtained from a population of healthy individual(s). The predictive algorithm may be trained by quantifying the amount of a glycan or glycopeptide in a sample obtained from a population of healthy individual(s). As used herein, the term “healthy individual” refers to an individual or group of individuals who are in a healthy state, e.g., patients who have not shown any symptoms of the disease, have not been diagnosed with the disease and/or are not likely to develop the disease. Preferably said healthy individual(s) is not on medication affecting the disease and has not been diagnosed with any other disease. The one or more healthy individuals may have a similar sex, age and body mass index (BMI) as compared with the test individual. The reference value may be determined by quantifying the amount of a glycan or glycopeptide in a sample obtained from a population of individual(s) suffering from the disease. The predictive algorithm may be trained by quantifying the amount of a marker in a sample obtained from a population of individual(s) suffering from the disease. More preferably such individual(s) may have similar sex, age and body mass index (BMI) as compared with the test individual. The reference value may be obtained from a population of individuals suffering from cancer. The predictive algorithm may be trained by quantifying the amount of a glycan or glycopeptide in a sample obtained from a population of individuals suffering from cancer. Once the characteristic glycan or glycopeptide profile of cancer is determined, the profile of markers from a biological sample obtained from an individual may be compared to this reference profile to determine whether the test subject also has cancer. Once the predictive algorithm is trained to classify cancer, the profile of markers from a biological sample obtained from an individual may be classified by the predictive algorithm to determine whether the test subject is also at that particular stage of cancer.

### Kits

In some examples, including any of the foregoing, set forth herein is a kit comprising a glycopeptide standard, a buffer, and one or more glycopeptides consisting of an amino acid sequence selected from the group consisting of SEQ ID NO: 101-131, 159-207, and 21-46.

In some examples, including any of the foregoing, set forth herein is a kit comprising a glycopeptide standard, a buffer, and one or more glycopeptides consisting essentially of an amino acid sequence selected from the group consisting of SEQ ID NO: 101-131, 159-207, and 21-46.

In some examples, set forth herein is a kit comprising a glycopeptide standard, a buffer, and one or more glycopeptides consisting of an amino acid sequence selected from the group consisting of SEQ ID NO: 101-131, 159-207, and 21-46, and combinations thereof. In some examples, set forth herein is a kit comprising a glycopeptide standard, a buffer, and one or more glycopeptides consisting essentially of an amino acid sequence selected from the group consisting of SEQ ID NO: 101-131. In some examples, set forth herein is a kit comprising a glycopeptide standard, a buffer, and one or more glycopeptides consisting essentially of an amino acid sequence selected from the group consisting of SEQ ID NO: 159-207. In some examples, set forth herein is a kit comprising a glycopeptide standard, a buffer, and one or more glycopeptides consisting essentially of an amino acid sequence selected from the group consisting of SEQ ID NO: 21-46.

In some examples, set forth herein is a kit for diagnosing or monitoring cancer in an individual wherein the glycan or glycopeptide profile of a sample from said individual is determined and the measured profile is compared with a profile of a normal patient or a profile of a patient with a family history of cancer. In some examples, the kit comprises one or more glycopeptides consisting of an amino acid sequence selected from the group consisting SEQ ID NO: 101-131, 159-207, and 21-46. In some examples, the kit comprises one or more glycopeptides consisting essentially of an amino acid sequence selected from the group consisting of SEQ ID NO: 101-131, 159-207, and 21-46.

In some examples, set forth herein is a kit comprising the reagents for quantification of the oxidized, nitrated, and/or glycated free adducts derived from glycopeptides.

### Clinical Assays

In some examples, the biomarkers, methods, and/or kits may be used in a clinical setting for diagnosing patients. In some of these examples, the analysis of samples includes the use of internal standards. These standards may include one or more glycopeptides consisting of an amino acid sequence selected from the group consisting of SEQ ID NO: 101-131, 159-207, and 21-46. These standards may include one or more glycopeptides consisting essentially of an amino acid sequence selected from the group consisting of SEQ ID NO: 101-131, 159-207, and 21-46.

In a clinical setting, samples may be prepared (e.g., by digestion) to include one or more glycopeptides consisting of an amino acid sequence selected from the group consisting of SEQ ID NO: 101-131, 159-207, and 21-46. In a clinical setting, samples may be prepared (e.g., by digestion) to include one or more glycopeptides consisting essentially of an amino acid sequence selected from the group consisting of SEQ ID NO: 101-131, 159-207, and 21-46. In some examples, the amount of a glycan or glycopeptide may be assessed by comparing the amount of one or more glycopeptides consisting of an amino acid sequence selected from the group consisting of SEQ ID NO: 101-131, 159-207, and 21-46 to the concentration of another biomarker. In some examples, the amount of a glycan or glycopeptide may be assessed by comparing the amount of one or more glycopeptides consisting essentially of an amino acid sequence selected from the group consisting of SEQ ID NO: 101-131, 159-207, and 21-46 to the concentration of another biomarker.

In some examples, the amount of a glycan or glycopeptide may be assessed by comparing the amount of one or more glycopeptides consisting of an amino acid sequence selected from the group consisting of SEQ ID NOs: 300-429 the amount of one or more glycopeptides consisting of an amino acid sequence selected from the group consisting of SEQ ID NOs: 300-429.

In some examples, the amount of a glycan or glycopeptide may be assessed by comparing the amount of one or more glycopeptides consisting essentially of an amino acid sequence selected from the group consisting of SEQ ID NO: 101-131, 159-207, and 21-46 to the amount of one or more glycopeptides consisting essentially of an amino acid sequence selected from the group consisting of SEQ ID NO: 101-131, 159-207, and 21-46.

In some examples, including any of the foregoing, the kit may include software for computing the normalization of a glycopeptide MRM transition signal.

In some examples, including any of the foregoing, the kit may include software for quantifying the amount of a glycopeptide consisting of, or consisting essentially of, an amino acid sequence selected from the group consisting of SEQ ID NO: 101-131, 159-207, and 21-46. In some examples, including any of the foregoing, the kit may include software for quantifying the relative amount of a glycopeptide consisting of, or consisting essentially of, an amino acid sequence selected from the group consisting of SEQ ID NO: 101-131, 159-207, and 21-46.

In some examples, including any of the foregoing, a trained model is stored on a server which is accessed by a clinician performing a method, set forth herein. In some examples, the clinician inputs the quantification of the MRM transition signals from a patient's sample into a trained model which are stored on a server. In some examples, the server is accessed by the internet, wireless communication, or other digital or telecommunication methods.

In some examples, including any of the foregoing, a trained model is stored on a server which is accessed by a clinician performing a method, set forth herein. In some examples, the clinician inputs the quantification of the glycopeptide or glycopeptides consisting of, or consisting essentially of, an amino acid sequence selected from the group consisting of SEQ ID NO: 101-131, 159-207, and 21-46 from a patient's sample into a trained model which are stored on a server.

In some examples, the server is accessed by the internet, wireless communication, or other digital or telecommunication

Individual KM curves may be plotted for the markers relevant in for the disease interest in four files. Hazard ratios and p-values on the plots are representative of the plotted high/low split at median biomarker expression. FIGS. 14A and 14B show progression-free survival (PFS) Kaplan-Meier curves of patients with metastatic melanoma for various glycopeptide fragments. FIGS. 15A and 15B show progression-free survival (PFS) Kaplan-Meier curves of patients with non-small-cell lung cancer (NSCLC) for various glycopeptide fragments. FIGS. 16-41 show overall survival (OS) Kaplan-Meier curves of patients with metastatic melanoma for various glycopeptide fragments of interest for melanoma. FIGS. 42-80 show progression-free survival (PFS) Kaplan-Meier curves of patients with metastatic melanoma for various glycopeptide fragments for melanoma.

IV. Additional Proteins and Glycopeptides

TABLE 12
Glycopeptides associated with melanoma

| | | | | Linking | |
|---|---|---|---|---|---|
| | | | | Site Pos. | Glycan |
| SEQ ID | Transition | Peptide Structure | | in Peptide | Structure |
| NO: | Number | (PS) NAME | Peptide Sequence | Sequence | GL NO. |
| SEQ ID | 101 | QUANTPEP.A2GL_ | DLLLPQPDLR | | N/A |
| NO: 101 | | DLLLPQPDLR | | | |
| SEQ ID | 102 | QUANTPEP.ANGT_ | SLDFTELDVAAEK | | N/A |
| NO: 102 | | SLDFTELDVAAEK | | | |
| SEQ ID | 103 | A1AT_70_5412 | QLAHQSNSTNIFFSPV | 1 | 5412 |
| NO: 103 | | | SIATAFAMLSLGTK | | |
| SEQ ID | 104 | HPT_184_5412 | MVSHHNLTTGATLIN | 1 | 5412 |
| NO: 104 | | | EQWLLTTAK | | |
| SEQ ID | 105 | HPT_241_6513 | VVLHPNYSQVDIGLIK | 3 | 6513 |
| NO: 105 | | | | | |
| SEQ ID | 106 | HEMO_187_5412 | SWPAVGNCSSALR | 2 | 5412 |
| NO: 106 | | | | | |
| SEQ ID | 107 | IC1_48_1102 | VATTVISK | 1 | 1102 |
| NO: 107 | | | | | |
| SEQ ID | 108 | HPT_184_6513 | MVSHHNLTTGATLIN | 1 | 6513 |
| NO: 108 | | | EQWLLTTAK | | |
| SEQ ID | 109 | APOC3_74_ | FSEFWDLDPEVRPTSA | 1 | N/A |
| NO: 109 | | NONGLYCOSYLATED | VAA | | |
| SEQ ID | 110 | IGM_209_5500 | GLTFQQNASSMCVPD | 2 | 5500 |
| NO: 110 | | | QDTAIR | | |
| SEQ ID | 112 | FETUA_156_5412 | VCQDCPLLAPLNDTR | 1 | 5412 |
| NO: 112 | | | | | |
| SEQ ID | 113 | QUANTPEP.B2M_ | VNHVTLSQPK | 1 | |
| NO: 113 | | VNHVTLSQPK | | | |
| SEQ ID | 114 | IC1_253_5412 | VLSNNSDANLELINT | 3 | 5412 |
| NO: 114 | | | WVAK | | |
| SEQ ID | 115 | CERU_138_5412 | EHEGAIYPDNTTDFQR | 1 | 5412 |
| NO: 115 | | | | | |
| SEQ ID | 116 | IGM_209_5501 | GLTFQQNASSMCVPD | 2 | 5501 |
| NO: 116 | | | QDTAIR | | |
| SEQ ID | 117 | THRB_416MC_5402 | WVLTAAHCLLYPPWD | 3 | 5402 |
| NO: 117 | | | KNFTENDLLVR | | |
| SEQ ID | 118 | TRFE_630_5412 | QQQHLFGSNVTDCSG | 2 | 5412 |
| NO: 118 | | | NFCLFR | | |
| SEQ ID | 119 | FETUA_176_6501 | AALAAFNAQNNGSNF | 2 | 6501 |
| NO: 119 | | | QLEEISR | | |
| SEQ ID | 120 | CO5_741_5412 | ANISHK | 1 | 5412 |
| NO: 120 | | | | | |
| SEQ ID | 121 | FETUA_176_5412 | AALAAFNAQNNGSNF | 2 | 5412 |
| NO: 121 | | | QLEEISR | | |
| SEQ ID | 122 | CFAH_911_5401 | ISEENETTCYMGK | 3 | 5401 |
| NO: 122 | | | | | |
| SEQ ID | 123 | IGG1_297_4511 | EEQYNSTYR | 1 | 4511 |
| NO: 123 | | | | | |
| SEQ ID | 124 | A2MG_247_5200 | IITILEEEMNVSVCGLY | 2 | 5200 |
| NO: 124 | | | TYGKPVPGHVTVSICR | | |
| SEQ ID | 125 | CERU_138_5402 | EHEGAIYPDNTTDFQR | 1 | 5402 |
| NO: 125 | | | | | |
| SEQ ID | 126 | IGA2_205_4510 | TPLTANITK | 1 | 4510 |
| NO: 126 | | | | | |
| SEQ ID | 127 | HRG_125_5402 | VIDFNCTTSSVSSALA | 1 | 5402 |
| NO: 127 | | | NTK | | |
| SEQ ID | 128 | HPT_207_121005 | NLFLNHSENATAK | 2 | 121005 |
| NO: 128 | | | | | |
| SEQ ID | 129 | AACT_106_7604 | FNLTETSEAEIHQSFQ | 1 | 7604 |
| NO: 129 | | | HLLR | | |
| SEQ ID | 130 | CERU_397_6503 | ENLTAPGSDSAVFFEQ | 3 | 6503 |
| NO: 130 | | | GTTR | | |
| SEQ ID | 131 | HPT_207_11904 | NLFLNHSENATAK | 2 | 11904 |
| NO: 131 | | | | | |

TABLE 13
Glycoproteins Associated with Melanoma

| SEQ | Protein | | Uniprot |
|---|---|---|---|
| ID NO. | Abbreviation | Protein Name | ID |
| SEQ ID | A2GL | Leucine-richAlpha- | P02750 |
| NO: 132 | | 2-glycoprotein | |
| SEQ ID | ANGT | P01019\| | P01019 |
| NO: 133 | | Angiotensinogen | |
| SEQ ID | HPT | Haptoglobin | P00738 |
| NO: 134 | | | |
| SEQ ID | HEMO | Hemopexin | P02790 |
| NO: 135 | | | |
| SEQ ID | IC1 | Plasma protease | P05155 |
| NO: 136 | | C1 inhibitor | |
| SEQ ID | HPT | Haptoglobin | P00738 |
| NO: 137 | | | |
| SEQ ID | APOC3 | Apolipoprotein | P02656 |
| NO: 138 | | C-III | |
| SEQ ID | IGM | Immunoglobulin | P01871 |
| NO: 139 | | heavy constant mu | |
| SEQ ID | FETUA | Alpha-2-HS- | P02765 |
| NO: 140 | | glycoprotein | |
| SEQ ID | B2M | Beta-2- | P61769 |
| NO: 141 | | microglobulin | |
| SEQ ID | IC1 | Plasma protease | P05155 |
| NO: 142 | | C1 inhibitor | |
| SEQ ID | CERU | Ceruloplasmin | P00450 |
| NO: 143 | | | |
| SEQ ID | IGM | Immunoglobulin | P01871 |
| NO: 144 | | heavy constant mu | |
| SEQ ID | THRB | Prothrombin | P00734 |
| NO: 145 | | | |
| SEQ ID | TRFE | Serotransferrin | P02787 |
| NO: 146 | | | |
| SEQ ID | FETUA | Alpha-2-HS- | P02765 |
| NO: 147 | | glycoprotein | |
| SEQ ID | CO5 | ComplementC5 | P01031 |
| NO: 148 | | | |
| SEQ ID | FETUA | Alpha-2-HS- | P02765 |
| NO: 149 | | glycoprotein | |
| SEQ ID | CFAH | ComplementFactorH | P08603 |
| NO: 150 | | | |
| SEQ ID | IGG1 | Immunoglobulin | P01857 |
| NO: 151 | | heavy constant | |
| | | gamma 1 | |
| SEQ ID | A2MG | Alpha-2- | P01023 |
| NO: 152 | | macroglobulin | |
| SEQ ID | CERU | Ceruloplasmin | P00450 |
| NO: 153 | | | |
| SEQ ID | IGA2 | Immunoglobulin | P01877 |
| NO: 154 | | heavy constant | |
| | | alpha 2 | |
| SEQ ID | HRG | Histidine-rich | P04196 |
| NO: 155 | | Glycoprotein | |
| SEQ ID | HPT | Haptoglobin | P00738 |
| NO: 156 | | | |
| SEQ ID | AACT | Alpha-1- | P01011 |
| NO: 157 | | antichymotrypsin | |
| SEQ ID | HPT | Haptoglobin | P00738 |
| NO: 158 | | | |

TABLE 14
Glycopeptides Associated with NSCLC

| | | | | Linking | |
|---|---|---|---|---|---|
| | | | | Site Pos. | Glycan |
| SEQ ID | Transition | Peptide Structure | | in Peptide | Structure |
| NO: | Number | (PS) NAME | Peptide Sequence | Sequence | GL NO. |
| SEQ ID | 159 | TRFE_630_6513 | QQQHLFGSNVTDCSG | 9 | 6513 |
| NO: 159 | | | NFCLFR | | |
| SEQ ID | 160 | AGP1_93_6503 | QDQCIYNTTYLNVQR | 7 | 6503 |
| NO: 160 | | | | | |
| SEQ ID | 161 | IGG2_297_5510 | EEQFNSTFR | 5 | 5510 |
| NO: 161 | | | | | |
| SEQ ID | 162 | IGGI_297_5410 | EEQYNSTYR | 5 | 5410 |
| NO: 162 | | | | | |
| SEQ ID | 163 | AACT_271_6502 | YTGNASALFILPDQDK | 4 | 6502 |
| NO: 163 | | | | | |
| SEQ ID | 164 | AGP1_103_6503 | ENGTISR | 2 | 6503 |
| NO: 164 | | | | | |
| SEQ ID | 165 | IGG1_297_3410 | EEQYNSTYR | 5 | 3410 |
| NO: 165 | | | | | |
| SEQ ID | 166 | IGG1_297_5510 | EEQYNSTYR | 5 | 5510 |
| NO: 166 | | | | | |
| SEQ ID | 167 | VTNC_86_6503 | NNATVHEQVGGPSLT | 2 | 6503 |
| NO: 167 | | | SDLQAQSK | | |
| SEQ ID | 168 | HPT_241_6513 | VVLHPNYSQVDIGLIK | 6 | 6513 |
| NO: 168 | | | | | |
| SEQ ID | 169 | CERU_762_6523 | ELHHLQEQNVSNAFL | 9 | 6523 |
| NO: 169 | | | DK | | |
| SEQ ID | 170 | HRG_345_5412 | HSHNNNSSDLHPHK | 6 | 5412 |
| NO: 170 | | | | | |
| SEQ ID | 171 | HPT_207_5401 | NLFLNHSENATAK | 5 | 5401 |
| NO: 171 | | | | | |
| SEQ ID | 172 | AGP1_93_8704 | QDQCIYNTTYLNVQR | 7 | 8704 |
| NO: 172 | | | | | |
| SEQ ID | 173 | HRG_125_5402 | VIDFNCTTSSVSSALA | 5 | 5402 |
| NO: 173 | | | NTK | | |
| SEQ ID | 174 | A1AT_271_5401 | YLGNATAIFFLPDEGK | 4 | 5401 |
| NO: 174 | | | | | |
| SEQ ID | 175 | KNG1_205_5412 | ITYSIVQTNCSK | 9 | 5412 |
| NO: 175 | | | | | |
| SEQ ID | 176 | TRFE_432_5401 | CGLVPVLAENYNK | 12 | 5401 |
| NO: 176 | | | | | |
| SEQ ID | 177 | IGG2_297_5410 | EEQFNSTFR | 5 | 5410 |
| NO: 177 | | | | | |
| SEQ ID | 178 | TRFE_630_5400 | QQQHLFGSNVTDCSG | 9 | 5400 |
| NO: 178 | | | NFCLFR | | |
| SEQ ID | 179 | AGP1_93_7603 | QDQCIYNTTYLNVQR | 7 | 7603 |
| NO: 179 | | | | | |
| SEQ ID | 180 | CERU_762_6512 | ELHHLQEQNVSNAFL | 9 | 6512 |
| NO: 180 | | | DK | | |
| SEQ ID | 181 | A1AT_107_6502 | ADTHDEILEGLNFNLT | 14 | 6502 |
| NO: 181 | | | EIPEAQIHEGFQELLR | | |
| SEQ ID | 182 | KLKB1_494_5400 | LQAPLNYTEFQKPICL | 6 | 5400 |
| NO: 182 | | | PSK | | |
| SEQ ID | 183 | IGC1_297_5411 | EEQYNSTYR | 5 | 5411 |
| NO: 183 | | | | | |
| SEQ ID | 184 | HPT_207_121005 | NLFLNHSENATAK | 5.9 | 121005 |
| NO: 184 | | | | | |
| SEQ ID | 185 | FETUA_176_5412 | AALAAFNAQNNGSNF | 11 | 5412 |
| NO: 185 | | | QLEEISR | | |
| SEQ ID | 186 | HPT_241_5412 | VVLHPNYSQVDIGLIK | 6 | 5412 |
| NO: 186 | | | | | |
| SEQ ID | 187 | CFAH_882_5401 | IPCSQPPQIEHGTINSSR | 15 | 5401 |
| NO: 187 | | | | | |
| SEQ ID | 188 | AGP1_93_6502 | QDQCIYNTTYLNVQR | 7 | 6502 |
| NO: 188 | | | | | |
| SEQ ID | 189 | IC1_352_5412 | VGQLQLSHNLSLVILV | 9 | 5412 |
| NO: 189 | | | PQNLK | | |
| SEQ ID | 190 | HEMO_187_NON- | SWPAVGNCSSALR | 7 | NONGLYCO- |
| NO: 190 | | GLYCOSYLATED | | | SYLATED |
| SEQ ID | 191 | KLKB1_396_5401 | IVGGTNSSWGEWPWQ | 6 | 5401 |
| NO: 191 | | | VSLQVK | | |
| SEQ ID | 192 | IGJ_71_5412 | ENISDPTSPLR | 2 | 5412 |
| NO: 192 | | | | | |
| SEQ ID | 193 | AGP12_72MC_7614 | SVQEIQATFFYFTPNK | 15 | 7614 |
| NO: 193 | | | TEDTIELR | | |
| SEQ ID | 194 | TRFE_630_5401 | QQQHLFGSNVTDCSG | 9 | 5401 |
| NO: 194 | | | NFCLFR | | |
| SEQ ID | 195 | TRFE_630_5411 | QQQHLFGSNVTDCSG | 9 | 5411 |
| NO: 195 | | | NFCLFR | | |
| SEQ ID | 196 | IGM_209_5512 | GLTFQQNASSMCVPD | 7 | 5512 |
| NO: 196 | | | QDTAIR | | |
| SEQ ID | 197 | KNG1_137_NON- | FSVATQTCQITPAEGPVVTAQY | N/A | NONGLYCO- |
| NO: 197 | | GLYCOSYLATED | DCLGCVHPISTQSPDLEPILR | | SYLATED |
| SEQ ID | 198 | FHR1_126_5402 | LQNNENNISCVER | 7 | 5402 |
| NO: 198 | | | | | |
| SEQ ID | 199 | IGG1_297_4500 | EEQYNSTYR | 5 | 4500 |
| NO: 199 | | | | | |
| SEQ ID | 200 | AGP1_93_7612 | QDQCIYNTTYLNVQR | 7 | 7612 |
| NO: 200 | | | | | |
| SEQ ID | 201 | A1AT_271_5402 | YLGNATAIFFLPDEGK | 4 | 5402 |
| NO: 201 | | | | | |
| SEQ ID | 202 | A1AT_271_6503 | YLGNATAIFFLPDEGK | 4 | 6503 |
| NO: 202 | | | | | |
| SEQ ID | 203 | KNG1_294_5412 | LNAENNATFYFK | 6 | 5412 |
| NO: 203 | | | | | |
| SEQ ID | 204 | CO2_621_6200 | QSVPAHFVALNGSK | 11 | 6200 |
| NO: 204 | | | | | |
| SEQ ID | 205 | HRG_271_2202 | SSTTKPPFKPHGSR | 1 | 2202 |
| NO: 205 | | | | | |
| SEQ ID | 206 | APOD_98_5412 | ADGTVNQIEGEATPVN | 16 | 5412 |
| NO: 206 | | | LTEPAK | | |
| SEQ ID | 207 | AEAM_33_5402 | DIENFNSTQK | 6 | 5402 |
| NO: 207 | | | | | |

TABLE 15
Glycoproteins associated with NSCLC

| SEQ ID | Protein | | |
|---|---|---|---|
| NO. | Abbreviation | Protein Name | Uniprot ID |
| SEQ ID | TRFE | Serotransferrin | P02787 |
| NO: 208 | | | |
| SEQ ID | AGP1 | Alpha-1-acid glycoprotein 1 | P02763 |
| NO: 209 | | | |
| SEQ ID | IGG2 | Immunoglobulin heavy constant gamma 2 | P01859 |
| NO: 210 | | | |
| SEQ ID | IGG1 | Immunoglobulin heavy constant gamma 1 | P01857 |
| NO: 211 | | | |
| SEQ ID | AACT | Alpha-1-antichymotrypsin | P01011 |
| NO: 212 | | | |
| SEQ ID | AGP1 | Alpha-1-acid glycoprotein 1 | P02763 |
| NO: 213 | | | |
| SEQ ID | IGG1 | Immunoglobulin heavy constant gamma 1 | P01857 |
| NO: 214 | | | |
| SEQ ID | VTNC | Vitronectin | P04004 |
| NO: 215 | | | |
| SEQ ID | HPT | Haptoglobin | P00738 |
| NO: 216 | | | |
| SEQ ID | CERU | Ceruloplasmin | P00450 |
| NO: 217 | | | |
| SEQ ID | HRG | Histidine-rich Glycoprotein | P04196 |
| NO: 218 | | | |
| SEQ ID | HPT | Haptoglobin | P00738 |
| NO: 219 | | | |
| SEQ ID | AGP1 | Alpha-1-acid glycoprotein 1 | P02763 |
| NO: 220 | | | |
| SEQ ID | HRG | Histidine-rich Glycoprotein | P04196 |
| NO: 221 | | | |
| SEQ ID | A1AT | Alpha-1-antitrypsin | P01009 |
| NO: 222 | | | |
| SEQ ID | KNG1 | Kininogen-1 | P01042 |
| NO: 223 | | | |
| SEQ ID | TRFE | Serotransferrin | P02787 |
| NO: 224 | | | |
| SEQ ID | IGG2 | Immunoglobulin heavy constant gamma 2 | P01859 |
| NO: 225 | | | |
| SEQ ID | TRFE | Serotransferrin | P02787 |
| NO: 226 | | | |
| SEQ ID | AGP1 | Alpha-1-acid glycoprotein 1 | P02763 |
| NO: 227 | | | |
| SEQ ID | CERU | Ceruloplasmin | P00450 |
| NO: 228 | | | |
| SEQ ID | A1AT | Alpha-1-antitrypsin | P01009 |
| NO: 229 | | | |
| SEQ ID | KLKB1 | Plasma Kallikrein | P03952 |
| NO: 230 | | | |
| SEQ ID | IGG1 | Immunoglobulin heavy constant gamma 1 | P01857 |
| NO: 231 | | | |
| SEQ ID | HPT | Haptoglobin | P00738 |
| NO: 232 | | | |
| SEQ ID | FETUA | Alpha-2-HS-glycoprotein | P02765 |
| NO: 233 | | | |
| SEQ ID | HPT | Haptoglobin | P00738 |
| NO: 234 | | | |
| SEQ ID | CFAH | Complement Factor H | P08603 |
| NO: 235 | | | |
| SEQ ID | AGP1 | Alpha-1-acid glycoprotein 1 | P02763 |
| NO: 236 | | | |
| SEQ ID | IC1 | Plasma protease C1 inhibitor | P05155 |
| NO: 237 | | | |
| SEQ ID | HEMO | Hemopexin | P02790 |
| NO: 238 | | | |
| SEQ ID | KLKB1 | Plasma Kallikrein | P03952 |
| NO: 239 | | | |
| SEQ ID | IGJ | Immunoglobulin J chain | P01591 |
| NO: 240 | | | |
| SEQ ID | AGP1 & 2 | Alpha-1-acid glycoprotein 1 & 2 | P02763 & |
| NO: 241 | | | P19652 |
| SEQ ID | TRFE | Serotransferrin | P02787 |
| NO: 242 | | | |
| SEQ ID | IGM | Immunoglobulin heavy constant mu | P01871 |
| NO: 243 | | | |
| SEQ ID | KNG1 | Kininogen-1 | P01042 |
| NO: 244 | | | |
| SEQ ID | FHR1 | Complement factor H-related protein 1 | Q03591 |
| NO: 245 | | | |
| SEQ ID | IGG1 | Immunoglobulin heavy constant gamma 1 | P01857 |
| NO: 246 | | | |
| SEQ ID | AGP1 | Alpha-1-acid glycoprotein 1 | P02763 |
| NO: 247 | | | |
| SEQ ID | A1AT | Alpha-1-antitrypsin\|A1AT | P01009 |
| NO: 248 | | | |
| SEQ ID | KNG1 | Kininogen-1\|KNG1 | P01042 |
| NO: 249 | | | |
| SEQ ID | CO2 | ComplementC2 | P06681 |
| NO: 250 | | | |
| SEQ ID | HRG | Histidine-rich Glycoprotein | P04196 |
| NO: 251 | | | |
| SEQ ID | APOD | Apolipoprotein D | P05090 |
| NO: 252 | | | |
| SEQ ID | AFAM | Afamin | P43652 |
| NO: 253 | | | |

TABLE 16
Glycopeptides

| | | | | | Linking | |
|---|---|---|---|---|---|---|
| Peptide | | | | | Site | Glycan |
| SEQ | | Protein | Protein | | Pos. in | Structure |
| ID | Peptide Structure | Abbre- | SEQ | | Protein | GL |
| NO: | (PS) Name | viation | ID | Peptide Sequence | Sequence | NO. |
| 300 | A1AT_70_5412 | A1AT | 430 | QLAHQSNSTNIFFSPVSI | 70 | 5412 |
| | | | | ATAFAMLSLGTK | | |
| 301 | AFAM_33_5402 | AFAM | 465 | DIENFNSTQK | 33 | 5402 |
| 302 | AGP1_93_6502 | AGP1 | 449 | QDQCIYNTTYLNVQR | 93 | 6502 |
| 303 | AGP12_72MC_7614 | AGP1 | 449 | SVQEIQATFFYFTPNKT | 72 | 7614 |
| | | AGP2 | 470 | EDTIFLR | | |
| 304 | CFAH_911_5401 | CFAH | 461 | ISEENETTCYMGK | 911 | 5401 |
| 305 | HPT_207_10803 | HPT | 436 | NLFLNHSENATAK | 207 | 10803 |
| 306 | HPT_207_5401 | HPT | 436 | NLFLNHSENATAK | 207 | 5401 |
| 307 | HPT_241_5412 | HPT | 436 | VVLHPNYSQVDIGLIK | 241 | 5412 |
| 308 | IGG1_297_5400 | IGG1 | 443 | EEQYNSTYR | 297 | 5400 |
| 309 | TRFE_630_5400 | TRFE | 451 | QQQHLFGSNVTDCSGN | 630 | 5400 |
| | | | | FCLFR | | |
| 310 | AGP1_33_6503 | AGP1 | 449 | QIPLCANLVPVPITNAT | 33 | 6503 |
| | | | | LDQITGK | | |
| 311 | UN13A_1005_7512 | | | ACLNSTYEYIFNNCHEL | 1005 | 7512 |
| | | | | YSR | | |
| 312 | A1AT_107_6502 | A1AT | 430 | ADTHDEILEGLNFNLTE | 107 | 6502 |
| | | | | IPEAQIHEGFQELLR | | |
| 313 | A1AT_271_5401 | A1AT | 430 | YLGNATAIFFLPDEGK | 271 | 5401 |
| 314 | A1AT_271_5402 | A1AT | 430 | YLGNATAIFFLPDEGK | 271 | 5402 |
| 315 | A1AT_271_6503 | A1AT | 430 | YLGNATAIFFLPDEGK | 271 | 6503 |
| 316 | A2MG_247_5200 | A2MG | 439 | IITILEEEMNVSVCGLYT | 247 | 5200 |
| | | | | YGKPVPGHVTVSICR | | |
| 317 | A2MG_869_5200 | A2MG | 439 | SLGNVNFTVSAEALESQ | 869 | 5200 |
| | | | | ELCGTEVPSVPEHGR | | |
| 318 | A2MG_869_6200 | A2MG | 439 | SLGNVNFTVSAEALESQ | 869 | 6200 |
| | | | | ELCGTEVPSVPEHGR | | |
| 319 | AACT_106_7604 | AACT | 437 | FNLTETSEAEIHQSFQH | 106 | 7604 |
| | | | | LLR | | |
| 320 | AACT_127_5401 | AACT | 437 | TLNQSSDELQLSMGNA | 127 | 5401 |
| | | | | MFVK | | |
| 321 | AACT_271_6502 | AACT | 437 | YTGNASALFILPDQDK | 271 | 6502 |
| 322 | AACT_271_7602 | AACT | 437 | YTGNASALFILPDQDK | 271 | 7602 |
| 323 | AACT_271_7603 | AACT | 437 | YTGNASALFILPDQDK | 271 | 7603 |
| 324 | AGP1_103_6503 | AGP1 | 449 | ENGTISR | 103 | 6503 |
| 325 | AGP1_33_6502 | AGP1 | 449 | QIPLCANLVPVPITNAT | 33 | 6502 |
| | | | | LDQITGK | | |
| 326 | AGP1_33_6503 | AGP1 | 449 | QIPLCANLVPVPITNAT | 33 | 6503 |
| | | | | LDQITGK | | |
| 327 | AGP1_93_6503 | AGP1 | 449 | QDQCIYNTTYLNVQR | 93 | 6503 |
| 328 | AGP1_93_7603 | AGP1 | 449 | QDQCIYNTTYLNVQR | 93 | 7603 |
| 329 | AGP1_93_7612 | AGP1 | 449 | QDQCIYNTTYLNVQR | 93 | 7612 |
| 330 | AGP1_93_8704 | AGP1 | 449 | QDQCIYNTTYLNVQR | 93 | 8704 |
| 331 | APOC3_74_NONGLYCOSYLATED | APOC3 | 447 | FSEFWDLDPEVRPTSAV | 74 | NON- |
| | | | | AA | | GLYCOSYLATED |
| 332 | APOD_98_5412 | APOD | 456 | ADGTVNQIEGEATPVN | 98 | 5412 |
| | | | | LTEPAK | | |
| 333 | CERU_138_5402 | CERU | 434 | EHEGAIYPDNTTDFQR | 138 | 5402 |
| 334 | CERU_138_5412 | CERU | 434 | EHEGAIYPDNTTDFQR | 138 | 5412 |
| 335 | CERU_397_6503 | CERU | 434 | ENLTAPGSDSAVFFEQG | 397 | 6503 |
| | | | | TTR | | |
| 336 | CERU_762_6512 | CERU | 434 | ELHHLQEQNVSNAFLD | 762 | 6512 |
| | | | | K | | |
| 337 | CERU_762_6523 | CERU | 434 | ELHHLQEQNVSNAFLD | 762 | 6523 |
| | | | | K | | |
| 338 | CFAH_1029_5401 | CFAH | 461 | MDGASNVTCINSR | 1029 | 5401 |
| 339 | CFAH_1029_5402 | CFAH | 461 | MDGASNVTCINSR | 1029 | 5402 |
| 340 | CFAH_882_5401 | CFAH | 461 | IPCSQPPQIEHGTINSSR | 882 | 5401 |
| 341 | CLUS_374_6501 | CLUS | 462 | LANLTQGEDQYYLR | 374 | 6501 |
| 342 | CO2_621_6200 | CO2 | 459 | QSVPAHFVALNGSK | 621 | 6200 |
| 343 | CO5_741_5412 | COS | 440 | ANISHK | 741 | 5412 |
| 344 | CO8B_243_6610 | CO8B | 460 | EYESYSDFERNVTEK | 243 | 6610 |
| 345 | CO8B_553_5410 | CO8B | 460 | WNCWSNWSSCSGR | 553 | 5410 |
| 346 | FETUA_156_5402 | FETUA | 450 | VCQDCPLLAPLNDTR | 156 | 5402 |
| 347 | FETUA_156_5412 | FETUA | 450 | VCQDCPLLAPLNDTR | 156 | 5412 |
| 348 | FETUA_176_5412 | FETUA | 450 | AALAAFNAQNNGSNFQ | 176 | 5412 |
| | | | | LEEISR | | |
| 349 | FETUA_176_6501 | FETUA | 450 | AALAAFNAQNNGSNFQ | 176 | 6501 |
| | | | | LEEISR | | |
| 350 | FHR1_126_5402 | FHR1 | 467 | LQNNENNISCVER | 126 | 5402 |
| 351 | HEMO_187_5412 | HEMO | 452 | SWPAVGNCSSALR | 187 | 5412 |
| 352 | HEMO_187_NONGLYCOSYLATED | HEMO | 452 | SWPAVGNCSSALR | 187 | NON- |
| | | | | | | GLYCOSYLATED |
| 353 | HEMO_453_5401 | HEMO | 452 | ALPQPQNVTSLLGCTH | 453 | 5401 |
| 354 | HPT_184_5412 | HPT | 436 | MVSHHNLTTGATLINE | 184 | 5412 |
| | | | | QWLLTTAK | | |
| 355 | HPT_184_5511 | HPT | 436 | MVSHHNLTTGATLINE | 184 | 5511 |
| | | | | QWLLTTAK | | |
| 356 | HPT_184_6513 | HPT | 436 | MVSHHNLTTGATLINE | 184 | 6513 |
| | | | | QWLLTTAK | | |
| 357 | HPT_207_11904 | HPT | 436 | NLFLNHSENATAK | 207 | 11904 |
| 358 | HPT_207_121005 | HPT | 436 | NLFLNHSENATAK | 207 | 121005 |
| 359 | HPT_241_5401 | HPT | 436 | VVLHPNYSQVDIGLIK | 241 | 5401 |
| 360 | HPT_241_6513 | HPT | 436 | VVLHPNYSQVDIGLIK | 241 | 6513 |
| 361 | HRG_125_5401 | HRG | 455 | VIDFNCTTSSVSSALAN | 125 | 5401 |
| | | | | TK | | |
| 362 | HRG_125_5402 | HRG | 455 | VIDFNCTTSSVSSALAN | 125 | 5402 |
| | | | | TK | | |
| 363 | HRG_271_2202 | HRG | 455 | SSTTKPPFKPHGSR | 271 | 2202 |
| 364 | HRG_345_5412 | HRG | 455 | HSHNNNSSDLHPHK | 345 | 5412 |
| 365 | IC1_253_5412 | IC1 | 457 | VLSNNSDANLELINTW | 253 | 5412 |
| | | | | VAK | | |
| 366 | IC1_352_5412 | IC1 | 457 | VGQLQLSHNLSLVILVP | 352 | 5412 |
| | | | | QNLK | | |
| 367 | IC1_48_1102 | IC1 | 457 | VATTVISK | 48 | 1102 |
| 368 | IGA12_144_3500 | IGA1 | 6 | LSLHRPALEDLLLGSEA | 144 | 3500 |
| | | IGA2 | 446 | NLTCTLTGLR | | |
| 369 | IGA12_144_4401 | IGA1 | 6 | LSLHRPALEDLLLGSEA | 144 | 4401 |
| | | IGA2 | 446 | NLTCTLTGLR | | |
| 370 | IGA12_144_4500 | IGA1 | 6 | LSLHRPALEDLLLGSEA | 144 | 4500 |
| | | IGA2 | 446 | NLTCTLTGLR | | |
| 371 | IGA12_144_5501 | IGA1 | 6 | LSLHRPALEDLLLGSEA | 144 | 5501 |
| | | IGA2 | 446 | NLTCTLTGLR | | |
| 372 | IGA12_144_5502 | IGA1 | | LSLHRPALEDLLLGSEA | 144 | 5502 |
| | | IGA2 | 446 | NLTCTLTGLR | | |
| 373 | IGA2_205_4510 | IGA2 | 446 | TPLTANITK | 205 | 4510 |
| 374 | IGG1_297_3410 | IGG1 | 443 | EEQYNSTYR | 297 | 3410 |
| 375 | IGG1_297_4400 | IGG1 | 443 | EEQYNSTYR | 297 | 4400 |
| 376 | IGG1_297_4500 | IGG1 | 443 | EEQYNSTYR | 297 | 4500 |
| 377 | IGG1_297_4510 | IGG1 | 443 | EEQYNSTYR | 297 | 4510 |
| 378 | IGG1_297_4511 | IGG1 | 443 | EEQYNSTYR | 297 | 4511 |
| 379 | IGG1_297_5410 | IGG1 | 443 | EEQYNSTYR | 297 | 5410 |
| 380 | IGG1_297_5411 | IGG1 | 443 | EEQYNSTYR | 297 | 5411 |
| 381 | IGG1_297_5510 | IGG1 | 443 | EEQYNSTYR | 297 | 5510 |
| 382 | IGG2_297_4411 | IGG2 | 210 | EEQFNSTFR | 297 | 4411 |
| 383 | IGG2_297_4500 | IGG2 | 210 | EEQFNSTFR | 297 | 4500 |
| 384 | IGG2_297_5410 | IGG2 | 210 | EEQFNSTFR | 297 | 5410 |
| 385 | IGG2_297_5411 | IGG2 | 210 | EEQFNSTFR | 297 | 5411 |
| 386 | IGG2_297_5510 | IGG2 | 210 | EEQFNSTFR | 297 | 5510 |
| 387 | IGG34_297_4410 | IGG3 | 14 | EEQYNSTFR | 297 | 4410 |
| | | IGG4 | 444 | | | |
| 388 | IGG34_297_4411 | IGG3 | 14 | EEQYNSTFR | 297 | 4411 |
| | | IGG4 | 444 | | | |
| 389 | IGL71_5412 | IGJ | 442 | ENISDPTSPLR | 71 | 5412 |
| 390 | IGM_209_5500 | IGM | 445 | GLTFQQNASSMCVPDQ | 209 | 5500 |
| | | | | DTAIR | | |
| 391 | IGM_209_5501 | IGM | 445 | GLTFQQNASSMCVPDQ | 209 | 5501 |
| | | | | DTAIR | | |
| 392 | IGM_209_5510 | IGM | 445 | GLTFQQNASSMCVPDQ | 209 | 5510 |
| | | | | DTAIR | | |
| 393 | IGM_209_5512 | IGM | 445 | GLTFQQNASSMCVPDQ | 209 | 5512 |
| | | | | DTAIR | | |
| 394 | ITIH4_517_5401 | ITIH4 | 468 | LPTQNITFQTESSVAEQ | 517 | 5401 |
| | | | | EAEFQSPK | | |
| 395 | KLKB1_127_5410 | KLKB1 | 453 | GVNFNVSK | 127 | 5410 |
| 396 | KLKB1_396_5401 | KLKB1 | 453 | IVGGTNSSWGEWPWQ | 396 | 5401 |
| | | | | VSLQVK | | |
| 397 | KLKB1_494_5400 | KLKB1 | 453 | LQAPLNYTEFQKPICLP | 494 | 5400 |
| | | | | SK | | |
| 398 | KLKB1_494_5410 | KLKB1 | 453 | LQAPLNYTEFQKPICLP | 494 | 5410 |
| | | | | SK | | |
| 399 | KNG1_137_NONGLYCOSYLATED | KNG1 | 441 | FSVATQTCQITPAEGPV | 137 | NON- |
| | | | | VTAQYDCLGCVHPIST | | GLYCOSYLATED |
| | | | | QSPDLEPILR | | |
| 400 | KNG1_205_5412 | KNG1 | 441 | ITYSIVQTNCSK | 205 | 5412 |
| 401 | KNG1_294_5412 | KNG1 | 441 | LNAENNATFYFK | 294 | 5412 |
| 402 | NEWQUANTPEP- | IGG3 | 14 | TPEVTCVVVDVSHEDP | N/A | N/A |
| | IGG3_TPEVTCVVVD | | | EVQFK | | |
| | VSHEDPEVQFK | | | | | |
| 403 | QUANTPEP.A1AT_A | A1AT | 430 | AVLTIDEK | N/A | N/A |
| | VLTIDEK | | | | | |
| 404 | QUANTPEP.A2GL_D | A2GL | 448 | DLLLPQPDLR | N/A | N/A |
| | LLLPQPDLR | | | | | |
| 405 | QUANTPEP.ANGT_S | ANGT | 438 | SLDFTELDVAAEK | N/A | N/A |
| | LDFTELDVAAEK | | | | | |
| 406 | QUANTPEP.B2M_VN | B2M | 466 | VNHVTLSQPK | N/A | N/A |
| | HVTLSQPK | | | | | |
| 407 | QUANTPEP.IGG4_TT | IGG4 | 444 | TTPPVLDSDGSFFLYSR | N/A | N/A |
| | PPVLDSDGSFFLYSR | | | | | |
| 408 | QUANTPEP.TRFE_D | TFRE | 451 | DDTVCLAK | N/A | N/A |
| | DTVCLAK | | | | | |
| 409 | THBG_36_5402THBG | THBG | 458 | VTACHSSQPNATLYK | 36 | 5402 |
| 410 | THRB_416MC_5402 | THRB | 433 | WVLTAAHCLLYPPWD | 416 | 5402 |
| | | | | KNFTENDLLVR | | |
| 411 | TRFE_432_5401 | TRFE | 451 | CGLVPVLAENYNK | 432 | 5401 |
| 412 | TRFE_630_5401 | TRFE | 451 | QQQHLFGSNVTDCSGN | 630 | 5401 |
| | | | | FCLFR | | |
| 413 | TRFE_630_5411 | TRFE | 451 | QQQHLFGSNVTDCSGN | 630 | 5411 |
| | | | | FCLFR | | |
| 414 | TRFE_630_5412 | TRFE | 451 | QQQHLFGSNVTDCSGN | 630 | 5412 |
| | | | | FCLFR | | |
| 415 | TRFE_630_6513 | TRFE | 451 | QQQHLFGSNVTDCSGN | 630 | 6513 |
| | | | | FCLFR | | |
| 416 | VTNC_169_5401 | VTNC | 454 | NGSLFAFR | 169 | 5401 |
| 417 | VTNC_86_6503 | VTNC | 454 | NNATVHEQVGGPSLTS | 86 | 6503 |
| | | | | DLQAQSK | | |
| 418 | PON1_324_6501 | PON1 | 464 | VTQVYAENGTVLQGST | 324 | 6501 |
| | | | | VASVYK | | |
| 419 | UN13A_1005_5431 | UN13A | 469 | ACLNSTYEYIFNNCHEL | 1005 | 5431 |
| | | | | YSR | | |
| 420 | CAN3_366_6513 | CAN3 | 463 | NPWGQVEWNGSWSDR | 366 | 6513 |
| 421 | UN13A_1005_7420 | UN13A | 469 | ACLNSTYEYIFNNCHEL | 1005 | 7420 |
| | | | | YSR | | |
| 422 | CAN3_366_6503 | CAN3 | 463 | NPWGQVEWNGSWSDR | 366 | 6503 |
| 423 | AACT_106_7604 | AACT | 437 | FNLTETSEAEIHQSFQH | 106 | 7604 |
| | | | | LLR | | |
| 424 | A1AT_107_5411 | A1AT | 430 | ADTHDEILEGLNFNLTE | 107 | 5411 |
| | | | | IPEAQIHEGFQELLR | | |
| 425 | AGP1_33_5402 | AGP1 | 449 | QIPLCANLVPVPITNAT | 33 | 5402 |
| | | | | LDQITGK | | |
| 426 | FETUA_176_7600 | FETUA | 450 | AALAAFNAQNNGSNFQ | 176 | 7600 |
| | | | | LEEISR | | |
| 427 | ITIH4_517_5420.5401 | ITIH5 | 468 | LPTQNITFQTESSVAEQ | 517 | 5420.5401 |
| | | | | EAEFQSPK | | |
| 428 | PON1_324_6502 | PON1 | 464 | VTQVYAENGTVLQGST | 324 | 6502 |
| | | | | VASVYK | | |
| 429 | AGP1_33_6502 | AGP1 | 449 | QIPLCANLVPVPITNAT | 33 | 6502 |
| | | | | LDQITGK | | |

TABLE 17
Glycoproteins

| SEQ | Protein | | Uniprot |
|---|---|---|---|
| ID NO: | Abbreviation | Protein Name | ID |
| 430 | A1AT | Alpha-1-antitrypsin | P01009 |
| 431 | A2MG | Alpha-2-macroglobulin | P01023 |
| 432 | KLKB1 | Plasma Kallikrein | P03952 |
| 433 | THRB | Prothrombin | P00734 |
| 434 | CERU | Ceruloplasmin | P00450 |
| 435 | THRB | Prothrombin | P00734 |
| 436 | HPT | Haptoglobin | P00738 |
| 437 | AACT | Alpha-1-antichymotrypsin | P01011 |
| 438 | ANGT | Angiotensinogen | P01019 |
| 439 | A2MG | Alpha-2-macroglobulin | P01023 |
| 440 | CO5 | ComplementC5 | P01031 |
| 441 | KNG1 | Kininogen-1 | P01042 |
| 442 | IGJ | Immunoglobulin J chain | P01591 |
| 443 | IGG1 | Immunoglobulin heavy constant | P01857 |
| | | gamma 1 | |
| 444 | IGG4 | Immunoglobulin heavy constant | P01861 |
| | | gamma 4 | |
| 445 | IGM | Immunoglobulin heavy constant | P01871 |
| | | mu | |
| 446 | IGA2 | Immunoglobulin heavy constant | P01877 |
| | | alpha 2 | |
| 447 | APOC3 | Apolipoprotein C-III | P02656 |
| 448 | A2GL | Leucine-richAlpha-2-glycoprotein | P02750 |
| 449 | AGP1 | Alpha-1-acid glycoprotein 1 | P02763 |
| 450 | FETUA | Alpha-2-HS-glycoprotein | P02765 |
| 451 | TREE | Serotransferrin | P02787 |
| 452 | HEMO | Hemopexin | P02790 |
| 453 | KLKB1 | Plasma Kallikrein | P03952 |
| 454 | VTNC | Vitronectin | P04004 |
| 455 | HRG | Histidine-rich Glycoprotein | P04196 |
| 456 | APOD | Apolipoprotein D | P05090 |
| 457 | IC1 | Plasma protease C1 inhibitor | P05155 |
| 458 | THBG | Thyroxine-bindingGlobulin | P05543 |
| 459 | CO2 | ComplementC2 | P06681 |
| 460 | CO8B | ComplementComponentC8BChain | P07358 |
| 461 | CFAH | ComplementFactorH | P08603 |
| 462 | CLUS | Clusterin | P10909 |
| 463 | CAN3 | Calpain-3 | P20807 |
| 464 | PON1 | Serum paraoxonase/arylesterase 1 | P27169 |
| 465 | AFAM | Afamin | P43652 |
| 466 | B2M | Beta-2-microglobulin | P61769 |
| 467 | FHR1 | Complement factor H-related | Q03591 |
| | | protein 1 | |
| 468 | ITIH4 | Inter-alpha-trypsin inhibitor heavy | Q14624 |
| | | chain H4 | |
| 469 | UN13A | Protein unc-13HomologA | Q9UPW8 |
| 470 | AGP2 | Alpha-1-acid glycoprotein 2 | P19652 |

TABLE 18
Protein abbreviation, glycosylation site, glycan structure, precursor ion m/z,
and product ion m/z for transitions associated with melanoma

| Transition | | | | Precursor | Product |
|---|---|---|---|---|---|
| Number | Protein | Site | Structure | m/z | m/z |
| 101 | A2GL | N/A | N/A | 590.3 | 725.4 |
| 102 | ANGT | N/A | N/A | 719.4 | 316.2 |
| 103 | A1AT | 70 | 5412 | 1107.7 | 366.1 |
| 104 | HPT | 184 | 5412 | 1258.7 | 366.1 |
| 105 | HPT | 241 | 6513 | 1201.5 | 366.1 |
| 106 | HEMO | 187 | 5412 | 1253.2 | 366.1 |
| 107 | IC1 | 48 | 1102 | 883.4 | 274.1 |
| 108 | HPT | 184 | 6513 | 1138.4 | 366.1 |
| 109 | APOC3 | 74 | NONGLYCOSYLATED | 1069.2 | 1097.59 |
| 110 | IGM | 209 | 5500 | 1042.4 | 366.1 |
| 112 | FETUA | 156 | 5412 | 1031.9 | 204.1 |
| 113 | B2M | N/A | N/A | 561.8 | 244.2 |
| 114 | IC1 | 253 | 5412 | 1114.2 | 204.1 |
| 115 | CERU | 138 | 5412 | 1062.2 | 366.1 |
| 116 | IGM | 209 | 5501 | 1115 | 366.1 |
| 117 | THRB | 416 | 5402 | 1076.5 | 274.1 |
| 118 | TRFE | 630 | 5412 | 1217.7 | 366.1 |
| 119 | FETUA | 176 | 6501 | 1161.7 | 366.1 |
| 120 | CO5 | 741 | 5412 | 1007.7 | 366.1 |
| 121 | FETUA | 176 | 5412 | 1180.2 | 366.1 |
| 122 | CFAH | 911 | 5401 | 1159.4 | 366.1 |
| 123 | IGG1 | 297 | 4511 | 1097.8 | 204.1 |
| 124 | A2MG | 247 | 5200 | 1239.1 | 1314.2 |
| 125 | CERU | 138 | 5402 | 1025.7 | 274.1 |
| 126 | IGA2 | 205 | 4510 | 923.5 | 366.1 |
| 127 | HRG | 125 | 5402 | 1056.2 | 366.1 |
| 128 | HPT | 207 | 121005 | 1378.9 | 366.1 |
| 129 | AACT | 106 | 7604 | 1184.9 | 274.1 |
| 130 | CERU | 397 | 6503 | 998.8 | 204.1 |
| 131 | HPT | 207 | 11904 | 1247.7 | 366.1 |

TABLE 19
Retention time, Δ retention time, and collision energy for
transitions associated with melanoma

| Transition | Retention Time | Delta Retention | Collision |
|---|---|---|---|
| Number | (min) | Time | Energy |
| 101 | 30.53 | 1.4 | 15 |
| 102 | 30.46 | 1.2 | 21 |
| 103 | 47.02 | 2 | 27 |
| 104 | 33.49 | 1.4 | 31 |
| 105 | 31.15 | 1.4 | 30 |
| 106 | 21.54 | 1.5 | 30 |
| 107 | 11.64 | 1.4 | 25 |
| 108 | 34.56 | 1.4 | 28 |
| 109 | 38.45 | N/A | N/A |
| 110 | 23.6 | 1.4 | 30 |
| 112 | 27.38 | 1.6 | 30 |
| 113 | 9.46 | 1.2 | 25 |
| 114 | 35.71 | 1.4 | 30 |
| 115 | 16.6 | 1.4 | 25 |
| 116 | 25.38 | 1.4 | 20 |
| 117 | 40.57 | 1.4 | 20 |
| 118 | 32.42 | 1.8 | 30 |
| 119 | 30.11 | 1.4 | 29 |
| 120 | 4.17 | 1.6 | 30 |
| 121 | 30.61 | 1.4 | 29 |
| 122 | 12.23 | 1.4 | 35 |
| 123 | 8.61 | 1.3 | 15 |
| 124 | 38.71 | 1.3 | 25 |
| 125 | 16.83 | 1.4 | 20 |
| 126 | 12.44 | 1.4 | 22 |
| 127 | 28.65 | 1.4 | 25 |
| 128 | 13.48 | 1.5 | 35 |
| 129 | 38.45 | 1.2 | 30 |
| 130 | 27.87 | 1.4 | 40 |
| 131 | 13.45 | 1.5 | 31 |

TABLE 20
Protein abbreviation, glycosylation site, glycan
structure, precursor ion m/z, and product ion m/z
for transitions associated with NSCLC

| Transition | | | | Precursor | Product |
|---|---|---|---|---|---|
| Number | Protein | Site | Structure | m/z | m/z |
| 159 | TRFE | 630 | 6513 | 1105.6 | 366.1 |
| 160 | AGP1 | 93 | 6503 | 1195.3 | 366.1 |
| 161 | IGG2 | 297 | 5510 | 1043.8 | 366.1 |
| 162 | IGG1 | 297 | 5410 | 987.1 | 366.1 |
| 163 | AACT | 271 | 6502 | 1441.6 | 366.1 |
| 164 | AGP1 | 103 | 6503 | 1213.3 | 366.1 |
| 165 | IGG1 | 297 | 3410 | 879 | 204.1 |
| 166 | IGG1 | 297 | 5510 | 1054.7 | 366.1 |
| 167 | VTNC | 86 | 6503 | 1311.8 | 366.1 |
| 168 | HPT | 241 | 6513 | 1201.5 | 366.1 |
| 169 | CERU | 762 | 6523 | 1295 | 274.1 |
| 170 | HRG | 345 | 5412 | 994.4 | 366.1 |
| 171 | HPT | 207 | 5401 | 1124.8 | 366.1 |
| 172 | AGP1 | 93 | 8704 | 967.4 | 366.1 |
| 173 | HRG | 125 | 5402 | 1056.2 | 366.1 |
| 174 | A1AT | 271 | 5401 | 1224.5 | 366.1 |
| 175 | KNG1 | 205 | 5412 | 942.4 | 274.1 |
| 176 | TRFE | 432 | 5401 | 1131.1 | 366.1 |
| 177 | IGG2 | 297 | 5410 | 976.1 | 366.1 |
| 178 | TRFE | 630 | 5400 | 1035.6 | 366.1 |
| 179 | AGP1 | 93 | 7603 | 1286.6 | 366.1 |
| 180 | CERU | 762 | 6512 | 1186 | 366.1 |
| 181 | A1AT | 107 | 6502 | 1253.6 | 366.1 |
| 182 | KLKB1 | 494 | 5400 | 968.2 | 366.1 |
| 183 | IGG1 | 297 | 5411 | 1084.1 | 366.1 |
| 184 | HPT | 207 | 121005 | 1378.9 | 366.1 |
| 185 | FETUA | 176 | 5412 | 1180.2 | 366.1 |
| 186 | HPT | 241 | 5412 | 1383 | 366.1 |
| 187 | CFAH | 882 | 5401 | 984.7 | 366.1 |
| 188 | AGP1 | 93 | 6502 | 1122.5 | 366.1 |
| 189 | IC1 | 352 | 5412 | 1167.3 | 366.1 |
| 190 | HEMO | 187 | NONGLYCOSYLATED | 703.3 | 566.3 |
| 191 | KLKB1 | 396 | 5401 | 1069.2 | 204.1 |
| 192 | IGJ | 71 | 5412 | 1193.8 | 366.1 |
| 193 | AGP12 | 72 | 7614 | 1313.1 | 366.1 |
| 194 | TRFE | 630 | 5401 | 1108.4 | 366.1 |
| 195 | TRFE | 630 | 5411 | 1144.9 | 366.1 |
| 196 | IGM | 209 | 5512 | 1224.1 | 366.1 |
| 197 | KNG1 | 137 | NONGLYCOSYLATED | 1190.6 | 1349.7 |
| 198 | FHR1 | 126 | 5402 | 1265.5 | 366.1 |
| 199 | IGG1 | 297 | 4500 | 951.7 | 204.1 |
| 200 | AGP1 | 93 | 7612 | 1250.3 | 366.1 |
| 201 | A1AT | 271 | 5402 | 991.2 | 366.1 |
| 202 | A1AT | 271 | 6503 | 1155.5 | 274.1 |
| 203 | KNG1 | 294 | 5412 | 946.9 | 204.1 |
| 204 | CO2 | 621 | 6200 | 945.1 | 829.4 |
| 205 | HRG | 271 | 2202 | 710.8 | 274.1 |
| 206 | APOD | 98 | 5412 | 1152.5 | 274.1 |
| 207 | AFAM | 33 | 5402 | 851.1 | 366.1 |

TABLE 21
Retention time, A retention time, and collision
energy for transitions associated with NSCLC

| | Retention | Delta | |
|---|---|---|---|
| Transition | Time | Retention | Collision |
| Number | (min) | Time | Energy |
| 159 | 33.44 | 1.4 | 27 |
| 160 | 23.77 | 1.4 | 25 |
| 161 | 12.99 | 1.2 | 25 |
| 162 | 7.9 | 1.3 | 24 |
| 163 | 30.91 | 1.4 | 35 |
| 164 | 5.97 | 1.6 | 30 |
| 165 | 8.01 | 1.3 | 21 |
| 166 | 8.09 | 1.3 | 20 |
| 167 | 19.44 | 1.4 | 37 |
| 168 | 31.27 | 1.4 | 30 |
| 169 | 20.77 | 1.4 | 25 |
| 170 | 6.55 | 1.4 | 25 |
| 171 | 14.38 | 1.5 | 30 |
| 172 | 23.81 | 1.4 | 23 |
| 173 | 28.7 | 1.4 | 25 |
| 174 | 37.62 | 1.4 | 30 |
| 175 | 16.93 | 1.4 | 20 |
| 176 | 26.42 | 1.4 | 28 |
| 177 | 12.87 | 1.2 | 20 |
| 178 | 30.62 | 1.4 | 25 |
| 179 | 23.61 | 1.4 | 25 |
| 180 | 19.97 | 1.4 | 36 |
| 181 | 42.79 | 1.6 | 30 |
| 182 | 30.4 | 1.4 | 30 |
| 183 | 8.35 | 1.3 | 27 |
| 184 | 13.41 | 1.5 | 35 |
| 185 | 30.64 | 1.4 | 29 |
| 186 | 30.52 | 1.4 | 35 |
| 187 | 14.9 | 1.6 | 25 |
| 188 | 23.17 | 1.4 | 28 |
| 189 | 39.45 | 1.5 | 30 |
| 190 | 21.83 | 1.4 | 20 |
| 191 | 39.8 | 1.4 | 25 |
| 192 | 16.04 | 1.4 | 25 |
| 193 | 41.23 | 1.4 | 27 |
| 194 | 31.54 | 1.8 | 27 |
| 195 | 30.97 | 1.6 | 30 |
| 196 | 26.21 | 1.4 | 30 |
| 197 | 38.59 | 1 | 30 |
| 198 | 11.55 | 1.5 | 30 |
| 199 | 8.13 | 1.3 | 23 |
| 200 | 22.84 | 1.4 | 31 |
| 201 | 38.43 | 1.4 | 24 |
| 202 | 38.74 | 1.4 | 30 |
| 203 | 22.96 | 1.4 | 20 |
| 204 | 16.26 | 1.4 | 25 |
| 205 | 6.74 | 1.4 | 15 |
| 206 | 24.42 | 1.4 | 30 |
| 207 | 11.62 | 1.2 | 20 |

In some embodiments, provided herein are methods for diagnosing a melanoma condition (metastatic melanoma) comprising detecting one or more biomarkers. In some embodiments, the one or more biomarkers comprise one or more glycopeptides. In some embodiments, the one or more biomarkers comprises one or more peptide structures set forth in Table 7. In some embodiments, the method comprises detecting one or more glycopeptides comprising a sequence set forth in SEQ ID NOs: 21-46. In some embodiments, the method comprises detecting one or more glycopeptides comprising a sequence set forth in SEQ ID NOs: 21-46. In some embodiments, the method comprises detecting one or more glycopeptides comprising a sequence set forth in SEQ ID NOs: 21-46. In some embodiments, the method comprises detecting one or more glycopeptides comprising a sequence set forth in SEQ ID NOs: 21-46. In some embodiments, the glycopeptide comprises a glycan with the structures in Table 7. In some embodiments, the glycopeptide is a glycopeptide of a glycoprotein provided in Table 8. In some embodiments, the glycopeptide is a glycopeptide provided in Table 16. In some embodiments the glycopeptide comprises a sequence set forth in SEQ ID NO:300-429. In some embodiments, the glycopeptide is a glycopeptide a glycoprotein comprising SEQ ID NO:1-20.

In some embodiments, the diagnosis is based upon presence and/or amount of at least one, at least two, at least three, at least four, at least five, at least six, at least seven or eight peptide structures from Table 7. In some embodiments, the diagnosis is based upon the presence and/or amount of one or more peptides comprising the amino acid sequence of SEQ ID NOs: 21-46. In some embodiments, the diagnosis is based upon the presence and/or amount of two or more peptides comprising the amino acid sequence of SEQ ID NOs: 21-46. In some embodiments, the diagnosis is based upon the presence and/or amount of three or more peptides comprising the amino acid sequence of SEQ ID NOs: 21-46. In some embodiments, the diagnosis is based upon the presence and/or amount of four or more peptides comprising the amino acid sequence of SEQ ID NOs: 21-46. In some embodiments, the diagnosis is based upon the presence and/or amount of five or more peptides comprising the amino acid sequence of SEQ ID NOs: 21-46. In some embodiments, the diagnosis is based upon the presence and/or amount of six or more peptides comprising the amino acid sequence of SEQ ID NOs: 21-46. In some embodiments, the diagnosis is based upon the presence and/or amount of seven or more peptides comprising the amino acid sequence of SEQ ID NOs: 21-46. In some embodiments, the diagnosis is based upon the presence and/or amount of each of the peptides comprising the amino acid sequence of SEQ ID NOs: 21-46. In some embodiments, the glycopeptide is a glycopeptide of a glycoprotein provided in Table 8. In some embodiments, the glycopeptide is a glycopeptide provided in Table 16. In some embodiments the glycopeptide comprises a sequence set forth in SEQ ID NO:300-429. In some embodiments, the glycopeptide is a glycopeptide a glycoprotein comprising SEQ ID NO:1-20.

In some embodiments, the diagnosis is based upon the presence and/or amount of one or more peptides consisting of the amino acid sequence of SEQ ID NOs: 21-46. In some embodiments, the diagnosis is based upon the presence and/or amount of two or more peptides consisting of the amino acid sequence of SEQ ID NOs: 21-46. In some embodiments, the diagnosis is based upon the presence and/or amount of three or more peptides consisting of the amino acid sequence of SEQ ID NOs: 21-46. In some embodiments, the diagnosis is based upon the presence and/or amount of four or more peptides consisting of the amino acid sequence of SEQ ID NOs: 21-46. In some embodiments, the diagnosis is based upon the presence and/or amount of five or more peptides consisting of the amino acid sequence of SEQ ID NOs: 21-46. In some embodiments, the diagnosis is based upon the presence and/or amount of six or more peptides consisting of the amino acid sequence of SEQ ID NOs: 21-46. In some embodiments, the diagnosis is based upon the presence and/or amount of seven or more peptides consisting of the amino acid sequence of SEQ ID NOs: 21-46. In some embodiments, the diagnosis is based upon the presence and/or amount of each of the peptides consisting of the amino acid sequence of SEQ ID NOs: 21-46. In some embodiments, the glycopeptide is a glycopeptide of a glycoprotein provided in Table 8. In some embodiments, the glycopeptide is a glycopeptide provided in Table 16. In some embodiments the glycopeptide comprises a sequence set forth in SEQ ID NO:300-429. In some embodiments, the glycopeptide is a glycopeptide a glycoprotein comprising SEQ ID NO:1-20.

In some embodiments, the diagnosis is based upon the presence and/or amount of one or more peptides comprising the amino acid sequence of SEQ ID NOs: 21-46. In some embodiments, the diagnosis is based upon the presence and/or amount of two or more peptides comprising the amino acid sequence of SEQ ID NOs: 21-46. In some embodiments, the diagnosis is based upon the presence and/or amount of three or more peptides comprising the amino acid sequence of SEQ ID NOs: 21-46. In some embodiments, the diagnosis is based upon the presence and/or amount of four or more peptides comprising the amino acid sequence of SEQ ID NOs: 21-46. In some embodiments, the diagnosis is based upon the presence and/or amount of five or more peptides comprising the amino acid sequence of SEQ ID NOs: 21-46. In some embodiments, the diagnosis is based upon the presence and/or amount of six or more peptides comprising the amino acid sequence of SEQ ID NOs: 21-46. In some embodiments, the diagnosis is based upon the presence and/or amount of seven or more peptides comprising the amino acid sequence of SEQ ID NOs: 21-46. In some embodiments, the diagnosis is based upon the presence and/or amount of each of the peptides comprising the amino acid sequence of SEQ ID NOs: 21-46. In some embodiments, the glycopeptide is a glycopeptide of a glycoprotein provided in Table 8. In some embodiments, the glycopeptide is a glycopeptide provided in Table 16. In some embodiments the glycopeptide comprises a sequence set forth in SEQ ID NO:300-429. In some embodiments, the glycopeptide is a glycopeptide a glycoprotein comprising SEQ ID NO:1-20.

In some embodiments, the diagnosis is based upon the presence and/or amount of one or more peptides consisting of the amino acid sequence of SEQ ID NOs: 21-46. In some embodiments, the diagnosis is based upon the presence and/or amount of two or more peptides consisting of the amino acid sequence of SEQ ID NOs: 21-46. In some embodiments, the diagnosis is based upon the presence and/or amount of three or more peptides consisting of the amino acid sequence of SEQ ID NOs: 21-46. In some embodiments, the diagnosis is based upon the presence and/or amount of four or more peptides consisting of the amino acid sequence of SEQ ID NOs: 21-46. In some embodiments, the diagnosis is based upon the presence and/or amount of five or more peptides consisting of the amino acid sequence of SEQ ID NOs: 21-46. In some embodiments, the diagnosis is based upon the presence and/or amount of six or more peptides consisting of the amino acid sequence of SEQ ID NOs: 21-46. In some embodiments, the diagnosis is based upon the presence and/or amount of seven or more peptides consisting of the amino acid sequence of SEQ ID NOs: 21-46. In some embodiments, the diagnosis is based upon the presence and/or amount of each of the peptides consisting of the amino acid sequence of SEQ ID NOs: 21-46. In some embodiments, the glycopeptide is a glycopeptide of a glycoprotein provided in Table 8. In some embodiments, the glycopeptide is a glycopeptide provided in Table 16. In some embodiments the glycopeptide comprises a sequence set forth in SEQ ID NO:300-429. In some embodiments, the glycopeptide is a glycopeptide a glycoprotein comprising SEQ ID NO:1-20.

In some embodiments, provided herein is a method of treating a melanoma condition (metastatic melanoma) in an individual based upon the presence, absence, or amount of one or more peptide structures set forth in Table 7. In some embodiments, one or more peptide structures set forth in SEQ ID NOs: 21-46 is detected. In some embodiments, the method further comprises delivering a therapeutic agent based upon the presence, absence, or amount of one or more peptide structures set forth in Table 7. In some embodiments, the method comprises selecting a therapeutic agent based upon the presence, absence, or amount of one or more peptide structures set forth in Table 7. In some embodiments, the therapeutic agent is a chemotherapeutic agent and/or a hormone therapy.

In some embodiments, provided herein are methods for diagnosing a melanoma condition (metastatic melanoma) comprising detecting one or more biomarkers. In some embodiments, the one or more biomarkers comprise one or more glycopeptides. In some embodiments, the one or more biomarkers comprises one or more peptide structures set forth in Table 12. In some embodiments, the method comprises detecting one or more glycopeptides comprising a sequence set forth in SEQ ID NOs: 101-131. In some embodiments, the method comprises detecting one or more glycopeptides comprising a sequence set forth in SEQ ID NOs: 101-131. In some embodiments, the method comprises detecting one or more glycopeptides comprising a sequence set forth in SEQ ID NOs: 101-131. In some embodiments, the method comprises detecting one or more glycopeptides comprising a sequence set forth in SEQ ID NOs: 101-131. In some embodiments, the glycopeptide comprises a glycan with the structures in Table 12. In some embodiments the glycopeptide is a glycopeptide of a glycoprotein provided in Table 13. In some embodiments, the glycopeptide is a glycopeptide of a glycoprotein provided in SEQ ID NO: 132-158.

In some embodiments, the diagnosis is based upon presence and/or amount of at least one, at least two, at least three, at least four, at least five, at least six, at least seven or eight peptide structures from Table 12. In some embodiments, the diagnosis is based upon the presence and/or amount of one or more peptides comprising the amino acid sequence of SEQ ID NOs: 101-131. In some embodiments, the diagnosis is based upon the presence and/or amount of two or more peptides comprising the amino acid sequence of SEQ ID NOs: 101-131. In some embodiments, the diagnosis is based upon the presence and/or amount of three or more peptides comprising the amino acid sequence of SEQ ID NOs: 101-131. In some embodiments, the diagnosis is based upon the presence and/or amount of four or more peptides comprising the amino acid sequence of SEQ ID NOs: 101-131. In some embodiments, the diagnosis is based upon the presence and/or amount of five or more peptides comprising the amino acid sequence of SEQ ID NOs: 101-131. In some embodiments, the diagnosis is based upon the presence and/or amount of six or more peptides comprising the amino acid sequence of SEQ ID NOs: 101-131. In some embodiments, the diagnosis is based upon the presence and/or amount of seven or more peptides comprising the amino acid sequence of SEQ ID NOs: 101-131. In some embodiments, the diagnosis is based upon the presence and/or amount of each of the peptides comprising the amino acid sequence of SEQ ID NOs: 101-131. In some embodiments the glycopeptide is a glycopeptide of a glycoprotein provided in Table 13. In some embodiments, the glycopeptide is a glycopeptide of a glycoprotein provided in SEQ ID NO: 132-158.

In some embodiments, the diagnosis is based upon the presence and/or amount of one or more peptides consisting of the amino acid sequence of SEQ ID NOs: 101-131. In some embodiments, the diagnosis is based upon the presence and/or amount of two or more peptides consisting of the amino acid sequence of SEQ ID NOs: 101-131. In some embodiments, the diagnosis is based upon the presence and/or amount of three or more peptides consisting of the amino acid sequence of SEQ ID NOs: 101-131. In some embodiments, the diagnosis is based upon the presence and/or amount of four or more peptides consisting of the amino acid sequence of SEQ ID NOs: 101-131. In some embodiments, the diagnosis is based upon the presence and/or amount of five or more peptides consisting of the amino acid sequence of SEQ ID NOs: 101-131. In some embodiments, the diagnosis is based upon the presence and/or amount of six or more peptides consisting of the amino acid sequence of SEQ ID NOs: 101-131. In some embodiments, the diagnosis is based upon the presence and/or amount of seven or more peptides consisting of the amino acid sequence of SEQ ID NOs: 101-131. In some embodiments, the diagnosis is based upon the presence and/or amount of each of the peptides consisting of the amino acid sequence of SEQ ID NOs: 101-131. In some embodiments the glycopeptide is a glycopeptide of a glycoprotein provided in Table 13. In some embodiments, the glycopeptide is a glycopeptide of a glycoprotein provided in SEQ ID NO: 132-158.

In some embodiments, the diagnosis is based upon the presence and/or amount of one or more peptides comprising the amino acid sequence of SEQ ID NOs: 101-131. In some embodiments, the diagnosis is based upon the presence and/or amount of two or more peptides comprising the amino acid sequence of SEQ ID NOs: 101-131. In some embodiments, the diagnosis is based upon the presence and/or amount of three or more peptides comprising the amino acid sequence of SEQ ID NOs: 101-131. In some embodiments, the diagnosis is based upon the presence and/or amount of four or more peptides comprising the amino acid sequence of SEQ ID NOs: 101-131. In some embodiments, the diagnosis is based upon the presence and/or amount of five or more peptides comprising the amino acid sequence of SEQ ID NOs: 101-131. In some embodiments, the diagnosis is based upon the presence and/or amount of six or more peptides comprising the amino acid sequence of SEQ ID NOs: 101-131. In some embodiments, the diagnosis is based upon the presence and/or amount of seven or more peptides comprising the amino acid sequence of SEQ ID NOs: 101-131. In some embodiments, the diagnosis is based upon the presence and/or amount of each of the peptides comprising the amino acid sequence of SEQ ID NOs: 101-131. In some embodiments the glycopeptide is a glycopeptide of a glycoprotein provided in Table 13. In some embodiments, the glycopeptide is a glycopeptide of a glycoprotein provided in SEQ ID NO: 132-158.

In some embodiments, the diagnosis is based upon the presence and/or amount of one or more peptides consisting of the amino acid sequence of SEQ ID NOs: 101-131. In some embodiments, the diagnosis is based upon the presence and/or amount of two or more peptides consisting of the amino acid sequence of SEQ ID NOs: 101-131. In some embodiments, the diagnosis is based upon the presence and/or amount of three or more peptides consisting of the amino acid sequence of SEQ ID NOs: 101-131. In some embodiments, the diagnosis is based upon the presence and/or amount of four or more peptides consisting of the amino acid sequence of SEQ ID NOs: 101-131. In some embodiments, the diagnosis is based upon the presence and/or amount of five or more peptides consisting of the amino acid sequence of SEQ ID NOs: 101-131. In some embodiments, the diagnosis is based upon the presence and/or amount of six or more peptides consisting of the amino acid sequence of SEQ ID NOs: 101-131. In some embodiments, the diagnosis is based upon the presence and/or amount of seven or more peptides consisting of the amino acid sequence of SEQ ID NOs: 101-131. In some embodiments, the diagnosis is based upon the presence and/or amount of each of the peptides consisting of the amino acid sequence of SEQ ID NOs: 101-131. In some embodiments the glycopeptide is a glycopeptide of a glycoprotein provided in Table 13. In some embodiments, the glycopeptide is a glycopeptide of a glycoprotein provided in SEQ ID NO: 132-158.

In some embodiments, provided herein is a method of treating a melanoma condition (metastatic melanoma) in an individual based upon the presence, absence, or amount of one or more peptide structures set forth in Table 12. In some embodiments, one or more peptide structures set forth in SEQ ID NOs: 101-131 is detected. In some embodiments, the method further comprises delivering a therapeutic agent based upon the presence, absence, or amount of one or more peptide structures set forth in Table 12. In some embodiments, the method comprises selecting a therapeutic agent based upon the presence, absence, or amount of one or more peptide structures set forth in Table 12. In some embodiments, the therapeutic agent is a chemotherapeutic agent and/or a hormone therapy. In some embodiments the glycopeptide is a glycopeptide of a glycoprotein provided in Table 13. In some embodiments, the glycopeptide is a glycopeptide of a glycoprotein provided in SEQ ID NO: 132-158.

In some embodiments, provided herein are methods for diagnosing non-small-cell lung cancer (NSCLC) comprising detecting one or more biomarkers. In some embodiments, the one or more biomarkers comprise one or more glycopeptides. In some embodiments, the one or more biomarkers comprises one or more peptide structures set forth in Table 14. In some embodiments, the method comprises detecting one or more glycopeptides comprising a sequence set forth in SEQ ID NOs: 159-207. In some embodiments, the method comprises detecting one or more glycopeptides comprising a sequence set forth in SEQ ID NOs: 159-207. In some embodiments, the method comprises detecting one or more glycopeptides comprising a sequence set forth in SEQ ID NOs: 159-207. In some embodiments, the method comprises detecting one or more glycopeptides comprising a sequence set forth in SEQ ID NOs: 159-207. In some embodiments, the glycopeptide comprises a glycan with the structures in Table 14. In some embodiments, the glycopeptide is a glycopeptide of a glycoprotein provided in Table 15. In some embodiments, the glycopeptide is a glycopeptide of a glycoprotein comprising a sequence set forth in SEQ ID NO: 208-253.

In some embodiments, the diagnosis is based upon presence and/or amount of at least one, at least two, at least three, at least four, at least five, at least six, at least seven or eight peptide structures from Table 14. In some embodiments, the diagnosis is based upon the presence and/or amount of one or more peptides comprising the amino acid sequence of SEQ ID NOs: 159-207. In some embodiments, the diagnosis is based upon the presence and/or amount of two or more peptides comprising the amino acid sequence of SEQ ID NOs: 159-207. In some embodiments, the diagnosis is based upon the presence and/or amount of three or more peptides comprising the amino acid sequence of SEQ ID NOs: 159-207. In some embodiments, the diagnosis is based upon the presence and/or amount of four or more peptides comprising the amino acid sequence of SEQ ID NOs: 159-207. In some embodiments, the diagnosis is based upon the presence and/or amount of five or more peptides comprising the amino acid sequence of SEQ ID NOs: 159-207. In some embodiments, the diagnosis is based upon the presence and/or amount of six or more peptides comprising the amino acid sequence of SEQ ID NOs: 159-207. In some embodiments, the diagnosis is based upon the presence and/or amount of seven or more peptides comprising the amino acid sequence of SEQ ID NOs: 159-207. In some embodiments, the diagnosis is based upon the presence and/or amount of each of the peptides comprising the amino acid sequence of SEQ ID NOs: 159-207. In some embodiments, the glycopeptide is a glycopeptide of a glycoprotein provided in Table 15. In some embodiments, the glycopeptide is a glycopeptide of a glycoprotein comprising a sequence set forth in SEQ ID NO: 208-253.

In some embodiments, the diagnosis is based upon the presence and/or amount of one or more peptides consisting of the amino acid sequence of SEQ ID NOs: 159-207. In some embodiments, the diagnosis is based upon the presence and/or amount of two or more peptides consisting of the amino acid sequence of SEQ ID NOs: 159-207. In some embodiments, the diagnosis is based upon the presence and/or amount of three or more peptides consisting of the amino acid sequence of SEQ ID NOs: 159-207. In some embodiments, the diagnosis is based upon the presence and/or amount of four or more peptides consisting of the amino acid sequence of SEQ ID NOs: 21-46. In some embodiments, the diagnosis is based upon the presence and/or amount of five or more peptides consisting of the amino acid sequence of SEQ ID NOs: 159-207. In some embodiments, the diagnosis is based upon the presence and/or amount of six or more peptides consisting of the amino acid sequence of SEQ ID NOs: 159-207. In some embodiments, the diagnosis is based upon the presence and/or amount of seven or more peptides consisting of the amino acid sequence of SEQ ID NOs: 159-207. In some embodiments, the diagnosis is based upon the presence and/or amount of each of the peptides consisting of the amino acid sequence of SEQ ID NOs: 159-207. In some embodiments, the glycopeptide is a glycopeptide of a glycoprotein provided in Table 15. In some embodiments, the glycopeptide is a glycopeptide of a glycoprotein comprising a sequence set forth in SEQ ID NO: 208-253.

In some embodiments, the diagnosis is based upon the presence and/or amount of one or more peptides comprising the amino acid sequence of SEQ ID NOs: 159-207. In some embodiments, the diagnosis is based upon the presence and/or amount of two or more peptides comprising the amino acid sequence of SEQ ID NOs: 159-207. In some embodiments, the diagnosis is based upon the presence and/or amount of three or more peptides comprising the amino acid sequence of SEQ ID NOs: 159-207. In some embodiments, the diagnosis is based upon the presence and/or amount of four or more peptides comprising the amino acid sequence of SEQ ID NOs: 159-207. In some embodiments, the diagnosis is based upon the presence and/or amount of five or more peptides comprising the amino acid sequence of SEQ ID NOs: 159-207. In some embodiments, the diagnosis is based upon the presence and/or amount of six or more peptides comprising the amino acid sequence of SEQ ID NOs: 159-207. In some embodiments, the diagnosis is based upon the presence and/or amount of seven or more peptides comprising the amino acid sequence of SEQ ID NOs: 159-207. In some embodiments, the diagnosis is based upon the presence and/or amount of each of the peptides comprising the amino acid sequence of SEQ ID NOs: 159-207. In some embodiments, the glycopeptide is a glycopeptide of a glycoprotein provided in Table 15. In some embodiments, the glycopeptide is a glycopeptide of a glycoprotein comprising a sequence set forth in SEQ ID NO: 208-253.

In some embodiments, the diagnosis is based upon the presence and/or amount of one or more peptides consisting of the amino acid sequence of SEQ ID NOs: 159-207. In some embodiments, the diagnosis is based upon the presence and/or amount of two or more peptides consisting of the amino acid sequence of SEQ ID NOs: 159-207. In some embodiments, the diagnosis is based upon the presence and/or amount of three or more peptides consisting of the amino acid sequence of SEQ ID NOs: 159-207. In some embodiments, the diagnosis is based upon the presence and/or amount of four or more peptides consisting of the amino acid sequence of SEQ ID NOs: 159-207. In some embodiments, the diagnosis is based upon the presence and/or amount of five or more peptides consisting of the amino acid sequence of SEQ ID NOs: 159-207. In some embodiments, the diagnosis is based upon the presence and/or amount of six or more peptides consisting of the amino acid sequence of SEQ ID NOs: 159-207. In some embodiments, the diagnosis is based upon the presence and/or amount of seven or more peptides consisting of the amino acid sequence of SEQ ID NOs: 159-207. In some embodiments, the diagnosis is based upon the presence and/or amount of each of the peptides consisting of the amino acid sequence of SEQ ID NOs: 159-207. In some embodiments, the glycopeptide is a glycopeptide of a glycoprotein provided in Table 15. In some embodiments, the glycopeptide is a glycopeptide of a glycoprotein comprising a sequence set forth in SEQ ID NO: 208-253.

In some embodiments, provided herein is a method of treating non-small-cell lung cancer (NSCLC) in an individual based upon the presence, absence, or amount of one or more peptide structures set forth in Table 14. In some embodiments, one or more peptide structures set forth in SEQ ID NOs: 159-207 is detected. In some embodiments, the method further comprises delivering a therapeutic agent based upon the presence, absence, or amount of one or more peptide structures set forth in Table 14. In some embodiments, the method comprises selecting a therapeutic agent based upon the presence, absence, or amount of one or more peptide structures set forth in Table 14. In some embodiments, the therapeutic agent is a chemotherapeutic agent and/or a hormone therapy. In some embodiments, the glycopeptide is a glycopeptide of a glycoprotein provided in Table 15. In some embodiments, the glycopeptide is a glycopeptide of a glycoprotein comprising a sequence set forth in SEQ ID NO: 208-253.

In the descriptions herein, it is understood that every description, variation, embodiment or aspect of a biomarker, peptide, glycopeptide, glycoprotein may be combined with every description, variation, embodiment or aspect of other biomarkers, peptides, glycopeptide, glycoproteins the same as if each and every combination of descriptions is specifically and individually listed.

### V. Examples

The following examples are included for illustrative purposes only and are not intended to limit the scope of the invention.

### Example 1: Glycoproteomics as Liquid Biopsy-Based Predictor of Checkpoint-Inhibitor Treatment Response in Patients with Metastatic Malignant Melanoma

Protein glycosylation is one of the most abundant and most complex form of post-translational protein modification. Glycosylation affects protein structure, conformation, and function. The elucidation of the potential role of differential protein glycosylation as biomarkers has so far been limited by the technical complexity of generating and interpreting this information. A novel, powerful platform has been recently established that combines ultra-high-performance liquid chromatography coupled to triple quadrupole mass spectrometry with a proprietary machine-learning and neural-network-based data processing engine that allows for high-throughput, highly scalable interrogation of the glycoproteome. This study assessed whether glycoproteomic biomarkers and signatures can predict which patients with metastatic malignant melanoma would respond to PD1/PDL1 checkpoint inhibitors.

Methods: this platform we interrogated 413 individual glycopeptide (GP) signatures derived from 69 abundant serum proteins in pretreatment blood samples from a cohort of 36 individuals (11 females, 25 males, age range 28 to 90 years) with metastatic malignant melanoma treated either with nivolumab plus ipilimumab (12 patients) or pembrolizumab (24 patients). Plasma samples were taken prior to beginning treatment, stored at −80 C, and run through InterVenn's targeted MRM panel.

The individual glycopeptide expression levels were associated with time from treatment initiation to progression/metastasis (progression-free survival, PFS) or death (overall survival, OS) in the patient cohorts.

In addition to assessing individual biomarker associations, multivariable models were built to predict PFS (Melanoma). The multivariate models were built by selecting a small subset of glycopeptides for modeling, proceeding to build a model with n−1 patients, predicting a survival score on the one holdout patient, and iterating over all patients as individual holdouts, to generate unbiased prediction scores for everyone (a leave-one-out cross-validation approach, LOOCV). The resulting scores were dichotomized at a cutoff which optimizes Harrell's C-index, and Kaplan-Meier (KM) curves were plotted.

Specifically, progression-free survival (PFS) data with follow-up of up to 3.7 years (median: 0.8 years) were used as clinical endpoint phenotype against which the predictive power of differential abundance of GPs was assessed. PFS data were analyzed using Cox Proportional Hazards models. Kaplan Meier curves were generated for GP markers that showed statistically significant differential abundances using a false discovery rate (FDR)-adjusted p-value of ≤0.1 as a cutoff. Hazard Ratio (HR) for PFS was calculated from a Cox Proportional Hazards model, representing the multiplicative increase in odds of progression for each increase of the biomarker by 1 unit. The p-value associated with the HR was analyzed, where p<0.01 was considered significant. The interaction p-value, the p-value associated with the biomarker x treatment interaction, was also analyzed, where significance indicates potential for use in treatment selection.

Further, as part of this example, an interrogation of 526 glycopeptide (GP) signatures derived from 75 serum proteins in pretreatment blood samples from a cohort of 205 individuals (66 females, 139 males, age range 24 to 97 years) with metastatic malignant melanoma treated either with nivolumab (N) with or without ipilimumab (I, 95 patients) or pembrolizumab (P, 110 patients) immune-checkpoint inhibitor (ICI) therapy.

In certain embodiments, FIGS. 14A, 14B illustrate the KM curves for a multivariable model, including the training phases and validation phases, respectively. Hazard ratios and p-values on the plots are representative of the high/low split at the risk score cut-off determined by optimizing for sensitivity for non-response. Study 1 KM curve of FIG. 14B labeled “Validation” contains patients from the validation and test data sets. In one example, the optimal model includes 6 biomarkers and a cutoff was selected in the validation set to optimize for sensitivity to response (e.g., test set 720 day performance: sensitivity=99.5%, specificity=25.6%) metrics/curves shown exclude Indeterminate calls (10% of patient set).

Results: 27 GPs with abundance differences at FDR p≤0.1 were identified, and among them 8 markers at p≤0.001. Using the latter 8 markers, a multivariable model for PFS was created by generating leave-one-out cross-validation (LOOCV) scores and determining an optimized cutoff value for these scores using Harrel's concordance index. Dichotomizing the LOOCV scores using this cutoff value demonstrated the model to yield a hazard ratio of 9.2 at a p-value of 10⁻⁵ for separating treatment responders and non-responders (70% vs. 0% PFS, respectively, at 18 months based on LOOCV score above/below cutoff), as compared to a hazard ratio of 1.5, p=0.5 for PDL1 expression. FIG. 1 shows a Kaplan-Meier curve of patients with metastatic melanoma treated either with a combination of ipilimumab and nivolumab or pembrolizumab alone, where progression-free survival (PFS) was 61% at 2.7 years in the Low Score group (black) as compared to PFS of 50% at 0.10 years in the High Score group (blue).

In an optimized assay containing 27 glycopeptides and 20 non-glycosylated peptides, we identified 14 GPs with abundance differences at FDR q≤0.05 with regard to PFS. Using 40% of the cohort as a training set and selecting 12 glycopeptide and non-glycosylated peptide biomarker features of the 47 total by LASSO shrinkage, we created a multivariable-model-based classifier for PFS that yielded a hazard ratio (HR) for prediction of likely ICI benefit of 7.5 at p<0.0001. This classifier was validated in the test set comprised of the held-out 60% of patients, yielding a HR of 4.7 at a similar p-value for separating patients likely benefiting from either single or combination ICI therapy and those likely not benefiting (50% PFS of 18 months vs. 3 months based on classifier score above/below cutoff). This classifier has a sensitivity of >99% to predict likely ICI benefit, while still performing at a specificity of 26%, thus helping to safely reduce ultimately unnecessary and non-beneficial exposure to these agents of one in four who otherwise would unnecessarily be exposed to them.

Conclusions: Our results indicate that glycoproteomics holds a strong promise as a response predictor to checkpoint inhibitor treatment that appears to significantly outperform other currently pursued biomarker approaches in this context.

### Example 2: Blood-Based Glycoprotein Signatures in Patients with Advanced Non-Small-Cell Lung Carcinoma (NSCLC) Receiving First-Line Immune Checkpoint Blockade

Background: Immune checkpoint blockade is an integral component of first-line therapy for most patients with advanced non-small cell lung cancer (NSCLC), however individual patient outcomes are highly variable and improved biomarkers are needed. Protein glycosylation is an emerging mechanism of immune evasion in cancer. Blood-based glycopeptide signatures were examined in a cohort of advanced NSCLC patients treated with first-line immune checkpoint blockade. This study assessed whether glycoproteomic biomarkers and signatures can predict which patients with NSCLC would respond to PD1/PDL1 checkpoint inhibitors.

Methods: In two independent studies, whether glycoproteomic biomarkers and signatures may predict which patients would respond to checkpoint inhibitor therapies was determined. For example, Study 1 included of n=205 patients with metastatic melanoma seen at Massachusetts General Hospital (MGH), treated either with Ipilimumab+Nivolumab (n=95) or Pembrolizumab (n=110). Plasma samples were taken prior to beginning treatment, stored at −80 C, and inputted to a targeted multiple reaction monitoring (MRM) panel. Study 2 included n=125 patients with metastatic non-small-cell lung cancer sourced from Tempus and treated with Pembrolizumab. Serum samples were taken prior to beginning treatment, stored at −80 C, and inputted to the targeted MRM panel. In both Study 1 and Study 2, individual glycopeptide expression levels were associated with time from treatment initiation to progression-free survival (PFS) (e.g., progression/metastasis) or overall survival (OS) in the patient cohorts.

In addition to assessing individual biomarker associations, multivariable models were built to predict OS (NSCLC) and PFS (Melanoma). The multivariable models were built to predict OS (NSCLC) and PFS (Melanoma) by selecting a small subset of glycopeptides through 5-fold repeated cross-validated LASSO regularization, proceeding to build a model with 40% of patients (allocated via balanced stratification on sex, age quartile, PFS/OS event), tuning hyperparameters in LASSO model in another 30% of patients, and predicting a survival score on the remaining 30% of holdout patients (to generate unbiased prediction scores). The resulting prediction scores were dichotomized at a cutoff which optimizes Harrell's C-index, and Kaplan-Meier (KM) curves were plotted final models for products were optimized for sensitivity for non-response. For example, in certain embodiments, FIGS. 15A and 15B illustrate the KM curves for a multivariable model, including the training phases and validation phases, respectively. Hazard ratios and p-values on the plots are representative of the high/low split at the risk score cut-off determined by optimizing for sensitivity for non-response. Study 2 KM curve of FIG. 15B labeled “Validation” contains patients only from the independent/unseen test set since there was no validation set. In one example, the optimal model includes 6 biomarkers and a cutoff was selected in the validation set to optimize for sensitivity to response (e.g., test set 720 day performance: sensitivity=99.5%, specificity=25.6%) metrics/curves shown exclude Indeterminate calls (10% of patient set).

Results: 30 GPs with abundance differences using a False Discovery Rate (FDR) threshold of 0.05 were identified. Using the 5 most predictive GP markers, a multivariable model for OS was created by generating leave-one-out cross-validation (LOOCV) scores and determining an optimized cutoff value of −0.83 (range: −2.2-3.4) for these scores using Harrell's concordance index. The median overall survival was 2.8 years for patients (n=14) whose GP classifier value was above the cutoff and 0.8 years for patients (n=32) whose GP classifier value was below the cutoff (HR 7.4, 95% CI 1.7-32.1, p=0.007). The model's performance was not affected by sex, age, or treatment regimen.

Conclusions: Blood-based glycopeptide signatures may represent novel, non-invasive biomarkers of clinical outcome to first-line immune checkpoint blockade in advanced NSCLC. These findings may be validated in larger cohorts and applied in clinical decision-making.

### V. Additional Considerations

Any headers and/or subheaders between sections and subsections of this document are included solely for the purpose of improving readability and do not imply that features cannot be combined across sections and subsection. Accordingly, sections and subsections do not describe separate embodiments.

While the present teachings are described in conjunction with various embodiments, it is not intended that the present teachings be limited to such embodiments. On the contrary, the present teachings encompass various alternatives, modifications, and equivalents, as will be appreciated by those of skill in the art. The present description provides preferred exemplary embodiments, and is not intended to limit the scope, applicability or configuration of the disclosure. Rather, the present description of the preferred exemplary embodiments will provide those skilled in the art with an enabling description for implementing various embodiments.

It is understood that various changes may be made in the function and arrangement of elements without departing from the spirit and scope as set forth in the appended claims. Thus, such modifications and variations are considered to be within the scope set forth in the appended claims. Further, the terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

Specific details are given in the present description to provide an understanding of the embodiments. However, it is understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

### Embodiments

Among the provided embodiments are:

1. A method for managing a treatment for a subject diagnosed with a melanoma condition, the method comprising:
receiving peptide structure data corresponding to a set of glycoproteins in a biological sample obtained from the subject;
computing a treatment score using quantification data identified from the peptide structure data for a set of peptide structures, wherein the set of peptide structures includes at least one peptide structure identified from a plurality of peptide structures listed in Table 1;
generating a treatment output that indicates a predicted response to the treatment for the subject using the treatment score.
   2. The method of Embodiment 1, wherein generating the treatment output comprises:
generating the predicted response to the treatment based on whether the treatment score is above a selected threshold.
   3. The method of Embodiment 2, wherein the selected threshold is 0.5.
   4. The method of Embodiment 2, wherein the generating the predicted response comprises:
identifying a first predicted response classification for the subject when the treatment score is above 0.5; and
identifying a second predicted response classification for the subject when the treatment score is not above 0.5.
   5. The method of Embodiment 4, wherein the first predicted response classification is sustained control and wherein the second predicted response classification is early disruption.
   6. The method of any one of Embodiments 1-5, wherein the treatment is pembrolizumab and wherein the set of peptide structures includes at least one peptide structure identified from a plurality of peptide structures listed in Table 2.
   7. The method of any one of Embodiments 1-6, wherein the treatment comprises a combination of nivolumab and ipilimumab and wherein the set of peptide structures includes at least one peptide structure identified from a plurality of peptide structures listed in Table 3.
   8. The method of any one of Embodiments 1-7, wherein the treatment outcome includes a recommendation to modify a treatment plan for the subject.
   9. The method of Embodiment 8, wherein the recommendation for modifying the treatment plan includes at least one of selecting a different treatment for the subject, altering a dosage for the treatment, or combining the treatment with at least one other treatment.
   10. The method of any one of Embodiments 1-9, wherein computing the treatment score comprises:
computing a proportion of the set of peptide structures having a selected abundance greater than a reference abundance.
   11. The method of Embodiment 10, wherein the reference abundance for a peptide structure of the set of peptide structures is a median of a plurality of abundances for the peptide structure across a sample population and wherein the selected abundance for a glycopeptide structure of the set of peptide structures is a relative abundance and the selected abundance for an aglycosylated peptide structure of the set of peptide structures is an absolute abundance.
   12. The method of any one of Embodiments 1-11, further comprising:
identifying the set of peptide structures using sample data and a statistical algorithm that identifies a relative significance for each peptide structure of a collection of peptide structures corresponding to the sample data.
   13. The method of Embodiment 12, wherein the statistical algorithm comprises a Wilcoxon rank-sum test.
   14. The method of Embodiment 12 or Embodiment 13, wherein identifying the set of peptide structures comprises:
performing a differential abundance analysis using the sample data to compare a first portion of the sample data corresponding to a first response classification for the treatment and a second portion of the sample data corresponding to a second response classification for the treatment to identify a selected N most differentiating peptide structures between the first response classification and the second response classification.
   15. The method of Embodiment 14, wherein the selected N most differentiating peptide structures is 20 peptide structures.
   16. The method of Embodiment 14 or Embodiment 15, wherein:
the first response classification is sustained control which indicates an absence of disruption events during a sustained period of time after treatment administration;
the second response classification is early disruption which indicates a presence of at least one disruption event during an initial period of time after treatment; and
the sustained period of time is longer than the initial period of time.
   17. The method of Embodiment 16, wherein the sustained period of time is 12 months and the initial period of time is 6 months.
   18. The method of any one of Embodiments 1-17, wherein the at least one peptide structure comprises a glycopeptide structure defined by a peptide sequence and a glycan structure linked to the peptide sequence at a linking site of the peptide sequence, as identified in Table 1, with the peptide sequence being one of SEQ ID NOS: 21-46 as defined in Table 7.
   19. The method of any one of Embodiments 1-18, wherein the quantification data for a peptide structure of the set of peptide structures comprises at least one of an adjusted abundance, a relative abundance, an absolute abundance, a normalized abundance, a relative quantity, an adjusted quantity, a normalized quantity, a relative concentration, an adjusted concentration, or a normalized concentration.
   20. The method of any one of Embodiments 1-19, wherein the peptide structure data is generated using multiple reaction monitoring mass spectrometry (MRM-MS).
   21. The method of any one of Embodiments 1-20, further comprising:
creating a sample from the biological sample; and preparing the sample using reduction, alkylation, and enzymatic digestion to form a prepared sample that includes a set of peptide structures.
   22. The method of Embodiment 21, further comprising:
generating the peptide structure data from the prepared sample using multiple reaction monitoring mass spectrometry (MRM-MS).
   23. The method of any one of Embodiments 1-22, wherein the treatment output comprises at least one of a design for the treatment or a therapeutic dosage for the treatment.
   24. The method of any one of Embodiments 1-23, further comprising:
sending the treatment output to a remote system.
   25. The method of any one of Embodiments 1-24, further comprising:
administering a therapeutic dosage of the treatment based on the predicted response being a predicted response classification that indicates the treatment will be successful.
   26. The method of any one of Embodiments 1-25, further comprising:
administering a therapeutic dosage of the treatment based on the predicted response being sustained control.
   27. A method for treatment management of a subject diagnosed with a melanoma condition, the method comprising:
receiving peptide structure data corresponding to a set of peptide structures associated with a set of glycoproteins in a biological sample obtained from the subject;
computing a plurality of treatment scores using quantification data identified from the peptide structure data for a plurality of subsets of the set of peptide structures, wherein each treatment score of the plurality of treatment scores corresponds to a different treatment of a plurality of treatments; wherein each subset of the plurality of subsets includes at least one peptide structure identified from a plurality of peptide structures listed in Table 1;
performing a comparison analysis of the plurality of treatment scores; and
generating a treatment output based on the comparison analysis, wherein the treatment output includes a recommended treatment plan for treating the subject.
   28. The method of Embodiment 27, wherein generating the treatment output comprises:
identifying a treatment of the plurality of treatments having a highest treatment score as a recommended treatment for treating the subject.
   29. The method of Embodiment 27 or Embodiment 28, wherein the plurality of treatments comprises a first treatment of pembrolizumab and a second treatment that is comprised of nivolumab and ipilimumab.
   30. The method of any one of Embodiments 27-29, wherein performing the comparison analysis comprises:
determining that a treatment of the plurality of treatments has a treatment score below a selected threshold; and
excluding the treatment from the comparison analysis.
   31. The method of Embodiment 30, wherein the selected threshold is 0.5.
   32. The method of any one of Embodiments 27-31, wherein the generating the treatment output comprises:
identifying a predicted response classification for the subject for each treatment of the plurality of treatments using a corresponding treatment score of the plurality of treatment scores.
   33. The method of Embodiment 32, wherein the predicted response classification is sustained control when the corresponding treatment score is above a selected threshold and is early disruption when the corresponding treatment score is not above the selected threshold.
   34. The method of Embodiment 33, wherein the selected threshold is 0.5.
   35. The method of any one of Embodiments 27-34, wherein generating the treatment output comprises:
identifying a treatment of the plurality of treatments having a highest treatment score;
determining that the highest treatment score is not above a selected threshold; and
generating the treatment output with the recommended treatment plan including a recommendation to modify an existing treatment plan for the subject.
   36. The method of Embodiment 35, wherein the recommendation for modifying the existing treatment plan includes at least one of selecting a different treatment for the subject, altering a dosage for a treatment that is part of the existing treatment plan, or combining the treatment with at least one other treatment.
   37. The method of any one of Embodiments 27-36, wherein generating the treatment output comprises:
identifying a treatment of the plurality of treatments having a highest treatment score as a highest-scored treatment;
determining that the highest treatment score is above a selected threshold; and
generating the treatment output with the recommended treatment plan identifying the highest-score treatment as a recommended treatment for treating the subject.
   38. The method of any one of Embodiments 27-37, wherein a first treatment of the plurality of treatments comprises pembrolizumab, wherein a second treatment of the plurality of treatments comprises a combination of nivolumab and ipilimumab, and wherein computing the plurality of treatment scores comprises:
computing a first treatment score for the first treatment using a first portion of the quantification data identified from the peptide structure data for a first subset of the plurality of subsets of the set of peptide structures, wherein the first subset includes at least one peptide structure identified from a plurality of peptide structures listed in Table 2; and
computing a second treatment score for the second treatment using a second portion of the quantification data identified from the peptide structure data for a second subset of the plurality of subsets of the set of peptide structures, wherein the second subset includes at least one peptide structure identified from a plurality of peptide structures listed in Table 3.
   39. The method of any one of Embodiments 27-38, wherein computing the plurality of treatment scores comprises:
computing a proportion of a subset of the plurality of subsets of the set of peptide structures having a selected abundance greater than a reference abundance as a treatment score of the plurality of treatment scores.
   40. The method of Embodiment 39, wherein the reference abundance for a peptide structure of the set of peptide structures is a median of a plurality of abundances for the peptide structure across a sample population and wherein the selected abundance for a glycopeptide structure of the set of peptide structures is a relative abundance and the selected abundance for an aglycosylated peptide structure of the set of peptide structures is an absolute abundance.
   41. The method of any one of Embodiments 27-40, further comprising:
identifying a subset of the plurality of subsets of the set of peptide structures using sample data and a statistical algorithm that identifies a relative significance for each peptide structure of a collection of peptide structures corresponding to the sample data with respect to a response to a selected treatment of the plurality of treatments.
   42. The method of Embodiment 41, wherein the statistical algorithm comprises a Wilcoxon rank-sum test.
   43. The method of Embodiment 41 or Embodiment 42, wherein identifying the subset comprises:
performing a differential abundance analysis using the sample data to compare a first portion of the sample data corresponding to a first response classification for the selected treatment and a second portion of the sample data corresponding to a second response classification for the selected treatment to identify a selected N most differentiating peptide structures between the first response classification and the second response classification.
   44. The method of Embodiment 43, wherein the selected N most differentiating peptide structures is 20 peptide structures.
   45. The method of Embodiment 43 or Embodiment 44, wherein:
the first response classification is sustained control which indicates an absence of disruption events during a sustained period of time after treatment administration;
the second response classification is early disruption which indicates a presence of at least one disruption event during an initial period of time after treatment; and
the sustained period of time is longer than the initial period of time.
   46. The method of Embodiment 45, wherein the sustained period of time is 12 months and the initial period of time is 6 months.
   47. The method of any one of Embodiments 27-46, wherein the at least one peptide structure comprises a glycopeptide structure defined by a peptide sequence and a glycan structure linked to the peptide sequence at a linking site of the peptide sequence, as identified in Table 1, with the peptide sequence being one of SEQ ID NOS: 21-46 as defined in Table 7.
   48. The method of any one of Embodiments 27-47, wherein the quantification data for a peptide structure of the set of peptide structures comprises at least one of an adjusted abundance, a relative abundance, an absolute abundance, a normalized abundance, a relative quantity, an adjusted quantity, a normalized quantity, a relative concentration, an adjusted concentration, or a normalized concentration.
   49. The method of claim any one of Embodiments 27-48, wherein the peptide structure data is generated using multiple reaction monitoring mass spectrometry (MRM-MS).
   50. The method of any one of Embodiments 27-49, further comprising:
creating a sample from the biological sample; and
   preparing the sample using reduction, alkylation, and enzymatic digestion to form a prepared sample that includes a set of peptide structures.
      51. The method of Embodiment 50, further comprising:
   generating the peptide structure data from the prepared sample using multiple reaction monitoring mass spectrometry (MRM-MS).
      52. The method of any one of Embodiments 27-51, wherein the recommended treatment plan identifies a recommended treatment and a therapeutic dosage for the recommended treatment.
      53. The method of Embodiment 52, further comprising:
   administering a therapeutic dosage of the recommended.
      54. The method of any one of Embodiments 27-53, further comprising:
   sending the treatment output to a remote system.
      55. A method for treatment management of a subject diagnosed with a melanoma condition, the method comprising:
   receiving peptide structure data corresponding to a set of peptide structures associated with a set of glycoproteins in a biological sample obtained from the subject;
   computing a first treatment score for a first treatment of pembrolizumab using first quantification data identified from the peptide structure data for a first subset of the set of peptide structures, wherein the first subset includes at least one peptide structure identified from a plurality of peptide structures listed in Table 2;
   computing a second treatment score for a second treatment comprised of nivolumab and ipilimumab using second quantification data identified from the peptide structure data for a second subset of the set of peptide structures, wherein the second subset includes at least one peptide structure identified from a plurality of peptide structures listed in Table 3;
   performing a comparison analysis of the first treatment score and the second treatment score; and
   generating a treatment output based on the comparison analysis, wherein the treatment output identifies one of the first treatment and the second treatment as a recommended treatment for the subject.
      56. The method of Embodiment 55, wherein computing the first treatment score comprises:
   computing a proportion of the first subset having a selected abundance greater than a reference abundance as the first treatment score.
      57. The method of Embodiment 55 or Embodiment 56, wherein computing the second treatment score comprises:
   computing a proportion of the second subset having a selected abundance greater than a reference abundance as the second treatment score.
      58. A method for treating a subject diagnosed with a melanoma condition, comprising:
   receiving peptide structure data corresponding to a set of glycoproteins in a biological sample obtained from the subject;
   computing a treatment score using quantification data identified from the peptide structure data for a set of peptide structures, wherein the set of peptide structures includes at least one peptide structure identified from a plurality of peptide structures listed in Table 1;
   generating a treatment output that indicates a predicted response to a treatment for the subject using the treatment score; and
   administering the treatment to the patient in response to the predicted response includes a positive response classification, the step of administering comprising at least one of intravenous or oral administration of the recommended treatment or a derivative thereof at a therapeutic dosage,
      wherein the treatment is selected as one from a group consisting of:
         a first treatment of pembrolizumab for which the therapeutic dosage of at least one of 200 mg every three weeks, 2 mg/kg every three weeks is administered, or 400 mg every 6 weeks; and
         a second treatment comprised of nivolumab and ipilimumab for which the therapeutic dosage of either 1 mg/kg nivolumab with 3 mg/kg ipilimumab or 3 mg/kg nivolumab with 1 mg/kg ipilimumab is administered.
            59. A method for treating a subject diagnosed with a melanoma condition, comprising:
   receiving peptide structure data corresponding to a set of peptide structures associated with a set of glycoproteins in a biological sample obtained from the subject;
   computing a plurality of treatment scores using quantification data identified from the peptide structure data for a plurality of subsets of the set of peptide structures, wherein each treatment score of the plurality of treatment scores corresponds to a different treatment of a plurality of treatments; wherein each subset of the plurality of subsets includes at least one peptide structure identified from a plurality of peptide structures listed in Table 1;
   performing a comparison analysis of the plurality of treatment scores;
   generating a treatment output based on the comparison analysis, wherein the treatment output includes a recommended treatment from the plurality of treatments for treating the subject; and
   administering the recommended treatment to the patient, the step of administering comprising at least one of intravenous or oral administration of the recommended treatment or a derivative thereof at a therapeutic dosage,
      wherein the plurality of treatments comprises:
         a first treatment of pembrolizumab for which the therapeutic dosage of at least one of 200 mg every three weeks, 2 mg/kg every three weeks is administered, or 400 mg every 6 weeks; and
         a second treatment comprised of nivolumab and ipilimumab for which the therapeutic dosage of either 1 mg/kg nivolumab with 3 mg/kg ipilimumab or 3 mg/kg nivolumab with 1 mg/kg ipilimumab is administered.
            60. A method for managing a treatment for a subject diagnosed with a melanoma condition, the method comprising:
   receiving sample data for a sample population, wherein the sample data characterizes responses of a plurality of sample subjects diagnosed with the melanoma condition to the treatment and includes sample peptide structure data for a collection of peptide structures for each subject of the plurality of sample subjects;
   grouping the sample data based on the responses of the plurality of sample subjects into a first group corresponding to a first response classification and a second group corresponding to a second response classification;
   performing a differential abundance analysis using the sample data to compare the first group of the sample data corresponding to the first response classification and the second group of the sample data corresponding to the second response classification to identify a set of peptide structures from the collection of peptide structures,
      wherein the set of peptide structures comprises a selected N most differentiating peptide structures between the first response classification and the second response classification; and
   receiving peptide structure data corresponding to a set of glycoproteins in a biological sample obtained from the subject;
   computing a treatment score for the treatment using quantification data identified from the peptide structure data for the set of peptide structures; and
   generating a treatment output that indicates a predicted response to the treatment for the subject using the treatment score.
      61. The method of Embodiment 60, wherein the set of peptide structures includes at least one peptide structure identified from a plurality of peptide structures listed in Table 1.
      62. The method of Embodiment 60 or claim 61, wherein the differential abundance analysis is performed using a Wilcoxon rank-sum test.
      63. The method of any one of Embodiments 60-62, wherein the selected N most differentiating peptide structures is 20 peptide structures.
      64. The method of any one of Embodiments 60-63, wherein:
   the first response classification is sustained control which indicates an absence of disruption events during a sustained period of time after treatment administration;
   the second response classification is early disruption which indicates a presence of at least one disruption event during an initial period of time after treatment; and
   the sustained period of time is longer than the initial period of time.
      65. The method of Embodiment 64, wherein the sustained period of time is 12 months and the initial period of time is 6 months.
      66. A method of treating melanoma in a subject, the method comprising:
   receiving peptide structure data corresponding to a set of glycoproteins in a biological sample obtained from the subject;
   computing a treatment score using quantification data identified from the peptide structure data for a set of peptide structures, wherein the set of peptide structures includes at least one peptide structure identified from a plurality of peptide structures listed in Table 1;
   generating a treatment output using the treatment score; and
   administering a pembrolizumab treatment to the subject if the treatment output includes at least one of a positive response classification for the pembrolizumab treatment or an identification of the pembrolizumab treatment as a recommended treatment.
      67. The method of Embodiment 66, wherein the administering comprises:
administering the pembrolizumab treatment at a dosage of 200 mg every 3 weeks.
   68. The method of Embodiment 66, wherein the administering comprises:
administering the pembrolizumab treatment at a dosage of 2 mg/kg mg every 3 weeks.
   69. The method of Embodiment 66, wherein the administering comprises:
administering the pembrolizumab treatment at a dosage of 400 mg every 3 weeks.
   70. The method of any one of Embodiments 66-69, wherein the administering comprises:
   administering the pembrolizumab treatment via an intravenous route of administration.
      71. The method of any one of Embodiments 66-70, wherein the administering comprises:
   administering the pembrolizumab treatment every three weeks for four doses.
      72. A method of treating melanoma in a subject, the method comprising:
   receiving peptide structure data corresponding to a set of glycoproteins in a biological sample obtained from the subject;
   computing a treatment score using quantification data identified from the peptide structure data for a set of peptide structures, wherein the set of peptide structures includes at least one peptide structure identified from a plurality of peptide structures listed in Table 1;
   generating a treatment output using the treatment score; and
   administering a combination treatment comprising a combination of nivolumab and ipilimumab to the subject if the treatment output includes at least one of a positive response classification for the combination treatment or an identification of the combination treatment as a recommended treatment.
      73. The method of Embodiment 72, wherein the administering comprises:
   administering the combination treatment to the subject at a dosage of 1 mg/kg of nivolumab with 3 mg/kg of ipilimumab.
      74. The method of Embodiment 72, wherein the administering comprises:
   administering the combination treatment to the subject at a dosage of 3 mg/kg of nivolumab with 1 mg/kg of ipilimumab.
      75. The method of any one of Embodiments 72-74, wherein the administering comprises:
   administering the combination treatment via an intravenous route of administration.
      76. The method of any one of Embodiments 72-75, wherein the administering comprises:
   administering the combination treatment every three weeks for four doses.
      77. A method of identifying patients with melanoma for treatment with a pembrolizumab treatment, the method comprising:
   receiving peptide structure data corresponding to a set of glycoproteins in a biological sample obtained from the subject;
   computing a treatment score using quantification data identified from the peptide structure data for a set of peptide structures, wherein the set of peptide structures includes at least one peptide structure identified from a plurality of peptide structures listed in Table 1; and
   generating a treatment output using the treatment score,
   wherein the patient is treated with the pembrolizumab treatment if the treatment output includes at least one of a positive response classification for the pembrolizumab treatment or an identification of the pembrolizumab treatment as a recommended treatment.
      78. The method of Embodiment 77, wherein the pembrolizumab treatment is administered at a dosage of 200 mg every 3 weeks.
      79. The method of Embodiment 77, wherein the pembrolizumab treatment is administered at a dosage of 2 mg/kg mg every 3 weeks.
      80. The method of Embodiment 77, wherein the pembrolizumab treatment is administered at a dosage of 400 mg every 3 weeks.
      81. The method of any one of Embodiments 77-80, wherein the pembrolizumab treatment is administered via an intravenous route of administration.
      82. The method of any one of Embodiments 77-81, wherein the pembrolizumab treatment is administered every three weeks for four doses.
      83. A method of identifying patients with melanoma for treatment with a combination treatment comprising nivolumab and ipilimumab, the method comprising:
   receiving peptide structure data corresponding to a set of glycoproteins in a biological sample obtained from the subject;
   computing a treatment score using quantification data identified from the peptide structure data for a set of peptide structures, wherein the set of peptide structures includes at least one peptide structure identified from a plurality of peptide structures listed in Table 1; and
   generating a treatment output using the treatment score,
   wherein the patient is treated with the combination treatment if the treatment output includes at least one of a positive response classification for the combination treatment or an identification of the combination treatment as a recommended treatment.
      84. The method of Embodiment 83, wherein the combination treatment is administered at a dosage of 1 mg/kg of nivolumab combined with 3 mg/kg of ipilimumab.
      85. The method of Embodiment 83, wherein the combination treatment is administered at a dosage of 3 mg/kg of nivolumab combined with 1 mg/kg of ipilimumab.
      86. The method of any one of Embodiments 83-85, wherein the combination treatment is administered via an intravenous route of administration.
      87. The method of any one of Embodiments 83-86, wherein the combination treatment is administered every three weeks for four doses.
      88. A method for analyzing a set of peptide structures in a sample from a patient, the method comprising:
(a) obtaining the sample from the patient;
   (b) preparing the sample to form a prepared sample comprising a set of peptide structures;
   (c) inputting the prepared sample into a reaction monitoring mass spectrometry system to detect a set of product ions associated with each peptide structure of the set of peptide structures,
      wherein the set of peptide structures includes at least one peptide structure selected from peptide structures PS-1 to PS-38 identified in Table 6;
      wherein the set of peptide structures includes a peptide structure that is characterized as having:
         (i) a precursor ion with a mass-charge (m/z) ratio within ±1.5 of the m/z ratio listed for the precursor ion in Table 6 as corresponding to the peptide structure; and
         (ii) a product ion having an m/z ratio within ±1.0 of the m/z ratio listed for the first product ion in Table 6 as corresponding to the peptide structure; and
   (d) generating quantification data for the set of product ions using the reaction monitoring mass spectrometry system.
      89. The method of Embodiment 88, wherein the mass-charge (m/z) ratio of the precursor ion is within ±1.0 of the m/z ratio listed for the precursor ion in Table 6 as corresponding to the peptide structure.
      90. The method of Embodiment 88, wherein the mass-charge (m/z) ratio of the precursor ion is within ±0.5 of the m/z ratio listed for the precursor ion in Table 6 as corresponding to the peptide structure.
      91. The method of any one of Embodiments 88-90, wherein the mass-charge (m/z) ratio of the product ion is within ±0.8 of the m/z ratio listed for the precursor ion in Table 6 as corresponding to the peptide structure.
      92. The method of any one of Embodiments 88-90, wherein the mass-charge (m/z) ratio of the product ion is within ±0.5 of the m/z ratio listed for the precursor ion in Table 6 as corresponding to the peptide structure.
      93. The method of any one of Embodiments 88-92, further comprising:
   generating a treatment output using the quantification data to treat a subject diagnosed with a melanoma condition.
      94. The method of any one of Embodiments 88-93, wherein the reaction monitoring mass spectrometry system uses at least one of multiple reaction monitoring mass spectrometry (MRM-MS), or selected reaction monitoring mass spectrometry (SRM-MS) to detect the set of product ions and generate the quantification data.
      95. The method of any one of Embodiments 88-94, wherein the sample comprises a plasma sample.
      96. The method of any one of Embodiments 88-94, wherein the sample comprises a serum sample.
      97. The method of any one of Embodiment 88-96, wherein preparing the sample comprises at least one of:
   denaturing one or more proteins in the sample to form one or more denatured proteins;
   reducing the one or more denatured proteins in the sample to form one or more reduced proteins;
   alkylating the one or more proteins in the sample using an alkylating agent to prevent reformation of disulfide bonds in the one or more reduced proteins to form one or more alkylated proteins; or
   digesting the one or more alkylated proteins in the sample using a proteolysis catalyst to form the prepared sample comprising the set of peptide structures.
      98. A composition comprising at least one of peptide structures PS-1 to PS-38 identified in Table 1.
      99. A composition comprising a peptide structure or a product ion, wherein:
   the peptide structure or product ion comprises the amino acid sequence having at least 90% sequence identity to any one of SEQ ID NOS: 21-46, corresponding to peptide structures PS-1 to PS-38 in Table 1; and
   the product ion is selected as one from a group consisting of product ions identified in Table 6 including product ions falling within an identified m/z range.
      100. A composition comprising a glycopeptide structure selected as one from a group consisting of peptide structures PS-1 to PS-38 identified in Table 6, wherein:
   the glycopeptide structure comprises:
      an amino acid peptide sequence identified in Table 5 as corresponding to the glycopeptide structure; and
      a glycan structure identified in Table 1 as corresponding to the glycopeptide structure in which the glycan structure is linked to a residue of the amino acid peptide sequence at a corresponding position identified in Table 1; and wherein the glycan structure has a glycan composition.
         101. The composition of Embodiment 100, wherein the glycan composition is identified in Table 7.
         102. The composition of Embodiment 100 or claim 101, wherein:
   the glycopeptide structure has a precursor ion having a charge identified in Table 6 as corresponding to the glycopeptide structure.
      103. The composition of any one of Embodiments 100-101, wherein:
   the glycopeptide structure has a precursor ion with an m/z ratio within ±1.5 of the m/z ratio listed for the precursor ion in Table 6 as corresponding to the glycopeptide structure.
      104. The composition of any one of Embodiments 100-101, wherein:
   the glycopeptide structure has a precursor ion with an m/z ratio within ±1.0 of the m/z ratio listed for the precursor ion in Table 6 as corresponding to the glycopeptide structure.
      105. The composition of any one of Embodiments 100-101, wherein:
   the glycopeptide structure has a precursor ion with an m/z ratio within ±0.5 of the m/z ratio listed for the precursor ion in Table 6 as corresponding to the glycopeptide structure.
      106. The composition of any one of Embodiments 100-105, wherein:
   the glycopeptide structure has a product ion with an m/z ratio within ±1.0 of the m/z ratio listed for the first product ion in Table 6 as corresponding to the glycopeptide structure.
      107. The composition of any one of Embodiments 100-105, wherein:
   the glycopeptide structure has a product ion with an m/z ratio within ±0.8 of the m/z ratio listed for the first product ion in Table 6 as corresponding to the glycopeptide structure.
      108. The composition of any one of Embodiments 100-105, wherein:
   the glycopeptide structure has a product ion with an m/z ratio within ±0.5 of the m/z ratio listed for the first product ion in Table 6 as corresponding to the glycopeptide structure.
      109. The composition of any one of Embodiments 100-108, wherein the glycopeptide structure has a monoisotopic mass identified in Table 1 as corresponding to the glycopeptide structure.
      110. A composition comprising a peptide structure selected as one from a plurality of peptide structures identified in Table 1, wherein:
   the peptide structure has a monoisotopic mass identified as corresponding to the peptide structure in Table 1; and
   the peptide structure comprises the amino acid sequence of SEQ ID NOs: 21-46 identified in Table 1 as corresponding to the peptide structure.
      111. The composition of Embodiment 110, wherein:
   the peptide structure has a precursor ion having a charge identified in Table 6 as corresponding to the peptide structure.
      112. The composition of Embodiment 110, wherein:
   the peptide structure has a precursor ion with an m/z ratio within ±1.5 of the m/z ratio listed for the precursor ion in Table 6 as corresponding to the peptide structure.
      113. The composition of Embodiment 110, wherein:
   the peptide structure has a precursor ion with an m/z ratio within ±1.0 of the m/z ratio listed for the precursor ion in Table 6 as corresponding to the peptide structure.
      114. The composition of Embodiment 110, wherein:
   the peptide structure has a precursor ion with an m/z ratio within ±0.5 of the m/z ratio listed for the precursor ion in Table 6 as corresponding to the peptide structure.
      115. The composition of any one of Embodiments 110-114, wherein:
   the peptide structure has a product ion with an m/z ratio within ±1.0 of the m/z ratio listed for the first product ion in Table 6 as corresponding to the peptide structure.
      116. The composition of any one of Embodiments 110-114, wherein:
   the peptide structure has a product ion with an m/z ratio within ±0.8 of the m/z ratio listed for the first product ion in Table 6 as corresponding to the peptide structure.
      117. The composition of any one of Embodiments 110-114, wherein:
   the peptide structure has a product ion with an m/z ratio within ±0.5 of the m/z ratio listed for the first product ion in Table 6 as corresponding to the peptide structure.
      118. A kit comprising at least one agent for quantifying at least one peptide structure identified in Table 1 to carry out at least a portion of the method of any one of claims 1-87.
      119. A kit comprising at least one of a glycopeptide standard, a buffer, or a set of peptide sequences to carry out at least a portion of the method of any one of claims 1-87, a peptide sequence of the set of peptide sequences identified by a corresponding one of SEQ ID NOS: 21-46, defined in Table 1.
      120. A system comprising:
   one or more data processors; and
      a non-transitory computer readable storage medium containing instructions which, when executed on the one or more data processors, cause the one or more data processors to perform part or all of any one of claims 1-87.
         121. A computer-program product tangibly embodied in a non-transitory machine-readable storage medium, including instructions configured to cause one or more data processors to perform part or all of any one of claims 1-87.
         122. A method for identifying one or more glycopeptide biomarkers predictive of a disease or a condition in a subject, the method comprising:
   obtaining from a subject a first sample at a first timepoint and a second sample at a second timepoint, wherein the first sample and the second sample comprise a glycoprotein;
   fragmenting the glycoprotein in the first sample or the second sample into one or more glycopeptides, wherein the one or more glycopeptides comprise one or more amino acid sequences selected from a group consisting of SEQ ID NO: 21-46, 101-131, and 159-207, and combinations thereof;
   determining an amount of the one or more glycopeptides using multiple reaction monitoring mass spectrometry (MRM-MS);
   associating the amount of the one or more glycopeptides with the first timepoint or the second timepoint, wherein the subject has a change in a disease or a condition from the first timepoint to the second timepoint; and
   identifying as glycopeptide biomarkers the glycopeptide where the amount of the one or more glycopeptides changed from the first timepoint to the second timepoint.
      123. A method for identifying one or more glycopeptide biomarkers predictive of a disease or a condition in a subject, the method comprising:
   obtaining, by a computer, data of an amount of one or more glycopeptides for a set (n) of subjects, wherein the one or more glycopeptides are generated by fragmenting a glycoprotein in a sample from a subject, the amount of one or more glycopeptides are determined using multiple reaction monitoring mass spectrometry (MRM-MS), and the data for each subject comprises data from samples taken at a plurality of timepoints;
   selecting, by the computer, a subset of the one or more glycopeptides to include in a predictive model;
   assessing, by the computer, the predictive model using a cross-validation with n−1 subjects to generate an outcome score for a holdout subject;
   iterating, by the computer, step (c) for each of n subjects as the holdout subject to generate an outcome score for each subject;
   dichotomizing, by the computer, the outcome scores for each subject at a cutoff outcome score as below or above the cutoff outcome score;
   analyzing, by the computer, the amount of one or more glycopeptides for subjects having outcome scores above the cutoff outcome score to the amount of one or more glycopeptides for subjects having outcome scores below the cutoff outcome score for each glycopeptide in the subset of the one or more glycopeptides to determine a hazard ratio and an interaction p-value for each glycopeptide;
   identifying, by the computer, the glycopeptide having the interaction p-value ≤0.05 as a glycopeptide biomarker for predicting the disease or the condition.
      124. A method for identifying one or more glycopeptide biomarkers predictive of a disease or a condition in a subject, the method comprising:
   obtaining, by a computer, data of an amount of one or more glycopeptides for a set (n) of subjects, wherein the one or more glycopeptides are generated by fragmenting a glycoprotein in a sample from a subject, the amount of one or more glycopeptides are determined using multiple reaction monitoring mass spectrometry (MRM-MS), and the data for each subject comprises data from samples taken at a plurality of timepoints;
   selecting, by the computer, a subset of the one or more glycopeptides to include in a predictive model;
   assessing, by the computer, the predictive model using a cross-validation with n−1 subjects to generate an outcome score for a holdout subject;
   iterating, by the computer, step (c) for each of n subjects as the holdout subject to generate an outcome score for each subject;
   dichotomizing, by the computer, the outcome scores for each subject at a cutoff outcome score as below or above the cutoff outcome score;
   analyzing, by the computer, the amount of one or more glycopeptides for subjects having outcome scores above the cutoff outcome score to the amount of one or more glycopeptides for subjects having outcome scores below the cutoff outcome score for each glycopeptide in the subset of the one or more glycopeptides to determine a hazard ratio and an interaction p-value for each glycopeptide;
   identifying, by the computer, the glycopeptide having the interaction p-value ≤0.05 as a glycopeptide biomarker for predicting the disease or the condition.
      125. A method for assessing a status of a condition and a treatment in a subject, the method comprising:
   fragmenting a glycoprotein in a sample from a subject into one or more glycopeptides, wherein the sample comprises one or more of glycoproteins, glycans, or glycopeptides;
   performing mass spectroscopy (MS) on the one or more glycopeptides using multiple reaction monitoring mass spectrometry (MRM-MS) to quantify an amount of the one or more glycopeptides in the sample, wherein the one or more glycopeptides comprise one or more amino acid sequences selected from a group consisting of SEQ ID NOs: 7, 9, 12, 15, 16, 18, 20, 30, 34, 37, 44, 59, 60, 61, 62, 66, 69, 70, 75, 77, 80, and 83, and combinations thereof;
   inputting data of the amount of the one or more glycopeptides into a trained model to generate an output probability, wherein the output probability is indicative of whether a treatment positively influences an outcome of the subject having a condition; and
   generating a treatment recommendation based on the output probability,
   wherein the condition is melanoma and the treatment comprises checkpoint inhibitors.
      126. A method for assessing a status of a condition and a treatment in a subject, the method comprising:
   fragmenting a glycoprotein in a sample from a subject into one or more glycopeptides, wherein the sample comprises one or more of glycoproteins, glycans, or glycopeptides;
   performing mass spectroscopy (MS) on the one or more glycopeptides using multiple reaction monitoring mass spectrometry (MRM-MS) to quantify an amount of the one or more glycopeptides in the sample, wherein the one or more glycopeptides comprise one or more amino acid sequences selected from a group consisting of SEQ ID NOs: 300-429, and combinations thereof;
   inputting data of the amount of the one or more glycopeptides into a trained model to generate an output probability, wherein the output probability is indicative of whether a treatment positively influences an outcome of the subject having a condition; and
   generating a treatment recommendation based on the output probability,
   wherein the condition is non-small cell lung cancer (NSCLC) and the treatment comprises checkpoint inhibitors.
      127. A glycopeptide comprising an amino acid sequence selected from a group consisting of SEQ ID NOs: 300-429, and combinations thereof.
      128. A kit comprising a glycopeptide standard comprising a glycopeptide comprising one or more amino acid sequences selected from a group consisting of SEQ ID NOs: 300-429, and an instruction for using the glycopeptide standard for treating cancer.
      129. The method of any one of Embodiments 122-125, wherein fragmenting comprises protease digestion.
      130. The method of any one of Embodiments 122-125, wherein fragmenting comprises applying a mechanical force.
      131. The method of any one of Embodiments 122-125, wherein the amount of one or more glycopeptides measures multiple reaction monitoring (MRM) transitions.
      132. The method of Embodiment 122, further generating a panel of glycopeptide biomarkers comprising one or more of the glycopeptide biomarkers identified in step (e).
      133. The method of Embodiment 123, wherein the cross-validation is leave-one-out cross-validation (LOOCV).
      134. The method of Embodiment 123, wherein the cutoff outcome score was determined to optimize Harrell's C-index.
      135. The method of Embodiment 123, wherein the interaction p-value is less than or equal to 0.01, 0.005, or 0.001 in step (g).
      136. The method of any one of Embodiments 125-126, wherein the outcome comprises overall survival time.
      137. The method of any one of Embodiments 125-126, wherein the outcome comprises progression-free survival time.
      138. The method of any one of Embodiments 125-126, wherein the treatment comprises one or more of ipilimumab, nivolumab, and pembrolizumab.
      139. The method of any one of Embodiments 125-126, wherein the treatment comprises one or more of PD-1-, PD-L1-, and CTLA-4-inhibitors.
      140. The method of any one of Embodiments 125-126, wherein the treatment comprises chemotherapy.
      141. The method of any one of Embodiments 125-126, wherein the chemotherapy comprises one or more of carboplatin and pemetrexed.
      142. The method of any one of Embodiments 125-126, wherein the recommendation comprises continuing the treatment if the output probability indicates the treatment positively influences the outcome.
      1. A method for managing a treatment for a subject diagnosed with a melanoma or non-small cell lung cancer condition, the method comprising:
   receiving peptide structure data corresponding to a set of glycoproteins in a biological sample obtained from the subject;
   computing a treatment score using quantification data identified from the peptide structure data for a set of peptide structures, wherein the set of peptide structures includes at least one peptide structure identified from a plurality of peptide structures listed in Table 7, Table 12, Table 14, or Table 16;
   generating a treatment output that indicates a predicted response to the treatment for the subject using the treatment score.
      2. The method of embodiment 1A, wherein generating the treatment output comprises:
   generating the predicted response to the treatment based on whether the treatment score is above a selected threshold.
      3. The method of embodiment 2A, wherein the selected threshold is 0.5.
      4. The method of embodiment 2A, wherein the generating the predicted response comprises:
   identifying a first predicted response classification for the subject when the treatment score is above 0.5; and
   identifying a second predicted response classification for the subject when the treatment score is not above 0.5.
      5. The method of embodiment 4A, wherein the first predicted response classification is sustained control and wherein the second predicted response classification is early disruption.
      6. The method of any one of embodiments 1A-5A, wherein the treatment is pembrolizumab and wherein the set of peptide structures includes at least one peptide structure identified from a plurality of peptide structures listed in Table 2.
      7. The method of any one of embodiments 1A-6A, wherein the condition is melanoma and the treatment comprises a combination of nivolumab and ipilimumab and wherein the set of peptide structures includes at least one peptide structure identified from a plurality of peptide structures listed in Table 3.
      8. The method of any one of embodiments 1A-7A, wherein the treatment outcome comprises a recommendation to modify a treatment plan for the subject.
      9. The method of embodiment 8A, wherein the recommendation for modifying the treatment plan includes at least one of selecting a different treatment for the subject, altering a dosage for the treatment, or combining the treatment with at least one other treatment.
      10. The method of any one of embodiments 1A-9A, wherein computing the treatment score comprises:
   computing a proportion of the set of peptide structures having a selected abundance greater than a reference abundance.
      11. The method of embodiment 10A, wherein the reference abundance for a peptide structure of the set of peptide structures is a median of a plurality of abundances for the peptide structure across a sample population and wherein the selected abundance for a glycopeptide structure of the set of peptide structures is a relative abundance and the selected abundance for an aglycosylated peptide structure of the set of peptide structures is an absolute abundance.
      12. The method of any one of embodiments 1A-11A, further comprising:
   identifying the set of peptide structures using sample data and a statistical algorithm that identifies a relative significance for each peptide structure of a collection of peptide structures corresponding to the sample data.
      13. The method of embodiment 12A, wherein the statistical algorithm comprises a Wilcoxon rank-sum test.
      14. The method of embodiment 12A or embodiment 13A, wherein identifying the set of peptide structures comprises:
   performing a differential abundance analysis using the sample data to compare a first portion of the sample data corresponding to a first response classification for the treatment and a second portion of the sample data corresponding to a second response classification for the treatment to identify a selected N most differentiating peptide structures between the first response classification and the second response classification.
      15. The method of embodiment 14A, wherein the selected N most differentiating peptide structures is 20 peptide structures.
      16. The method of embodiment 14A or embodiment 15A, wherein:
   the first response classification is sustained control which indicates an absence of disruption events during a sustained period of time after treatment administration;
   the second response classification is early disruption which indicates a presence of at least one disruption event during an initial period of time after treatment; and
   the sustained period of time is longer than the initial period of time.
      17. The method of embodiment 16A, wherein the sustained period of time is 12 months and the initial period of time is 6 months.
      18. The method of any one of embodiments 1A-17A, wherein the at least one peptide structure comprises a glycopeptide structure defined by a peptide sequence and a glycan structure linked to the peptide sequence at a linking site of the peptide sequence, as identified in Table 1, with the peptide sequence being one of SEQ ID NOS: 21-46 as defined in Table 7.
      19. The method of any one of embodiments 1A-18A, wherein the quantification data for a peptide structure of the set of peptide structures comprises at least one of an adjusted abundance, a relative abundance, an absolute abundance, a normalized abundance, a relative quantity, an adjusted quantity, a normalized quantity, a relative concentration, an adjusted concentration, or a normalized concentration.
      20. The method of any one of embodiments 1A-19A, wherein the peptide structure data is generated using multiple reaction monitoring mass spectrometry (MRM-MS).
      21. The method of any one of embodiments 1A-20A, further comprising:
   creating a sample from the biological sample; and preparing the sample using reduction, alkylation, and enzymatic digestion to form a prepared sample that includes a set of peptide structures.
      22. The method of embodiment 21A, further comprising:
   generating the peptide structure data from the prepared sample using multiple reaction monitoring mass spectrometry (MRM-MS).
      23. The method of any one of embodiments 1A-22A, wherein the treatment output comprises at least one of a design for the treatment or a therapeutic dosage for the treatment.
      24. The method of any one of embodiments 1A-23A, further comprising:
   sending the treatment output to a remote system.
      25. The method of any one of embodiments 1A-24A, further comprising:
   administering a therapeutic dosage of the treatment based on the predicted response being a predicted response classification that indicates the treatment will be successful.
      26. The method of any one of embodiments 1A-25A, further comprising:
   administering a therapeutic dosage of the treatment based on the predicted response being sustained control.
      27. A method for treatment management of a subject diagnosed with a melanoma or non-small cell lung cancer condition, the method comprising:
   receiving peptide structure data corresponding to a set of peptide structures associated with a set of glycoproteins in a biological sample obtained from the subject;
   computing a plurality of treatment scores using quantification data identified from the peptide structure data for a plurality of subsets of the set of peptide structures, wherein each treatment score of the plurality of treatment scores corresponds to a different treatment of a plurality of treatments; wherein each subset of the plurality of subsets includes at least one peptide structure identified from a plurality of peptide structures listed in Table 1, Table 12, Table 14, or Table 16;
   performing a comparison analysis of the plurality of treatment scores; and
   generating a treatment output based on the comparison analysis, wherein the treatment output includes a recommended treatment plan for treating the subject.
      28. The method of embodiment 27A, wherein generating the treatment output comprises:
   identifying a treatment of the plurality of treatments having a highest treatment score as a recommended treatment for treating the subject.
      29. The method of embodiment 27A or embodiment 28A, wherein the condition is melanoma and the plurality of treatments comprises a first treatment of pembrolizumab and a second treatment that is comprised of nivolumab and ipilimumab.
      30. The method of any one of embodiments 27A-29A, wherein performing the comparison analysis comprises:
   determining that a treatment of the plurality of treatments has a treatment score below a selected threshold; and
   excluding the treatment from the comparison analysis.
      31. The method of embodiment 30A, wherein the selected threshold is 0.5.
      32. The method of any one of embodiments 27A-31A, wherein the generating the treatment output comprises:
   identifying a predicted response classification for the subject for each treatment of the plurality of treatments using a corresponding treatment score of the plurality of treatment scores.
      33. The method of embodiment 32A, wherein the predicted response classification is sustained control when the corresponding treatment score is above a selected threshold and is early disruption when the corresponding treatment score is not above the selected threshold.
      34. The method of embodiment 33A, wherein the selected threshold is 0.5.
      35. The method of any one of embodiments 27A-34A, wherein generating the treatment output comprises:
   identifying a treatment of the plurality of treatments having a highest treatment score;
   determining that the highest treatment score is not above a selected threshold; and
   generating the treatment output with the recommended treatment plan including a recommendation to modify an existing treatment plan for the subject.
      36. The method of embodiment 35A, wherein the recommendation for modifying the existing treatment plan includes at least one of selecting a different treatment for the subject, altering a dosage for a treatment that is part of the existing treatment plan, or combining the treatment with at least one other treatment.
      37. The method of any one of embodiments 27A-36A, wherein generating the treatment output comprises:
   identifying a treatment of the plurality of treatments having a highest treatment score as a highest-scored treatment;
   determining that the highest treatment score is above a selected threshold; and
   generating the treatment output with the recommended treatment plan identifying the highest-score treatment as a recommended treatment for treating the subject.
      38. The method of any one of embodiments 27A-37A, wherein the condition is melanoma and wherein a first treatment of the plurality of treatments comprises pembrolizumab, wherein a second treatment of the plurality of treatments comprises a combination of nivolumab and ipilimumab, and wherein computing the plurality of treatment scores comprises:
   computing a first treatment score for the first treatment using a first portion of the quantification data identified from the peptide structure data for a first subset of the plurality of subsets of the set of peptide structures, wherein the first subset includes at least one peptide structure identified from a plurality of peptide structures listed in Table 2; and
   computing a second treatment score for the second treatment using a second portion of the quantification data identified from the peptide structure data for a second subset of the plurality of subsets of the set of peptide structures, wherein the second subset includes at least one peptide structure identified from a plurality of peptide structures listed in Table 3.
      39. The method of any one of embodiments 27A-38A, wherein computing the plurality of treatment scores comprises:
   computing a proportion of a subset of the plurality of subsets of the set of peptide structures having a selected abundance greater than a reference abundance as a treatment score of the plurality of treatment scores.
      40. The method of embodiment 39A, wherein the reference abundance for a peptide structure of the set of peptide structures is a median of a plurality of abundances for the peptide structure across a sample population and wherein the selected abundance for a glycopeptide structure of the set of peptide structures is a relative abundance and the selected abundance for an aglycosylated peptide structure of the set of peptide structures is an absolute abundance.
      41. The method of any one of embodiments 27A-40A, further comprising:
   identifying a subset of the plurality of subsets of the set of peptide structures using sample data and a statistical algorithm that identifies a relative significance for each peptide structure of a collection of peptide structures corresponding to the sample data with respect to a response to a selected treatment of the plurality of treatments.
      42. The method of embodiment 41A, wherein the statistical algorithm comprises a Wilcoxon rank-sum test.
      43. The method of embodiment 41A or embodiment 42A, wherein identifying the subset comprises:
   performing a differential abundance analysis using the sample data to compare a first portion of the sample data corresponding to a first response classification for the selected treatment and a second portion of the sample data corresponding to a second response classification for the selected treatment to identify a selected N most differentiating peptide structures between the first response classification and the second response classification.
      44. The method of embodiment 43A, wherein the selected N most differentiating peptide structures is 20 peptide structures.
      45. The method of embodiment 43A or embodiment 44A, wherein:
   the first response classification is sustained control which indicates an absence of disruption events during a sustained period of time after treatment administration;
   the second response classification is early disruption which indicates a presence of at least one disruption event during an initial period of time after treatment; and
   the sustained period of time is longer than the initial period of time.
      46. The method of embodiment 45, wherein the sustained period of time is 12 months and the initial period of time is 6 months.
      47. The method of any one of embodiments 27A-46A, wherein the at least one peptide structure comprises a glycopeptide structure defined by a peptide sequence and a glycan structure linked to the peptide sequence at a linking site of the peptide sequence, as identified in Table 1, with the peptide sequence being one of SEQ ID NOS: 21-46 as defined in Table 7.
      48. The method of any one of embodiments 27A-47A, wherein the quantification data for a peptide structure of the set of peptide structures comprises at least one of an adjusted abundance, a relative abundance, an absolute abundance, a normalized abundance, a relative quantity, an adjusted quantity, a normalized quantity, a relative concentration, an adjusted concentration, or a normalized concentration.
      49. The method of claim any one of embodiments 27A-48A, wherein the peptide structure data is generated using multiple reaction monitoring mass spectrometry (MRM-MS).
      50. The method of any one of embodiments 27A-49A, further comprising:
creating a sample from the biological sample; and
   preparing the sample using reduction, alkylation, and enzymatic digestion to form a prepared sample that includes a set of peptide structures.
      51. The method of embodiment 50A, further comprising:
   generating the peptide structure data from the prepared sample using multiple reaction monitoring mass spectrometry (MRM-MS).
      52. The method of any one of embodiments 27A-51A, wherein the recommended treatment plan identifies a recommended treatment and a therapeutic dosage for the recommended treatment.
      53. The method of embodiment 52A, further comprising:
   administering a therapeutic dosage of the recommended.
      54. The method of any one of embodiments 27A-53A, further comprising:
   sending the treatment output to a remote system.
      55. A method for treatment management of a subject diagnosed with a melanoma condition, the method comprising:
   receiving peptide structure data corresponding to a set of peptide structures associated with a set of glycoproteins in a biological sample obtained from the subject;
   computing a first treatment score for a first treatment of pembrolizumab using first quantification data identified from the peptide structure data for a first subset of the set of peptide structures, wherein the first subset includes at least one peptide structure identified from a plurality of peptide structures listed in Table 2;
   computing a second treatment score for a second treatment comprised of nivolumab and ipilimumab using second quantification data identified from the peptide structure data for a second subset of the set of peptide structures, wherein the second subset includes at least one peptide structure identified from a plurality of peptide structures listed in Table 3;
   performing a comparison analysis of the first treatment score and the second treatment score; and
   generating a treatment output based on the comparison analysis, wherein the treatment output identifies one of the first treatment and the second treatment as a recommended treatment for the subject.
      56. The method of embodiment 55A, wherein computing the first treatment score comprises:
   computing a proportion of the first subset having a selected abundance greater than a reference abundance as the first treatment score.
      57. The method of embodiment 55A or embodiment 56A, wherein computing the second treatment score comprises:
   computing a proportion of the second subset having a selected abundance greater than a reference abundance as the second treatment score.
      58. A method for treating a subject diagnosed with a melanoma or non-small cell lung cancer condition, comprising:
   receiving peptide structure data corresponding to a set of glycoproteins in a biological sample obtained from the subject;
   computing a treatment score using quantification data identified from the peptide structure data for a set of peptide structures, wherein the set of peptide structures includes at least one peptide structure identified from a plurality of peptide structures listed in Table 1, Table 12, Table 14, or Table 16;
   generating a treatment output that indicates a predicted response to a treatment for the subject using the treatment score; and
   administering the treatment to the patient in response to the predicted response includes a positive response classification, the step of administering comprising at least one of intravenous or oral administration of the recommended treatment or a derivative thereof at a therapeutic dosage,
      wherein the treatment is selected as one from a group consisting of:
         a first treatment of pembrolizumab for which the therapeutic dosage of at least one of 200 mg every three weeks, 2 mg/kg every three weeks is administered, or 400 mg every 6 weeks; and
         a second treatment comprised of nivolumab and ipilimumab for which the therapeutic dosage of either 1 mg/kg nivolumab with 3 mg/kg ipilimumab or 3 mg/kg nivolumab with 1 mg/kg ipilimumab is administered.
            59. A method for treating a subject diagnosed with a melanoma condition, comprising:
   receiving peptide structure data corresponding to a set of peptide structures associated with a set of glycoproteins in a biological sample obtained from the subject;
   computing a plurality of treatment scores using quantification data identified from the peptide structure data for a plurality of subsets of the set of peptide structures, wherein each treatment score of the plurality of treatment scores corresponds to a different treatment of a plurality of treatments; wherein each subset of the plurality of subsets includes at least one peptide structure identified from a plurality of peptide structures listed in Table 1, Table 12, Table 14, or Table 16;
   performing a comparison analysis of the plurality of treatment scores;
   generating a treatment output based on the comparison analysis, wherein the treatment output includes a recommended treatment from the plurality of treatments for treating the subject; and
   administering the recommended treatment to the patient, the step of administering comprising at least one of intravenous or oral administration of the recommended treatment or a derivative thereof at a therapeutic dosage,
      wherein the plurality of treatments comprises:
         a first treatment of pembrolizumab for which the therapeutic dosage of at least one of 200 mg every three weeks, 2 mg/kg every three weeks is administered, or 400 mg every 6 weeks; and
         a second treatment comprised of nivolumab and ipilimumab for which the therapeutic dosage of either 1 mg/kg nivolumab with 3 mg/kg ipilimumab or 3 mg/kg nivolumab with 1 mg/kg ipilimumab is administered.
            60. A method for managing a treatment for a subject diagnosed with a melanoma or non-small cell lung cancer condition, the method comprising:
   receiving sample data for a sample population, wherein the sample data characterizes responses of a plurality of sample subjects diagnosed with the melanoma or non-small cell lung cancer condition to the treatment and includes sample peptide structure data for a collection of peptide structures for each subject of the plurality of sample subjects;
   grouping the sample data based on the responses of the plurality of sample subjects into a first group corresponding to a first response classification and a second group corresponding to a second response classification;
   performing a differential abundance analysis using the sample data to compare the first group of the sample data corresponding to the first response classification and the second group of the sample data corresponding to the second response classification to identify a set of peptide structures from the collection of peptide structures,
      wherein the set of peptide structures comprises a selected N most differentiating peptide structures between the first response classification and the second response classification; and
   receiving peptide structure data corresponding to a set of glycoproteins in a biological sample obtained from the subject;
   computing a treatment score for the treatment using quantification data identified from the peptide structure data for the set of peptide structures; and
   generating a treatment output that indicates a predicted response to the treatment for the subject using the treatment score.
      61. The method of embodiment 60A, wherein the set of peptide structures includes at least one peptide structure identified from a plurality of peptide structures listed in Table 1, Table 12, Table 14, or Table 16.
      62. The method of embodiment 60A or embodiment 61A, wherein the differential abundance analysis is performed using a Wilcoxon rank-sum test.
      63. The method of any one of embodiments 60A-62A, wherein the selected N most differentiating peptide structures is 20 peptide structures.
      64. The method of any one of embodiment 60A-63A, wherein:
   the first response classification is sustained control which indicates an absence of disruption events during a sustained period of time after treatment administration;
   the second response classification is early disruption which indicates a presence of at least one disruption event during an initial period of time after treatment; and
   the sustained period of time is longer than the initial period of time.
      65. The method of embodiment 64A, wherein the sustained period of time is 12 months and the initial period of time is 6 months.
      66. A method of treating melanoma or non-small cell lung cancer in a subject, the method comprising:
   receiving peptide structure data corresponding to a set of glycoproteins in a biological sample obtained from the subject;
   computing a treatment score using quantification data identified from the peptide structure data for a set of peptide structures, wherein the set of peptide structures includes at least one peptide structure identified from a plurality of peptide structures listed in Table 1, Table 12, Table 14, or Table 16;
   generating a treatment output using the treatment score; and
   administering a pembrolizumab treatment to the subject if the treatment output includes at least one of a positive response classification for the pembrolizumab treatment or an identification of the pembrolizumab treatment as a recommended treatment.
      67. The method of embodiment 66A, wherein the administering comprises:
administering the pembrolizumab treatment at a dosage of 200 mg every 3 weeks.
   68. The method of embodiment 66A, wherein the administering comprises:
administering the pembrolizumab treatment at a dosage of 2 mg/kg mg every 3 weeks.
   69. The method of embodiment 66A, wherein the administering comprises:
administering the pembrolizumab treatment at a dosage of 400 mg every 3 weeks.
   70. The method of any one of embodiments 66A-69A, wherein the administering comprises:
   administering the pembrolizumab treatment via an intravenous route of administration.
      71. The method of any one of embodiments 66A-70A, wherein the administering comprises:
   administering the pembrolizumab treatment every three weeks for four doses.
      72. A method of treating melanoma or non-small cell lung cancer in a subject, the method comprising:
   receiving peptide structure data corresponding to a set of glycoproteins in a biological sample obtained from the subject;
   computing a treatment score using quantification data identified from the peptide structure data for a set of peptide structures, wherein the set of peptide structures includes at least one peptide structure identified from a plurality of peptide structures listed in Table 1, Table 12, Table 14, or Table 16;
   generating a treatment output using the treatment score; and
   administering a combination treatment comprising a combination of nivolumab and ipilimumab to the subject if the treatment output includes at least one of a positive response classification for the combination treatment or an identification of the combination treatment as a recommended treatment.
      73. The method of embodiment 72A, wherein the administering comprises:
   administering the combination treatment to the subject at a dosage of 1 mg/kg of nivolumab with 3 mg/kg of ipilimumab.
      74. The method of embodiment 72A, wherein the administering comprises:
   administering the combination treatment to the subject at a dosage of 3 mg/kg of nivolumab with 1 mg/kg of ipilimumab.
      75. The method of any one of embodiments 72A-74A, wherein the administering comprises:
   administering the combination treatment via an intravenous route of administration.
      76. The method of any one of embodiments 72A-75A, wherein the administering comprises:
   administering the combination treatment every three weeks for four doses.
      77. A method of identifying patients with melanoma or non-small cell lung cancer for treatment with a pembrolizumab treatment, the method comprising:
   receiving peptide structure data corresponding to a set of glycoproteins in a biological sample obtained from the subject;
   computing a treatment score using quantification data identified from the peptide structure data for a set of peptide structures, wherein the set of peptide structures includes at least one peptide structure identified from a plurality of peptide structures listed in Table 1, Table 12, Table 14, or Table 16; and
   generating a treatment output using the treatment score,
   wherein the patient is treated with the pembrolizumab treatment if the treatment output includes at least one of a positive response classification for the pembrolizumab treatment or an identification of the pembrolizumab treatment as a recommended treatment.
      78. The method of embodiment 77A, wherein the pembrolizumab treatment is administered at a dosage of 200 mg every 3 weeks.
      79. The method of embodiment 77A, wherein the pembrolizumab treatment is administered at a dosage of 2 mg/kg mg every 3 weeks.
      80. The method of embodiment 77A, wherein the pembrolizumab treatment is administered at a dosage of 400 mg every 3 weeks.
      81. The method of any one of embodiments 77A-80A, wherein the pembrolizumab treatment is administered via an intravenous route of administration.
      82. The method of any one of embodiment 77A-81A, wherein the pembrolizumab treatment is administered every three weeks for four doses.
      83. A method of identifying patients with melanoma for treatment with a combination treatment comprising nivolumab and ipilimumab, the method comprising:
   receiving peptide structure data corresponding to a set of glycoproteins in a biological sample obtained from the subject;
   computing a treatment score using quantification data identified from the peptide structure data for a set of peptide structures, wherein the set of peptide structures includes at least one peptide structure identified from a plurality of peptide structures listed in Table 1, Table 12, Table 14, or Table 16; and
   generating a treatment output using the treatment score,
   wherein the patient is treated with the combination treatment if the treatment output includes at least one of a positive response classification for the combination treatment or an identification of the combination treatment as a recommended treatment.
      84. The method of embodiment 83A, wherein the combination treatment is administered at a dosage of 1 mg/kg of nivolumab combined with 3 mg/kg of ipilimumab.
      85. The method of embodiment 83a, wherein the combination treatment is administered at a dosage of 3 mg/kg of nivolumab combined with 1 mg/kg of ipilimumab.
      86. The method of any one of embodiments 83A-85A, wherein the combination treatment is administered via an intravenous route of administration.
      87. The method of any one of embodiments 83A-86A, wherein the combination treatment is administered every three weeks for four doses.
      88. A method for analyzing a set of peptide structures in a sample from a patient, the method comprising:
   (a) obtaining the sample from the patient;
      (b) preparing the sample to form a prepared sample comprising a set of peptide structures;
      (c) inputting the prepared sample into a reaction monitoring mass spectrometry system to detect a set of product ions associated with each peptide structure of the set of peptide structures,
         wherein the set of peptide structures includes at least one peptide structure selected from peptide structures PS-1 to PS-38 identified in Table 6;
         wherein the set of peptide structures includes a peptide structure that is characterized as having:
            (i) a precursor ion with a mass-charge (m/z) ratio within ±1.5 of the m/z ratio listed for the precursor ion in Table 6 as corresponding to the peptide structure; and
            (ii) a product ion having an m/z ratio within ±1.0 of the m/z ratio listed for the first product ion in Table 6 as corresponding to the peptide structure; and
      (d) generating quantification data for the set of product ions using the reaction monitoring mass spectrometry system.
         89. The method of embodiment 88A, wherein the mass-charge (m/z) ratio of the precursor ion is within ±1.0 of the m/z ratio listed for the precursor ion in Table 6 as corresponding to the peptide structure.
         90. The method of embodiment 88A, wherein the mass-charge (m/z) ratio of the precursor ion is within ±0.5 of the m/z ratio listed for the precursor ion in Table 6 as corresponding to the peptide structure.
         91. The method of any one of embodiments 88A-90A, wherein the mass-charge (m/z) ratio of the product ion is within ±0.8 of the m/z ratio listed for the precursor ion in Table 6 as corresponding to the peptide structure.
         92. The method of any one of embodiments 88A-90A, wherein the mass-charge (m/z) ratio of the product ion is within ±0.5 of the m/z ratio listed for the precursor ion in Table 6 as corresponding to the peptide structure.
         93. The method of any one of embodiments 88A-92A, further comprising:
   generating a treatment output using the quantification data to treat a subject diagnosed with a melanoma condition.
      94. The method of any one of embodiments 88A-93A, wherein the reaction monitoring mass spectrometry system uses at least one of multiple reaction monitoring mass spectrometry (MRM-MS), or selected reaction monitoring mass spectrometry (SRM-MS) to detect the set of product ions and generate the quantification data.
      95. The method of any one of embodiments 88A-94A, wherein the sample comprises a plasma sample.
      96. The method of any one of embodiments 88A-94A, wherein the sample comprises a serum sample.
      97. The method of any one of embodiments 88A-96A, wherein preparing the sample comprises at least one of:
   denaturing one or more proteins in the sample to form one or more denatured proteins;
   reducing the one or more denatured proteins in the sample to form one or more reduced proteins;
   alkylating the one or more proteins in the sample using an alkylating agent to prevent reformation of disulfide bonds in the one or more reduced proteins to form one or more alkylated proteins; or
   digesting the one or more alkylated proteins in the sample using a proteolysis catalyst to form the prepared sample comprising the set of peptide structures.
      98. A composition comprising at least one of peptide structures identified in Table 1, Table 12, Table 14, or Table 16.
      99. A composition comprising a peptide structure or a product ion, wherein:
   the peptide structure or product ion comprises the amino acid sequence having at least 90% sequence identity to any one of SEQ ID NOS: 21-46, 101-131, and 159-257; and
   the product ion is selected as one from a group consisting of product ions identified in Table 6, 18, or 20 including product ions falling within an identified m/z range.
      100. A composition comprising a glycopeptide structure selected as one from a group consisting of peptide structures PS-1 to PS-38 identified in Table 6, peptide structures from Table 18, or peptide structures from Table 20, wherein:
   the glycopeptide structure comprises:
      an amino acid peptide sequence identified in Table 5 as corresponding to the glycopeptide structure; and
      a glycan structure identified in Table 1 as corresponding to the glycopeptide structure in which the glycan structure is linked to a residue of the amino acid peptide sequence at a corresponding position identified in Table 1; and
         wherein the glycan structure has a glycan composition.
            101. The composition of embodiment 100A, wherein the glycan composition is identified in Table 7.
            102. The composition of embodiment 100A or embodiment 101A, wherein:
   the glycopeptide structure has a precursor ion having a charge identified in Table 6, Table 18, or Table 20 as corresponding to the glycopeptide structure.
      103. The composition of any one of embodiments 100A-101A, wherein:
   the glycopeptide structure has a precursor ion with an m/z ratio within ±1.5 of the m/z ratio listed for the precursor ion in Table 6, Table 18, or Table 20 as corresponding to the glycopeptide structure.
      104. The composition of any one of embodiments 100A-101A, wherein:
   the glycopeptide structure has a precursor ion with an m/z ratio within ±1.0 of the m/z ratio listed for the precursor ion in Table 6, Table 18, or Table 20 as corresponding to the glycopeptide structure.
      105. The composition of any one of embodiments 100A-101A, wherein:
   the glycopeptide structure has a precursor ion with an m/z ratio within ±0.5 of the m/z ratio listed for the precursor ion in Table 6, Table 18, or Table 20 as corresponding to the glycopeptide structure.
      106. The composition of any one of embodiments 100A-105A, wherein:
   the glycopeptide structure has a product ion with an m/z ratio within ±1.0 of the m/z ratio listed for the first product ion in Table 6, Table 18, or Table 20 as corresponding to the glycopeptide structure.
      107. The composition of any one of embodiments 100A-105A, wherein:
   the glycopeptide structure has a product ion with an m/z ratio within ±0.8 of the m/z ratio listed for the first product ion in Table 6, Table 18, or Table 20 as corresponding to the glycopeptide structure.
      108. The composition of any one of embodiments 100A-105A, wherein:
   the glycopeptide structure has a product ion with an m/z ratio within ±0.5 of the m/z ratio listed for the first product ion in Table 6 as corresponding to the glycopeptide structure.
      109. The composition of any one of embodiments 100A-108A, wherein the glycopeptide structure has a monoisotopic mass identified in Table 1 as corresponding to the glycopeptide structure.
      110. A composition comprising a peptide structure selected as one from a plurality of peptide structures identified in Table 1, wherein:
   the peptide structure has a monoisotopic mass identified as corresponding to the peptide structure in Table 1; and
   the peptide structure comprises the amino acid sequence of SEQ ID NOs: 21-46 as corresponding to the peptide structure.
      111. The composition of embodiment 110A, wherein:
   the peptide structure has a precursor ion having a charge identified in Table 6 as corresponding to the peptide structure.
      112. The composition of embodiment 110A, wherein:
   the peptide structure has a precursor ion with an m/z ratio within ±1.5 of the m/z ratio listed for the precursor ion in Table 6 as corresponding to the peptide structure.
      113. The composition of embodiment 110A, wherein:
   the peptide structure has a precursor ion with an m/z ratio within ±1.0 of the m/z ratio listed for the precursor ion in Table 6 as corresponding to the peptide structure.
      114. The composition of embodiment 110A, wherein:
   the peptide structure has a precursor ion with an m/z ratio within ±0.5 of the m/z ratio listed for the precursor ion in Table 6 as corresponding to the peptide structure.
      115. The composition of any one of embodiments 110A-114A, wherein:
   the peptide structure has a product ion with an m/z ratio within ±1.0 of the m/z ratio listed for the first product ion in Table 6 as corresponding to the peptide structure.
      116. The composition of any one of embodiments 110A-114A, wherein:
   the peptide structure has a product ion with an m/z ratio within ±0.8 of the m/z ratio listed for the first product ion in Table 6 as corresponding to the peptide structure.
      117. The composition of any one of embodiments 110A-114A, wherein:
   the peptide structure has a product ion with an m/z ratio within ±0.5 of the m/z ratio listed for the first product ion in Table 6 as corresponding to the peptide structure.
      118. A kit comprising at least one agent for quantifying at least one peptide structure identified in Table 1, Table 12, Table 14, or Table 16 to carry out at least a portion of the method of any one of claims 1-87.
      119. A kit comprising at least one of a glycopeptide standard, a buffer, or a set of peptide sequences to carry out at least a portion of the method of any one of claims 1-87, a peptide sequence of the set of peptide sequences identified by a corresponding one of SEQ ID NOS: 21-46, 101-131, 159-207, or 300-429.
      120. A system comprising:
one or more data processors; and
   a non-transitory computer readable storage medium containing instructions which, when executed on the one or more data processors, cause the one or more data processors to perform part or all of any one of claims 1-87.
      121. A computer-program product tangibly embodied in a non-transitory machine-readable storage medium, including instructions configured to cause one or more data processors to perform part or all of any one of embodiments 1A-87A.
      122. The method of any one of embodiments 1A-82A, wherein the subject has melanoma and wherein the set of peptide structures comprises at least one peptide structure from table 12.
      123. The method of embodiment 122A, wherein the set of peptide structures comprises at least two, at least three, at least 4, at least 5, at least 6, at least 7, at least 8, at least 9, at least 10, at least 15 or at least 20 peptide structures from table 12.
      124. The method of embodiment 122A or 123A, wherein the subject has advanced melanoma and/or malignant melanoma.
      125. The method any one of embodiments 1A-87A, wherein the subject has non-small cell lung cancer, and wherein the set of peptide structures comprises at least one peptide structure from table 14.
      126. The method of embodiment 125A, wherein the set of peptide structures comprises at least two, at least three, at least 4, at least 5, at least 6, at least 7, at least 8, at least 9, at least 10, at least 15 or at least 20 peptide structures from table 14.
      127. The method any one of embodiments 1A-87A, wherein the subject has non-small cell lung cancer, and wherein the set of peptide structures comprises at least one peptide structure from table 16.
      128. The method of embodiment 125A, wherein the set of peptide structures comprises at least two, at least three, at least 4, at least 5, at least 6, at least 7, at least 8, at least 9, at least 10, at least 15 or at least 20 peptide structures from table 16.
      129. The method of any one of embodiments 27A-28A, wherein if the treatment output indicates that the subject is not likely to respond to pembrolizumab or nivolumab and ipilimumab, the recommended treatment plan comprises an alternative therapy selected from the group consisting of standard non-checkpoint immunotherapy, standard chemotherapy, combination chemotherapy and non-checkpoint immunotherapy, targeted therapy, radiation therapy, a new generation checkpoint inhibitor alone or in combination, a LAG-3 inhibitor, a recommend for participation in a clinical trial for an oncotherapeutic, laser therapy or photodynamic therapy.
      130. The method of any one of embodiments 27A-28A, wherein if the treatment output indicates that the subject is not likely to respond to pembrolizumab or nivolumab and ipilimumab, further comprising administering an alternative therapy selected from the group consisting of standard non-checkpoint immunotherapy, standard chemotherapy, combination chemotherapy and non-checkpoint immunotherapy, targeted therapy, radiation therapy, a new generation checkpoint inhibitor alone or in combination, a LAG-3 inhibitor, a recommend for participation in a clinical trial for an oncotherapeutic, laser therapy or photodynamic therapy.
      131. The method of any one of embodiments 27A-28A, wherein the subject has melanoma, and wherein if the treatment output indicates that the subject is not likely to respond to pembrolizumab or nivolumab and ipilimumab, the recommended treatment plan comprises an alternative therapy selected from the group consisting of other immunotherapy, injection of T-VEC (talimogene laherparepvec) vaccine, Bacille Clamette-Guerin vaccine, imiquimod cream, IL-2 immunotherapy, chemotherapy, dacarbazine and temozolomide either alone or in combination with other drugs, combination of BRAF inhibitor and MEK inhibitor for subjects with BRAF gene change, imatinib or nilotinib for subjects with changes to c-KIT gene, and radiation therapy.
      132. The method of any one of embodiments 27A-28A, wherein the subject has melanoma, and wherein if the treatment output indicates that the subject is not likely to respond to pembrolizumab or nivolumab and ipilimumab, further comprising administering an alternative therapy selected from the group consisting of other immunotherapy, injection of T-VEC (talimogene laherparepvec) vaccine, Bacille Clamette-Guerin vaccine, imiquimod cream, IL-2 immunotherapy, chemotherapy, dacarbazine and temozolomide either alone or in combination with other drugs, combination of BRAF inhibitor and MEK inhibitor for subjects with BRAF gene change, imatinib or nilotinib for subjects with changes to c-KIT gene, and radiation therapy.
      133. The method of any one of embodiments 27A-28A, wherein the subject has non-small cell lung cancer, and wherein if the treatment output indicates that the subject is not likely to respond to nivolumab and ipilimumab, the recommended treatment plan comprises an alternative therapy selected from the group consisting of adjuvant treatment with osimertinib for subject with EGFR mutations, targeted therapy for patients with certain gene mutations such as anti-angiogenic agents, drugs that target cells with KRAS gene changes, drugs that target cells with EGFR changes, drugs that target cells with ALK gene changes, drugs that target cells with ROS1 gene changes, drugs that target cells with BRAF gene changes, chemotherapy, cisplatin, carboplatin, paclitaxel, albumin-bound paclitaxel, docetaxel, gemcitabine, vinorelbine, etoposide, pemetrexed, chemotherapy combined with radiation therapy (chemoradiation) and chemoradiation followed by durvalumab.
      134. The method of any one of embodiments 27A-28A, wherein the subject has non-small cell lung cancer, and wherein if the treatment output indicates that the subject is not likely to respond to nivolumab and ipilimumab, administering an alternative therapy selected from the group consisting of adjuvant treatment with osimertinib for subject with EGFR mutations, targeted therapy for patients with certain gene mutations such as anti-angiogenic agents, drugs that target cells with KRAS gene changes, drugs that target cells with EGFR changes, drugs that target cells with ALK gene changes, drugs that target cells with ROS1 gene changes, drugs that target cells with BRAF gene changes, chemotherapy, cisplatin, carboplatin, paclitaxel, albumin-bound paclitaxel, docetaxel, gemcitabine, vinorelbine, etoposide, pemetrexed, chemotherapy combined with radiation therapy (chemoradiation) and chemoradiation followed by durvalumab.
      135. The method of any one of embodiments 129A-134A, wherein the alternative therapy is a first-line therapy.
      136. The method of any one of embodiments 129A-134A, wherein the subject has received a first-line therapy, wherein the alternative therapy is a second-line therapy, and wherein the alternative therapy is different from the first-line therapy.
      137. The method of any one of embodiments 8A-26A, wherein the recommendation for modifying the existing treatment plan comprises selecting a different treatment for the subject selected from the group consisting of standard non-checkpoint immunotherapy, standard chemotherapy, combination chemotherapy and non-checkpoint immunotherapy, targeted therapy, radiation therapy, a new generation checkpoint inhibitor alone or in combination, a LAG-3 inhibitor, a recommend for participation in a clinical trial for an oncotherapeutic, laser therapy or photodynamic therapy.
      138. The method of any one of embodiments 8A-26A, wherein the subject has melanoma, and wherein the recommendation for modifying the existing treatment plan comprises selecting a different treatment for the subject selected from the group consisting of other immunotherapy, injection of T-VEC (talimogene laherparepvec) vaccine, Bacille Clamette-Guerin vaccine, imiquimod cream, IL-2 immunotherapy, chemotherapy, dacarbazine and temozolomide either alone or in combination with other drugs, combination of BRAF inhibitor and MEK inhibitor for subjects with BRAF gene change, imatinib or nilotinib for subjects with changes to c-KIT gene, and radiation therapy.
      139. The method of any one of embodiments 8A-26A, wherein the subject has non-small cell lung cancer and wherein the recommendation for modifying the existing treatment plan comprises selecting a different treatment for the subject selected from the group consisting of adjuvant treatment with osimertinib for subject with EGFR mutations, targeted therapy for patients with certain gene mutations such as anti-angiogenic agents, drugs that target cells with KRAS gene changes, drugs that target cells with EGFR changes, drugs that target cells with ALK gene changes, drugs that target cells with ROS1 gene changes, drugs that target cells with BRAF gene changes, chemotherapy, cisplatin, carboplatin, paclitaxel, albumin-bound paclitaxel, docetaxel, gemcitabine, vinorelbine, etoposide, pemetrexed, chemotherapy combined with radiation therapy (chemoradiation) and chemoradiation followed by durvalumab.
      140. The method of any one of embodiment 137A-139A, further comprising administering the selected different treatment.
      141. The method of embodiment 136A or embodiment 137A, wherein the subject has received a previous therapy and wherein the recommendation for modifying the existing treatment plan comprises selecting a therapy other than the previous therapy.
      142. A method for identifying a subject that is unlikely to respond to treatment with pembrolizumab or nivolumab and ipilimumab comprising
   receiving peptide structure data corresponding to a set of glycoproteins in a biological sample obtained from the subject;
   computing a treatment score using quantification data identified from the peptide structure data for a set of peptide structures, wherein the set of peptide structures includes at least one peptide structure identified from a plurality of peptide structures listed in Table 7, Table 12, Table 14, or Table 16;
   generating a treatment output that indicates a predicted response to the pembrolizumab or nivolumab and ipilimumab for the subject using the treatment score.
      143. The method of embodiment 142A, wherein generating the treatment output comprises: generating the predicted response to the treatment based on whether the treatment score is above a selected threshold.
      144. The method of embodiment 143A, wherein if predicted response to treatment indicates that the subject is unlikely to respond to treatment with pembrolizumab or nivolumab and ipilimumab, the subject is administered other immunotherapy, injection of T-VEC (talimogene laherparepvec) vaccine, Bacille Clamette-Guerin vaccine, imiquimod cream, IL-2 immunotherapy, chemotherapy, dacarbazine and temozolomide either alone or in combination with other drugs, combination of BRAF inhibitor and MEK inhibitor for subjects with BRAF gene change, imatinib or nilotinib for subjects with changes to c-KIT gene, radiation therapy, osimertinib for subject with EGFR mutations, targeted therapy for patients with certain gene mutations such as anti-angiogenic agents, drugs that target cells with KRAS gene changes, drugs that target cells with EGFR changes, drugs that target cells with ALK gene changes, drugs that target cells with ROS1 gene changes, drugs that target cells with BRAF gene changes, chemotherapy, cisplatin, carboplatin, paclitaxel, albumin-bound paclitaxel, docetaxel, gemcitabine, vinorelbine, etoposide, pemetrexed, chemotherapy combined with radiation therapy (chemoradiation) and chemoradiation followed by durvalumab, non-checkpoint immunotherapy, standard chemotherapy, combination chemotherapy and non-checkpoint immunotherapy, targeted therapy, radiation therapy, a new generation checkpoint inhibitor alone or in combination, a LAG-3 inhibitor, a recommend for participation in a clinical trial for an oncotherapeutic, laser therapy and photodynamic therapy.

What is claimed is:

1. A method for managing a treatment for a subject diagnosed with a melanoma or non-small cell lung cancer condition, the method comprising:
receiving peptide structure data corresponding to a set of glycoproteins in a biological sample obtained from the subject;
computing a treatment score using quantification data identified from the peptide structure data for a set of peptide structures, wherein the set of peptide structures includes at least one peptide structure identified from a plurality of peptide structures listed in Table 1, Table 12, Table 14, or Table 16; and
generating a treatment output that indicates a predicted response to the treatment for the subject using the treatment score.

2. The method of claim 1, wherein generating the treatment output comprises:
generating the predicted response to the treatment based on whether the treatment score is above a selected threshold.

3. (canceled)

4. The method of claim 2, wherein the generating the predicted response comprises:
identifying a first predicted response classification for the subject when the treatment score is above 0.5; and
identifying a second predicted response classification for the subject when the treatment score is not above 0.5.

5. The method of claim 4, wherein the first predicted response classification is sustained control and wherein the second predicted response classification is early disruption.

6. The method of claim 1, wherein the treatment is pembrolizumab and wherein the set of peptide structures includes at least one peptide structure identified from a plurality of peptide structures listed in Table 2.

7. The method of claim 1, wherein the condition is melanoma and the treatment comprises a combination of nivolumab and ipilimumab and wherein the set of peptide structures includes at least one peptide structure identified from a plurality of peptide structures listed in Table 3.

8. The method of claim 1, wherein the treatment outcome comprises a recommendation to modify a treatment plan for the subject.

9. (canceled)

10. The method of claim 1, wherein computing the treatment score comprises:
computing a proportion of the set of peptide structures having a selected abundance greater than a reference abundance.

11. The method of claim 10, wherein the reference abundance for a peptide structure of the set of peptide structures is a median of a plurality of abundances for the peptide structure across a sample population and wherein the selected abundance for a glycopeptide structure of the set of peptide structures is a relative abundance and the selected abundance for an aglycosylated peptide structure of the set of peptide structures is an absolute abundance.

12. The method of claim 1, further comprising:
identifying the set of peptide structures using sample data and a statistical algorithm that identifies a relative significance for each peptide structure of a collection of peptide structures corresponding to the sample data.

13.-15. (canceled)

16. The method of claim 14, wherein:
the first response classification is sustained control which indicates an absence of disruption events during a sustained period of time after treatment administration;
the second response classification is early disruption which indicates a presence of at least one disruption event during an initial period of time after treatment; and
the sustained period of time is longer than the initial period of time.

17. (canceled)

18. The method of claim 1, wherein the at least one peptide structure comprises a glycopeptide structure defined by a peptide sequence and a glycan structure linked to the peptide sequence at a linking site of the peptide sequence, as identified in Table 1, with the peptide sequence being one of SEQ ID NOS: 21-46 as defined in Table 7.

19. The method of claim 1, wherein the quantification data for a peptide structure of the set of peptide structures comprises at least one of an adjusted abundance, a relative abundance, an absolute abundance, a normalized abundance, a relative quantity, an adjusted quantity, a normalized quantity, a relative concentration, an adjusted concentration, or a normalized concentration.

20. The method of claim 1, wherein the peptide structure data is generated using multiple reaction monitoring mass spectrometry (MRM-MS).

21. The method of claim 1, further comprising:
creating a sample from the biological sample; and
preparing the sample using reduction, alkylation, and enzymatic digestion to form a prepared sample that includes a set of peptide structures.

22. (canceled)

23. The method of claim 1, wherein the treatment output comprises at least one of a design for the treatment or a therapeutic dosage for the treatment.

24. (canceled)

25. The method of claim 1, further comprising:
administering a therapeutic dosage of the treatment based on the predicted response being a predicted response classification that indicates the treatment will be successful.

26. The method of claim 1, further comprising:
administering a therapeutic dosage of the treatment based on the predicted response being sustained control.

27.-57. (canceled)

58. A method for treating a subject diagnosed with a melanoma or non-small cell lung cancer condition, comprising:
receiving peptide structure data corresponding to a set of glycoproteins in a biological sample obtained from the subject;
computing a treatment score using quantification data identified from the peptide structure data for a set of peptide structures, wherein the set of peptide structures includes at least one peptide structure identified from a plurality of peptide structures listed in Table 1, Table 12, Table 14, or Table 16;
generating a treatment output that indicates a predicted response to a treatment for the subject using the treatment score; and
administering the treatment to the patient in response to the predicted response includes a positive response classification, the step of administering comprising at least one of intravenous or oral administration of the recommended treatment or a derivative thereof at a therapeutic dosage,
wherein the treatment is selected as one from a group consisting of:
a first treatment of pembrolizumab for which the therapeutic dosage of at least one of 200 mg every three weeks, 2 mg/kg every three weeks is administered, or 400 mg every 6 weeks; and
a second treatment comprised of nivolumab and ipilimumab for which the therapeutic dosage of either 1 mg/kg nivolumab with 3 mg/kg ipilimumab or 3 mg/kg nivolumab with 1 mg/kg ipilimumab is administered.

59.-65. (canceled)

66. A method of treating melanoma or non-small cell lung cancer in a subject, the method comprising:
receiving peptide structure data corresponding to a set of glycoproteins in a biological sample obtained from the subject;
computing a treatment score using quantification data identified from the peptide structure data for a set of peptide structures, wherein the set of peptide structures includes at least one peptide structure identified from a plurality of peptide structures listed in Table 1, Table 12, Table 14, or Table 16;
generating a treatment output using the treatment score; and
administering a pembrolizumab treatment to the subject if the treatment output includes at least one of a positive response classification for the pembrolizumab treatment or an identification of the pembrolizumab treatment as a recommended treatment.

67.-71. (canceled)

72. A method of treating melanoma or non-small cell lung cancer in a subject, the method comprising:
receiving peptide structure data corresponding to a set of glycoproteins in a biological sample obtained from the subject;
computing a treatment score using quantification data identified from the peptide structure data for a set of peptide structures, wherein the set of peptide structures includes at least one peptide structure identified from a plurality of peptide structures listed in Table 1, Table 12, Table 14, or Table 16;
generating a treatment output using the treatment score; and
administering a combination treatment comprising a combination of nivolumab and ipilimumab to the subject if the treatment output includes at least one of a positive response classification for the combination treatment or an identification of the combination treatment as a recommended treatment.

73.-76. (canceled)

77. A method of identifying patients with melanoma or non-small cell lung cancer for treatment with a pembrolizumab treatment, the method comprising:
receiving peptide structure data corresponding to a set of glycoproteins in a biological sample obtained from the subject;
computing a treatment score using quantification data identified from the peptide structure data for a set of peptide structures, wherein the set of peptide structures includes at least one peptide structure identified from a plurality of peptide structures listed in Table 1, Table 12, Table 14, or Table 16; and
generating a treatment output using the treatment score,
wherein the patient is treated with the pembrolizumab treatment if the treatment output includes at least one of a positive response classification for the pembrolizumab treatment or an identification of the pembrolizumab treatment as a recommended treatment.

78.-82. (canceled)

83. A method of identifying patients with melanoma for treatment with a combination treatment comprising nivolumab and ipilimumab, the method comprising:
receiving peptide structure data corresponding to a set of glycoproteins in a biological sample obtained from the subject;
computing a treatment score using quantification data identified from the peptide structure data for a set of peptide structures, wherein the set of peptide structures includes at least one peptide structure identified from a plurality of peptide structures listed in Table 1, Table 12, Table 14, or Table 16; and
generating a treatment output using the treatment score,
wherein the patient is treated with the combination treatment if the treatment output includes at least one of a positive response classification for the combination treatment or an identification of the combination treatment as a recommended treatment.

84.-87. (canceled)

88. A method for analyzing a set of peptide structures in a sample from a patient, the method comprising:
(a) obtaining the sample from the patient;
(b) preparing the sample to form a prepared sample comprising a set of peptide structures;
(c) inputting the prepared sample into a reaction monitoring mass spectrometry system to detect a set of product ions associated with each peptide structure of the set of peptide structures,
wherein the set of peptide structures includes at least one peptide structure selected from peptide structures PS-1 to PS-38 identified in Table 6;
wherein the set of peptide structures includes a peptide structure that is characterized as having:
(i) a precursor ion with a mass-charge (m/z) ratio within ±1.5 of the m/z ratio listed for the precursor ion in Table 6 as corresponding to the peptide structure; and
(ii) a product ion having an m/z ratio within ±1.0 of the m/z ratio listed for the first product ion in Table 6 as corresponding to the peptide structure; and
(d) generating quantification data for the set of product ions using the reaction monitoring mass spectrometry system.

89.-144. (canceled)
